SEQUENCE LISTING (320 sequences)

SEQ ID NO: 1 - protein, 1257 aa, Homo sapiens
MVVALRYVWPLLLCSPCLLIQIPEEYEGHHVMEPPVITEQSPRRLVVFPTDDISLKCEASGKPEVQFRWTRDGVHFKPKEELGVTVYQSPHSGSFTITGNNSNFAQRFQGIYRCFASNKLGTAMSHEIRLMAEGAPKWPKETVKPVEVEEGESVVLPCNPPPSAEPLRIYWMNSKILHIKQDERVTMGQNGNLYFANVLTSDNHSDYICHAHFPGTRTIIQKEPIDLRVKATNSMIDRKPRLLFPTNSSSHLVALQGQPLVLECIAEGFPTPTIKWLRPSGPMPADRVTYQNHNKTLQLLKVGEEDDGEYRCLAENSLGSARHAYYVTVEAAPYWLHKPQSHLYGPGETARLDCQVQGRPQPEVTWRINGIPVEELAKDQKYRIQRGALILSNVQPSDTMVTQCEARNRHGLLLANAYIYVVQLPAKILTADNQTYMAVQGSTAYLLCKAFGAPVPSVQWLDEDGTTVLQDERFFPYANGTLGIRDLQANDTGRYFCLAANDQNNVTIMANLKVKDATQITQGPRSTIEKKGSRVTFTCQASFDPSLQPSITWRGDGRDLQELGDSDKYFIEDGRLVIHSLDYSDQGNYSCVASTELDVVESRAQLLVVGSPGPVPRLVLSDLHLLTQSQVRVSWSPAEDHNAPIEKYDIEFEDKEMAPEKWYSLGKVPGNQTSTTLKLSPYVHYTFRVTAINKYGPGEPSPVSETVVTPEAAPEKNPVDVKGEGNETTNMVITWKPLRWMDWNAPQVQYRVQWRPQGTRGPWQEQIVSDPFLVVSNTSTFVPYEIKVQAVNSQGKGPEPQVTIGYSGEDYPQAIPELEGIEILNSSAVLVKWRPVDLAQVKGHLRGYNVTYWREGSQRKHSKRHIHKDHVVVPANTTSVILSGLRPYSSYHLEVQAFNGRGSGPASEFTFSTPEGVPGHPEALHLECQSNTSLLLRWQPPLSHNGVLTGYVLSYHPLDEGGKGQLSFNLRDPELRTHNLTDLSPHLRYRFQLQATTKEGPGEAIVREGGTMALSGISDFGNISATAGENYSVVSWVPKEGQCNFRFHILFKALGEEKGGASLSPQYVSYNQSSYTQWDLQPDTDYEIHLFKERMFRHQMAVKTNGTGRVRLPPAGFATEGWFIGFVSAIILLLLVLLILCFIKRSKGGKYSVKDKEDTQVDSEARPMKDETFGEYRSLESDNEEKAFGSSQPSLNGDIKPLGSDDSLADYGGSVDVQFNEDGSFIGQYSGKKEKEAAGGNDSSGATSPINPAVALE

SEQ ID NO: 2 - protein, 8 aa, synthetic construct
GFIFSSYN

SEQ ID NO: 3 - protein, 8 aa, synthetic construct
ISSSSSTT

SEQ ID NO: 4 - protein, 17 aa, synthetic construct
ASLYSGYDYYYYYGMDV

SEQ ID NO: 5 - protein, 11 aa, synthetic construct
QSLVHSDGNTY

SEQ ID NO: 6 - skipped

SEQ ID NO: 7 - protein, 9 aa, synthetic construct
MQATQFPFT

SEQ ID NO: 8 - protein, 124 aa, synthetic construct
EVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSS

SEQ ID NO: 9 - protein, 112 aa, synthetic construct
DIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIK

SEQ ID NO: 10 - protein, 454 aa, synthetic construct
EVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 219 aa, synthetic construct
DIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 8 aa, synthetic construct
GYTFTNYY

SEQ ID NO: 13 - protein, 8 aa, synthetic construct
IGPGSGSP

SEQ ID NO: 14 - protein, 9 aa, synthetic construct
ARWLLPFDY

SEQ ID NO: 15 - protein, 5 aa, synthetic construct
SSVNY

SEQ ID NO: 16 - skipped

SEQ ID NO: 17 - protein, 9 aa, synthetic construct
QQFSSDPLT

SEQ ID NO: 18 - protein, 116 aa, synthetic construct
QVQLVQSGAEVKKPGSSVKVSCKASGYTFTNYYIHWVRQAPGQGLEWMGKIGPGSGSPYYQGMFKDRVTITADESTSTAYMELSSLRSEDTAVYYCARWLLPFDYWGQGTLVTVSS

SEQ ID NO: 19 - protein, 106 aa, synthetic construct
EIVLTQSPGTLSLSPGERATLSCRASSSVNYMHWYQQKPGQAPRLLIYATSILASGIPDRFSGSGSGTDYTLTISRLEPEDFAVYYCQQFSSDPLTFGGGTKVEIK

SEQ ID NO: 20 - protein, 446 aa, synthetic construct
QVQLVQSGAEVKKPGSSVKVSCKASGYTFTNYYIHWVRQAPGQGLEWMGKIGPGSGSPYYQGMFKDRVTITADESTSTAYMELSSLRSEDTAVYYCARWLLPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 213 aa, synthetic construct
EIVLTQSPGTLSLSPGERATLSCRASSSVNYMHWYQQKPGQAPRLLIYATSILASGIPDRFSGSGSGTDYTLTISRLEPEDFAVYYCQQFSSDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 116 aa, synthetic construct
QVQLVQSGAEVKKPGASVKVSCKASGYTFTNYYIHWVRQAPGQGLEWMGKIGPGSGSPYYNGMFKDRVTMTRDTSTSTVYMELSSLRSEDTAVYYCARWLLPFDYWGQGTLVTVSS

SEQ ID NO: 23 - protein, 106 aa, synthetic construct
EIVLTQSPATLSLSPGERATLSCRASSSVNYMHWYQQKPGQAPRLLIYATSILASGIPARFSGSGSGTDYTLTISSLEPEDFAVYYCQQFSSDPLTFGGGTKVEIK

SEQ ID NO: 24 - protein, 116 aa, synthetic construct
QVQLVQSGAEVKKPGASVKVSCKASGYTFTNYYIHWVRQAPGQGLEWMGKIGPGSGSPYYNGMFKDRVTSTRDTSISTAYMELSRLRSDDTVVYYCARWLLPFDYWGQGTLVTVSS

SEQ ID NO: 25 - protein, 116 aa, synthetic construct
QVQLVESGGGVVQPGRSLRLSCAASGYTFTNYYIHWVRQAPGKGLEWVAKIGPGSGSPYYNGMFKDRFTISRDNSKNTLYLQMNSLRAEDTAVYYCARWLLPFDYWGQGTLVTVSS

SEQ ID NO: 26 - protein, 9 aa, synthetic construct
AKWLLPFDY

SEQ ID NO: 27 - protein, 116 aa, synthetic construct
EVQLLESGGGLVQPGGSLRLSCAASGYTFTNYYIHWVRQAPGKGLEWVSKIGPGSGSPYYNGMFKDRFTISVDNSKNTLYLQMNSLRAEDTAVYYCAKWLLPFDYWGQGTLVTVSS

SEQ ID NO: 28 - protein, 116 aa, synthetic construct
QVQLVQSGAEVKKPGSSVKVSCKASGYTFTNYYIHWVRQAPGQGLEWMGKIGPGSGSPYYNGMFKDRVTITADESTSTAYMELSSLRSEDTAVYYCARWLLPFDYWGQGTLVTVSS

SEQ ID NO: 29 - protein, 116 aa, synthetic construct
EVQLLESGGGLVQPGGSLRLSCAASGYTFTNYYIHWVRQAPGKGLEWVSKIGPGSGSPYYADSVKGRFTISVDNSKNTLYLQMNSLRAEDTAVYYCAKWLLPFDYWGQGTLVTVSS

SEQ ID NO: 30 - protein, 106 aa, synthetic construct
EIVMTQSPATLSVSPGERATLSCRASSSVNYMHWYQQKPGQAPRLLIYATSILASGIPARFSGSGSGTEYTLTISSLQSEDFAVYYCQQFSSDPLTFGGGTKVEIK

SEQ ID NO: 31 - protein, 106 aa, synthetic construct
DIQMTQSPSTLSASVGDRVTITCRASSSVNYMHWYQQKPGKAPKLLIYATSILASGVPSRFSGSGSGTEYTLTISSLQPDDFATYYCQQFSSDPLTFGGGTKVEIK

SEQ ID NO: 32 - protein, 106 aa, synthetic construct
DIQMTQSPSSVSASVGDRVTITCRASSSVNYMHWYQQKPGKAPKPLIYATSILASGVPSRFSGSGSGTDYTLTISSLQPEDFATYYCQQFSSDPLTFGGGTKVEIK

SEQ ID NO: 33 - protein, 116 aa, synthetic construct
QVQLRQSGAELVRPGTSVKMSCKASGYTFTNYYIHWVKQRPGQGLEWIGKIGPGSGSPYYNGMFKDKATLTVDTSSSTAYMQLSSLTSEDSAVYFCARWLLPFDYWGQGTTLTVSS

SEQ ID NO: 34 - protein, 106 aa, synthetic construct
QIVLSQSPGILSASPGEKVTMTCRASSSVNYMHWYQQKSGSSPKPWIFATSILASGVPPRFSGSGSGTSYSLTISSVEAEDTATYYCQQFSSDPLTFGAGTKLELK

SEQ ID NO: 35 - protein, 8 aa, synthetic construct
GGSINSYY

SEQ ID NO: 36 - protein, 7 aa, synthetic construct
FYTSGST

SEQ ID NO: 37 - protein, 16 aa, synthetic construct
AREWSGSFYGGDAFDI

SEQ ID NO: 38 - protein, 12 aa, synthetic construct
QSVLNSSNNKNY

SEQ ID NO: 39 - skipped

SEQ ID NO: 40 - protein, 9 aa, synthetic construct
QQYYSTPYT

SEQ ID NO: 41 - protein, 122 aa, synthetic construct
QVQLQESGPGLVKPSETLSLTCTVSGGSINSYYWSWIRQPAGKGLEWIGRFYTSGSTNHNPSLKSRVTMSVDTSKNQFSLKLTSVTAADTAVYYCAREWSGSFYGGDAFDIWGQGTMVTVSS

SEQ ID NO: 42 - protein, 113 aa, synthetic construct
DIVMTQSPDSLAVSLGERATINCKSSQSVLNSSNNKNYLTWYQQRPGQPPNLLIYWASTRESGVPDRFSGSGSGTDFTLTISSLQAEDVAVYYCQQYYSTPYTFGQGTKLEIE

SEQ ID NO: 43 - protein, 8 aa, synthetic construct
GYPFTGYY

SEQ ID NO: 44 - protein, 8 aa, synthetic construct
INPNSGDT

SEQ ID NO: 45 - protein, 13 aa, synthetic construct
AREMGTTKGYFDL

SEQ ID NO: 46 - protein, 6 aa, synthetic construct
QGISSY

SEQ ID NO: 47 - skipped

SEQ ID NO: 48 - protein, 9 aa, synthetic construct
QQLNSYPDT

SEQ ID NO: 49 - protein, 120 aa, synthetic construct
QVQLVQSGAEVKKPGASVRVSCKASGYPFTGYYMHWVRQAPGQGLEWMGWINPNSGDTDYVQKFQGRVTMTRDTSISTAYMELSRLRSDDTAVYYCAREMGTTKGYFDLWGRGTLVTVSS

SEQ ID NO: 50 - protein, 107 aa, synthetic construct
DIQLTQSPSFLSASVGDRVTITCRASQGISSYLAWFQQKPGKAPRLLIYAASTLQSGVPSRFSGSRSGTEFTLTISSLQPEDFATYYCQQLNSYPDTFGQGTKVDIK

SEQ ID NO: 51 - protein, 12 aa, synthetic construct
QTILYSPNNKNY

SEQ ID NO: 52 - skipped

SEQ ID NO: 53 - protein, 113 aa, synthetic construct
DIVMTQSPDSLAVSLGERATINCKSSQTILYSPNNKNYLAWYQQKPGQPPKLLIYLTSTRESGVPDRFSGSGSGTDFTLTISSLQAEDVAVYYCQQYYSTPYTFGQGTKLEIK

SEQ ID NO: 54 - protein, 8 aa, synthetic construct
GYTFIGYY

SEQ ID NO: 55 - protein, 8 aa, synthetic construct
INPNSGGT

SEQ ID NO: 56 - protein, 13 aa, synthetic construct
ARGGLERRDAFDI

SEQ ID NO: 57 - protein, 6 aa, synthetic construct
QSISNW

SEQ ID NO: 58 - skipped

SEQ ID NO: 59 - protein, 8 aa, synthetic construct
QQYDTYRT

SEQ ID NO: 60 - protein, 120 aa, synthetic construct
QVQLVQSGAEVKKPGASVKVSCKASGYTFIGYYLHWVRQAPGQGLEWMGWINPNSGGTNSAQKFQGRVTMTRDTSISTTYMDLSRLRSDDTAVYYCARGGLERRDAFDIWGQGTLVTVSS

SEQ ID NO: 61 - protein, 106 aa, synthetic construct
DIQMTQSPSTLSASVGDRVTITCRASQSISNWLAWYQQKPGKAPNLLIYKASSLESGVPSRFSGSGSGTEFTLTISSLQPDDFATYYCQQYDTYRTFGQGTKVEIK

SEQ ID NO: 62 - protein, 8 aa, synthetic construct
GFTFSHYW

SEQ ID NO: 63 - protein, 8 aa, synthetic construct
IMQDGSEK

SEQ ID NO: 64 - protein, 12 aa, synthetic construct
ARDRGVTTLFDY

SEQ ID NO: 65 - protein, 6 aa, synthetic construct
QSISSW

SEQ ID NO: 66 - protein, 8 aa, synthetic construct
QQYYSYGT

SEQ ID NO: 67 - protein, 119 aa, synthetic construct
EVQLVESGGGLVQPGGSLRLSCGVSGFTFSHYWMNWVRQAPGKGLEWVANIMQDGSEKYYVDSVKGRFTISRDNAKNSLYLQMNSLRVEDTAVYYCARDRGVTTLFDYWGQGILVTVSS

SEQ ID NO: 68 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYYSYGTFGQGTKVEIK

SEQ ID NO: 69 - protein, 8 aa, synthetic construct
GYIFISYW

SEQ ID NO: 70 - protein, 8 aa, synthetic construct
INPRNGRT

SEQ ID NO: 71 - protein, 12 aa, synthetic construct
ARGGDYGGSFDY

SEQ ID NO: 72 - protein, 10 aa, synthetic construct
ESVDSYGNSF

SEQ ID NO: 73 - skipped

SEQ ID NO: 74 - protein, 9 aa, synthetic construct
QQSNEDPWT

SEQ ID NO: 75 - protein, 119 aa, synthetic construct
QVQLQQPGAELVKPGASVKLSCKASGYIFISYWMHWVKQRPGQGLEWIGEINPRNGRTNYNEKFKSKATLTVDKSSSTAYMQLTSLTSEDSAVYYCARGGDYGGSFDYWGQGTTLTVSS

SEQ ID NO: 76 - protein, 111 aa, synthetic construct
DIVLTQSPASLAVSLGQRATISCRASESVDSYGNSFMHWYQQKPGQPPKLLIYRASNLESGIPARFSGSGSRTDFTLTVNPVEADDVATYYCQQSNEDPWTFGGGTKLEIK

SEQ ID NO: 77 - protein, 245 aa, synthetic construct
QVQLQQPGAELVKPGASVKLSCKASGYIFISYWMHWVKQRPGQGLEWIGEINPRNGRTNYNEKFKSKATLTVDKSSSTAYMQLTSLTSEDSAVYYCARGGDYGGSFDYWGQGTTLTVSSGGGGSGGGGSGGGGSDIVLTQSPASLAVSLGQRATISCRASESVDSYGNSFMHWYQQKPGQPPKLLIYRASNLESGIPARFSGSGSRTDFTLTVNPVEADDVATYYCQQSNEDPWTFGGGTKLEIK

SEQ ID NO: 78 - protein, 245 aa, synthetic construct
DIVLTQSPASLAVSLGQRATISCRASESVDSYGNSFMHWYQQKPGQPPKLLIYRASNLESGIPARFSGSGSRTDFTLTVNPVEADDVATYYCQQSNEDPWTFGGGTKLEIKGGGGSGGGGSGGGGSQVQLQQPGAELVKPGASVKLSCKASGYIFISYWMHWVKQRPGQGLEWIGEINPRNGRTNYNEKFKSKATLTVDKSSSTAYMQLTSLTSEDSAVYYCARGGDYGGSFDYWGQGTTLTVSS

SEQ ID NO: 79 - DNA, 735 bp, synthetic construct
  source 1..735: mol_type = other DNA
caggtacagctccagcaaccgggggcggagttggtaaagccgggtgcgtccgtaaaattgtcatgcaaggccagcggctatatcttcattagctactggatgcactgggttaagcaacggcccggccagggtcttgagtggatcggggaaattaatccgaggaacggccggacgaactacaacgaaaaattcaaatctaaagctactctcaccgtggataagtcttcttctacagcttatatgcagttgaccagtctcaccagtgaagacagtgcggtatattactgtgcaaggggtggagactatgggggctcattcgattattgggggcaaggtactacactgactgtatcttccggcggtggtggctcaggcggcggaggatccgggggggggggatccgacatagtactcactcagagcccagcttcactggctgtgtctttgggacaacgcgcaactatatcctgccgggcgtcagaatctgtagatagctatggaaacagttttatgcattggtatcagcaaaaacccggtcagccccctaagttgctcatttatagagcctcaaacctcgaatcagggattccggcacgcttcagtgggtctggtagtagaacggatttcacacttacagtcaatccagtcgaggccgacgatgtggctacatactactgccagcaatccaatgaagatccttggacatttggtggcgggacaaagctggaaataaag

SEQ ID NO: 80 - DNA, 735 bp, synthetic construct
  source 1..735: mol_type = other DNA
gacatagtactcactcagagcccagcttcactggctgtgtctttgggacaacgcgcaactatatcctgccgggcgtcagaatctgtagatagctatggaaacagttttatgcattggtatcagcaaaaacccggtcagccccctaagttgctcatttatagagcctcaaacctcgaatcagggattccggcacgcttcagtgggtctggtagtagaacggatttcacacttacagtcaatccagtcgaggccgacgatgtggctacatactactgccagcaatccaatgaagatccttggacatttggtggcgggacaaagctggaaataaagggcggtggtggctcaggcggcggaggatccgggggggggggatcccaggtacagctccagcaaccgggggcggagttggtaaagccgggtgcgtccgtaaaattgtcatgcaaggccagcggctatatcttcattagctactggatgcactgggttaagcaacggcccggccagggtcttgagtggatcggggaaattaatccgaggaacggccggacgaactacaacgaaaaattcaaatctaaagctactctcaccgtggataagtcttcttctacagcttatatgcagttgaccagtctcaccagtgaagacagtgcggtatattactgtgcaaggggtggagactatgggggctcattcgattattgggggcaaggtactacactgactgtatcttcc

SEQ ID NO: 81 - protein, 8 aa, synthetic construct
GFIFSSYN

SEQ ID NO: 82 - protein, 8 aa, synthetic construct
ISSSSSTT

SEQ ID NO: 83 - protein, 17 aa, synthetic construct
ASLYSGYDYYYYYGMDV

SEQ ID NO: 84 - protein, 11 aa, synthetic construct
QSLVHSDGNTY

SEQ ID NO: 85 - skipped

SEQ ID NO: 86 - protein, 9 aa, synthetic construct
MQATQFPFT

SEQ ID NO: 87 - protein, 124 aa, synthetic construct; identical to SEQ ID NO: 8

SEQ ID NO: 88 - protein, 112 aa, synthetic construct; identical to SEQ ID NO: 9

SEQ ID NO: 89 - protein, 251 aa, synthetic construct
EVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSSGGGGSGGGGSGGGGSDIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIK

SEQ ID NO: 90 - protein, 252 aa, synthetic construct
DIVMTQTPLSSPVSLGQPASISCRSSQSLVHSDGNTYLSWLQQRPGQPPRVLIYKISKRFSGVPDRFSGSGAGTDFTLKISRVEAEDVGVYYCMQATQFPFTFGPGTKVDIKGGGGSGGGGSGGGGSEEVQMVESGGGLVQPGGSLRLSCAAPGFIFSSYNMNWVRQAPGKGLEWVSYISSSSSTTYYADSVKGRFTISRDNAKNSLYLQMNSLRDEDTAVYYCASLYSGYDYYYYYGMDVWGQGTTVTVSS

SEQ ID NO: 91 - DNA, 753 bp, synthetic construct
  source 1..753: mol_type = other DNA
gaagtccagatggtggagtcaggagggggtctggtccaaccggggggtagcctgaggttgtcctgcgcggcaccaggatttatcttctcaagctataacatgaactgggtacgccaagcacctggcaaaggactggaatgggtaagttacatttcttccagttcatcaacaacttactatgcagacagtgtgaaaggccggtttactataagccgggacaatgctaagaactcactctatctccaaatgaactccctccgcgacgaggacacagctgtctattactgtgctagtctctactctggctacgattactattactattatggaatggatgtgtgggggcaaggcactactgtcacagtctcaagcggcggtggtggctcaggcggcggaggatccgggggggggggatccgatattgttatgacgcaaacgcccttgtcatcacctgtatcattgggtcagccggcttccatatcctgccgatctagtcaatccttggtgcactcagacgggaatacctacctttcatggctccagcagagacccgggcaaccaccgagagtgttgatctataaaatttctaaacggtttagcggggtccctgacagattctccggttccggggctggaactgattttacacttaaaatatcccgagtcgaagcggaagacgtgggggtgtattattgcatgcaagcaactcaatttcccttcacatttgggcctggtacaaaagtggacatcaag

SEQ ID NO: 92 - DNA, 753 bp, synthetic construct
  source 1..753: mol_type = other DNA
gatattgttatgacgcaaacgcccttgtcatcacctgtatcattgggtcagccggcttccatatcctgccgatctagtcaatccttggtgcactcagacgggaatacctacctttcatggctccagcagagacccgggcaaccaccgagagtgttgatctataaaatttctaaacggtttagcggggtccctgacagattctccggttccggggctggaactgattttacacttaaaatatcccgagtcgaagcggaagacgtgggggtgtattattgcatgcaagcaactcaatttcccttcacatttgggcctggtacaaaagtggacatcaagggcggtggtggctcaggcggcggaggatccgggggggggggatccgaagtccagatggtggagtcaggagggggtctggtccaaccggggggtagcctgaggttgtcctgcgcggcaccaggatttatcttctcaagctataacatgaactgggtacgccaagcacctggcaaaggactggaatgggtaagttacatttcttccagttcatcaacaacttactatgcagacagtgtgaaaggccggtttactataagccgggacaatgctaagaactcactctatctccaaatgaactccctccgcgacgaggacacagctgtctattactgtgctagtctctactctggctacgattactattactattatggaatggatgtgtgggggcaaggcactactgtcacagtctcaagc

SEQ ID NO: 93 - protein, 8 aa, synthetic construct
GFSLTSYG

SEQ ID NO: 94 - protein, 7 aa, synthetic construct
IWGDGST

SEQ ID NO: 95 - protein, 11 aa, synthetic construct
ATSTVGYAVDY

SEQ ID NO: 96 - protein, 6 aa, synthetic construct
ENIYSN

SEQ ID NO: 97 - skipped

SEQ ID NO: 98 - protein, 9 aa, synthetic construct
QHFWGSPYT

SEQ ID NO: 99 - protein, 117 aa, synthetic construct
QVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSS

SEQ ID NO: 100 - protein, 107 aa, synthetic construct
DIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIK

SEQ ID NO: 101 - protein, 239 aa, synthetic construct
QVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSSGGGGSGGGGSGGGGSDIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIK

SEQ ID NO: 102 - protein, 239 aa, synthetic construct
DIQMTQSPASLSVSVGETVTITCRASENIYSNLAWYQQRQGKSPQLLVYAATNLADGVPSRFSGSGSGTQYSLKINSLQSEDFGSYYCQHFWGSPYTFGGGTKLEIKGGGGSGGGGSGGGGSQVQLKESGPGLVAPSQSLSITCTISGFSLTSYGVHWVRQPPGKGLEWLVVIWGDGSTSYNSALKSRLSISKDNSKSQVFLKMNSLQTDDTAMYFCATSTVGYAVDYWGQGTSVTVSS

SEQ ID NO: 103 - DNA, 717 bp, synthetic construct
  source 1..717: mol_type = other DNA
caagtccagctcaaggagagcggtccaggactcgtagcaccctctcaatctttgtctatcacttgtaccataagcggtttttccttgacaagttatggcgtacattgggtgagacaaccgccaggtaaaggactcgaatggttggttgttatctggggggatgggagtaccagctacaacagcgcactcaagtctcgcctttcaatttctaaggacaacagtaaatcacaggtttttctgaagatgaatagcctgcaaaccgacgacacggcgatgtatttctgtgcgacttctacggtaggttatgccgtcgactattggggtcagggtacgtcagttaccgtaagtagcggcggtggtggctcaggcggcggaggatccgggggggggggatccgacatccagatgacccagtcaccggccagtttgtccgtttccgtaggcgagaccgtaaccatcacatgccgagcaagcgagaatatctattccaacctggcctggtatcagcagagacaaggcaaaagtccgcaacttttggtttatgctgccactaatttggccgatggggtcccgagtcgattttctggctctggctccgggacgcagtattccctgaagataaactctttgcagtcagaagattttggttcatattattgtcagcatttctggggcagcccgtacacatttggaggtgggaccaaattggaaataaaa

SEQ ID NO: 104 - DNA, 717 bp, synthetic construct
  source 1..717: mol_type = other DNA
gacatccagatgacccagtcaccggccagtttgtccgtttccgtaggcgagaccgtaaccatcacatgccgagcaagcgagaatatctattccaacctggcctggtatcagcagagacaaggcaaaagtccgcaacttttggtttatgctgccactaatttggccgatggggtcccgagtcgattttctggctctggctccgggacgcagtattccctgaagataaactctttgcagtcagaagattttggttcatattattgtcagcatttctggggcagcccgtacacatttggaggtgggaccaaattggaaataaaaggcggtggtggctcaggcggcggaggatccgggggggggggatcccaagtccagctcaaggagagcggtccaggactcgtagcaccctctcaatctttgtctatcacttgtaccataagcggtttttccttgacaagttatggcgtacattgggtgagacaaccgccaggtaaaggactcgaatggttggttgttatctggggggatgggagtaccagctacaacagcgcactcaagtctcgcctttcaatttctaaggacaacagtaaatcacaggtttttctgaagatgaatagcctgcaaaccgacgacacggcgatgtatttctgtgcgacttctacggtaggttatgccgtcgactattggggtcagggtacgtcagttaccgtaagtagc

SEQ ID NO: 105 - protein, 8 aa, synthetic construct
GDSISSYY

SEQ ID NO: 106 - protein, 7 aa, synthetic construct
IYTSGTT

SEQ ID NO: 107 - protein, 13 aa, synthetic construct
ASHSSGWYDAFDI

SEQ ID NO: 108 - protein, 12 aa, synthetic construct
QSVLYSSNNKNY

SEQ ID NO: 109 - protein, 9 aa, synthetic construct
QQYYSTPPT

SEQ ID NO: 110 - protein, 119 aa, synthetic construct
QVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSS

SEQ ID NO: 111 - protein, 113 aa, synthetic construct
DIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIK

SEQ ID NO: 112 - protein, 247 aa, synthetic construct
QVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSSGGGGSGGGGSGGGGSDIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIK

SEQ ID NO: 113 - protein, 247 aa, synthetic construct
DIVMTQSPDSLAVSLGERATINCKSSQSVLYSSNNKNYLAWYQQKPGQPPKLFIYWASTRESGVPDRFSGSGSGTDFTLTISSLQAEDVAVYYCQQYYSTPPTFGQGTKVEIKGGGGSGGGGSGGGGSQVQLQESGPGLVKPSETLSLTCTVSGDSISSYYWTWIRQPAGKGLEWIGRIYTSGTTNYNPSLKSRVTMSVDTSKNQFSLKLSSVTAADTAVYYCASHSSGWYDAFDIWGQGTMVTVSS

SEQ ID NO: 114 - DNA, 741 bp, synthetic construct
  source 1..741: mol_type = other DNA
caggtacagcttcaagaaagcggtccaggacttgtgaagccgagcgagacgctttcactgacttgcacagtcagtggggacagtattagttcctattactggacatggatacgacaaccagcagggaaaggtctggaatggattgggcgcatttacaccagtggaactacaaattacaacccatccctcaaatccagggttacgatgagcgtcgacacgagtaagaatcagttttccctgaagctctccagtgtgacggctgcagatactgcagtgtattattgcgcttcccactcaagtggttggtacgatgctttcgacatctgggggcagggtactatggtgaccgtgtcctcaggcggtggtggctcaggcggcggaggatccgggggggggggatccgacatcgtaatgacccaatcacctgattctctggctgtgagccttggtgagagggcaacaataaactgcaagagctctcagtccgtactttatagtagcaacaacaagaactatttggcgtggtaccagcagaagccgggtcagccgccaaagctgtttatatattgggcctctacccgcgaatctggagtaccggacaggtttagtggtagtggctcaggtacggattttacgctcaccattagctctctgcaagcagaggacgtagccgtatattattgccagcaatattattctacgccacccacctttggtcaggggacgaaagttgagatcaag

SEQ ID NO: 115 - DNA, 741 bp, synthetic construct
  source 1..741: mol_type = other DNA
gacatcgtaatgacccaatcacctgattctctggctgtgagccttggtgagagggcaacaataaactgcaagagctctcagtccgtactttatagtagcaacaacaagaactatttggcgtggtaccagcagaagccgggtcagccgccaaagctgtttatatattgggcctctacccgcgaatctggagtaccggacaggtttagtggtagtggctcaggtacggattttacgctcaccattagctctctgcaagcagaggacgtagccgtatattattgccagcaatattattctacgccacccacctttggtcaggggacgaaagttgagatcaagggcggtggtggctcaggcggcggaggatccgggggggggggatcccaggtacagcttcaagaaagcggtccaggacttgtgaagccgagcgagacgctttcactgacttgcacagtcagtggggacagtattagttcctattactggacatggatacgacaaccagcagggaaaggtctggaatggattgggcgcatttacaccagtggaactacaaattacaacccatccctcaaatccagggttacgatgagcgtcgacacgagtaagaatcagttttccctgaagctctccagtgtgacggctgcagatactgcagtgtattattgcgcttcccactcaagtggttggtacgatgctttcgacatctgggggcagggtactatggtgaccgtgtcctca

SEQ ID NO: 116 - protein, 8 aa, synthetic construct
GYTFTSYW

SEQ ID NO: 117 - protein, 8 aa, synthetic construct
VYPGSGST

SEQ ID NO: 118 - protein, 11 aa, synthetic construct
TRPGNYEGFAY

SEQ ID NO: 119 - protein, 10 aa, synthetic construct
ESVDSYGSSF

SEQ ID NO: 120 - skipped

SEQ ID NO: 121 - protein, 9 aa, synthetic construct
QQNNEDPWT

SEQ ID NO: 122 - protein, 118 aa, synthetic construct
QVQLQQPGSDLVRPGTSVKLSCKASGYTFTSYWMHWVKQRPGQGLEWIGNVYPGSGSTKYDEKFKTKATLTVDTSSNTAYMELSSLTSEDSAVYYCTRPGNYEGFAYWGQGTLVTVSA

SEQ ID NO: 123 - protein, 111 aa, synthetic construct
NIVLTQSPASLAVSLGQRATISCRASESVDSYGSSFMHWYQQKPGQPPKLLIYLASNLESGVPARFSGSGSRTDFTLTIDPVEADDAATYYCQQNNEDPWTFGGGTKLEIK

SEQ ID NO: 124 - protein, 244 aa, synthetic construct
QVQLQQPGSDLVRPGTSVKLSCKASGYTFTSYWMHWVKQRPGQGLEWIGNVYPGSGSTKYDEKFKTKATLTVDTSSNTAYMELSSLTSEDSAVYYCTRPGNYEGFAYWGQGTLVTVSAGGGGSGGGGSGGGGSNIVLTQSPASLAVSLGQRATISCRASESVDSYGSSFMHWYQQKPGQPPKLLIYLASNLESGVPARFSGSGSRTDFTLTIDPVEADDAATYYCQQNNEDPWTFGGGTKLEIK

SEQ ID NO: 125 - protein, 244 aa, synthetic construct
NIVLTQSPASLAVSLGQRATISCRASESVDSYGSSFMHWYQQKPGQPPKLLIYLASNLESGVPARFSGSGSRTDFTLTIDPVEADDAATYYCQQNNEDPWTFGGGTKLEIKGGGGSGGGGSGGGGSQVQLQQPGSDLVRPGTSVKLSCKASGYTFTSYWMHWVKQRPGQGLEWIGNVYPGSGSTKYDEKFKTKATLTVDTSSNTAYMELSSLTSEDSAVYYCTRPGNYEGFAYWGQGTLVTVSA

SEQ ID NO: 126 - DNA, 732 bp, synthetic construct
  source 1..732: mol_type = other DNA
caggtgcaacttcagcagcccggctctgatcttgtgcgccctggtacttctgtaaagcttagctgtaaagcttctggttatacgttcacgagttattggatgcactgggtaaagcagcggcctggacagggattggagtggatcggaaacgtgtaccctggcagtggttctacaaagtatgacgagaaatttaagacaaaagcaacactcactgtcgatacgtcttcaaacacagcgtacatggagttgagttcattgacctccgaggacagcgctgtatattactgcacacgccctggaaactacgaaggtttcgcgtattgggggcaggggaccttggtcacagtgtcagccggcggtggtggctcaggcggcggaggatccgggggggggggatccaacatagtccttacgcaaagtccagcttcactggcagtatctctgggtcaacgcgctaccattagttgtcgcgcaagcgaatccgtcgatagctatgggtcttcctttatgcactggtatcagcagaagccgggacagccgcccaagctgctgatctacctcgcgtccaacttggagtcaggcgttcccgcacggtttagtggaagcggaagtagaactgattttactcttacgattgatcctgtcgaggctgatgatgctgctacatattattgtcagcagaataatgaggatccttggaccttcggcgggggaaccaaattggaaattaaa

SEQ ID NO: 127 - DNA, 732 bp, synthetic construct
  source 1..732: mol_type = other DNA
aacatagtccttacgcaaagtccagcttcactggcagtatctctgggtcaacgcgctaccattagttgtcgcgcaagcgaatccgtcgatagctatgggtcttcctttatgcactggtatcagcagaagccgggacagccgcccaagctgctgatctacctcgcgtccaacttggagtcaggcgttcccgcacggtttagtggaagcggaagtagaactgattttactcttacgattgatcctgtcgaggctgatgatgctgctacatattattgtcagcagaataatgaggatccttggaccttcggcgggggaaccaaattggaaattaaaggcggtggtggctcaggcggcggaggatccgggggggggggatcccaggtgcaacttcagcagcccggctctgatcttgtgcgccctggtacttctgtaaagcttagctgtaaagcttctggttatacgttcacgagttattggatgcactgggtaaagcagcggcctggacagggattggagtggatcggaaacgtgtaccctggcagtggttctacaaagtatgacgagaaatttaagacaaaagcaacactcactgtcgatacgtcttcaaacacagcgtacatggagttgagttcattgacctccgaggacagcgctgtatattactgcacacgccctggaaactacgaaggtttcgcgtattgggggcaggggaccttggtcacagtgtcagcc

SEQ ID NO: 128 - protein, 118 aa, synthetic construct
QVQLVQSGAEVKKPGASVKVSCKASGYTFTSYWMHWVRQAPGQGLEWMGNVYPGSGSTKYAQKFQGRVTMTRDTSTSTVYMELSSLRSEDTAVYYCARPGNYEGFAYWGQGTLVTVSS

SEQ ID NO: 129 - protein, 111 aa, synthetic construct
NIQLTQSPSSLSASVGDRVTITCRASESVDSYGSSFMHWYQQKPGKPPKLLIYLASNLESGVPSRFSGSGSGTDFTLTISSLQPEDFATYYCQQNNEDPWTFGQGTKLEIK

SEQ ID NO: 130 - protein, 118 aa, synthetic construct
QVQLVQSGAEVKKPGASVKVSCKASGYTFTSYWMHWVRQAPGQGLEWIGNVYPGSGSTKYAQKFQGRATLTVDTSTSTVYMELSSLRSEDTAVYYCARPGNYEGFAYWGQGTLVTVSS

SEQ ID NO: 131 - protein, 111 aa, synthetic construct
DIQLTQSPSSLSASVGDRVTITCRASESVDSYGSSFMHWYQQKPGKPPKLLIYLASNLESGVPSRFSGSGSGTDFTLTISSLQPEDFATYYCQQNNEDPWTFGQGTKLEIK

SEQ ID NO: 132 - protein, 111 aa, synthetic construct
DIQLTQSPSSLSASVGDRVTITCRASESVDSYGSSFMHWYQQKPGKPPKLLIYLASNLESGVPSRFSGSGSRTDFTLTISSLQPEDFATYYCQQNNEDPWTFGQGTKLEIK

SEQ ID NO: 133 - protein, 111 aa, synthetic construct; identical to SEQ ID NO: 129

SEQ ID NO: 134 - protein, 118 aa, synthetic construct
QVQLVQSGAEVKKPGASVKVSCKASGYTFTSYWMHWVRQAPGQGLEWIGNVYPGSGSTKYAQKFQGRATLTVDTSTSTAYMELSSLRSEDTAVYYCTRPGNYEGFAYWGQGTLVTVSS

SEQ ID NO: 135 - protein, 118 aa, synthetic construct
QVQLVQSGAEVKKPGASVKVSCKASGYTFTSYWMHWVRQAPGQGLEWIGNVYPGSGSTKYAQKFQGRATLTVDTSTNTAYMELSSLRSEDTAVYYCTRPGNYEGFAYWGQGTLVTVSS

SEQ ID NO: 136 - protein, 111 aa, synthetic construct
DIQMTQSPSSLSASVGDRVTITCRASESVDSYGSSFMHWYQQKPGKAPKLLIYLASNLESGVPSRFSGSGSGTDFTLTISSLQPEDFATYYCQQNNEDPWTFGQGTKLEIK

SEQ ID NO: 137 - skipped

SEQ ID NO: 138 - protein, 19 aa, synthetic construct
GGGGSGGGGSGGGSGGGGS

SEQ ID NO: 139 - protein, 15 aa, synthetic construct
GGGGSGGGGSGGGGS

SEQ ID NO: 140 - protein, 24 aa, synthetic construct
GGGGSGGGGSGGGGSGGGSGGGGS

SEQ ID NO: 141 - protein, 29 aa, synthetic construct
GGGGSGGGGSGGGGSGGGGSGGGSGGGGS

SEQ ID NO: 142 - protein, 5 aa, synthetic construct
GGGGS

SEQ ID NO: 143 - protein, 10 aa, synthetic construct
GGGGSGGGGS

SEQ ID NO: 144 - protein, 330 aa, synthetic construct [sequence omitted]

SEQ ID NO: 145 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 146 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 147 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 107 aa, synthetic construct [sequence omitted]

SEQ ID NO: 149 - protein, 15 aa, Homo sapiens
KQDERVTMGQNGNLY

SEQ ID NO: 150 - protein, 6 aa, Homo sapiens
SMIDRK

SEQ ID NO: 151 - protein, 11 aa, Homo sapiens
KVGEEDDGEYR

SEQ ID NO: 152 - protein, 18 aa, Homo sapiens
KYDIEFEDKEMAPEKWYS

SEQ ID NO: 153 - protein, 17 aa, Homo sapiens
RWMDWNAPQVQYRVQWR

SEQ ID NO: 154 - protein, 15 aa, synthetic construct
NYYIHWVRQAPGQGL

SEQ ID NO: 155 - protein, 15 aa, synthetic construct
YYIHWVRQAPGQGLE

SEQ ID NO: 156 - protein, 15 aa, synthetic construct
YIHWVRQAPGQGLEW

SEQ ID NO: 157 - protein, 15 aa, synthetic construct
IHWVRQAPGQGLEWM

SEQ ID NO: 158 - protein, 15 aa, synthetic construct
ESTSTAYMELSSLRS

SEQ ID NO: 159 - protein, 15 aa, synthetic construct
STSTAYMELSSLRSE

SEQ ID NO: 160 - protein, 15 aa, synthetic construct
TSTAYMELSSLRSED

SEQ ID NO: 161 - protein, 15 aa, synthetic construct
STAYMELSSLRSEDT

SEQ ID NO: 162 - protein, 15 aa, synthetic construct
TAYMELSSLRSEDTA

SEQ ID NO: 163 - protein, 15 aa, synthetic construct
AYMELSSLRSEDTAV

SEQ ID NO: 164 - protein, 15 aa, synthetic construct
YMELSSLRSEDTAVY

SEQ ID NO: 165 - protein, 15 aa, synthetic construct
WGQGTLVTVSSASTK

SEQ ID NO: 166 - protein, 15 aa, synthetic construct
GQGTLVTVSSASTKG

SEQ ID NO: 167 - protein, 15 aa, synthetic construct
QGTLVTVSSASTKGP

SEQ ID NO: 168 - protein, 15 aa, synthetic construct
GTLVTVSSASTKGPS

SEQ ID NO: 169 - protein, 15 aa, synthetic construct
LTSGVHTFPAVLQSS

SEQ ID NO: 170 - protein, 15 aa, synthetic construct
TSGVHTFPAVLQSSG

SEQ ID NO: 171 - protein, 15 aa, synthetic construct
HTFPAVLQSSGLYSL

SEQ ID NO: 172 - protein, 15 aa, synthetic construct
TFPAVLQSSGLYSLS

SEQ ID NO: 173 - protein, 15 aa, synthetic construct
FPAVLQSSGLYSLSS

SEQ ID NO: 174 - protein, 15 aa, synthetic construct
VLQSSGLYSLSSVVT

SEQ ID NO: 175 - protein, 15 aa, synthetic construct
LQSSGLYSLSSVVTV

SEQ ID NO: 176 - protein, 15 aa, synthetic construct
QSSGLYSLSSVVTVP

SEQ ID NO: 177 - protein, 15 aa, synthetic construct
SSGLYSLSSVVTVPS

SEQ ID NO: 178 - protein, 15 aa, synthetic construct
SGLYSLSSVVTVPSS

SEQ ID NO: 179 - protein, 15 aa, synthetic construct
GLYSLSSVVTVPSSS

SEQ ID NO: 180 - protein, 15 aa, synthetic construct
LYSLSSVVTVPSSSL

SEQ ID NO: 181 - protein, 15 aa, synthetic construct
YSLSSVVTVPSSSLG

SEQ ID NO: 182 - protein, 15 aa, synthetic construct
SLSSVVTVPSSSLGT

SEQ ID NO: 183 - protein, 15 aa, synthetic construct
LSSVVTVPSSSLGTQ

SEQ ID NO: 184 - protein, 15 aa, synthetic construct
SSVVTVPSSSLGTQT

SEQ ID NO: 185 - protein, 15 aa, synthetic construct
CPAPELLGGPSVFLF

SEQ ID NO: 186 - protein, 15 aa, synthetic construct
PAPELLGGPSVFLFP

SEQ ID NO: 187 - protein, 15 aa, synthetic construct
APELLGGPSVFLFPP

SEQ ID NO: 188 - protein, 15 aa, synthetic construct
EQYNSTYRVVSVLTV

SEQ ID NO: 189 - protein, 15 aa, synthetic construct
QYNSTYRVVSVLTVL

SEQ ID NO: 190 - protein, 15 aa, synthetic construct
YNSTYRVVSVLTVLH

SEQ ID NO: 191 - protein, 15 aa, synthetic construct
NSTYRVVSVLTVLHQ

SEQ ID NO: 192 - protein, 15 aa, synthetic construct
STYRVVSVLTVLHQD

SEQ ID NO: 193 - protein, 15 aa, synthetic construct
TYRVVSVLTVLHQDW

SEQ ID NO: 194 - protein, 15 aa, synthetic construct
DGSFFLYSKLTVDKS

SEQ ID NO: 195 - protein, 15 aa, synthetic construct
GSFFLYSKLTVDKSR

SEQ ID NO: 196 - protein, 15 aa, synthetic construct
SFFLYSKLTVDKSRW

SEQ ID NO: 197 - protein, 15 aa, synthetic construct
FFLYSKLTVDKSRWQ

SEQ ID NO: 198 - protein, 15 aa, synthetic construct
FLYSKLTVDKSRWQQ

SEQ ID NO: 199 - protein, 15 aa, synthetic construct
EALHNHYTQKSLSLS

SEQ ID NO: 200 - protein, 15 aa, synthetic construct
ALHNHYTQKSLSLSP

SEQ ID NO: 201 - protein, 15 aa, synthetic construct
LHNHYTQKSLSLSPG

SEQ ID NO: 202 - protein, 15 aa, synthetic construct
HNHYTQKSLSLSPGK

SEQ ID NO: 203 - protein, 15 aa, synthetic construct
SPGKNHYTQKSLSLS

SEQ ID NO: 204 - protein, 15 aa, synthetic construct
PGKNHYTQKSLSLSP

SEQ ID NO: 205 - protein, 15 aa, synthetic construct
GKNHYTQKSLSLSPG

SEQ ID NO: 206 - protein, 15 aa, synthetic construct
KNHYTQKSLSLSPGK

SEQ ID NO: 207 - protein, 9 aa, synthetic construct
WVRQAPGQG

SEQ ID NO: 208 - protein, 9 aa, synthetic construct
VRQAPGQGL

SEQ ID NO: 209 - protein, 9 aa, synthetic construct
YMELSSLRS

SEQ ID NO: 210 - protein, 9 aa, synthetic construct
LVTVSSAST

SEQ ID NO: 211 - protein, 9 aa, synthetic construct
VHTFPAVLQ

SEQ ID NO: 212 - protein, 9 aa, synthetic construct
VLQSSGLYS

SEQ ID NO: 213 - protein, 9 aa, synthetic construct
LYSLSSVVT

SEQ ID NO: 214 - protein, 9 aa, synthetic construct
LSSVVTVPS

SEQ ID NO: 215 - protein, 9 aa, synthetic construct
VVTVPSSSL

SEQ ID NO: 216 - protein, 9 aa, synthetic construct
LLGGPSVFL

SEQ ID NO: 217 - protein, 9 aa, synthetic construct
YRVVSVLTV

SEQ ID NO: 218 - protein, 9 aa, synthetic construct
FLYSKLTVD

SEQ ID NO: 219 - protein, 9 aa, synthetic construct
YSKLTVDKS

SEQ ID NO: 220 - protein, 9 aa, synthetic construct
HYTQKSLSL

SEQ ID NO: 221 - protein, 15 aa, synthetic construct
EIVLTQSPGTLSLSP

SEQ ID NO: 222 - protein, 15 aa, synthetic construct
IVLTQSPGTLSLSPG

SEQ ID NO: 223 - protein, 15 aa, synthetic construct
GQAPRLLIYATSILA

SEQ ID NO: 224 - protein, 15 aa, synthetic construct
QAPRLLIYATSILAS

SEQ ID NO: 225 - protein, 15 aa, synthetic construct
APRLLIYATSILASG

SEQ ID NO: 226 - protein, 15 aa, synthetic construct
PRLLIYATSILASGI

SEQ ID NO: 227 - protein, 15 aa, synthetic construct
RLLIYATSILASGIP

SEQ ID NO: 228 - protein, 15 aa, synthetic construct
LLIYATSILASGIPD

SEQ ID NO: 229 - protein, 15 aa, synthetic construct
LIYATSILASGIPDR

SEQ ID NO: 230 - protein, 15 aa, synthetic construct
GTKVEIKRTVAAPSV

SEQ ID NO: 231 - protein, 15 aa, synthetic construct
TKVEIKRTVAAPSVF

SEQ ID NO: 232 - protein, 15 aa, synthetic construct
KVEIKRTVAAPSVFI

SEQ ID NO: 233 - protein, 15 aa, synthetic construct
VEIKRTVAAPSVFIF

SEQ ID NO: 234 - protein, 15 aa, synthetic construct
EIKRTVAAPSVFIFP

SEQ ID NO: 235 - protein, 15 aa, synthetic construct
IKRTVAAPSVFIFPP

SEQ ID NO: 236 - protein, 15 aa, synthetic construct
DEQLKSGTASVVCLL

SEQ ID NO: 237 - protein, 15 aa, synthetic construct
EQLKSGTASVVCLLN

SEQ ID NO: 238 - protein, 15 aa, synthetic construct
DSKDSTYSLSSTLTL

SEQ ID NO: 239 - protein, 15 aa, synthetic construct
SKDSTYSLSSTLTLS

SEQ ID NO: 240 - protein, 15 aa, synthetic construct
KDSTYSLSSTLTLSK

SEQ ID NO: 241 - protein, 15 aa, synthetic construct
DSTYSLSSTLTLSKA

SEQ ID NO: 242 - protein, 15 aa, synthetic construct
STYSLSSTLTLSKAD

SEQ ID NO: 243 - protein, 15 aa, synthetic construct
TYSLSSTLTLSKADY

SEQ ID NO: 244 - protein, 9 aa, synthetic construct
LTQSPGTLS

SEQ ID NO: 245 - protein, 9 aa, synthetic construct
LIYATSILA

SEQ ID NO: 246 - protein, 9 aa, synthetic construct
IKRTVAAPS

SEQ ID NO: 247 - protein, 9 aa, synthetic construct
TVAAPSVFI

SEQ ID NO: 248 - protein, 9 aa, synthetic construct
LKSGTASVV

SEQ ID NO: 249 - protein, 9 aa, synthetic construct
YSLSSTLTL

SEQ ID NO: 250 - protein, 15 aa, synthetic construct
SGGGLVQPGGSLRLS

SEQ ID NO: 251 - protein, 15 aa, synthetic construct
GGGLVQPGGSLRLSC

SEQ ID NO: 252 - protein, 15 aa, synthetic construct
GGLVQPGGSLRLSCA

SEQ ID NO: 253 - protein, 15 aa, synthetic construct
GLVQPGGSLRLSCAA

SEQ ID NO: 254 - protein, 15 aa, synthetic construct
QPGGSLRLSCAAPGF

SEQ ID NO: 255 - protein, 15 aa, synthetic construct
PGGSLRLSCAAPGFI

SEQ ID NO: 256 - protein, 15 aa, synthetic construct
GGSLRLSCAAPGFIF

SEQ ID NO: 257 - protein, 15 aa, synthetic construct
GSLRLSCAAPGFIFS

SEQ ID NO: 258 - protein, 15 aa, synthetic construct
SLRLSCAAPGFIFSS

SEQ ID NO: 259 - protein, 15 aa, synthetic construct
NMNWVRQAPGKGLEW

SEQ ID NO: 260 - protein, 15 aa, synthetic construct
MNWVRQAPGKGLEWV

SEQ ID NO: 261 - protein, 15 aa, synthetic construct
KGLEWVSYISSSSST

SEQ ID NO: 262 - protein, 15 aa, synthetic construct
GLEWVSYISSSSSTT

SEQ ID NO: 263 - protein, 15 aa, synthetic construct
LEWVSYISSSSSTTY

SEQ ID NO: 264 - protein, 15 aa, synthetic construct
EWVSYISSSSSTTYY

SEQ ID NO: 265 - protein, 15 aa, synthetic construct
WVSYISSSSSTTYYA

SEQ ID NO: 266 - protein, 15 aa, synthetic construct
VSYISSSSSTTYYAD

SEQ ID NO: 267 - protein, 15 aa, synthetic construct
SYISSSSSTTYYADS

SEQ ID NO: 268 - protein, 15 aa, synthetic construct
DNAKNSLYLQMNSLR

SEQ ID NO: 269 - protein, 15 aa, synthetic construct
NAKNSLYLQMNSLRD

SEQ ID NO: 270 - protein, 15 aa, synthetic construct
AKNSLYLQMNSLRDE

SEQ ID NO: 271 - protein, 15 aa, synthetic construct
KNSLYLQMNSLRDED

SEQ ID NO: 272 - protein, 15 aa, synthetic construct
NSLYLQMNSLRDEDT

SEQ ID NO: 273 - protein, 15 aa, synthetic construct
SLYLQMNSLRDEDTA

SEQ ID NO: 274 - protein, 15 aa, synthetic construct
LYLQMNSLRDEDTAV

SEQ ID NO: 275 - protein, 9 aa, synthetic construct
VQPGGSLRL

SEQ ID NO: 276 - protein, 9 aa, synthetic construct
LRLSCAAPG

SEQ ID NO: 277 - protein, 9 aa, synthetic construct
VRQAPGKGL

SEQ ID NO: 278 - protein, 9 aa, synthetic construct
WVSYISSSS

SEQ ID NO: 279 - protein, 9 aa, synthetic construct
YISSSSSTT

SEQ ID NO: 280 - protein, 9 aa, synthetic construct
SLYLQMNSL

SEQ ID NO: 281 - protein, 9 aa, synthetic construct
YLQMNSLRD

SEQ ID NO: 282 - protein, 15 aa, synthetic construct
DIVMTQTPLSSPVSL

SEQ ID NO: 283 - protein, 15 aa, synthetic construct
AEDVGVYYCMQATQF

SEQ ID NO: 284 - protein, 15 aa, synthetic construct
EDVGVYYCMQATQFP

SEQ ID NO: 285 - protein, 15 aa, synthetic construct
DVGVYYCMQATQFPF

SEQ ID NO: 286 - protein, 15 aa, synthetic construct
VGVYYCMQATQFPFT

SEQ ID NO: 287 - protein, 15 aa, synthetic construct
GVYYCMQATQFPFTF

SEQ ID NO: 288 - protein, 15 aa, synthetic construct
VYYCMQATQFPFTFG

SEQ ID NO: 289 - protein, 15 aa, synthetic construct
YYCMQATQFPFTFGP

SEQ ID NO: 290 - protein, 15 aa, synthetic construct
ATQFPFTFGPGTKVD

SEQ ID NO: 291 - protein, 15 aa, synthetic construct
TQFPFTFGPGTKVDI

SEQ ID NO: 292 - protein, 15 aa, synthetic construct
QFPFTFGPGTKVDIK

SEQ ID NO: 293 - protein, 15 aa, synthetic construct
FPFTFGPGTKVDIKR

SEQ ID NO: 294 - protein, 15 aa, synthetic construct
GTKVDIKRTVAAPSV

SEQ ID NO: 295 - protein, 15 aa, synthetic construct
TKVDIKRTVAAPSVF

SEQ ID NO: 296 - protein, 15 aa, synthetic construct
KVDIKRTVAAPSVFI

SEQ ID NO: 297 - protein, 15 aa, synthetic construct
VDIKRTVAAPSVFIF

SEQ ID NO: 298 - protein, 15 aa, synthetic construct
DIKRTVAAPSVFIFP

SEQ ID NO: 299 - protein, 15 aa, synthetic construct
PRVLIYKISKRFSGV

SEQ ID NO: 300 - protein, 15 aa, synthetic construct
PGQPPRVLIYKISKR

SEQ ID NO: 301 - protein, 15 aa, synthetic construct
GQPPRVLIYKISKRF

SEQ ID NO: 302 - protein, 15 aa, synthetic construct
QPPRVLIYKISKRFS

SEQ ID NO: 303 - protein, 15 aa, synthetic construct
PPRVLIYKISKRFSG

SEQ ID NO: 304 - protein, 15 aa, synthetic construct
RVLIYKISKRFSGVP

SEQ ID NO: 305 - protein, 15 aa, synthetic construct
VLIYKISKRFSGVPD

SEQ ID NO: 306 - protein, 15 aa, synthetic construct
LIYKISKRFSGVPDR

SEQ ID NO: 307 - protein, 15 aa, synthetic construct
IYKISKRFSGVPDRF

SEQ ID NO: 308 - protein, 9 aa, synthetic construct
IVMTQTPLS

SEQ ID NO: 309 - protein, 9 aa, synthetic construct
YYCMQATQF

SEQ ID NO: 310 - protein, 9 aa, synthetic construct
YCMQATQFP

SEQ ID NO: 311 - protein, 9 aa, synthetic construct
FTFGPGTKV

SEQ ID NO: 312 - protein, 9 aa, synthetic construct
LIYKISKRF

SEQ ID NO: 313 - protein, 9 aa, synthetic construct
VLIYKISKR

SEQ ID NO: 314 - protein, 9 aa, synthetic construct
IYKISKRFS

SEQ ID NO: 315 - protein, 9 aa, synthetic construct
YKISKRFSG

SEQ ID NO: 316 - protein, 6 aa, synthetic construct
HHHHHH

SEQ ID NO: 317 - protein, 63 aa, synthetic construct
KILHIKQDERVTMGQNGNLYFIDLRVKATNSMIDRKPRLLFPKTLQLLKVGEEDDGEYRCLAE

SEQ ID NO: 318 - protein, 42 aa, synthetic construct
IEKYDIEFEDKEMAPEKWYSLWKPLRWMDWNAPQVQYRVQWR

SEQ ID NO: 319 - protein, 5 aa, synthetic construct
  MOD_RES 5: note = Citrulline
EEEVX

SEQ ID NO: 320 - protein, 4 aa, synthetic construct
EGGG

(19) United States
(12) Patent Application Publication
(10) Pub. No.: US 2025/0340636 A1
(43) Pub. Date: Nov. 6, 2025

(54) ANTIBODIES TARGETING L1CAM AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(72) Inventors: Joan Massague, New York, NY (US); Karuna Ganesh, New York, NY (US); Ivo Lorenz, New York, NY (US); Paul Balderes, New York, NY (US); Abdul Khan, New York, NY (US); Sreekumar Kodangattil, New York, NY (US); Manuel Baca, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., Box 300, NY (US)

(21) Appl. No.: 19/258,450

(22) Filed: Jul. 2, 2025

Related U.S. Application Data
(63) Continuation of application No. PCT/US2024/010618, filed on Jan. 8, 2024.
(60) Provisional application No. 63/478,829, filed on Jan. 6, 2023.

(51) Int. Cl. C07K 16/28; A61K 47/68; A61P 35/00; G01N 33/68

(52) U.S. Cl. CPC C07K 16/2803; A61K 47/6803; A61K 47/6849; A61K 47/6889; A61P 35/00; G01N 33/6854; C07K 2317/622; G01N 2333/70503

(57) ABSTRACT

The presently disclosed subject matter provides antibodies or antigen-binding fragments thereof that bind to L1CAM and methods of using such antibodies or antigen-binding fragments thereof. The presently disclosed subject further provides immunoconjugates comprising anti-L1CAM antibodies or antigen-binding fragments thereof and methods of using such immunoconjugates.

### CROSS-REFERENCE TO RELATED APPLICATION

This application is a continuation application of International Patent Application No. PCT/US2024/010618, filed on Jan. 8, 2024, which claims priority to U.S. Provisional Application No. 63/478,829, filed on Jan. 6, 2023, the contents of each of which is hereby incorporated by reference in their entireties, and to each of which priority is claimed.

### SEQUENCE LISTING

The instant application contains a Sequence Listing which has been submitted herewith and is hereby incorporated by reference in its entirety. Said .xml copy, created on Jul. 2, 2025, is named 0727341785.xml, and is 290,608 bytes in size.

### TECHNICAL FIELD

The presently disclosed subject matter relates to antibodies that bind to L1CAM, immunoconjugates comprising such antibodies and methods of using such antibodies and immunoconjugates.

### BACKGROUND

Advanced solid tumors remain mostly incurable due to the emergence of tumor subclones. Termed Metastasis Stem Cells (MetSCs), these tumor subclones are capable of self-renewal, slow cell-cycling, tumor re-initiation, and therapy resistance. The cell adhesion molecule L1CAM was identified as a critical target specifically on MetSCs. In multiple solid cancers, including colorectal, lung, breast, ovarian, pancreas, prostate, and hepatobiliary cancers, L1CAM⁺ MetSCs are enriched following therapy, regenerating more aggressive metastatic cancers, which are the principal cause of cancer mortality. High L1CAM expression in tumors is almost universally associated with poor prognosis.

L1CAM is a crucial marker and mediator of quiescence-capable, chemoresistant, stem-like metastasis regenerating cells. As a result, L1CAM is an attractive target for the treatment of advanced solid tumors. Further, L1CAM is critical for the persistence of multiple solid tumors, including the three types that most prominently cause cancer death (e.g., lung, breast, and colorectal cancer), in multiple metastatic organ sites such as the bone, lung, liver, and brain. L1CAM is critical for the outgrowth of aggressive cancer cells right after freshly seeding a target organ, as well as indolent cancer cells that emerge from dormancy. Thus, new therapeutic approaches are needed to target this dependency of metastasis stem cells and to treat advanced solid tumors.

### SUMMARY

The presently disclosed subject matter provides antibodies or antigen-binding fragments thereof that specifically bind to L1CAM, immunoconjugates comprising such antibodies, and methods of using the antibodies or antigen-binding fragments thereof.

The presently disclosed subject matter provides an anti-L1CAM antibody or an antigen-binding fragment thereof, comprising
(a) a heavy chain variable region comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, a CDR3 comprising the amino acid sequence set forth in SEQ ID NO: 4 or a conservative modification thereof; and a light chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
(b) a heavy chain variable region comprising CDR1 comprising the amino acid sequence set forth in SEQ ID NO: 12 or a conservative modification thereof, a CDR2 comprising an amino acid sequence set forth in SEQ ID NO: 13 or a conservative modification thereof, a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof;
(c) a heavy chain variable region comprising CDR1 comprising the amino acid sequence set forth in SEQ ID NO: 12 or a conservative modification thereof, a CDR2 comprising an amino acid sequence set forth in SEQ ID NO: 13 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof;
(d) a heavy chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, a CDR3 comprising the amino acid sequence set forth in SEQ ID NO: 37 or a conservative modification thereof; and a light chain variable region comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof;
(e) a heavy chain variable region comprising CDR1 comprising the amino acid sequence set forth in SEQ ID NO: 43 or a conservative modification thereof, a CDR2 comprising an amino acid sequence set forth in SEQ ID NO: 44 or a conservative modification thereof, a CDR3 comprising the amino acid sequence set forth in SEQ ID NO: 45 or a conservative modification thereof; and a light chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof;
(f) a heavy chain variable region comprising CDR1 comprising the amino acid sequence set forth in SEQ ID NO: 43 or a conservative modification thereof, a CDR2 comprising an amino acid sequence set forth in SEQ ID NO: 44 or a conservative modification thereof, a CDR3 comprising the amino acid sequence set forth in SEQ ID NO: 45 or a conservative modification thereof; and a light chain variable region comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof;
(g) a heavy chain variable region comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, a CDR3 comprising the amino acid sequence set forth in SEQ ID NO: 56 or a conservative modification thereof; and a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(h) a heavy chain variable region comprising CDR1 comprising the amino acid sequence set forth in SEQ ID NO: 62 or a conservative modification thereof, a CDR2 comprising an amino acid sequence set forth in SEQ ID NO: 63 or a conservative modification thereof, a CDR3 comprising the amino acid sequence set forth in SEQ ID NO: 64 or a conservative modification thereof; and a light chain variable region comprising a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof;
(i) a heavy chain variable region comprising CDR1 comprising the amino acid sequence set forth in SEQ ID NO: 69 or a conservative modification thereof, a CDR2 comprising an amino acid sequence set forth in SEQ ID NO: 70 or a conservative modification thereof, a CDR3 comprising the amino acid sequence set forth in SEQ ID NO: 71 or a conservative modification thereof; and a light chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof;
(j) a heavy chain variable region comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, a CDR3 comprising the amino acid sequence set forth in SEQ ID NO: 83 or a conservative modification thereof; and a light chain variable region comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof;
(k) a heavy chain variable region comprising CDR1 comprising the amino acid sequence set forth in SEQ ID NO: 93 or a conservative modification thereof, a CDR2 comprising an amino acid sequence set forth in SEQ ID NO: 94 or a conservative modification thereof, a CDR3 comprising the amino acid sequence set forth in SEQ ID NO: 95 or a conservative modification thereof; and a light chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof;
(l) a heavy chain variable region comprising CDR1 comprising the amino acid sequence set forth in SEQ ID NO: 105 or a conservative modification thereof, a CDR2 comprising an amino acid sequence set forth in SEQ ID NO: 106 or a conservative modification thereof, a CDR3 comprising the amino acid sequence set forth in SEQ ID NO: 107 or a conservative modification thereof; and a light chain variable region comprising a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof; or
(m) a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a light chain variable region comprising a CDR1 comprising the amino acid sequence set forth in SEQ ID NO: 119 or a conservative modification thereof, a CDR2 comprising the amino acid sequence set forth in SEQ ID NO: 120, and a CDR3 comprising the amino acid sequence set forth in SEQ ID NO: 121 or a conservative modification thereof.

In certain embodiments,
(a) the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) the heavy chain variable region comprises CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 26; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(d) the heavy chain variable region comprises a CDR1 comprising the amino acid sequence set forth in SEQ ID NO: 35, a CDR2 comprising the amino acid sequence set forth in SEQ ID NO: 36, a CDR3 comprising the amino acid sequence set forth in SEQ ID NO: 37; and the light chain variable region comprises a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40v;
(e) the heavy chain variable region comprises CDR1 comprising the amino acid sequence set forth in SEQ ID NO: 43, a CDR2 comprising an amino acid sequence set forth in SEQ ID NO: 44, a CDR3 comprising the amino acid sequence set forth in SEQ ID NO: 45; and the light chain variable region comprises a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(f) the heavy chain variable region comprises CDR1 comprising the amino acid sequence set forth in SEQ ID NO: 43, a CDR2 comprising an amino acid sequence set forth in SEQ ID NO: 44, a CDR3 comprising the amino acid sequence set forth in SEQ ID NO: 45; and the light chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40;
(g) the heavy chain variable region comprises a CDR1 comprising the amino acid sequence set forth in SEQ ID NO: 54, a CDR2 comprising the amino acid sequence set forth in SEQ ID NO: 55, a CDR3 comprising the amino acid sequence set forth in SEQ ID NO: 56; and the light chain variable region comprises a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) the heavy chain variable region comprises CDR1 comprising the amino acid sequence set forth in SEQ ID NO: 62, a CDR2 comprising an amino acid sequence set forth in SEQ ID NO: 63, a CDR3 comprising the amino acid sequence set forth in SEQ ID NO: 64; and the light chain variable region comprises a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66;
(i) the heavy chain variable region comprises CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(j) the heavy chain variable region comprises a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
(k) the heavy chain variable region comprises CDR1 comprising the amino acid sequence set forth in SEQ ID NO: 93, a CDR2 comprising an amino acid sequence set forth in SEQ ID NO: 94, a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
(l) the heavy chain variable region comprises CDR1 comprising the amino acid sequence set forth in SEQ ID NO: 105, a CDR2 comprising an amino acid sequence set forth in SEQ ID NO: 106, a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109; or
(m) the heavy chain variable region comprises a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and the light chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments,
(a) the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprising a CDR1 comprises the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or
(c) the heavy chain variable region comprises a CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprising a CDR1 comprises the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the heavy chain variable region comprises a CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the heavy chain variable region comprises a CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the anti-L1CAM antibody or an antigen-binding fragment thereof comprises
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 22, SEQ ID NO: 24, SEQ ID NO: 25, SEQ ID NO: 27, SEQ ID NO: 28, SEQ ID NO: 29, SEQ ID NO: 33, SEQ ID NO: 41, SEQ ID NO: 49, SEQ ID NO: 60, SEQ ID NO: 67, SEQ ID NO: 75, SEQ ID NO: 87, SEQ ID NO: 99, SEQ ID NO: 110, SEQ ID NO: 122, SEQ ID NO: 128, SEQ ID NO: 130, SEQ ID NO: 134, or SEQ ID NO: 135; or
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 9, SEQ ID NO: 19, SEQ ID NO: 23, SEQ ID NO: 30, SEQ ID NO: 31, SEQ ID NO: 32, SEQ ID NO: 34, SEQ ID NO: 42, SEQ ID NO: 50, SEQ ID NO: 53, SEQ ID NO: 61, SEQ ID NO: 68, SEQ ID NO: 76, SEQ ID NO: 88, SEQ ID NO: 100, SEQ ID NO: 111, SEQ ID NO: 123, SEQ ID NO: 129, SEQ ID NO: 131, SEQ ID NO: 132, or SEQ ID NO: 133.

In certain embodiments, the anti-L1CAM antibody or an antigen-binding fragment thereof comprises
(a) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 9;
(b) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 18, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(c) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(d) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(e) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(f) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(g) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(h) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(i) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(j) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(k) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(l) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(m) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(n) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(o) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(p) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(q) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(r) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(s) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(t) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(u) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(v) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(w) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(x) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(y) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(z) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (aa) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ab) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ac) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ad) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ae) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(af) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ag) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 33, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 34;
(ah) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 41, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 42;
(ai) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 50;
(aj) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 53;
(ak) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 60, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 61;
(al) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 67, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 68;
(am) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 75, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 76;
(an) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 87, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 88;
(ao) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 99, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 100;
(ap) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 110, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 111;
(aq) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 122, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 123;
(ar) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 128, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 129;
(as) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131;
(at) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132;
(au) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133;
(av) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131;
(aw) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132;
(ax) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133;
(ay) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131;
(az) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132;
(ba) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133; and
(bb) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 136.

In certain embodiments,
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 22, and the light chain variable region comprises the amino acid sequence set forth in SEQ ID NO: 23;
(d) the heavy chain variable region comprises the amino acid sequence set forth in SEQ ID NO: 24, and the light chain variable region comprises the amino acid sequence set forth in SEQ ID NO: 23;
(e) the heavy chain variable region comprises the amino acid sequence set forth in SEQ ID NO: 25, and the light chain variable region comprises the amino acid sequence set forth in SEQ ID NO: 23;
(f) the heavy chain variable region comprises the amino acid sequence set forth in SEQ ID NO: 27, and the light chain variable region comprises the amino acid sequence set forth in SEQ ID NO: 23;
(g) the heavy chain variable region comprises the amino acid sequence set forth in SEQ ID NO: 28, and the light chain variable region comprises the amino acid sequence set forth in SEQ ID NO: 23;
(h) the heavy chain variable region comprises the amino acid sequence set forth in SEQ ID NO: 29, and the light chain variable region comprises the amino acid sequence set forth in SEQ ID NO: 23;
(i) the heavy chain variable region comprises the amino acid sequence set forth in SEQ ID NO: 22, and the light chain variable region comprises the amino acid sequence set forth in SEQ ID NO: 30;
j) the heavy chain variable region comprises the amino acid sequence set forth in SEQ ID NO: 24, and the light chain variable region comprises the amino acid sequence set forth in SEQ ID NO: 30;
(k) the heavy chain variable region comprises the amino acid sequence set forth in SEQ ID NO: 25, and the light chain variable region comprises the amino acid sequence set forth in SEQ ID NO: 30;
(l) the heavy chain variable region comprises the amino acid sequence set forth in SEQ ID NO: 27, and the light chain variable region comprises the amino acid sequence set forth in SEQ ID NO: 30;
(m) the heavy chain variable region comprises the amino acid sequence set forth in SEQ ID NO: 28, and the light chain variable region comprises the amino acid sequence set forth in SEQ ID NO: 30;
(n) the heavy chain variable region comprises the amino acid sequence set forth in SEQ ID NO: 29, and the light chain variable region comprises the amino acid sequence set forth in SEQ ID NO: 30;
(o) the heavy chain variable region comprises the amino acid sequence set forth in SEQ ID NO: 22, and the light chain variable region comprises the amino acid sequence set forth in SEQ ID NO: 31;
(p) the heavy chain variable region comprises the amino acid sequence set forth in SEQ ID NO: 24, and the light chain variable region comprises the amino acid sequence set forth in SEQ ID NO: 31;
(q) the heavy chain variable region comprises the amino acid sequence set forth in SEQ ID NO: 25, and the light chain variable region comprises the amino acid sequence set forth in SEQ ID NO: 31;
(r) the heavy chain variable region comprises the amino acid sequence set forth in SEQ ID NO: 27, and the light chain variable region comprises the amino acid sequence set forth in SEQ ID NO: 31;
(s) the heavy chain variable region comprises the amino acid sequence set forth in SEQ ID NO: 28, and the light chain variable region comprises the amino acid sequence set forth in SEQ ID NO: 31;
(t) the heavy chain variable region comprises the amino acid sequence set forth in SEQ ID NO: 29, and the light chain variable region comprises the amino acid sequence set forth in SEQ ID NO: 31;
(u) the heavy chain variable region comprises the amino acid sequence set forth in SEQ ID NO: 22, and the light chain variable region comprises the amino acid sequence set forth in SEQ ID NO: 32;
(v) the heavy chain variable region comprises the amino acid sequence set forth in SEQ ID NO: 24, and the light chain variable region comprises the amino acid sequence set forth in SEQ ID NO: 32;
(w) the heavy chain variable region comprises the amino acid sequence set forth in SEQ ID NO: 25, and the light chain variable region comprises the amino acid sequence set forth in SEQ ID NO: 32;
(x) the heavy chain variable region comprises the amino acid sequence set forth in SEQ ID NO: 27, and the light chain variable region comprises the amino acid sequence set forth in SEQ ID NO: 32;
(y) the heavy chain variable region comprises the amino acid sequence set forth in SEQ ID NO: 28, and the light chain variable region comprises the amino acid sequence set forth in SEQ ID NO: 32;
(z) the heavy chain variable region comprises the amino acid sequence set forth in SEQ ID NO: 29, and the light chain variable region comprises the amino acid sequence set forth in SEQ ID NO: 32;
(aa) the heavy chain variable region comprises the amino acid sequence set forth in SEQ ID NO: 22, and the light chain variable region comprises the amino acid sequence set forth in SEQ ID NO: 19;
(ab) the heavy chain variable region comprises the amino acid sequence set forth in SEQ ID NO: 24, and the light chain variable region comprises the amino acid sequence set forth in SEQ ID NO: 19;
(ac) the heavy chain variable region comprises the amino acid sequence set forth in SEQ ID NO: 25, and the light chain variable region comprises the amino acid sequence set forth in SEQ ID NO: 19;
(ad) the heavy chain variable region comprises the amino acid sequence set forth in SEQ ID NO: 27, and the light chain variable region comprises the amino acid sequence set forth in SEQ ID NO: 19;
(ae) the heavy chain variable region comprises the amino acid sequence set forth in SEQ ID NO: 28, and the light chain variable region comprises the amino acid sequence set forth in SEQ ID NO: 19;
(af) the heavy chain variable region comprises the amino acid sequence set forth in SEQ ID NO: 29, and the light chain variable region comprises the amino acid sequence set forth in SEQ ID NO: 19;
(ag) the heavy chain variable region comprising the amino acid sequence set forth in SEQ ID NO: 33, and the light chain variable region comprising the amino acid sequence set forth in SEQ ID NO: 34;
(ah) the heavy chain variable region comprising the amino acid sequence set forth in SEQ ID NO: 41, and the light chain variable region comprising the amino acid sequence set forth in SEQ ID NO: 42;
(ai) the heavy chain variable region comprising the amino acid sequence set forth in SEQ ID NO: 49, and the light chain variable region comprising the amino acid sequence set forth in SEQ ID NO: 50;
(aj) the heavy chain variable region comprising the amino acid sequence set forth in SEQ ID NO: 49, and the light chain variable region comprising the amino acid sequence set forth in SEQ ID NO: 53;
(ak) the heavy chain variable region comprising the amino acid sequence set forth in SEQ ID NO: 60, and the light chain variable region comprising the amino acid sequence set forth in SEQ ID NO: 61;
(al) the heavy chain variable region comprising the amino acid sequence set forth in SEQ ID NO: 67, and the light chain variable region comprising the amino acid sequence set forth in SEQ ID NO: 68;
(am) the heavy chain variable region comprising the amino acid sequence set forth in SEQ ID NO: 75, and the light chain variable region comprising the amino acid sequence set forth in SEQ ID NO: 76;
(an) the heavy chain variable region comprising the amino acid sequence set forth in SEQ ID NO: 87, and the light chain variable region comprising the amino acid sequence set forth in SEQ ID NO: 88;
(ao) the heavy chain variable region comprising the amino acid sequence set forth in SEQ ID NO: 99, and the light chain variable region comprising the amino acid sequence set forth in SEQ ID NO: 100;
(ap) the heavy chain variable region comprising the amino acid sequence set forth in SEQ ID NO: 110, and the light chain variable region comprising the amino acid sequence set forth in SEQ ID NO: 111;
(aq) the heavy chain variable region comprising the amino acid sequence set forth in SEQ ID NO: 122, and the light chain variable region comprising the amino acid sequence set forth in SEQ ID NO: 123;
(ar) the heavy chain variable region comprising the amino acid sequence set forth in SEQ ID NO: 128, and the light chain variable region comprising the amino acid sequence set forth in SEQ ID NO: 129;
(as) the heavy chain variable region comprising the amino acid sequence set forth in SEQ ID NO: 130, and the light chain variable region comprising the amino acid sequence set forth in SEQ ID NO: 131;
(at) the heavy chain variable region comprising the amino acid sequence set forth in SEQ ID NO: 130, and the light chain variable region comprising the amino acid sequence set forth in SEQ ID NO: 132;
(au) the heavy chain variable region comprising the amino acid sequence set forth in SEQ ID NO: 130, and the light chain variable region comprising the amino acid sequence set forth in SEQ ID NO: 133;
(av) the heavy chain variable region comprising the amino acid sequence set forth in SEQ ID NO: 134, and the light chain variable region comprising the amino acid sequence set forth in SEQ ID NO: 131;
(aw) the heavy chain variable region comprising the amino acid sequence set forth in SEQ ID NO: 134, and the light chain variable region comprising the amino acid sequence set forth in SEQ ID NO: 132;
(ax) the heavy chain variable region comprising the amino acid sequence set forth in SEQ ID NO: 134, and the light chain variable region comprising the amino acid sequence set forth in SEQ ID NO: 133;
(ay) the heavy chain variable region comprising the amino acid sequence set forth in SEQ ID NO: 135, and the light chain variable region comprising the amino acid sequence set forth in SEQ ID NO: 131;
(az) the heavy chain variable region comprising the amino acid sequence set forth in SEQ ID NO: 135, and the light chain variable region comprising the amino acid sequence set forth in SEQ ID NO: 132;
(ba) the heavy chain variable region comprising the amino acid sequence set forth in SEQ ID NO: 135, and the light chain variable region comprising the amino acid sequence set forth in SEQ ID NO: 133; or
(bb) the heavy chain variable region comprising the amino acid sequence set forth in SEQ ID NO: 135, and the light chain variable region comprising the amino acid sequence set forth in SEQ ID NO: 136.

In certain embodiments, the antibody comprises a comprises a heavy chain constant region and/or a light chain constant region. In certain embodiments,
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence set forth in SEQ ID NO: 144, SEQ ID NO: 145, SEQ ID NO: 146, or SEQ ID NO: 147; and/or
(b) the light chain constant region comprises an amino acid sequence that is at least about 80%, at least about 85%, at least about 90%, at least about 95%, at least about 96%, at least about 97%, at least about 98%, at least about 99% at least about 100% identical to the amino acid sequence set forth in SEQ ID NO: 148.

In certain embodiments,
(a) the heavy chain constant region comprises the amino acid sequence set forth in SEQ ID NO: 144, SEQ ID NO: 145, SEQ ID NO: 146, or SEQ ID NO: 147; and/or
(b) the light chain constant region comprises the amino acid sequence set forth in SEQ ID NO: 148.

In certain embodiments, the antibody comprises a human variable region framework region. In certain embodiments, the antibody of antigen-fragment thereof is a fully human or an antigen-binding fragment thereof. In certain embodiments, the antibody or antigen-binding fragment thereof is a chimeric antibody or an antigen-binding fragment thereof. In certain embodiments, the antibody or antigen-binding fragment thereof is a humanized antibody or an antigen-binding fragment thereof. In certain embodiments, the antigen-binding fragment is a Fab, Fab′, F(ab′)2, variable fragment (Fv), or single chain variable region (scFv). In certain embodiments, the antigen-binding fragment is an scFv.

The presently disclosed subject matter also provides an anti-L1CAM antibody or an antigen-binding fragment thereof comprising
(a) a heavy chain variable region comprising a CDR1, a CDR2, and a CDR3 of the heavy chain variable region comprising the amino acid sequence set forth in SEQ ID NO: 8, and a light chain variable region comprising a CDR1, a CDR2, and a CDR3 of the light chain variable region comprising the amino acid sequence set forth in SEQ ID NO: 9;
(b) a heavy chain variable region comprising a CDR1, a CDR2, and a CDR3 of the heavy chain variable region comprising the amino acid sequence set forth in SEQ ID NO: 18, and a light chain variable region comprising a CDR1, a CDR2, and a CDR3 of the light chain variable region comprising the amino acid sequence set forth in SEQ ID NO: 19;
(c) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 23;
(d) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 23;
(e) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 23;
(f) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 23;
(g) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 23;
(h) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 23;
(i) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 30;
(j) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 30;
(k) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 30;
(l) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 30;
(m) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 30;
(n) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 30;
(o) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 31;
(p) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 31;
(q) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 31;
(r) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 31;
(s) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 31;
(t) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 31;
(u) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 32;
(v) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 32;
(w) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 32;
(x) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 32;
(y) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 32;
(z) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 32;
(aa) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 19;
(ab) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 19;
(ac) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 19;
(ad) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 19;
(ae) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 19;
(af) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 19;
(ag) the heavy chain variable region comprising the amino acid sequence set forth in SEQ ID NO: 33, and the light chain variable region comprising the amino acid sequence set forth in SEQ ID NO: 34;
(ah) the heavy chain variable region comprising the amino acid sequence set forth in SEQ ID NO: 41, and the light chain variable region comprising the amino acid sequence set forth in SEQ ID NO: 42;
(ai) the heavy chain variable region comprising the amino acid sequence set forth in SEQ ID NO: 49, and the light chain variable region comprising the amino acid sequence set forth in SEQ ID NO: 50;
(aj) the heavy chain variable region comprising the amino acid sequence set forth in SEQ ID NO: 49, and the light chain variable region comprising the amino acid sequence set forth in SEQ ID NO: 53;
(ak) the heavy chain variable region comprising the amino acid sequence set forth in SEQ ID NO: 60, and the light chain variable region comprising the amino acid sequence set forth in SEQ ID NO: 61;
(al) the heavy chain variable region comprising the amino acid sequence set forth in SEQ ID NO: 67, and the light chain variable region comprising the amino acid sequence set forth in SEQ ID NO: 68;
(am) the heavy chain variable region comprising the amino acid sequence set forth in SEQ ID NO: 75, and the light chain variable region comprising the amino acid sequence set forth in SEQ ID NO: 76;
(an) the heavy chain variable region comprising the amino acid sequence set forth in SEQ ID NO: 87, and the light chain variable region comprising the amino acid sequence set forth in SEQ ID NO: 88;
(ao) the heavy chain variable region comprising the amino acid sequence set forth in SEQ ID NO: 99, and the light chain variable region comprising the amino acid sequence set forth in SEQ ID NO: 100;
(ap) the heavy chain variable region comprising the amino acid sequence set forth in SEQ ID NO: 110, and the light chain variable region comprising the amino acid sequence set forth in SEQ ID NO: 111;
(aq) the heavy chain variable region comprising the amino acid sequence set forth in SEQ ID NO: 122, and the light chain variable region comprising the amino acid sequence set forth in SEQ ID NO: 123;
(ar) the heavy chain variable region comprising the amino acid sequence set forth in SEQ ID NO: 128, and the light chain variable region comprising the amino acid sequence set forth in SEQ ID NO: 129;
(as) the heavy chain variable region comprising the amino acid sequence set forth in SEQ ID NO: 130, and the light chain variable region comprising the amino acid sequence set forth in SEQ ID NO: 131;
(at) the heavy chain variable region comprising the amino acid sequence set forth in SEQ ID NO: 130, and the light chain variable region comprising the amino acid sequence set forth in SEQ ID NO: 132;
(au) the heavy chain variable region comprising the amino acid sequence set forth in SEQ ID NO: 130, and the light chain variable region comprising the amino acid sequence set forth in SEQ ID NO: 133;
(av) the heavy chain variable region comprising the amino acid sequence set forth in SEQ ID NO: 134, and the light chain variable region comprising the amino acid sequence set forth in SEQ ID NO: 131;
(aw) the heavy chain variable region comprising the amino acid sequence set forth in SEQ ID NO: 134, and the light chain variable region comprising the amino acid sequence set forth in SEQ ID NO: 132;
(ax) the heavy chain variable region comprising the amino acid sequence set forth in SEQ ID NO: 134, and the light chain variable region comprising the amino acid sequence set forth in SEQ ID NO: 133;
(ay) the heavy chain variable region comprising the amino acid sequence set forth in SEQ ID NO: 135, and the light chain variable region comprising the amino acid sequence set forth in SEQ ID NO: 131;
(az) the heavy chain variable region comprising the amino acid sequence set forth in SEQ ID NO: 135, and the light chain variable region comprising the amino acid sequence set forth in SEQ ID NO: 132;
(ba) the heavy chain variable region comprising the amino acid sequence set forth in SEQ ID NO: 135, and the light chain variable region comprising the amino acid sequence set forth in SEQ ID NO: 133; or
(bb) the heavy chain variable region comprising the amino acid sequence set forth in SEQ ID NO: 135, and the light chain variable region comprising the amino acid sequence set forth in SEQ ID NO: 136.

The presently disclosed subject matter also provides an anti-L1CAM antibody or an antigen-binding fragment thereof comprising:
(a) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 77 or SEQ ID NO: 78;
(b) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 89 or SEQ ID NO: 90;
(c) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 101 or SEQ ID NO: 102;
(d) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 112 or SEQ ID NO: 113; or
(e) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 124 or SEQ ID NO: 125.

In certain embodiments,
(a) the scFv comprises the amino acid sequence set forth in SEQ ID NO: 77 or SEQ ID NO: 78;
(b) the scFv comprises the amino acid sequence set forth in SEQ ID NO: 89 or SEQ ID NO: 90;
(c) the scFv comprises the amino acid sequence set forth in SEQ ID NO: 101 or SEQ ID NO: 102;
(d) the scFv comprises the amino acid sequence set forth in SEQ ID NO: 112 or SEQ ID NO: 113; or
(e) the scFv comprises the amino acid sequence set forth in SEQ ID NO: 124 or SEQ ID NO: 125.

The presently disclosed subject matter further provides an anti-L1CAM antibody or an antigen-binding fragment thereof comprising a heavy chain variable region comprising a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

The presently disclosed subject matter also provides an anti-L1CAM antibody or an antigen-binding fragment thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

The presently disclosed subject matter also provides an anti-L1CAM antibody or an antigen-binding fragment thereof comprising a heavy chain variable region comprising a CDR1 comprising the amino acid sequence set forth in SEQ ID NO: 69, a CDR2 comprising the amino acid sequence set forth in SEQ ID NO: 70, a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

The presently disclosed subject matter also provides an anti-L1CAM antibody or an antigen-binding fragment thereof comprising a heavy chain variable region comprising a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

The presently disclosed subject matter also provides an anti-L1CAM antibody or an antigen-binding fragment thereof comprising a heavy chain variable region comprising a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 95; and a light chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

The presently disclosed subject matter also provides an anti-L1CAM antibody or an antigen-binding fragment thereof comprising a heavy chain variable region comprising a heavy chain variable region comprising the amino acid sequence set forth in SEQ ID NO: 8, and a light chain variable region comprising the amino acid sequence set forth in SEQ ID NO: 9.

The presently disclosed subject matter also provides an anti-L1CAM antibody or an antigen-binding fragment thereof comprising a heavy chain variable region comprising a heavy chain variable region comprising the amino acid sequence set forth in SEQ ID NO: 18, and a light chain variable region comprising the amino acid sequence set forth in SEQ ID NO: 19.

The presently disclosed subject matter also provides an anti-L1CAM antibody or an antigen-binding fragment thereof comprising a heavy chain variable region comprising a heavy chain variable region comprising the amino acid sequence set forth in SEQ ID NO: 75, and a light chain variable region comprising the amino acid sequence set forth in SEQ ID NO: 76.

The presently disclosed subject matter also provides an anti-L1CAM antibody or an antigen-binding fragment thereof comprising a heavy chain variable region comprising a heavy chain variable region comprising the amino acid sequence set forth in SEQ ID NO: 87, and a light chain variable region comprising the amino acid sequence set forth in SEQ ID NO: 88.

The presently disclosed subject matter also provides an anti-L1CAM antibody or an antigen-binding fragment thereof comprising a heavy chain variable region comprising a heavy chain variable region comprising the amino acid sequence set forth in SEQ ID NO: 99, and a light chain variable region comprising the amino acid sequence set forth in SEQ ID NO: 100.

The presently disclosed subject matter also provides an anti-L1CAM antibody or an antigen-binding fragment thereof comprising the scFv comprises the amino acid sequence set forth in SEQ ID NO: 77 or SEQ ID NO: 78.

The presently disclosed subject matter also provides an anti-L1CAM antibody or an antigen-binding fragment thereof comprising the scFv comprises the amino acid sequence set forth in SEQ ID NO: 89 or SEQ ID NO: 90.

The presently disclosed subject matter also provides an anti-L1CAM antibody or an antigen-binding fragment thereof comprising the scFv comprises the amino acid sequence set forth in SEQ ID NO: 101 or SEQ ID NO: 102.

In certain embodiments, the presently disclosed subject matter provides an antibody or an antigen-binding fragment thereof that cross-competes for binding to or binds to the same epitope region on L1CAM with an anti-L1CAM antibody or an antigen-binding fragment thereof disclosed herein.

Further, the presently disclosed subject matter provides for an immunoconjugate comprising an anti-L1CAM antibody or antigen-binding fragment thereof disclosed herein. In certain embodiments, the anti-L1CAM antibody or antigen-binding fragment thereof is linked to a therapeutic agent. In certain embodiments, the therapeutic agent is a drug, a cytotoxin, or a radioactive isotope. In certain embodiments, the therapeutic agent is a compound selected from the following classes of compounds: dolastatins, maytansinoids, duocarmycins, anthracyclines, camptothecins, and amatoxins. In certain embodiments, the therapeutic agent is selected from the group consisting of monomethyl auristatin E, ABZ038, duocarmycin TM, PNU159682, SN38, and α-Amanitin. In certain embodiments, the therapeutic agent is PNU159682.

In certain embodiments, the immunoconjugate comprises a linker. In certain embodiments, the linker is a cleavable linker or a non-cleavable linker. In certain embodiments, the immunoconjugate comprises a molecule of formula:

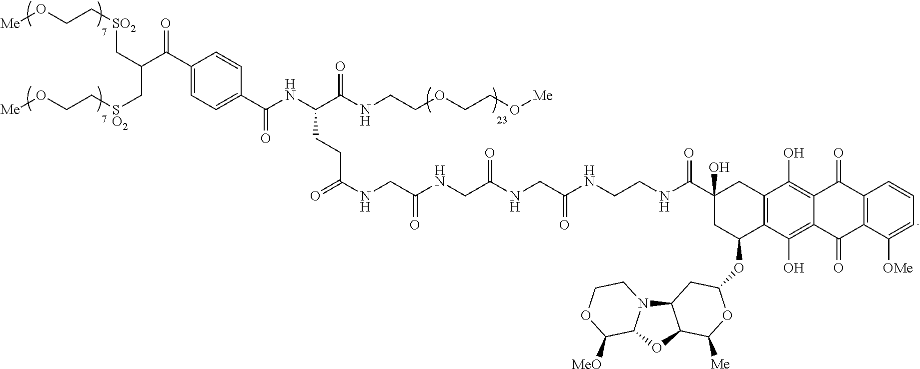

In certain embodiments, the immunoconjugate comprises a molecule of formula:

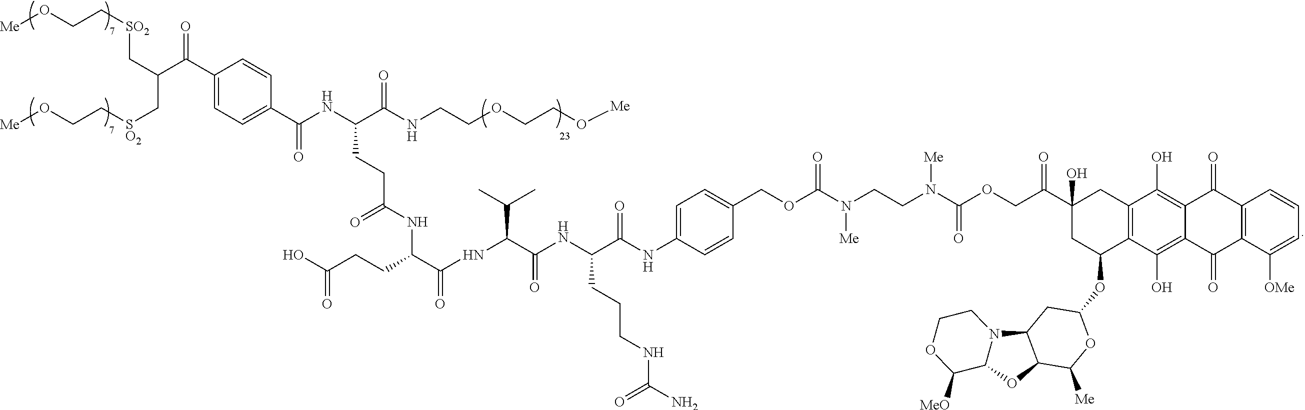

Moreover, the presently disclosed subject matter provides a multi-specific molecule comprising an anti-L1CAM antibody or antigen-binding fragment thereof disclosed herein, linked to one or more functional moieties. In certain embodiments, the one or more functional moieties have a different binding specificity than the anti-L1CAM antibody or antigen-binding fragment thereof.

Also, the presently disclosed subject matter provides a composition comprising an anti-L1CAM antibody or antigen-binding fragment thereof, an immunoconjugate, or a multi-specific molecule disclosed herein. In certain embodiments, the composition is a pharmaceutical composition that further comprises a pharmaceutically acceptable carrier.

The presently disclosed subject matter provides a nucleic acid that encodes an anti-L1CAM antibody or antigen-binding fragment thereof, a heavy chain variable region of an anti-L1CAM antibody or antigen-binding fragment thereof, and/or a light chain variable region of an anti-L1CAM antibody or antigen-binding fragment thereof disclosed herein. In certain embodiments, the presently disclosed subject matter provides a vector or a host cell comprising the nucleic acid disclosed herein.

Further, the presently disclosed subject matter provides a method for detecting L1CAM in a whole cell, a tissue, or a blood sample. In certain embodiments, the method comprises contacting a cell, tissue, or blood sample with an anti-L1CAM antibody or antigen-binding fragment thereof disclosed herein. In certain embodiments, the anti-L1CAM antibody or antigen-binding fragment thereof comprises a detectable label. In certain embodiments, the method comprises determining the amount of the labeled antibody or antigen-binding fragment thereof bound to the cell, tissue, or blood sample by measuring the amount of detectable label associated with said cell or tissue, wherein the amount of bound antibody or antigen-binding fragment thereof indicates the amount of L1CAM in the cell, tissue or blood sample.

The presently disclosed subject matter provides a method of treating or ameliorating a disease or disorder associated with L1CAM in a subject, and/or reducing tumor burden in a subject, and/or treating and/or preventing a tumor in a subject, and/or increasing or lengthening survival of a subject having a tumor. In certain embodiments, the method comprises administering to the subject an anti-L1CAM antibody or antigen-binding fragment thereof, an immunoconjugate, a multi-specific molecule, or a composition disclosed herein.

In certain embodiments, the disease or disorder is a tumor. In certain embodiments, the method reduces the number of the tumor cells, reduces the tumor size, and/or eradicates the tumor in the subject. In certain embodiments, the method reduces or eradicates tumor burden in the subject. In certain embodiments, the tumor is cancer. In certain embodiments, the tumor is a metastatic cancer. In certain embodiments, metastatic cancer is an advanced metastatic cancer. In certain embodiments, the subject is a human.

The presently disclosed subject matter further provides a kit for treating or ameliorating a disease or disorder in a subject, reducing tumor burden in a subject, treating and/or preventing a tumor in a subject, and/or increasing or lengthening survival of a subject having a tumor, comprising an anti-L1CAM antibody or antigen-binding fragment thereof, an immunoconjugate, a multi-specific molecule, or a composition disclosed herein. In certain embodiments, the kit further comprises written instructions for using the antibody or antigen-binding fragment thereof, immunoconjugate, multi-specific molecule, or composition disclosed herein.

### BRIEF DESCRIPTION OF THE FIGURES

The following Detailed Description, given by way of example, but not intended to limit the invention to specific embodiments described, may be understood in conjunction with the accompanying drawings.

FIGS. 1A-1F depict effects of L1CAM on metastatic outgrowth. FIG. 1A shows L1CAM mediating cancer cell spreading. FIG. 1B shows L1CAM expression is associated with poor survival in CRC. FIG. 1C shows doxycycline-inducible L1CAM knockdown after intracardiac injection of lung adenocarcinoma cells reduces metastatic growth. FIG. 1D shows YAP nuclear localization in H2030-BrM lung adenocarcinoma cells spreading on vascular basal lamina is reduced by L1CAM knockdown. FIG. 1E shows reduction in brain metastasis by L1CAM knockdown is rescued by active YAP5SA mutant but not by inactive YAPS94A mutant. FIG. 1F shows a representative model of metastatic outgrowth: L1CAM amplifies Integrin-ILK signaling and triggers a YAP transcriptional program for metastatic colonization.

FIGS. 2A-2C depict L1CAM expression in human lung and colorectal cancer. FIG. 2A shows L1CAM enrichment in post-therapy residual disease in lung. FIG. 2B shows L1CAM enrichment in post-therapy residual disease in colorectal cancer. FIG. 2C shows L1CAM⁺ cells express low levels of the proliferation marker Ki67.

FIGS. 3A-3C depict distinct L1CAM⁺ population driving CRC organoid and metastatic regeneration. FIG. 3A shows L1CAM⁺ cells FACS-sorted from resected patient CRC liver metastases regenerate organoids more efficiently than L1CAM⁻ cells. FIG. 3B shows L1CAM⁺ cells FACS-sorted from resected patient CRC liver metastases regenerate xenograft tumors more efficiently than L1CAM⁻ cells. FIG. 3C shows L1CAM CRISPR knockout cells have diminished organoid regeneration capacity. FIGS. 3D and 3E show single-cell mRNA sequencing of primary tumor/liver metastasis CRC patient-derived organoids having differential abundance of LGR5⁺ tumor-initiating cells and L1CAM⁺ metastasis stem cells.

FIG. 4 shows L1CAM⁺ cells in normal and metastatic regeneration.

FIGS. 5A-5J depict plasticity of the L1CAM⁺ MetSC phenotype. FIG. 5A shows L1CAM expression is dynamically regulated during organoid growth. Progressive restriction of L1CAM immuno-histochemistry staining to cells at the periphery, and overall decrease in L1CAM expression, with increasing organoid size. FIG. 5B shows L1CAM is induced by dissociation of normal, primary, and metastatic human CRC organoids. FIG. 5C shows a time course of regenerating organoids derived from L1CAMhigh (left) or L1CAMlow (right) sorted from 21-day MSK107Li (top) or MSK121Li (bottom) organoids. FIGS. 5D and 5E show dynamic induction of the L1CAMhigh phenotype by a subset of pre-existing L1CAMlow cells. FIG. 5D shows percentage of tdTomato and GFP-expressing cells. FIG. 5E shows relative proportions in the population at the indicated timepoints following mixing. FIG. 5F shows distribution of cells derived from tdTomato+GFP-L1CAMhigh and tdTomato-GFP+L1CAMlow precursors. FIG. 5G shows L1CAM expression of cells derived from tdTomato+GFP-L1CAMhigh and tdTomato-GFP+L1CAMlow precursors. FIGS. 5H-5J show combination of L1CAM inhibition with chemotherapy impairs tumor growth in vivo to a greater extent than chemotherapy alone.

FIG. 6 depicts screening funnel and hit summary for hybridoma supernatants. The steps of the screening funnel are shown in sequence on the left. For each step, utilized method, selection criteria and number of hits for wild-type mice (Balb/c & A/J) and transgenic mice (AlivaMab®) are indicated.

FIG. 7 depicts internalization of L1CAM lead candidate mAbs. A Fab-ZAP assay was used to determine the potency of internalization for the L1CAM lead candidate mAbs in HEK293T cells overexpressing human L1CAM. The L1CAM mAbs and a mouse IgG1 isotype control were pre-incubated at various concentrations with a saporin-conjugated Fab fragment that binds mouse Fc. Five days later, cell viability was measured and plotted against mAb concentrations.

FIG. 8 depicts K_{D} determination by BLI of humanized variants.

FIG. 9 depicts binding of humanized variants of the presently disclosed antibodies to cells expressing L1CAM by FACS analysis.

FIG. 10 depicts interaction between human L1CAM (huL1CAM) and TDI-Y-004. SEQ ID NO: 317 is shown in FIG. 10.

FIGS. 11A-11E depict 3D rendering of interaction between human L1CAM (huL1CAM) and TDI-Y-004. A PDB structure of huL1CAM was generated using Swissmodel software and was identified on the epitope site. Identified amino acids are corresponding to 155-169 (KQDERVTMGQNGNLY; SEQ ID NO: 149), 209-214 (SMIDRK; SEQ ID NO: 150), and 276-286 (KVGEEDDGEYR; SEQ ID NO: 151) of huL1CAM. FIG. 11A shows ribbon representation of front view. FIG. 11B shows back view. FIG. 11C shows first side view. FIG. 11D shows second side view. FIG. 11E shows top view.

FIG. 12 depicts interaction between human L1CAM (huL1CAM) and TDI-Y-005. SEQ ID NO: 318 is shown in FIG. 10.

FIGS. 13A-13E depict 3D rendering of interaction between human L1CAM (huL1CAM) and TDI-Y-005. A PDB structure of huL1CAM was generated using Swissmodel software and was identified on the epitope site. Identified amino acids are corresponding to 622-639 (KYDIEFEDKEMAPEKWYS; SEQ ID NO: 152) and 714-730 (RWMDWNAPQVQYRVQWR; SEQ ID NO: 153) of huL1CAM. FIG. 13A shows ribbon representation of front view. FIG. 13B shows back view. FIG. 13C shows first side view. FIG. 13D shows second side view. FIG. 13E shows top view.

FIGS. 14A-14C depict binding of TDI-Y-004 and TDI-Y-005 to overexpressed and endogenous L1CAM on cells. FIG. 14A shows HEK293T cells overexpressing L1CAM stained with TDI-Y-004 and TDI-Y-005 at the indicated concentrations. FIG. 14B shows MCF-7 cells overexpressing L1CAM stained with TDI-Y-004 and TDI-Y-005 at the indicated concentrations. FIG. 14C shows MDA-MB-231 cells expressing endogenous L1CAM stained with TDI-Y-004 and TDI-Y-005 at the indicated concentrations. MFI values are normalized to secondary antibody-only controls (MFI ratio).

FIG. 15 depicts the ThioBridge® ADC Platform. Interchain disulfide bonds between the heavy and light chains and the hinge regions of IgGs are reduced to produce pairs of cysteine residues with free thiols, which serve as acceptor sites for a bis-sulfone bis-alkylating attachment unit to covalently rebridge the disulfides. A spacer comprising a polymer chain is included to increase the hydrophilicity of the ADC. The toxic drug is conjugated via a self-immolative release linker.

FIGS. 16A and 16B depict structures of linker-payloads used for conjugation to anti-L1CAM human IgG1 antibodies h14A10, hz143G03 (N61Q), and isotype control antibody hIgG1.

FIG. 16A shows ThioBridge® non-cleavable PNU159682. FIG. 16B shows ThioBridge® ‘Glu-Val-Cit-PAB’ cleavable PNU159682.

FIG. 17 depicts cytotoxicity of Antibody-Drug Conjugates (ADCs). ADCs with six different toxic payloads conjugated to 143G03, 14A10 and an isotype control mouse IgG1 were screened for their killing potency on various cell types as indicated. Free drug was included where available.

FIG. 18 depicts cytotoxicity of TDI-Y-004 and TDI-Y-005 ADCs with PNU-159682. ADCs generated with the lead mAbs (TDI-Y-004 and TDI-Y-005) plus a human IgG1 isotype control (hIgG1) with PNU-159682 and a cleavable (top) or non-cleavable (bottom) linker was added to HEK293T-L1CAM, MCF-7 or MDA-MB-231 cells as indicated. The killing curve for free PNU-159682 is included for each construct. RLU, relative luciferase units.

FIGS. 19A-19V depict analytical data of antibody-drug conjugates. FIG. 19A shows deconvoluted LC-MS spectrum for h14A10 ThioBridge®-Glu[-Glu(OH)-Val-Cit-PAB-DMEA-PNU159682]-PEG(24) conjugate (upper spectrum) and mAb h14A10 (lower spectrum). FIG. 19B shows hydrophobic interaction chromatogram (A=280 nm) for h14A10 ThioBridge®-Glu[-Glu(OH)-Val-Cit-PAB-DMEA-PNU159682]-PEG(24) and mAb h13A10. FIG. 19C shows size exclusion chromatogram (A=280 nm) for h14A10 ThioBridge®-Glu[-Glu(OH)-Val-Cit-PAB-DMEA-PNU159682]-PEG(24) and PBS blank. FIG. 19D shows non-reducing SDS-PAGE analysis of the samples (1) Novex Sharp MW Markers (2) mAb h14A10 (3) mAb h14A10 reduced with TCEP (6 eq. per mAb) for 1 h at 40° C. (4) h14A10 ThioBridge®-Glu[-Glu(OH)-Val-Cit-PAB-DMEA-PNU159682]-PEG(24) (5) mAb hz143G03 (N61Q) (6) mAb hz143G03 (N61Q) reduced with TCEP (6 eq. per mAb) for 1 h at 40° C. (7) hz143G03 (N61Q) ThioBridge®-Glu[-Glu(OH)-Val-Cit-PAB-DMEA-PNU159682]-PEG(24) (8) mAb hIgG1 (9) mAb hIgG1 reduced with TCEP (6 eq. per mAb) for 1 h at 40° C. (10) hIgG1 ThioBridge®-Glu[-Glu(OH)-Val-Cit-PAB-DMEA-PNU159682]-PEG(24). FIG. 19E shows size exclusion chromatogram (A=495 nm) for h14A10 ThioBridge®-Glu[-Glu(OH)-Val-Cit-PAB-DMEA-PNU159682]-PEG(24) (TDI100-JN006), hz143G03 (N61Q) ThioBridge®-Glu[-Glu(OH)-Val-Cit-PAB-DMEA-PNU159682]-PEG(24) (TDI100-JN007), hIgG1 ThioBridge®-Glu[-Glu(OH)-Val-Cit-PAB-DMEA-PNU159682]-PEG(24) (TDI100-JN008) and PBS blank. FIG. 19F shows deconvoluted LC-MS spectrum for h143G03 ThioBridge®-Glu[-Glu(OH)-Val-Cit-PAB-DMEA-PNU159682]-PEG(24) conjugate (upper spectrum) and mAb hz143G03 (N61Q) (lower spectrum). FIG. 19G shows hydrophobic interaction chromatogram (A=280 nm) for hz143G03 (N61Q) ThioBridge®-Glu[-Glu(OH)-Val-Cit-PAB-DMEA-PNU159682]-PEG(24) and mAb hz143G03 (N61Q). FIG. 19H shows size exclusion chromatogram (A=280 nm) for hz143G03 (N61Q) ThioBridge-GluEGlu(OH)-Val-Cit-PAB-DMEA-PNU1596821-PEG(24) and PBS blank. FIG. 19I shows deconvoluted LC-MS spectrum for hIgG1 ThioBridge®-Glu[-Glu(OH)-Val-Cit-PAB-DMEA-PNU159682]-PEG(24) conjugate (upper spectrum) and mAb hIgG1 (lower spectrum). FIG. 19J shows hydrophobic interaction chromatogram (A=280 nm) for hIgG1 ThioBridge®-Glu[-Glu(OH)-Val-Cit-PAB-DMEA-PNU159682]-PEG(24) and mAb hIgG1. FIG. 19K shows size exclusion chromatogram (A=280 nm) for hIgG1 ThioBridge-GluEGlu(OH)-Val-Cit-PAB-DMEA-PNU159682]-PEG(24) and PBS blank. FIG. 19L shows deconvoluted LC-MS spectrum h14A10 ThioBridge6-Glu(-Gly3-EDA-PNU159682)-PEG(24u) (upper spectrum) and mAb h14A10 (lower spectrum). FIG. 19M shows hydrophobic interaction chromatogram (A=280 nm) for h14A10 ThioBridge®-Glu(-Gly3-EDA-PNU159682)-PEG(24u) and mAb h14A10. FIG. 19N shows size exclusion chromatogram (A=280 nm) for h14A10 ThioBridge®-Glu(-Gly3-EDA-PNU159682)-PEG(24u) and PBS blank. FIG. 19O shows non-reducing SDS-PAGE analysis of the samples (1) Novex Sharp MW Markers (2) mAb h14A10 (3) mAb h14A10 reduced with TCEP (6 eq. per mAb) for 1 h at 40° C. (4) h14A10 ThioBridge®-Glu(-Gly3-EDA-PNU159682)-PEG(24u) (5) mAb hz143G03 (N61Q) (6) mAb hz143G03 (N61Q) reduced with TCEP (6 eq. per mAb) for 1 h at 40° C. (7) hz143G03 (N61Q) ThioBridge®-Glu(-Gly3-EDA-PNU159682)-PEG(24u) (8) mAb hIgG1 (9) mAb hIgG1 reduced with TCEP (6 eq. per mAb) for 1 h at 40° C. (10) hIgG1 ThioBridge®-Glu(-Gly3-EDA-PNU159682)-PEG(24u). FIG. 19P shows size exclusion chromatogram (A=495 nm) for h14A10 ThioBridge®-Glu(-Gly3-EDA-PNU159682)-PEG(24u) (TDI100-JN004), h143G03 ThioBridge®-Glu(-Gly3-EDA-PNU159682)-PEG(24u) (TDI100-JN005), hIgG1 ThioBridge®-Glu(-Gly3-EDA-PNU159682)-PEG(24u) (TDI100-JN003) and PBS blank. FIG. 19Q shows deconvoluted LC-MS spectrum hz143G03 (N61Q) ThioBridge®-Glu(-Gly3-EDA-PNU159682)-PEG(24u) conjugate (upper spectrum) and mAb hz143G03 (N61Q) (lower spectrum). FIG. 19R shows hydrophobic interaction chromatogram (A=280 nm) for hz143G03 (N61Q)ThioBridge®-Glu(-Gly3-EDA-PNU159682)-PEG(24u) and mAb hz143G03 (N61Q). FIG. 19S shows size exclusion chromatogram (A=280 nm) for hz143G03 (N61Q) ThioBridge®-Glu(-Gly3-EDA-PNU159682)-PEG(24u) and PBS blank. FIG. 19T shows deconvoluted LC-MS spectrum hIgG1 ThioBridge®-Glu(-Gly3-EDA-PNU159682)-PEG(24u) conjugate (upper spectrum) and mAb hIgG1 (lower spectrum). FIG. 19U shows hydrophobic interaction chromatogram (A=280 nm) for hIgG1 ThioBridge®-Glu(-Gly3-EDA-PNU159682)-PEG(24u) and mAb hIgG1. FIG. 19V shows size exclusion chromatogram (λ=280 nm) for hlgG1 ThioBridge®-Glu(-Gly3-EDA-PNU159682)-PEG(24u) and PBS blank. Amino acid sequence EEEVX is set forth as SEQ ID NO: 319. Amino acid sequence EGGG is set forth as SEQ ID NO: 320.

FIGS. 20A-20E depict HIC chromatograms of tested antibody-drug conjugates. FIG. 20A shows HIC chromatograms of ThioBridge® non-cleavable PNU159682 ADCs incubated in human serum for 96 h. FIG. 20B shows HIC chromatograms of different lots of ThioBridge® ‘Glu-Val-Cit-PAB’ PNU159682 ADCs incubated in human serum for 96 h. FIG. 20C shows HIC chromatograms of ThioBridge® non-cleavable PNU159682 ADCs incubated in mouse serum for 96 h. FIG. 20D shows HIC chromatograms of different lots of ThioBridge® ‘Glu-Val-Cit-PAB’ PNU159682 ADCs incubated in mouse serum for 96 h. FIG. 20E shows HIC chromatograms of Blank mouse serum and hz143G03 (N61Q) ThioBridge® ‘Glu-Val-Cit-PAB’ cleavable PNU159682 incubated in mouse serum for 96 h.

FIG. 21 depicts affinity capture method development. Deconvoluted LC-MS spectra of (1) (ADC starting material—h14A10 ThioBridge® ‘Glu-Val-Cit-PAB’ cleavable PNU159682); (2) ADC captured from human serum; (3) ADC captured from mouse serum; (4) ADC captured from PBS.

FIGS. 22A and 22B depict analysis of human and mouse sera. FIG. 22A shows h14A10 ThioBridge® ‘Glu-Val-Cit-PAB’ cleavable PNU159682 affinity captured from human serum and analyzed by LC-MS, after reduction with DTT. FIG. 22B shows h14A10 ThioBridge® ‘Glu-Val-Cit-PAB’ cleavable PNU159682 affinity captured from mouse serum and analyzed by LC-MS, after reduction with DTT.

FIG. 23 depicts reagent-related species investigated for ThioBridge® non-cleavable PNU159682 reagent ADCs.

FIG. 24 shows reagent-related species investigated for ThioBridge® ‘Glu-Val-Cit-PAB’ cleavable PNU159682 reagent ADCs.

FIGS. 25A-25D depict cytotoxicity of TDI-Y-004 and TDI-Y-005 ADCs on organoid models. FIG. 25A shows a schematic of methods to prepare organoid models. FIG. 25B shows growth of organoids in presence of the indicated molecules. FIG. 25C shows modulation of organoid growth with L1CAM antibodies. FIG. 25D shows organoid growth with L1CAM antibody-drug conjugates.

FIGS. 26A-26C depict in vivo anti-tumor efficacy of TDI-Y-004 and TDI-Y-005 ADCs. Athymic mice were transplanted subcutaneously with MDA-MB-231-LM2 breast cancer cells. Once tumors measuring 100 mm3 were established, animals were administered 4 weekly doses of ADCs generated with the L1CAM lead mAbs (TDI-Y-004 and TDI-Y-005) plus a human IgG1 isotype control (IgG) conjugated to PNU-159682 (PNU) with a cleavable (C) linker (0.3 mg/kg) or non-cleavable (NC) linker (1 mg/kg), or vehicle control. FIGS. 26A and 26B show tumor growth monitored on a weekly basis. FIG. 26C shows overall survival monitored on a weekly basis.

FIGS. 27A-27C depict in vivo anti-lung metastasis efficacy of TDI-Y-004 and TDI-Y-005 ADCs. MDA-MB-231-LM2 breast cancer cells were introduced via tail vein injection into athymic mice to generate lung metastasis. Animals were administered 4 weekly doses of ADCs generated with the L1CAM lead mAbs (TDI-Y-004 and TDI-Y-005) plus a human IgG1 isotype control (IgG) conjugated to PNU-159682 (PNU) with a cleavable (C) linker (0.3 mg/kg) or non-cleavable (NC) linker (1 mg/kg), or vehicle control. FIGS. 27A and 27B show tumor growth monitored on a weekly basis. FIG. 27C shows overall survival monitored on a weekly basis.

FIGS. 28A-28C depict in vivo anti-tumor efficacy of TDI-Y-005 ADCs. Athymic mice were transplanted subcutaneously with CRC107 cancer cells. Once tumors were established, animals were administered 4 weekly doses of ADCs generated with the L1CAM lead TDI-Y-005 plus a human IgG1 isotype control (IgG) conjugated to PNU-159682 (PNU) with a cleavable (C) linker (0.3 mg/kg) or non-cleavable (NC) linker (1 mg/kg), or vehicle control. In certain cohorts, mice were also administered with irinotecan. FIG. 28A shows tumor volume monitored on a weekly basis. FIGS. 28B and 28C show tumor volume at 4 weeks and 7 weeks of treatment.

FIGS. 29A-29C depict manufacturability studies of TDI-Y-004. FIG. 29A shows HPLC-SEC chromatogram. FIG. 29B shows a non-reduced CE-SDS chromatogram. FIG. 29C shows a reduced CE-SDS chromatogram.

FIGS. 30A-30C depict manufacturability studies of TDI-Y-005. FIG. 30A shows HPLC-SEC chromatogram. FIG. 30B shows a non-reduced CE-SDS chromatogram. FIG. 30C shows a reduced CE-SDS chromatogram.

FIGS. 31A and 31B depict structures of the cleavable and non-cleavable linkers utilized for L1CAM ADC generation. FIG. 31A shows structure of the cleavable linker (ThioBridge®-Glu-(Glu-Val-Cit-PAB-DMAE-PNU-159682)-PEG(24u)). FIG. 31B shows structure of the non-cleavable linker (ThioBridge®-Glu-(Gly3-EDA-PNU-159682)-PEG(24u)). The ADCs had an average drug-to-antibody ratio (DAR) of 4.

FIGS. 32A and 32B illustrate binding of humanized 76H12 Fab variants. FIG. 32A shows flow cytometry for binding to 293T cells engineered to overexpress L1CAM. FIG. 32B shows kinetic data of tested humanized variants binding to L1CAM-Fc fusion protein.

FIGS. 33A and 33B illustrate in vitro and in vivo efficacy of the anti-L1CAM 13F04 ADC against human metastatic lung adenocarcinoma (LUAD) cells. FIG. 33A shows cell viability was assessed 96 h later using CellTiterGlo reagent and was normalized to DMSO control. Data are averages of triplicate samples from three independent experiments. Bars represent standard deviation. FIG. 33B shows tumor growth monitored using BLI measurements. Bars represent standard deviation. n=10 mice for 13F04 and 9 mice for IgG ADC group. ** P<0.01

### DETAILED DESCRIPTION

Metastatic cancer is the second-leading cause of death in the US (nearly 600,000 deaths each year). There is an unmet need for drugs capable of targeting and eliminating therapy-resistant disease in patients with advanced solid tumors. The presently disclosed subject matter is based, in part, on the generation of novel anti-L1CAM antibodies that allow the elimination of quiescence-capable metastasis stem cells when conjugated to a therapeutic agent. These antibodies or immunoconjugates of such antibodies can be complementary to chemotherapy and therapies that target rapidly proliferating cells. This novel concept in cancer therapeutics can address the plasticity of advanced cancer driving metastatic relapse after therapy. Non-limiting embodiments of the present disclosure are described by the present specification and Examples.

For purposes of clarity of disclosure and not by way of limitation, the detailed description is divided into the following subsections:
1. Definitions;
2. L1CAM;
3. Anti-L1CAM Antibodies;
4. Immunoconjugates;
5. Nucleic Acids encoding the Antibodies or Antigen-binding Fragments;
6. Pharmaceutical Compositions and Methods of Treatment;
7. Diagnostic and Prognostic Methods;
8. Kits; and
9. Exemplary Embodiments.

### 1. Definitions

In the description that follows, certain conventions will be followed as regards the usage of terminology. Generally, terms used herein are intended to be interpreted consistently with the meaning of those terms as they are known to those of skill in the art.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is Composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies includes one or more fragments of an antibody that retain the ability to specifically bind to an antigen (e.g., a L1CAM polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to L1CAM” is intended to refer to an antibody that binds to L1CAM (e.g., human L1CAM) with a dissociation constant (K_{D}) of about 1×10⁻⁸ M or less, about 5×10⁻⁹ M or less, about 1×10⁻⁹ M or less, about 5×10⁻¹¹ M or less, about 1×10⁻¹ M or less, about 5×10⁻¹¹ M or less, or about 1×10⁻¹¹ M or less.

An “antibody that competes for binding” or “antibody that cross-competes for binding” with a reference antibody for binding to an antigen, e.g., L1CAM, refers to an antibody that blocks binding of the reference antibody to the antigen (e.g., L1CAM) in a competition assay by 50% or more, and conversely, the reference antibody blocks binding of the antibody to the antigen (e.g., L1CAM) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen“are used interchangeably herein with the term” an antibody which binds specifically to an antigen (e.g., a L1CAM polypeptide).”

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers, e.g., for use in generating an scFv, are disclosed in Shen et al., Anal Chem (2008); 80(6):1910-1917 and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 138, which is provided below:

[SEQID NO: 138]
GGGGSGGGGSGGGSGGGGS

In certain embodiments, the linker comprises or consists of the amino acid sequence set forth in SEQ ID NO: 139, which is provided below:

[SEQ ID NO: 139]
GGGGSGGGGSGGGGS

In certain embodiments, the linker comprises or consists of the amino acid sequence set forth in SEQ ID NO: 140, which is provided below:

[SEQ ID NO: 140]
GGGGSGGGGSGGGGSGGGSGGGGS

In certain embodiments, the linker comprises or consists of the amino acid sequence set forth in SEQ ID NO: 141, which is provided below:

[SEQ ID NO: 141]
GGGGSGGGGSGGGGSGGGGSGGGSGGGGS

In certain embodiments, the linker comprises or consists of the amino acid sequence set forth in SEQ ID NO: 142, which is provided below:

[SEQ ID NO: 142]
GGGGS

In certain embodiments, the linker comprises or consists of the amino acid sequence set forth in SEQ ID NO: 143, which is provided below:

[SEQ ID NO: 143]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 August 12; Shieh et al., J Imunol 2009; 183(4):2277-85; Giomarelli et al., Thromb Haemost 2007; 97(6):955-63; Fife eta., J Clin Invst 2006; 116(8):2252-61; Brocks et al., Immunotechnology 1997; 3(3):173-84; Moosmayer et al., Ther Immunol 1995; 2(10:31-40). Agonistic scFvs having stimulatory activity have been described (see, e.g., Peter et al., J Bio. Chem 2003; 25278(38):36740-7; Xie et al., Nat Biotech 1997; 15(8):768-71; Ledbetter et al., Crit Rev Immunol 1997; 17(5-6):427-55; Ho et al., BioChim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-L1CAM antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters; guinea pigs; rabbits; dogs; cats; sheep; pigs; goats; cattle; horses; and non-human primates such as apes and monkeys.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor, e.g., a tumor associated with L1CAM.

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 2. L1CAM

L1CAM is a transmembrane protein member of the L1 protein family, encoded by the L1CAM gene. This protein, of about 200 to about 220 kDa, is a neuronal cell adhesion molecule with a strong implication in cell migration, adhesion, neurite outgrowth, myelination and neuronal differentiation (Samatov et al., Prog Histochem Cytochem. 2016 August; 51(2):25-32). L1CAM is composed of six IgG domains and three fibronectin type III domains and a short cytoplasmic domain. The extracellular domain forms homophilic interactions and heterophilic interactions with integrins and several extracellular matrix proteins. Via its cytoplasmic tail, L1CAM binds ezrin, ankyrin, and other signaling adaptor proteins. L1CAM is predominantly expressed by developing neurons, in which it regulates axon extension and synaptogenesis, and is also found in certain hematological and endothelial cell types. Notably, cancer cells exclusively express non-neuronal splice forms of L1CAM, and it was recently discovered that these isoforms are also naturally expressed in pericytes, the contractile mesenchymal cells that wrap around capillaries to regulate blood flow. L1CAM is involved in metastatic outgrowth of multiple solid tumors, including the three most prominent causes of cancer death (e.g., lung, breast, and colorectal cancer), as well as in metastatic outgrowth in multiple organ sites (e.g., bone, lung, liver, and brain). Further, L1CAM is involved in the outgrowth of aggressive cancer cells right after freshly seeding a target organ as well as indolent cancer cells that emerge from dormancy. L1CAM is required for the initiation of micrometastases, maintenance and expansion of established metastases, and for the re-initiation of metastatic growth by dormant micrometastases.

In certain embodiments, the presently disclosed anti-L1CAM antibodies or antigen-binding fragments thereof bind to human L1CAM. In certain embodiments, the human L1CAM comprises or consists of the amino acid sequence with a UniProt Reference No: P32004 (SEQ ID NO: 1) or a fragment thereof. SEQ ID NO: 1 is provided below. In certain embodiments, the human L1CAM comprises an extracellular domain, a transmembrane domain, and a cytoplasmic domain. In certain embodiments, the extracellular domain comprises or consists of amino acids 20 to 1120 of SEQ ID NO: 1. In certain embodiments, the transmembrane domain comprises or consists of amino acids 1121 to 1143 of SEQ ID NO: 1. In certain embodiments, the cytoplasmic domain comprises or consists of amino acids 1144 to 1257 of SEQ ID NO: 1.

[SEQ ID NO: 1]
MVVALRYVWPLLLCSPCLLIQIPEEYEGHHVMEPPVITEQSPRRLVVFPT
DDISLKCEASGKPEVQFRWTRDGVHFKPKEELGVTVYQSPHSGSFTITGN
NSNFAQRFQGIYRCFASNKLGTAMSHEIRLMAEGAPKWPKETVKPVEVEE
GESVVLPCNPPPSAEPLRIYWMNSKILHIKQDERVIMGQNGNLYFANVLT
SDNHSDYICHAHFPGTRTIIQKEPIDLRVKATNSMIDRKPRLLFPTNSSS
HLVALQGQPLVLECIAEGFPTPTIKWLRPSGPMPADRVTYQNHNKTLQLL
KVGEEDDGEYRCLAENSLGSARHAYYVTVEAAPYWLHKPQSHLYGPGETA
RLDCQVQGRPQPEVTWRINGIPVEELAKDQKYRIQRGALILSNVQPSDTM
VTQCEARNRHGLLLANAYIYVVQLPAKILTADNQTYMAVQGSTAYLLCKA
FGAPVPSVQWLDEDGTTVLQDERFFPYANGTLGIRDLQANDTGRYFCLAA
NDQNNVTIMANLKVKDATQITQGPRSTIEKKGSRVTFTCQASFDPSLQPS
ITWRGDGRDLQELGDSDKYFIEDGRLVIHSLDYSDQGNYSCVASTELDVV
ESRAQLLVVGSPGPVPRLVLSDLHLLTQSQVRVSWSPAEDHNAPIEKYDI
EFEDKEMAPEKWYSLGKVPGNQTSTTLKLSPYVHYTFRVTAINKYGPGEP
SPVSETVVTPEAAPEKNPVDVKGEGNETTNMVITWKPLRWMDWNAPQVQY
RVQWRPQGTRGPWQEQIVSDPFLVVSNTSTFVPYEIKVQAVNSQGKGPEP
QVTIGYSGEDYPQAIPELEGIEILNSSAVLVKWRPVDLAQVKGHLRGYNV
TYWREGSQRKHSKRHIHKDHVVVPANTTSVILSGLRPYSSYHLEVQAFNG
RGSGPASEFTFSTPEGVPGHPEALHLECQSNTSLLLRWQPPLSHNGVLTG
YVLSYHPLDEGGKGQLSFNLRDPELRTHNLTDLSPHLRYRFQLQATTKEG
PGEAIVREGGTMALSGISDFGNISATAGENYSVVSWVPKEGQCNFRFHIL
FKALGEEKGGASLSPQYVSYNQSSYTQWDLQPDTDYEIHLFKERMFRHQM
AVKTNGTGRVRLPPAGFATEGWFIGFVSAIILLLLVLLILCFIKRSKGGK
YSVKDKEDTQVDSEARPMKDETFGEYRSLESDNEEKAFGSSQPSLNGDIK
PLGSDDSLADYGGSVDVQFNEDGSFIGQYSGKKEKEAAGGNDSSGATSPI
NPAVALE

In certain embodiments, the L1CAM comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 1 or a fragment thereof.

In certain embodiments, the presently disclosed anti-L1CAM antibodies or antigen-binding fragments thereof bind to a portion of human L1CAM. In certain embodiments, the presently disclosed anti-L1CAM antibodies or antigen-binding fragments thereof bind to the extracellular domain of L1CAM. In certain embodiments, the presently disclosed anti-L1CAM antibodies or antigen-binding fragments thereof bind to amino acids 20 to 1120 of SEQ ID NO: 1.

### 3. Anti-L1CAM Antibodies

The antibodies of the presently disclosed subject matter are characterized by particular functional features or properties of the antibodies. For example, the antibodies bind specifically to L1CAM (e.g., bind to human L1CAM).

In certain embodiments, a presently disclosed antibody or antigen-binding fragment binds to L1CAM (e.g., human L1CAM) with a binding affinity, for example with a dissociation constant (K_{D}) of 1×10⁻⁸ M or less, e.g., about 1×10⁻⁸ M or less, about 5×10⁻⁹ M or less, about 1×10⁻⁹ M or less, about 5×10⁻¹⁰ M or less, about 1×10⁻¹⁰ M or less, or about 1×10⁻¹¹ M or less. In certain embodiments, the presently disclosed anti-L1CAM antibody or antigen-binding fragment thereof binds to L1CAM (e.g., human L1CAM) with a K_{D} of about 5×10⁻⁹ M or less. In certain embodiments, the presently disclosed anti-L1CAM antibody or antigen-binding fragment thereof binds to L1CAM (e.g., human L1CAM) with a K_{D} of about 1×10⁻⁹ M or less. In certain embodiments, the presently disclosed anti-L1CAM antibody or antigen-binding fragment thereof binds to L1CAM (e.g., human L1CAM) with a K_{D} of about 1×10⁻¹⁰ M or less. In certain embodiments, the presently disclosed anti-L1CAM antibody or antigen-binding fragment thereof binds to L1CAM (e.g., human L1CAM) with a K_{D} of between about 1×10⁻⁹ M and about 1×10⁻¹⁰ M. In certain embodiments, the presently disclosed anti-L1CAM antibody or antigen-binding fragment thereof binds to L1CAM (e.g., human L1CAM) with a K_{D} of between about 1×10⁻¹⁰ M and about 1×10⁻¹¹ M. In certain embodiments, the presently disclosed anti-L1CAM antibody or antigen-binding fragment thereof binds to L1CAM (e.g., human L1CAM) with a K_{D} of about 1×10⁻¹⁰ M. In certain embodiments, the presently disclosed anti-L1CAM antibody or antigen-binding fragment thereof binds to L1CAM (e.g., human L1CAM) with a K_{D} of about 2×10¹¹ M.

In certain embodiments, a presently disclosed antibody or antigen-binding fragment binds to a cell expressing L1CAM (e.g., a metastatic cell expressing L1CAM) with a Half maximal effective concentration (EC₅₀) value of from about 1 nM to about 50 nM, from about 5 nM to about 50 nM, from about 10 nM to about 50 nM, from about 20 nM to about 50 nM, from about 30 nM to about 50 nM, from about 40 nM to about 50 nM, or greater than about 50 nM. In certain embodiments, a presently disclosed antibody or antigen-binding fragment binds to a cell expressing L1CAM (e.g., a metastatic cell expressing L1CAM) with an EC₅₀ value from about 1 nM to about 5 nM. In certain embodiments, a presently disclosed antibody or antigen-binding fragment binds to a cell expressing L1CAM (e.g., a metastatic cell expressing L1CAM) with an EC₅₀ value of about 1 nM. In certain embodiments, a presently disclosed antibody or antigen-binding fragment binds to a cell expressing L1CAM (e.g., a metastatic cell expressing L1CAM) with an EC₅₀ value of about 4.8 nM.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE. In certain embodiments, the antibody heavy chain constant region is chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda. In certain embodiments, the antibody light chain constant region is kappa.

In constructing a recombinant immunoglobulin, appropriate amino acid sequences for constant regions of various immunoglobulin isotypes and methods for the production of a wide array of antibodies are known to those of skill in the art.

3.1. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications. In certain embodiments, the Fc portion is conjugated to a cytotoxin.

The results presented here highlight the specificity, sensitivity and utility of the presently disclosed antibodies or antigen-binding fragments in targeting a L1CAM polypeptide (e.g., a human L1CAM polypeptide).

In certain embodiments, the anti-L1CAM antibody is an scFv, an scFv-Fc fusion protein or a full-length human IgG (or antibody fragment thereof) with V_{H} and V_{L} regions or CDRs selected from Table 1. In certain embodiments, the anti-L1CAM scFv comprises a V_{H} comprising the amino acid sequence set forth in SEQ ID NO: 8. In certain embodiments, the anti-L1CAM scFv comprises a V_{L} comprising the amino acid sequence set forth in SEQ ID NO: 9. SEQ ID NO: 8 and 9 are provided in Table 1. In certain embodiments, the scFv is designated as “scFv-Y-005”.

In certain embodiments, the anti-L1CAM scFv comprises a V_{H} comprising the amino acid sequence set forth in SEQ ID NO: 8 and a V_{L} comprising the amino acid sequence set forth in SEQ ID NO: 9.

In certain embodiments, the anti-L1CAM scFv comprises a V_{H} Comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof. SEQ ID NOs: 2-4 are provided in Table 1.

In certain embodiments, the anti-L1CAM scFv comprises a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOs: 4-6 are provided in Table 1.

In certain embodiments, the anti-L1CAM scFv comprises a V_{H} Comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, a CDR3 comprising the amino acid sequence set forth in SEQ ID NO: 4 or a conservative modification thereof; and a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification, and a CDR3 comprising the amino acid sequence set forth in SEQ ID NO: 7 or a conservative modification thereof.

In certain embodiments, the anti-L1CAM scFv comprises a V_{H} Comprising a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and a V_{L} comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the anti-L1CAM scFv comprises a V_{H} comprising the amino acid sequence set forth in SEQ ID NO: 8, and a V_{L} comprising the amino acid sequence set forth in SEQ ID NO: 9. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

In certain embodiments, the anti-L1CAM antibody is an antibody with a heavy chain and a light chain selected from Table 1. In certain embodiments, the anti-L1CAM antibody comprises a heavy chain comprising the amino acid sequence set forth in SEQ ID NO: 10. In certain embodiments, the anti-L1CAM antibody comprises a light chain comprising the amino acid sequence set forth in SEQ ID NO: 11. SEQ ID NO: 10 and 11 are provided in Table 1. In certain embodiments, the anti-L1CAM antibody is designated as “TDI-Y-005”.

TABLE 1

| CDRs | 1 | 2 | 3 |
|---|---|---|---|
| VH | GFIFSSYN | ISSSSSTT | ASLYSGYDYYY |
| | [SEQ ID | [SEQ ID | YYGMDV |
| | NO: 2] | NO: 3] | [SEQ ID |
| | | | NO: 4] |
| VL | QSLVHSDGNTY | KIS | MQATQFPFT |
| | [SEQ ID | [SEQ ID | [SEQ ID |
| | NO: 5] | NO: 6] | NO: 7] |

| | |
|---|---|
| Full | EVQMVESGGGLVQPGGSLRLSCAAPGFIFSSYNM |
| VH | NWVRQAPGKGLEWVSYISSSSSTTYYADSVKGRF |
| | TISRDNAKNSLYLQMNSLRDEDTAVYYCASLYSG |
| | YDYYYYYGMDVWGQGTTVTVSS |
| | [SEQ ID NO: 8] |
| Full | DIVMTQTPLSSPVSLGQPASISCRSSQSLVHSDG |
| VL | NTYLSWLQQRPGQPPRVLIYKISKRFSGVPDRES |
| | GSGAGTDFTLKISRVEAEDVGVYYCMQATQFPFT |
| | FGPGTKVDIK |
| | [SEQ ID NO: 9] |
| Heavy | EVQMVESGGGLVQPGGSLRLSCAAPGFIFSSYNM |
| Chain | NWVRQAPGKGLEWVSYISSSSSTTYYADSVKGRF |
| | TISRDNAKNSLYLQMNSLRDEDTAVYYCASLYSG |
| | YDYYYYYGMDVWGQGTTVTVSSASTKGPSVFPLA |
| | PSSKSTSGGTAALGCLVKDYFPEPVTVSWNSGAL |
| | TSGVHTFPAVLQSSGLYSLSSVVTVPSSSLGTQT |
| | YICNVNHKPSNTKVDKKVEPKSCDKTHTCPPCPA |
| | PELLGGPSVFLFPPKPKDTLMISRTPEVTCVVVD |
| | VSHEDPEVKFNWYVDGVEVHNAKTKPREEQYNST |
| | YRVVSVLTVLHQDWLNGKEYKCKVSNKALPAPIE |
| | KTISKAKGQPREPQVYTLPPSRDELTKNQVSLTC |
| | LVKGFYPSDIAVEWESNGQPENNYKTTPPVLDSD |
| | GSFFLYSKLTVDKSRWQQGNVFSCSVMHEALHNH |
| | YTQKSLSLSPGK |
| | [SEQ ID NO: 10] |
| Light | DIVMTQTPLSSPVSLGQPASISCRSSQSLVHSDGN |
| Chain | TYLSWLQQRPGQPPRVLIYKISKRFSGVPDRFSGS |
| | GAGTDFTLKISRVEAEDVGVYYCMQATQFPFTFGP |
| | GTKVDIKRTVAAPSVFIFPPSDEQLKSGTASVVCL |
| | LNNFYPREAKVQWKVDNALQSGNSQESVTEQDSKD |
| | STYSLSSTLTLSKADYEKHKVYACEVTHQGLSSPV |
| | TKSFNRGEC |
| | [SEQ ID NO: 11] |

In certain embodiments, the anti-L1CAM antibody is an scFv, an scFv-Fc fusion protein or a full-length human IgG (or antibody fragment thereof) with V_{H} and V_{L} regions or CDRs selected from Table 2. In certain embodiments, the anti-L1CAM scFv comprises a V_{H} comprising the amino acid sequence set forth in SEQ ID NO: 18. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 18 and a V_{L} comprising the amino acid sequence set forth in SEQ ID NO: 19. SEQ ID NO: 18 and 19 are provided in Table 2. In certain embodiments, the scFv is designated as “scFv-Y-004”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 2.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 2.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 18, and a V_{L} comprising the amino acid sequence set forth in SEQ ID NO: 19. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

In certain embodiments, the anti-L1CAM antibody is an antibody with a heavy chain and a light chain selected from Table 2. In certain embodiments, the anti-L1CAM antibody comprises a heavy chain comprising the amino acid sequence set forth in SEQ ID NO: 20. In certain embodiments, the anti-L1CAM antibody comprises a light chain comprising the amino acid sequence set forth in SEQ ID NO: 21. SEQ ID NO: 20 and 21 are provided in Table 2. In certain embodiments, the anti-L1CAM antibody is designated as “TDI-Y-004”.

TABLE 2

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGSSVKVSCKASGYTFTNYYIH |
| V_{H} | WVRQAPGQGLEWMGKIGPGSGSPYYQGMFKDRVTI |
| | TADESTSTAYMELSSLRSEDTAVYYCARWLLPFDY |
| | WGQGTLVTVSS |
| | [SEQ ID NO: 18] |
| Full | EIVLTQSPGTLSLSPGERATLSCRASSSVNYMHWY |
| V_{L} | QQKPGQAPRLLIYATSILASGIPDRFSGSGSGTDY |
| | TLTISRLEPEDFAVYYCQQFSSDPLTFGGGTKVEI |
| | K |
| | [SEQ ID NO: 19] |
| Heavy | QVQLVQSGAEVKKPGSSVKVSCKASGYTFTNYYIH |
| Chain | WVRQAPGQGLEWMGKIGPGSGSPYYQGMFKDRVTI |
| | TADESTSTAYMELSSLRSEDTAVYYCARWLLPFDY |
| | WGQGTLVTVSSASTKGPSVFPLAPSSKSTSGGTAA |
| | LGCLVKDYFPEPVTVSWNSGALTSGVHTFPAVLQS |
| | SGLYSLSSVVTVPSSSLGTQTYICNVNHKPSNTKV |
| | DKKVEPKSCDKTHTCPPCPAPELLGGPSVFLFPPK |
| | PKDTLMISRTPEVTCVVVDVSHEDPEVKFNWYVDG |
| | VEVHNAKTKPREEQYNSTYRVVSVLTVLHQDWLNG |
| | KEYKCKVSNKALPAPIEKTISKAKGQPREPQVYTL |
| | PPSRDELTKNQVSLTCLVKGFYPSDIAVEWESNGQ |
| | PENNYKTTPPVLDSDGSFFLYSKLTVDKSRWQQGN |
| | VFSCSVMHEALHNHYTQKSLSLSPGK |
| | [SEQ ID NO: 20] |
| Light | EIVLTQSPGTLSLSPGERATLSCRASSSVNYMHWY |
| Chain | QQKPGQAPRLLIYATSILASGIPDRFSGSGSGTDY |
| | TLTISRLEPEDFAVYYCQQFSSDPLTFGGGTKVEI |
| | KRTVAAPSVFIFPPSDEQLKSGTASVVCLLNNFYP |
| | REAKVQWKVDNALQSGNSQESVTEQDSKDSTYSLS |
| | STLTLSKADYEKHKVYACEVTHQGLSSPVTKSFNR |
| | GEC |
| | [SEQ ID NO: 21] |

In certain embodiments, the anti-L1CAM antibody is an scFv, an scFv-Fc fusion protein or a full-length human IgG (or antibody fragment thereof) with V_{H} and V_{L} regions or CDRs selected from Table 3. In certain embodiments, the anti-L1CAM scFv comprises a V_{H} comprising the amino acid sequence set forth in SEQ ID NO: 22. In certain embodiments, the anti-L1CAM scFv comprises a V_{L} comprising the amino acid sequence set forth in SEQ ID NO: 23. In certain embodiments, the anti-L1CAM scFv comprises a V_{H} comprising the amino acid sequence set forth in SEQ ID NO: 22 and a V_{L} comprising the amino acid sequence set forth in SEQ ID NO: 23. SEQ ID NO: 22 and 23 are provided in Table 3. In certain embodiments, the scFv is designated as “k1-h1”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 3.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 3.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 22, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 3

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGASVKVSCKASGYTFTNYYIH |
| V_{H} | WVRQAPGQGLEWMGKIGPGSGSPYYNGMFKDRVTM |
| | TRDTSTSTVYMELSSLRSEDTAVYYCARWLLPFDY |
| | WGQGTLVTVSS |
| | [SEQ ID NO: 22] |
| Full | EIVLTQSPATLSLSPGERATLSCRASSSVNYMHWY |
| V_{L} | QQKPGQAPRLLIYATSILASGIPARFSGSGSGTDY |
| | TLTISSLEPEDFAVYYCQQFSSDPLTFGGGTKVEI |
| | K |
| | [SEQ ID NO: 23] |

In certain embodiments, the anti-L1CAM antibody is an scFv, an scFv-Fc fusion protein or a full-length human IgG (or antibody fragment thereof) with V_{H} and V_{L} regions or CDRs selected from Table 4. In certain embodiments, the anti-L1CAM scFv comprises a V_{H} comprising the amino acid sequence set forth in SEQ ID NO: 24. In certain embodiments, the anti-L1CAM scFv comprises a V_{L} comprising the amino acid sequence set forth in SEQ ID NO: 23. In certain embodiments, the anti-L1CAM scFv comprises a V_{H} comprising the amino acid sequence set forth in SEQ ID NO: 24 and a V_{L} comprising the amino acid sequence set forth in SEQ ID NO: 23. SEQ ID NO: 24 and 23 are provided in Table 4. In certain embodiments, the scFv is designated as “k1-h2”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 4.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 4.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 24, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 4

| CDRS | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVQSGAEVKKPGASVKVSCKASGYTFTNYYIH |
| V_{H} | WVRQAPGQGLEWMGKIGPGSGSPYYNGMFKDRVTS |
| | TRDTSISTAYMELSRLRSDDTVVYYCARWLLPFDY |
| | WGQGTLVTVSS |
| | [SEQ ID NO: 24] |
| Full | EIVLTQSPATLSLSPGERATLSCRASSSVNYMHWY |
| V_{L} | QQKPGQAPRLLIYATSILASGIPARFSGSGSGTDY |
| | TLTISSLEPEDFAVYYCQQFSSDPLTFGGGTKVEI |
| | K |
| | [SEQ ID NO: 23] |

In certain embodiments, the anti-L1CAM antibody is an scFv, an scFv-Fc fusion protein or a full-length human IgG (or antibody fragment thereof) with V_{H} and V_{L} regions or CDRs selected from Table 5. In certain embodiments, the anti-L1CAM scFv comprises a V_{H} comprising the amino acid sequence set forth in SEQ ID NO: 25. In certain embodiments, the anti-L1CAM scFv comprises a V_{L} comprising the amino acid sequence set forth in SEQ ID NO: 23. In certain embodiments, the anti-L1CAM scFv comprises a V_{H} comprising the amino acid sequence set forth in SEQ ID NO: 25 and a V_{L} comprising the amino acid sequence set forth in SEQ ID NO: 23. SEQ ID NO: 25 and 23 are provided in Table 5. In certain embodiments, the scFv is designated as “k1-h3”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 5.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 5.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 25, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 5

| CDRs | 1 | 2 | 3 |
|---|---|---|---|
| V_{H} | GYTFTNYY | IGPGSGSP | ARWLLPFDY |
| | [SEQ ID | [SEQ ID | [SEQ ID |
| | NO: 12] | NO: 13] | NO: 14] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | QVQLVESGGGVVQPGRSLRLSCAASGYTFTNYYIH |
| V_{H} | WVRQAPGKGLEWVAKIGPGSGSPYYNGMFKDRFTI |
| | SRDNSKNTLYLQMNSLRAEDTAVYYCARWLLPFDY |
| | WGQGTLVTVSS |
| | [SEQ ID NO: 25] |
| Full | EIVLTQSPATLSLSPGERATLSCRASSSVNYMHWY |
| V_{L} | QQKPGQAPRLLIYATSILASGIPARFSGSGSGTDY |
| | TLTISSLEPEDFAVYYCQQFSSDPLTFGGGTKVEI |
| | K |
| | [SEQ ID NO: 23] |

In certain embodiments, the anti-L1CAM antibody is an scFv, an scFv-Fc fusion protein or a full-length human IgG (or antibody fragment thereof) with V_{H} and V_{L} regions or CDRs selected from Table 6. In certain embodiments, the anti-L1CAM scFv comprises a V_{H} comprising the amino acid sequence set forth in SEQ ID NO: 27. In certain embodiments, the anti-L1CAM scFv comprises a V_{L} comprising the amino acid sequence set forth in SEQ ID NO: 23. In certain embodiments, the anti-L1CAM scFv comprises a V_{H} comprising the amino acid sequence set forth in SEQ ID NO: 27 and a V_{L} comprising the amino acid sequence set forth in SEQ ID NO: 23. SEQ ID NO: 27 and 23 are provided in Table 6. In certain embodiments, the scFv is designated as “k1-h4”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 6.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 6.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 27, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 6

| CDRs | 1 | 2 | 3 |
|---|---|---|---|
| V_{H} | GYTFTNYY | IGPGSGSP | AKWLLPFDY |
| | [SEQ ID | [SEQ ID | [SEQ ID |
| | NO: 12] | NO: 13] | NO: 26] |
| V_{L} | SSVNY | ATS | QQFSSDPLT |
| | [SEQ ID | [SEQ ID | [SEQ ID |
| | NO: 15] | NO: 16] | NO: 17] |

| | |
|---|---|
| Full | EVQLLESGGGLVQPGGSLRLSCAASGYTFTNYYIH |
| V_{H} | WVRQAPGKGLEWVSKIGPGSGSPYYNGMFKDRFTI |
| | SVDNSKNTLYLQMNSLRAEDTAVYYCAKWLLPFDY |
| | WGQGTLVTVSS |
| | [SEQ ID NO: 27] |
| Full | EIVLTQSPATLSLSPGERATLSCRASSSVNYMHWY |
| V_{L} | QQKPGQAPRLLIYATSILASGIPARFSGSGSGTDY |
| | TLTISSLEPEDFAVYYCQQFSSDPLTFGGGTKVEI |
| | K |
| | [SEQ ID NO: 23] |

In certain embodiments, the anti-L1CAM antibody is an scFv, an scFv-Fc fusion protein or a full-length human IgG (or antibody fragment thereof) with V_{H} and V_{L} regions or CDRs selected from Table 7. In certain embodiments, the anti-L1CAM scFv comprises a V_{H} comprising the amino acid sequence set forth in SEQ ID NO: 28. In certain embodiments, the anti-L1CAM scFv comprises a V_{L} comprising the amino acid sequence set forth in SEQ ID NO: 23. In certain embodiments, the anti-L1CAM scFv comprises a V_{H} comprising the amino acid sequence set forth in SEQ ID NO: 28 and a V_{L} comprising the amino acid sequence set forth in SEQ ID NO: 23. SEQ ID NO: 28 and 23 are provided in Table 7. In certain embodiments, the scFv is designated as “k1-h5”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 7.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 7.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 28, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 7

| CDRS | 1 | 2 | 3 |
|---|---|---|---|
| V_{H} | GYTFTNYY | IGPGSGSP | ARWLLPEDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| V_{H} | SPYYNGMFKDRVTITADESTSTAYMELSSLRSEDTAVYYCARWLLPFDYWGQGTLV |
| | TVSS |
| | [SEQ ID NO: 28] |
| Full | EIVLTQSPATLSLSPGERATLSCRASSSVNYMHWYQQKPGQAPRLLIYATSILASG |
| V_{L} | IPARFSGSGSGTDYTLTISSLEPEDFAVYYCQQFSSDPLTFGGGTKVEIK |
| | [SEQ ID NO: 23] |

In certain embodiments, the anti-L1CAM antibody is an scFv, an scFv-Fc fusion protein or a full-length human IgG (or antibody fragment thereof) with V_{H} and V_{L} regions or CDRs selected from Table 8. In certain embodiments, the anti-L1CAM scFv comprises a V_{H} comprising the amino acid sequence set forth in SEQ ID NO: 29. In certain embodiments, the anti-L1CAM scFv comprises a V_{L} comprising the amino acid sequence set forth in SEQ ID NO: 23. In certain embodiments, the anti-L1CAM scFv comprises a V_{H} comprising the amino acid sequence set forth in SEQ ID NO: 29 and a V_{L} comprising the amino acid sequence set forth in SEQ ID NO: 23. SEQ ID NO: 29 and 23 are provided in Table 8. In certain embodiments, the scFv is designated as “k1-h6”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 8.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 8.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 29, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 8

| CDRs | 1 | 2 | 3 |
|---|---|---|---|
| V_{H} | GYTFTNYY | IGPGSGSP | AKWLLPEDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYADSVKGRFTI SVDNSKNTLYLQMNSLRAEDTAVYYCAKWLLPFDYWGQGTLV |
| | TVSS |
| | [SEQ ID NO: 29] |
| Full | EIVLTQSPATLSLSPGERATLSCRASSSVNYMHWYQQKPGQAPRLLIYATSILASG |
| V_{L} | IPARFSGSGSGTDYTLTISSLEPEDFAVYYCQQFSSDPLTFGGGTKVEIK |
| | [SEQ ID NO: 23] |

In certain embodiments, the anti-L1CAM antibody is an scFv, an scFv-Fc fusion protein or a full-length human IgG (or antibody fragment thereof) with V_{H} and V_{L} regions or CDRs selected from Table 9. In certain embodiments, the anti-L1CAM scFv comprises a V_{H} comprising the amino acid sequence set forth in SEQ ID NO: 22. In certain embodiments, the anti-L1CAM scFv comprises a V_{L} comprising the amino acid sequence set forth in SEQ ID NO: 30. In certain embodiments, the anti-L1CAM scFv comprises a V_{H} comprising the amino acid sequence set forth in SEQ ID NO: 22 and a V_{L} comprising the amino acid sequence set forth in SEQ ID NO: 30. SEQ ID NO: 22 and 23 are provided in Table 9. In certain embodiments, the scFv is designated as “k2-h1”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 9.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 9.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 22, and a V_{L} comprising the amino acid sequence set forth in SEQ ID NO: 30. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 9

| CDRs | 1 | 2 | 3 |
|---|---|---|---|
| V_{H} | GYTFTNYY | IGPGSGSP | ARWLLPEDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS |
| | [SEQ ID NO: 22] |
| Full | EIVMTQSPATLSVSPGERATLSCRASSSVNYMHWYQQKPGQAPRLLIYATSILASG |
| V_{L} | I PARFSGSGSGTEYTLTISSLQSEDFAVYYCQQFSSDPLTFGGGTKVEIK |
| | [SEQ ID NO: 30] |

In certain embodiments, the anti-L1CAM antibody is an scFv, an scFv-Fc fusion protein or a full-length human IgG (or antibody fragment thereof) with V_{H} and V_{L} regions or CDRs selected from Table 10. In certain embodiments, the anti-L1CAM scFv comprises a V_{H} comprising the amino acid sequence set forth in SEQ ID NO: 24. In certain embodiments, the anti-L1CAM scFv comprises a V_{L} comprising the amino acid sequence set forth in SEQ ID NO: 30. In certain embodiments, the anti-L1CAM scFv comprises a V_{H} comprising the amino acid sequence set forth in SEQ ID NO: 24 and a V_{L} comprising the amino acid sequence set forth in SEQ ID NO: 30. SEQ ID NO: 24 and 23 are provided in Table 10. In certain embodiments, the scFv is designated as “k2-h2”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 10.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 10.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 24, and a V_{L} comprising the amino acid sequence set forth in SEQ ID NO: 30. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 10

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS |
| | [SEQ ID NO: 24] |
| Full | EIVMTQSPATLSVSPGERATLSCRASSSVNYMHWYQQKPGQAPRLLIYATSILASG |
| V_{L} | IPARFSGSGSGTEYTLTISSLQSEDFAVYYCQQFSSDPLTFGGGTKVEIK |
| | [SEQ ID NO: 30] |

In certain embodiments, the anti-L1CAM antibody is an scFv, an scFv-Fc fusion protein or a full-length human IgG (or antibody fragment thereof) with V_{H} and V_{L} regions or CDRs selected from Table 11. In certain embodiments, the anti-L1CAM scFv comprises a V_{H} comprising the amino acid sequence set forth in SEQ ID NO: 25. In certain embodiments, the anti-L1CAM scFv comprises a V_{L} comprising the amino acid sequence set forth in SEQ ID NO: 30. In certain embodiments, the anti-L1CAM scFv comprises a V_{H} comprising the amino acid sequence set forth in SEQ ID NO: 25 and a V_{L} comprising the amino acid sequence set forth in SEQ ID NO: 30. SEQ ID NO: 25 and 30 are provided in Table 11. In certain embodiments, the scFv is designated as “k2-h3”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 11.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 11.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 25, and a V_{L} comprising the amino acid sequence set forth in SEQ ID NO: 30. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 11

| CDRS | 1 | 2 | 3 |
|---|---|---|---|
| V_{H} | GYTFTNYY | IGPGSGSP | ARWLLPEDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVESGGGVVQPGRSLRLSCAASGYTFTNYYIHWVRQAPGKGLEWVAKIGPGSG |
| V_{H} | SPYYNGMFKDRFTISRDNSKNTLYLQMNSLRAEDTAVYYCARWLLPFDYWGQGTLV |
| | TVSS |
| | [SEQ ID NO: 25] |
| Full | EIVMTQSPATLSVSPGERATLSCRASSSVNYMHWYQQKPGQAPRLLIYATSILASG |
| V_{L} | IPARFSGSGSGTEYTLTISSLQSEDFAVYYCQQFSSDPLTFGGGTKVEIK |
| | [SEQ ID NO: 30] |

In certain embodiments, the anti-L1CAM antibody is an scFv, an scFv-Fc fusion protein or a full-length human IgG (or antibody fragment thereof) with V_{H} and V_{L} regions or CDRs selected from Table 12. In certain embodiments, the anti-L1CAM scFv comprises a V_{H} comprising the amino acid sequence set forth in SEQ ID NO: 27. In certain embodiments, the anti-L1CAM scFv comprises a V_{L} comprising the amino acid sequence set forth in SEQ ID NO: 30. In certain embodiments, the anti-L1CAM scFv comprises a V_{H} comprising the amino acid sequence set forth in SEQ ID NO: 27 and a V_{L} comprising the amino acid sequence set forth in SEQ ID NO: 30. SEQ ID NO: 27 and 30 are provided in Table 12. In certain embodiments, the scFv is designated as “k2-h4”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 12.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 12.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 27, and a V_{L} comprising the amino acid sequence set forth in SEQ ID NO: 30. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 12

| CDRS | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYNGMFKDRFTI SVDNSKNTLYLQMNSLRAEDTAVYYCAKWLLPFDYWGQGTLV |
| | TVSS |
| | [SEQ ID NO: 27] |
| Full | EIVMTQSPATLSVSPGERATLSCRASSSVNYMHWYQQKPGQAPRLLIYATSILASG |
| V_{L} | IPARFSGSGSGTEYTLTISSLQSEDFAVYYCQQFSSDPLTFGGGTKVEIK |
| | [SEQ ID NO: 30] |

In certain embodiments, the anti-L1CAM antibody is an scFv, an scFv-Fc fusion protein or a full-length human IgG (or antibody fragment thereof) with V_{H} and V_{L} regions or CDRs selected from Table 13. In certain embodiments, the anti-L1CAM scFv comprises a V_{H} comprising the amino acid sequence set forth in SEQ ID NO: 28. In certain embodiments, the anti-L1CAM scFv comprises a V_{L} comprising the amino acid sequence set forth in SEQ ID NO: 30. In certain embodiments, the anti-L1CAM scFv comprises a V_{H} comprising the amino acid sequence set forth in SEQ ID NO: 28 and a V_{L} comprising the amino acid sequence set forth in SEQ ID NO: 30. SEQ ID NO: 28 and 30 are provided in Table 13. In certain embodiments, the scFv is designated as “k2-h5”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 13.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 13.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 28, and a V_{L} comprising the amino acid sequence set forth in SEQ ID NO: 30. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 13

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| V_{H} | SPYYNGMFKDRVTITADESTSTAYMELSSLRSEDTAVYYCARWLLPFDYWGQGTLV |
| | TVSS |
| | [SEQ ID NO: 28] |
| Full | EIVMTQSPATLSVSPGERATLSCRASSSVNYMHWYQQKPGQAPRLLIYATSILASG |
| V_{L} | IPARFSGSGSGTEYTLTISSLQSEDFAVYYCQQFSSDPLTFGGGTKVEIK |
| | [SEQ ID NO: 30] |

In certain embodiments, the anti-L1CAM antibody is an scFv, an scFv-Fc fusion protein or a full-length human IgG (or antibody fragment thereof) with V_{H} and V_{L} regions or CDRs selected from Table 14. In certain embodiments, the anti-L1CAM scFv comprises a V_{H} comprising the amino acid sequence set forth in SEQ ID NO: 29. In certain embodiments, the anti-L1CAM scFv comprises a V_{L} comprising the amino acid sequence set forth in SEQ ID NO: 30. In certain embodiments, the anti-L1CAM scFv comprises a V_{H} comprising the amino acid sequence set forth in SEQ ID NO: 29 and a V_{L} comprising the amino acid sequence set forth in SEQ ID NO: 30. SEQ ID NO: 29 and 30 are provided in Table 14. In certain embodiments, the scFv is designated as “k2-h6”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 14.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 14.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 29, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 14

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYADSVKGRFTI SVDNSKNTLYLQMNSLRAEDTAVYYCAKWLLPFDYWGQGTLV |
| | TVSS |
| | [SEQ ID NO: 29] |
| Full | EIVMTQSPATLSVSPGERATLSCRASSSVNYMHWYQQKPGQAPRLLIYATSILASG |
| V_{L} | IPARFSGSGSGTEYTLTISSLQSEDFAVYYCQQFSSDPLTFGGGTKVEIK |
| | [SEQ ID NO: 30] |

In certain embodiments, the anti-L1CAM antibody is an scFv, an scFv-Fc fusion protein or a full-length human IgG (or antibody fragment thereof) with V_{H} and V_{L} regions or CDRs selected from Table 15. In certain embodiments, the anti-L1CAM scFv comprises a V_{H} comprising the amino acid sequence set forth in SEQ ID NO: 22. In certain embodiments, the anti-L1CAM scFv comprises a V_{L} comprising the amino acid sequence set forth in SEQ ID NO: 31. In certain embodiments, the anti-L1CAM scFv comprises a V_{H} comprising the amino acid sequence set forth in SEQ ID NO: 22 and a V_{L} comprising the amino acid sequence set forth in SEQ ID NO: 31. SEQ ID NO: 22 and 31 are provided in Table 15. In certain embodiments, the scFv is designated as “k3-h1”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 15.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 15.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 22, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 15

| CDRS | 1 | 2 | 3 |
|---|---|---|---|
| V_{H} | GYTFTNYY | IGPGSGSP | ARWLLPEDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS |
| | [SEQ ID NO: 22] |
| Full | DIQMTQSPSTLSASVGDRVTITCRASSSVNYMHWYQQKPGKAPKLLIYATSILASG |
| V_{L} | VPSRFSGSGSGTEYTLTISSLQPDDFATYYCQQFSSDPLTFGGGTKVEIK |
| | [SEQ ID NO: 31] |

In certain embodiments, the anti-L1CAM antibody is an scFv, an scFv-Fc fusion protein or a full-length human IgG (or antibody fragment thereof) with V_{H} and V_{L} regions or CDRs selected from Table 16. In certain embodiments, the anti-L1CAM scFv comprises a V_{H} comprising the amino acid sequence set forth in SEQ ID NO: 24. In certain embodiments, the anti-L1CAM scFv comprises a V_{L} comprising the amino acid sequence set forth in SEQ ID NO: 31. In certain embodiments, the anti-L1CAM scFv comprises a V_{H} comprising the amino acid sequence set forth in SEQ ID NO: 24 and a V_{L} comprising the amino acid sequence set forth in SEQ ID NO: 31. SEQ ID NO: 24 and 31 are provided in Table 16. In certain embodiments, the scFv is designated as “k3-h2”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 16.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 16.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 24, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 16

| CDRs | 1 | 2 | 3 |
|---|---|---|---|
| V_{H} | GYTFTNYY | IGPGSGSP | ARWLLPEDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS |
| | [SEQ ID NO: 24] |
| Full | DIQMTQSPSTLSASVGDRVTITCRASSSVNYMHWYQQKPGKAPKLLIYATSILASG |
| V_{L} | VPSRESGSGSGTEYTLTISSLQPDDFATYYCQQFSSDPLTFGGGTKVEIK |
| | [SEQ ID NO: 31] |

In certain embodiments, the anti-L1CAM antibody is an scFv, an scFv-Fc fusion protein or a full-length human IgG (or antibody fragment thereof) with V_{H} and V_{L} regions or CDRs selected from Table 17. In certain embodiments, the anti-L1CAM scFv comprises a V_{H} comprising the amino acid sequence set forth in SEQ ID NO: 25. In certain embodiments, the anti-L1CAM scFv comprises a V_{L} comprising the amino acid sequence set forth in SEQ ID NO: 31. In certain embodiments, the anti-L1CAM scFv comprises a V_{H} comprising the amino acid sequence set forth in SEQ ID NO: 25 and a V_{L} comprising the amino acid sequence set forth in SEQ ID NO: 31. SEQ ID NO: 25 and 31 are provided in Table 17. In certain embodiments, the scFv is designated as “k3-h3”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 17.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 17.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 25, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 17

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVESGGGVVQPGRSLRLSCAASGYTFTNYYIHWVRQAPGKGLEWVAKIGPGSG |
| V_{H} | SPYYNGMFKDRFTISRDNSKNTLYLQMNSLRAEDTAVYYCARWLLPFDYWGQGTLV |
| | TVSS |
| | [SEQ ID NO: 25] |
| Full | DIQMTQSPSTLSASVGDRVTITCRASSSVNYMHWYQQKPGKAPKLLIYATSILASG |
| V_{L} | VPSRESGSGSGTEYTLTISSLQPDDFATYYCQQFSSDPLTFGGGTKVEIK |
| | [SEQ ID NO: 31] |

In certain embodiments, the anti-L1CAM antibody is an scFv, an scFv-Fc fusion protein or a full-length human IgG (or antibody fragment thereof) with V_{H} and V_{L} regions or CDRs selected from Table 18. In certain embodiments, the anti-L1CAM scFv comprises a V_{H} comprising the amino acid sequence set forth in SEQ ID NO: 27. In certain embodiments, the anti-L1CAM scFv comprises a V_{L} comprising the amino acid sequence set forth in SEQ ID NO: 31. In certain embodiments, the anti-L1CAM scFv comprises a V_{H} comprising the amino acid sequence set forth in SEQ ID NO: 27 and a V_{L} comprising the amino acid sequence set forth in SEQ ID NO: 31. SEQ ID NO: 27 and 31 are provided in Table 18. In certain embodiments, the scFv is designated as “k3-h4”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 18.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 18.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 27, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 18

| CDRS | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYNGMFKDRFTI SVDNSKNTLYLQMNSLRAEDTAVYYCAKWLLPFDYWGQGTLV |
| | TVSS |
| | [SEQ ID NO: 27] |
| Full | DIQMTQSPSTLSASVGDRVTITCRASSSVNYMHWYQQKPGKAPKLLIYATSILASG |
| V_{L} | VPSRFSGSGSGTEYTLTISSLQPDDFATYYCQQFSSDPLTFGGGTKVEIK |
| | [SEQ ID NO: 31] |

In certain embodiments, the anti-L1CAM antibody is an scFv, an scFv-Fc fusion protein or a full-length human IgG (or antibody fragment thereof) with V_{H} and V_{L} regions or CDRs selected from Table 19. In certain embodiments, the anti-L1CAM scFv comprises a V_{H} comprising the amino acid sequence set forth in SEQ ID NO: 28. In certain embodiments, the anti-L1CAM scFv comprises a V_{L} comprising the amino acid sequence set forth in SEQ ID NO: 31. In certain embodiments, the anti-L1CAM scFv comprises a V_{H} comprising the amino acid sequence set forth in SEQ ID NO: 28 and a V_{L} comprising the amino acid sequence set forth in SEQ ID NO: 31. SEQ ID NO: 28 and 31 are provided in Table 19. In certain embodiments, the scFv is designated as “k3-h5”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 19.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 19.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 28, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 19

| CDRS | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| V_{H} | SPYYNGMFKDRVTITADESTSTAYMELSSLRSEDTAVYYCARWLLPFDYWGQGTLV |
| | TVSS |
| | [SEQ ID NO: 28] |
| Full | DIQMTQSPSTLSASVGDRVTITCRASSSVNYMHWYQQKPGKAPKLLIYATSILASG |
| V_{L} | VPSRFSGSGSGTEYTLTISSLQPDDFATYYCQQFSSDPLTFGGGTKVEIK |
| | [SEQ ID NO: 31] |

In certain embodiments, the anti-L1CAM antibody is an scFv, an scFv-Fc fusion protein or a full-length human IgG (or antibody fragment thereof) with V_{H} and V_{L} regions or CDRs selected from Table 20. In certain embodiments, the anti-L1CAM scFv comprises a V_{H} comprising the amino acid sequence set forth in SEQ ID NO: 29. In certain embodiments, the anti-L1CAM scFv comprises a V_{L} comprising the amino acid sequence set forth in SEQ ID NO: 31. In certain embodiments, the anti-L1CAM scFv comprises a V_{H} comprising the amino acid sequence set forth in SEQ ID NO: 29 and a V_{L} comprising the amino acid sequence set forth in SEQ ID NO: 31. SEQ ID NO: 29 and 31 are provided in Table 20. In certain embodiments, the scFv is designated as “k3-h6”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 20.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 20.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 29, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 20

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYADSVKGRFTI SVDNSKNTLYLQMNSLRAEDTAVYYCAKWLLPFDYWGQGTLV |
| | TVSS |
| | [SEQ ID NO: 29] |
| Full | DIQMTQSPSTLSASVGDRVTITCRASSSVNYMHWYQQKPGKAPKLLIYATSILASG |
| V_{L} | VPSRFSGSGSGTEYTLTISSLQPDDFATYYCQQFSSDPLTFGGGTKVEIK |
| | [SEQ ID NO: 31] |

In certain embodiments, the anti-L1CAM antibody is an scFv, an scFv-Fc fusion protein or a full-length human IgG (or antibody fragment thereof) with V_{H} and V_{L} regions or CDRs selected from Table 21. In certain embodiments, the anti-L1CAM scFv comprises a V_{H} comprising the amino acid sequence set forth in SEQ ID NO: 22. In certain embodiments, the anti-L1CAM scFv comprises a V_{L} comprising the amino acid sequence set forth in SEQ ID NO: 32. In certain embodiments, the anti-L1CAM scFv comprises a V_{H} comprising the amino acid sequence set forth in SEQ ID NO: 22 and a V_{L} comprising the amino acid sequence set forth in SEQ ID NO: 32. SEQ ID NO: 22 and 32 are provided in Table 21. In certain embodiments, the scFv is designated as “k4-h1”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 21.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 21.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 22, and a V_{L} comprising the amino acid sequence set forth in SEQ ID NO: 32. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 21

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS |
| | [SEQ ID NO: 22] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the anti-L1CAM antibody is an scFv, an scFv-Fc fusion protein or a full-length human IgG (or antibody fragment thereof) with V_{H} and V_{L} regions or CDRs selected from Table 22. In certain embodiments, the anti-L1CAM scFv comprises a V_{H} comprising the amino acid sequence set forth in SEQ ID NO: 24. In certain embodiments, the anti-L1CAM scFv comprises a V_{L} comprising the amino acid sequence set forth in SEQ ID NO: 32. In certain embodiments, the anti-L1CAM scFv comprises a V_{H} comprising the amino acid sequence set forth in SEQ ID NO: 24 and a V_{L} comprising the amino acid sequence set forth in SEQ ID NO: 32. SEQ ID NO: 24 and 32 are provided in Table 22. In certain embodiments, the scFv is designated as “k4-h2”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 22.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 22.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 24, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 22

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS |
| | [SEQ ID NO: 24] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the anti-L1CAM antibody is an scFv, an scFv-Fc fusion protein or a full-length human IgG (or antibody fragment thereof) with V_{H} and V_{L} regions or CDRs selected from Table 23. In certain embodiments, the anti-L1CAM scFv comprises a V_{H} comprising the amino acid sequence set forth in SEQ ID NO: 25. In certain embodiments, the anti-L1CAM scFv comprises a V_{L} comprising the amino acid sequence set forth in SEQ ID NO: 32. In certain embodiments, the anti-L1CAM scFv comprises a V_{H} comprising the amino acid sequence set forth in SEQ ID NO: 25 and a V_{L} comprising the amino acid sequence set forth in SEQ ID NO: 32. SEQ ID NO: 25 and 23 are provided in Table 23. In certain embodiments, the scFv is designated as “k4-h3”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 23.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 23.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 25, and a V_{L} comprising the amino acid sequence set forth in SEQ ID NO: 32. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 23

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVESGGGVVQPGRSLRLSCAASGYTFTNYYIHWVRQAPGKGLEWVAKIGPGSG |
| V_{H} | SPYYNGMFKDRFTISRDNSKNTLYLQMNSLRAEDTAVYYCARWLLPFDYWGQGTLV |
| | TVSS |
| | [SEQ ID NO: 25] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the anti-L1CAM antibody is an scFv, an scFv-Fc fusion protein or a full-length human IgG (or antibody fragment thereof) with V_{H} and V_{L} regions or CDRs selected from Table 24. In certain embodiments, the anti-L1CAM scFv comprises a V_{H} comprising the amino acid sequence set forth in SEQ ID NO: 27. In certain embodiments, the anti-L1CAM scFv comprises a V_{L} comprising the amino acid sequence set forth in SEQ ID NO: 32. In certain embodiments, the anti-L1CAM scFv comprises a V_{H} comprising the amino acid sequence set forth in SEQ ID NO: 27 and a V_{L} comprising the amino acid sequence set forth in SEQ ID NO: 32. SEQ ID NO: 27 and 32 are provided in Table 24. In certain embodiments, the scFv is designated as “k4-h4”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 24.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 24.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 27, and a V_{L} comprising the amino acid sequence set forth in SEQ ID NO: 32. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 24

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYNGMFKDRFTI SVDNSKNTLYLQMNSLRAEDTAVYYCAKWLLPFDYWGQGTLV |
| | TVSS |
| | [SEQ ID NO: 27] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the anti-L1CAM antibody is an scFv, an scFv-Fc fusion protein or a full-length human IgG (or antibody fragment thereof) with V_{H} and V_{L} regions or CDRs selected from Table 25. In certain embodiments, the anti-L1CAM scFv comprises a V_{H} comprising the amino acid sequence set forth in SEQ ID NO: 28. In certain embodiments, the anti-L1CAM scFv comprises a V_{L} comprising the amino acid sequence set forth in SEQ ID NO: 32. In certain embodiments, the anti-L1CAM scFv comprises a V_{H} comprising the amino acid sequence set forth in SEQ ID NO: 28 and a V_{L} comprising the amino acid sequence set forth in SEQ ID NO: 32. SEQ ID NO: 28 and 32 are provided in Table 25. In certain embodiments, the scFv is designated as “k4-h5”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 25.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 25.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 28, and a V_{L} comprising the amino acid sequence set forth in SEQ ID NO: 32. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 25

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| V_{H} | SPYYNGMFKDRVTITADESTSTAYMELSSLRSEDTAVYYCARWLLPFDYWGQGTLV |
| | TVSS |
| | [SEQ ID NO: 28] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the anti-L1CAM antibody is an scFv, an scFv-Fc fusion protein or a full-length human IgG (or antibody fragment thereof) with V_{H} and V_{L} regions or CDRs selected from Table 26. In certain embodiments, the anti-L1CAM scFv comprises a V_{H} comprising the amino acid sequence set forth in SEQ ID NO: 29. In certain embodiments, the anti-L1CAM scFv comprises a V_{L} comprising the amino acid sequence set forth in SEQ ID NO: 32. In certain embodiments, the anti-L1CAM scFv comprises a V_{H} comprising the amino acid sequence set forth in SEQ ID NO: 29 and a V_{L} comprising the amino acid sequence set forth in SEQ ID NO: 32. SEQ ID NO: 29 and 32 are provided in Table 26. In certain embodiments, the scFv is designated as “k4-h6”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 26.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 26.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 29, and a V_{L} comprising the amino acid sequence set forth in SEQ ID NO: 32. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 26

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYADSVKGRFTISVDNSKNTLYLQMNSLRAEDTAVYYCAKWLLPFDYWGQGTLV |
| | TVSS |
| | [SEQ ID NO: 29] |
| Full | DIQMTQSPSSVSASVGDRVTITCRASSSVNYMHWYQQKPGKAPKPLIYATSILASG |
| V_{L} | VPSRFSGSGSGTDYTLTISSLQPEDFATYYCQQFSSDPLTFGGGTKVEIK |
| | [SEQ ID NO: 32] |

In certain embodiments, the anti-L1CAM antibody is an scFv, an scFv-Fc fusion protein or a full-length human IgG (or antibody fragment thereof) with V_{H} and V_{L} regions or CDRs selected from Table 27. In certain embodiments, the anti-L1CAM scFv comprises a V_{H} comprising the amino acid sequence set forth in SEQ ID NO: 22. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 22 and a V_{L} comprising the amino acid sequence set forth in SEQ ID NO: 19. SEQ ID NO: 22 and 19 are provided in Table 27. In certain embodiments, the scFv is designated as “k5-h1”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 27.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 27.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 22, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus. V_{L}-V_{H}.

TABLE 27

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTMTRDTSTSTVYMELSSLRSEDTAVYYCARWLLPFDYWGQGTLV |
| | TVSS |
| | [SEQ ID NO: 22] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK |
| | [SEQ ID NO: 19] |

In certain embodiments, the anti-L1CAM antibody is an scFv, an scFv-Fc fusion protein or a full-length human IgG (or antibody fragment thereof) with V_{H} and V_{L} regions or CDRs selected from Table 28. In certain embodiments, the anti-L1CAM scFv comprises a V_{H} comprising the amino acid sequence set forth in SEQ ID NO: 24. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 24 and a V_{L} comprising the amino acid sequence set forth in SEQ ID NO: 19. SEQ ID NO: 24 and 19 are provided in Table 28. In certain embodiments, the scFv is designated as “k5-h2”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 28.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 28.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 24, and a V_{L} comprising the amino acid sequence set forth in SEQ ID NO: 19. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 28

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGASVKVSCKASGYTFTNYYIHWVRQAPGQGLEWMGKIGPGSG |
| V_{H} | SPYYNGMFKDRVTSTRDTSISTAYMELSRLRSDDTVVYYCARWLLPFDYWGQGTLV |
| | TVSS |
| | [SEQ ID NO: 24] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK |
| | [SEQ ID NO: 19] |

In certain embodiments, the anti-L1CAM antibody is an scFv, an scFv-Fc fusion protein or a full-length human IgG (or antibody fragment thereof) with V_{H} and V_{L} regions or CDRs selected from Table 29. In certain embodiments, the anti-L1CAM scFv comprises a V_{H} comprising the amino acid sequence set forth in SEQ ID NO: 25. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 25 and a V_{L} comprising the amino acid sequence set forth in SEQ ID NO: 19. SEQ ID NO: 25 and 19 are provided in Table 29. In certain embodiments, the scFv is designated as “k5-h3”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 29.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 29.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 25, and a V_{L} comprising the amino acid sequence set forth in SEQ ID NO: 19. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 29

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVESGGGVVQPGRSLRLSCAASGYTFTNYYIHWVRQAPGKGLEWVAKIGPGSG |
| V_{H} | SPYYNGMFKDRFTISRDNSKNTLYLQMNSLRAEDTAVYYCARWLLPFDYWGQGTLV |
| | TVSS |
| | [SEQ ID NO: 25] |
| Full | EIVLTQSPGTLSLSPGERATLSCRASSSVNYMHWYQQKPGQAPRLLIYATSILASG |
| V_{L} | IPDRESGSGSGTDYTLTI SRLEPEDFAVYYCQQFSSDPLTFGGGTKVEIK |
| | [SEQ ID NO: 19] |

In certain embodiments, the anti-L1CAM antibody is an scFv, an scFv-Fc fusion protein or a full-length human IgG (or antibody fragment thereof) with V_{H} and V_{L} regions or CDRs selected from Table 30. In certain embodiments, the anti-L1CAM scFv comprises a V_{H} comprising the amino acid sequence set forth in SEQ ID NO: 27. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 27 and a V_{L} comprising the amino acid sequence set forth in SEQ ID NO: 19. SEQ ID NO: 27 and 19 are provided in Table 30. In certain embodiments, the scFv is designated as “k5-h4”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 30.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 30.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 27, and a V_{L} comprising the amino acid sequence set forth in SEQ ID NO: 19. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 30

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYNGMFKDRFTISVDNSKNTLYLQMNSLRAEDTAVYYCAKWLLPFDYWGQGTLV |
| | TVSS |
| | [SEQ ID NO: 27] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK |
| | [SEQ ID NO: 19] |

In certain embodiments, the anti-L1CAM antibody is an scFv, an scFv-Fc fusion protein or a full-length human IgG (or antibody fragment thereof) with V_{H} and V_{L} regions or CDRs selected from Table 31. In certain embodiments, the anti-L1CAM scFv comprises a V_{H} comprising the amino acid sequence set forth in SEQ ID NO: 28. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 28 and a V_{L} comprising the amino acid sequence set forth in SEQ ID NO: 19. SEQ ID NO: 28 and 19 are provided in Table 31. In certain embodiments, the scFv is designated as “k5-h5”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 31.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 31.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 28, and a V_{L} comprising the amino acid sequence set forth in SEQ ID NO: 19. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 31

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | QVQLVQSGAEVKKPGSSVKVSCKASGYTFTNYYIHWVRQAPGQGLEWMGKIGPGSG |
| V_{H} | SPYYNGMFKDRVTITADESTSTAYMELSSLRSEDTAVYYCARWLLPFDYWGQGTLV |
| | TVSS |
| | [SEQ ID NO: 28] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK |
| | [SEQ ID NO: 19] |

In certain embodiments, the anti-L1CAM antibody is an scFv, an scFv-Fc fusion protein or a full-length human IgG (or antibody fragment thereof) with V_{H} and V_{L} regions or CDRs selected from Table 32. In certain embodiments, the anti-L1CAM scFv comprises a V_{H} comprising the amino acid sequence set forth in SEQ ID NO: 29. In certain embodiments, the anti-L1CAM scFv comprises a V_{L} comprising the amino acid sequence set forth in SEQ ID NO: 19. In certain embodiments, the anti-L1CAM scFv comprises a V_{H} comprising the amino acid sequence set forth in SEQ ID NO: 29 and a V_{L} comprising the amino acid sequence set forth in SEQ ID NO: 19. SEQ ID NO: 29 and 19 are provided in Table 32. In certain embodiments, the scFv is designated as “k5-h6”.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 32.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 32.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 29, and a V_{L} comprising the amino acid sequence set forth in SEQ ID NO: 32. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 32

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNY | ATS | QQFSSDPLT |
| | [SEQ ID NO: 15] | [SEQ ID NO: 16] | [SEQ ID NO: 17] |

| | |
|---|---|
| Full | EVQLLESGGGLVQPGGSLRLSCAASGYTFTNYYIHWVRQAPGKGLEWVSKIGPGSG |
| V_{H} | SPYYADSVKGRFTISVDNSKNTLYLQMNSLRAEDTAVYYCAKWLLPFDYWGQGTLV |
| | TVSS |
| | [SEQ ID NO: 29] |
| Full | EIVLTQSPGTLSLSPGERATLSCRASSSVNYMHWYQQKPGQAPRLLIYATSILASG |
| V_{L} | IPDRFSGSGSGTDYTLTISRLEPEDFAVYYCQQFSSDPLTFGGGTKVEIK |
| | [SEQ ID NO: 19] |

In certain embodiments, the anti-L1CAM antibody is an scFv, an scFv-Fc fusion protein or a full-length human IgG (or antibody fragment thereof) with V_{H} and V_{L} regions or CDRs selected from Table 33. In certain embodiments, the anti-L1CAM scFv comprises a V_{H} comprising the amino acid sequence set forth in SEQ ID NO: 33. In certain embodiments, the anti-L1CAM scFv comprises a V_{L} comprising the amino acid sequence set forth in SEQ ID NO: 34. SEQ ID NO: 33 and 34 are provided in Table 33. In certain embodiments, the scFv is designated as “143G03”.

In certain embodiments, the anti-L1CAM scFv comprises a V_{H} comprising the amino acid sequence set forth in SEQ ID NO: 33 and a V_{L} comprising the amino acid sequence set forth in SEQ ID NO: 34.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 26 or a conservative modification thereof. SEQ ID NOs: 12, 13, and 26 are provided in Table 33.

In certain embodiments, the anti-L1CAM scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 33.

In certain embodiments, the anti-L1CAM scFv comprises a V_{H} Comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification, and a CDR3 comprising the amino acid sequence set forth in SEQ ID NO: 17 or a conservative modification thereof.

In certain embodiments, the anti-L1CAM scFv comprises a V_{H} Comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 26; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-L1CAM scFv comprises a V_{H} comprising the amino acid sequence set forth in SEQ ID NO: 33, and a V_{L} comprising the amino acid sequence set forth in SEQ ID NO: 34. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 33

| CDRs | 1 | 2 | 3 |
|---|---|---|---|
| V_{H} | GYTFTNYY | IGPGSGSP | AKWLLPFDY |
| | [SEQ ID NO: 12] | [SEQ ID NO: 13] | [SEQ ID NO: 26] |
| V_{L} | SSVNYMH | ATS | QQFSSDPLT |
| | [SEQ ID NO: 15] | [SEQ ID NO: 16] | [SEQ ID NO: 17] |

| | |
|---|---|
| Full | QVQLRQSGAELVRPGTSVKMSCKASGYTFTNYYIHWVKQRPGQGLEWIGKIGPGSG |
| V_{H} | SPYYNGMFKDKATLTVDTSSSTAYMQLSSLTSEDSAVYFCARWLLPFDYWGQGTTL |
| | TVSS |
| | [SEQ ID NO: 33] |
| Full | QIVLSQSPGILSASPGEKVTMTCRASSSVNYMHWYQQKSGSSPKPWIFATSILASG |
| V_{L} | VPPRFSGSGSGTSYSLTISSVEAEDTATYYCQQFSSDPLTFGAGTKLELK |
| | [SEQ ID NO: 34] |

In certain embodiments, the anti-L1CAM antibody is an scFv, an scFv-Fc fusion protein or a full-length human IgG (or antibody fragment thereof) with V_{H} and V_{L} regions or CDRs selected from Table 34. In certain embodiments, the anti-L1CAM scFv comprises a V_{H} comprising the amino acid sequence set forth in SEQ ID NO: 41. In certain embodiments, the anti-L1CAM scFv comprises a V_{L} comprising the amino acid sequence set forth in SEQ ID NO: 42. SEQ ID NO: 41 and 42 are provided in Table 34. In certain embodiments, the scFv is designated as “04B06”.

In certain embodiments, the anti-L1CAM scFv comprises a V_{H} comprising the amino acid sequence set forth in SEQ ID NO: 41 and a V_{L} comprising the amino acid sequence set forth in SEQ ID NO: 42.

In certain embodiments, the anti-L1CAM scFv comprises a V_{H} Comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof. SEQ ID NOs: 35-37 are provided in Table 34.

In certain embodiments, the anti-L1CAM scFv comprises a V_{L} comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof. SEQ ID NOs: 38-40 are provided in Table 34.

In certain embodiments, the anti-L1CAM scFv comprises a V_{H} Comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, a CDR3 comprising the amino acid sequence set forth in SEQ ID NO: 37 or a conservative modification thereof; and a V_{L} comprising a CDR1 comprising the amino acid sequence set forth in SEQ ID NO: 38 or a conservative modification thereof, a CDR2 comprising the amino acid sequence set forth in SEQ ID NO: 39 or a conservative modification, and a CDR3 comprising the amino acid sequence set forth in SEQ ID NO: 40 or a conservative modification thereof.

In certain embodiments, the anti-L1CAM scFv comprises a V_{H} Comprising a CDR1 comprising the amino acid sequence set forth in SEQ ID NO: 35, a CDR2 comprising the amino acid sequence set forth in SEQ ID NO: 36, a CDR3 comprising the amino acid sequence set forth in SEQ ID NO: 37; and a V_{L} comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40.

In certain embodiments, the anti-L1CAM scFv comprises a V_{H} comprising the amino acid sequence set forth in SEQ ID NO: 41, and a V_{L} comprising the amino acid sequence set forth in SEQ ID NO: 42. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 34

| CDRs | 1 | 2 | 3 |
|---|---|---|---|
| V_{H} | GGSINSYY | FYTSGST | AREWSGSFYGGDAFDI |
| | [SEQ ID NO: 35] | [SEQ ID NO: 36] | [SEQ ID NO: 37] |
| V_{L} | QSVLNSSNNKNY | WAS | QQYYSTPYT |
| | [SEQ ID NO: 38] | [SEQ ID NO: 39] | [SEQ ID NO: 40] |

| | |
|---|---|
| Full | QVQLQESGPGLVKPSETLSLTCTVSGGSINSYYWSWIRQPAGKGLEWIGRFYTSGS |
| V_{H} | TNHNPSLKSRVTMSVDTSKNQFSLKLTSVTAADTAVYYCAREWSGSFYGGDAFDIW |
| | GQGTMVTVSS |
| | [SEQ ID NO: 41] |
| Full | DIVMTQSPDSLAVSLGERATINCKSSQSVLNSSNNKNYLTWYQQRPGQPPNLLIYW |
| V_{L} | ASTRESGVPDRFSGSGSGTDFTLTISSLQAEDVAVYYCQQYYSTPYTFGQGTKLEI |
| | E |
| | [SEQ ID NO: 42] |

In certain embodiments, the anti-L1CAM antibody is an scFv, an scFv-Fc fusion protein or a full-length human IgG (or antibody fragment thereof) with V_{H} and V_{L} regions or CDRs selected from Table 35. In certain embodiments, the anti-L1CAM scFv comprises a V_{H} comprising the amino acid sequence set forth in SEQ ID NO: 49. In certain embodiments, the anti-L1CAM scFv comprises a V_{L} comprising the amino acid sequence set forth in SEQ ID NO: 50. SEQ ID NO: 49 and 50 are provided in Table 35. In certain embodiments, the scFv is designated as “12D10v1”.

In certain embodiments, the anti-L1CAM scFv comprises a V_{H} comprising the amino acid sequence set forth in SEQ ID NO: 49 and a V_{L} comprising the amino acid sequence set forth in SEQ ID NO: 50.

In certain embodiments, the anti-L1CAM scFv comprises a V_{H} Comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof. SEQ ID NOs: 43-45 are provided in Table 35.

In certain embodiments, the anti-L1CAM scFv comprises a V_{L} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof. SEQ ID NOs: 46-48 are provided in Table 35.

In certain embodiments, the anti-L1CAM scFv comprises a V_{H} Comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, a CDR3 comprising the amino acid sequence set forth in SEQ ID NO: 45 or a conservative modification thereof; and a V_{L} comprising a CDR1 comprising the amino acid sequence set forth in SEQ ID NO: 46 or a conservative modification thereof, a CDR2 comprising the amino acid sequence set forth in SEQ ID NO: 47 or a conservative modification, and a CDR3 comprising the amino acid sequence set forth in SEQ ID NO: 48 or a conservative modification thereof.

In certain embodiments, the anti-L1CAM scFv comprises a V_{H} Comprising a CDR1 comprising the amino acid sequence set forth in SEQ ID NO: 43, a CDR2 comprising the amino acid sequence set forth in SEQ ID NO: 44, a CDR3 comprising the amino acid sequence set forth in SEQ ID NO: 45; and a V_{L} comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48.

In certain embodiments, the anti-L1CAM scFv comprises a V_{H} comprising the amino acid sequence set forth in SEQ ID NO: 49, and a V_{L} comprising the amino acid sequence set forth in SEQ ID NO: 50. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 35

| CDRs | 1 | 2 | 3 |
|---|---|---|---|
| V_{H} | GYPFTGYY | INPNSGDT | AREMGTTKGYFDL |
| | [SEQ ID NO: 43] | [SEQ ID NO: 44] | [SEQ ID NO: 45] |
| V_{L} | QGISSY | AAS | QQLNSYPDT |
| | [SEQ ID NO: 46] | [SEQ ID NO: 47] | [SEQ ID NO: 48] |

| | |
|---|---|
| Full | QVQLVQSGAEVKKPGASVRVSCKASGYPFTGYYMHWVRQAPGQGLEWMGWINPNSG |
| V_{H} | DTDYVQKFQGRVTMTRDTSISTAYMELSRLRSDDTAVYYCAREMGTTKGYFDLWGR |
| | GTLVTVSS |
| | [SEQ ID NO: 49] |
| Full | DIQLTQSPSFLSASVGDRVTITCRASQGISSYLAWFQQKPGKAPRLLIYAASTLQS |
| V_{L} | GVPSRFSGSRSGTEFTLTISSLQPEDFATYYCQQLNSYPDTFGQGTKVDIK |
| | [SEQ ID NO: 50] |

In certain embodiments, the anti-L1CAM antibody is an scFv, an scFv-Fc fusion protein or a full-length human IgG (or antibody fragment thereof) with V_{H} and V_{L} regions or CDRs selected from Table 36. In certain embodiments, the anti-L1CAM scFv comprises a V_{H} comprising the amino acid sequence set forth in SEQ ID NO: 49. In certain embodiments, the anti-L1CAM scFv comprises a V_{L} comprising the amino acid sequence set forth in SEQ ID NO: 53. SEQ ID NO: 49 and 53 are provided in Table 36. In certain embodiments, the scFv is designated as “12D10v2”.

In certain embodiments, the anti-L1CAM scFv comprises a V_{H} comprising the amino acid sequence set forth in SEQ ID NO: 49 and a V_{L} comprising the amino acid sequence set forth in SEQ ID NO: 53.

In certain embodiments, the anti-L1CAM scFv comprises a V_{H} Comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof. SEQ ID NOs: 43-45 are provided in Table 36.

In certain embodiments, the anti-L1CAM scFv comprises a V_{L} comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof. SEQ ID NOs: 40, 51, and 52 are provided in Table 36.

In certain embodiments, the anti-L1CAM scFv comprises a V_{H} Comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, a CDR3 comprising the amino acid sequence set forth in SEQ ID NO: 45 or a conservative modification thereof; and a V_{L} comprising a CDR1 comprising the amino acid sequence set forth in SEQ ID NO: 51 or a conservative modification thereof, a CDR2 comprising the amino acid sequence set forth in SEQ ID NO: 52 or a conservative modification, and a CDR3 comprising the amino acid sequence set forth in SEQ ID NO: 40 or a conservative modification thereof.

In certain embodiments, the anti-L1CAM scFv comprises a V_{H} Comprising a CDR1 comprising the amino acid sequence set forth in SEQ ID NO: 43, a CDR2 comprising the amino acid sequence set forth in SEQ ID NO: 44, a CDR3 comprising the amino acid sequence set forth in SEQ ID NO: 45; and a V_{L} comprising a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40.

In certain embodiments, the anti-L1CAM scFv comprises a V_{H} comprising the amino acid sequence set forth in SEQ ID NO: 49, and a V_{L} comprising the amino acid sequence set forth in SEQ ID NO: 53. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 36

| CDRs | 1 | 2 | 3 |
|---|---|---|---|
| V_{H} | GYPFTGYY | INPNSGDT | AREMGTTKGYFDL |
| | [SEQ ID NO: 43] | [SEQ ID NO: 44] | [SEQ ID NO: 45] |
| V_{L} | QTILYSPNNKNY | LTS | QQYYSTPYT |
| | [SEQ ID NO: 51] | [SEQ ID NO: 52] | [SEQ ID NO: 40] |

| | |
|---|---|
| Full | QVQLVQSGAEVKKPGASVRVSCKASGYPFTGYYMHWVRQAPGQGLEWMGWINPNSG |
| V_{H} | DTDYVQKFQGRVTMTRDTSISTAYMELSRLRSDDTAVYYCAREMGTTKGYFDLWGR |
| | GTLVTVSS |
| | [SEQ ID NO: 49] |
| Full | DIVMTQSPDSLAVSLGERATINCKSSQTILYSPNNKNYLAWYQQKPGQPPKLLIYL |
| V_{L} | TSTRESGVPDRFSGSGSGTDFTLTISSLQAEDVAVYYCQQYYSTPYTFGQGTKLEI |
| | K |
| | [SEQ ID NO: 53] |

In certain embodiments, the anti-L1CAM antibody is an scFv, an scFv-Fc fusion protein or a full-length human IgG (or antibody fragment thereof) with V_{H} and V_{L} regions or CDRs selected from Table 37. In certain embodiments, the anti-L1CAM scFv comprises a V_{H} comprising the amino acid sequence set forth in SEQ ID NO: 60. In certain embodiments, the anti-L1CAM scFv comprises a V_{L} comprising the amino acid sequence set forth in SEQ ID NO: 61. SEQ ID NO: 60 and 61 are provided in Table 37. In certain embodiments, the scFv is designated as “13G04”.

In certain embodiments, the anti-L1CAM scFv comprises a V_{H} comprising the amino acid sequence set forth in SEQ ID NO: 60 and a V_{L} comprising the amino acid sequence set forth in SEQ ID NO: 61.

In certain embodiments, the anti-L1CAM scFv comprises a V_{H} Comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof. SEQ ID NOs: 54-56 are provided in Table 37.

In certain embodiments, the anti-L1CAM scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 58-59 are provided in Table 37.

In certain embodiments, the anti-L1CAM scFv comprises a V_{H} Comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification, and a CDR3 comprising the amino acid sequence set forth in SEQ ID NO: 59 or a conservative modification thereof.

In certain embodiments, the anti-L1CAM scFv comprises a V_{H} Comprising a CDR1 comprising the amino acid sequence set forth in SEQ ID NO: 54, a CDR2 comprising the amino acid sequence set forth in SEQ ID NO: 55, a CDR3 comprising the amino acid sequence set forth in SEQ ID NO: 56; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59.

In certain embodiments, the anti-L1CAM scFv comprises a V_{H} comprising the amino acid sequence set forth in SEQ ID NO: 60, and a V_{L} comprising the amino acid sequence set forth in SEQ ID NO: 61. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 37

| CDRs | 1 | 2 | 3 |
|---|---|---|---|
| V_{H} | GYTFIGYY | INPNSGGT | ARGGLERRDAFDI |
| | [SEQ ID | [SEQ ID | [SEQ ID NO: 56] |
| | NO: 54] | NO: 55] | |
| V_{L} | QSISNW | KAS | QQYDTYRT |
| | [SEQ ID | [SEQ ID | [SEQ ID NO: 59] |
| | NO: 57] | NO: 58] | |

| | |
|---|---|
| Full | QVQLVQSGAEVKKPGASVKVSCKASGYTFIGYYLHWVRQAP |
| V_{H} | GQGLEWMGWINPNSGGTNSAQKFQGRVTMTRDTSISTTYMD |
| | LSRLRSDDTAVYYCARGGLERRDAFDIWGQGTLVTVSS |
| | [SEQ ID NO: 60] |
| Full | DIQMTQSPSTLSASVGDRVTITCRASQSISNWLAWYQQKPG |
| V_{L} | KAPNLLIYKASSLESGVPSRFSGSGSGTEFTLTISSLQPDD |
| | FATYYCQQYDTYRTFGQGTKVEIK |
| | [SEQ ID NO: 61] |

In certain embodiments, the anti-L1CAM antibody is an scFv, an scFv-Fc fusion protein or a full-length human IgG (or antibody fragment thereof) with V_{H} and V_{L} regions or CDRs selected from Table 38. In certain embodiments, the anti-L1CAM scFv comprises a V_{H} comprising the amino acid sequence set forth in SEQ ID NO: 67. In certain embodiments, the anti-L1CAM scFv comprises a V_{L} comprising the amino acid sequence set forth in SEQ ID NO: 68. SEQ ID NO: 67 and 68 are provided in Table 38. In certain embodiments, the scFv is designated as “15G02”.

In certain embodiments, the anti-L1CAM scFv comprises a V_{H} comprising the amino acid sequence set forth in SEQ ID NO: 67 and a V_{L} comprising the amino acid sequence set forth in SEQ ID NO: 68.

In certain embodiments, the anti-L1CAM scFv comprises a V_{H} Comprising a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof. SEQ ID NOs: 62-64 are provided in Table 38.

In certain embodiments, the anti-L1CAM scFv comprises a V_{L} comprising a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof. SEQ ID NOs: 58, 65, and 66 are provided in Table 38.

In certain embodiments, the anti-L1CAM scFv comprises a V_{H} Comprising a CDR1 comprising the amino acid sequence set forth in SEQ ID NO: 62 or a conservative modification thereof, a CDR2 comprising the amino acid sequence set forth in SEQ ID NO: 63 or a conservative modification thereof, a CDR3 comprising the amino acid sequence set forth in SEQ ID NO: 64 or a conservative modification thereof; and a V_{L} comprising a CDR1 comprising the amino acid sequence set forth in SEQ ID NO: 65 or a conservative modification thereof, a CDR2 comprising the amino acid sequence set forth in SEQ ID NO: 58 or a conservative modification, and a CDR3 comprising the amino acid sequence set forth in SEQ ID NO: 66 or a conservative modification thereof.

In certain embodiments, the anti-L1CAM scFv comprises a V_{H} Comprising a CDR1 comprising the amino acid sequence set forth in SEQ ID NO: 62, a CDR2 comprising the amino acid sequence set forth in SEQ ID NO: 63, a CDR3 comprising the amino acid sequence set forth in SEQ ID NO: 64; and a V_{L} comprising a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66.

In certain embodiments, the anti-L1CAM scFv comprises a V_{H} comprising the amino acid sequence set forth in SEQ ID NO: 67, and a V_{L} comprising the amino acid sequence set forth in SEQ ID NO: 68. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 38

| CDRs | 1 | 2 | 3 |
|---|---|---|---|
| V_{H} | GFTFSHYW | IMQDGSEK | ARDRGVTTLFDY |
| | [SEQ ID | [SEQ ID | [SEQ ID NO: 64] |
| | NO: 62] | NO: 63] | |
| V_{L} | QSISSW | KAS | QQYYSYGT |
| | [SEQ ID | [SEQ ID | [SEQ ID NO: 66] |
| | NO: 65] | NO: 58] | |

| | |
|---|---|
| Full | EVQLVESGGGLVQPGGSLRLSCGVSGFTFSHYWMNWVRQAP |
| V_{H} | GKGLEWVANIMQDGSEKYYVDSVKGRFTISRDNAKNSLYLQ |
| | MNSLRVEDTAVYYCARDRGVTTLFDYWGQGILVTVSS |
| | [SEQ ID NO: 67] |
| Full | DIQMTQSPSTLSASVGDRVTITCRASQSISSWLAWYQQKPG |
| V_{L} | KAPKLLIYKASSLESGVPSRFSGSGSGTEFTLTISSLQPDD |
| | FATYYCQQYYSYGTFGQGTKVEIK |
| | [SEQ ID NO: 68] |

In certain embodiments, the anti-L1CAM antibody is an scFv, an scFv-Fc fusion protein or a full-length human IgG (or antibody fragment thereof) with V_{H} and V_{L} regions or CDRs selected from Table 39. In certain embodiments, the anti-L1CAM scFv comprises a V_{H} comprising the amino acid sequence set forth in SEQ ID NO: 75. In certain embodiments, the anti-L1CAM scFv comprises a V_{L} comprising the amino acid sequence set forth in SEQ ID NO: 76. SEQ ID NO: 75 and 76 are provided in Table 39. In certain embodiments, the scFv is designated as “13F04”.

In certain embodiments, the anti-L1CAM scFv comprises a V_{H} comprising the amino acid sequence set forth in SEQ ID NO: 75 and a V_{L} comprising the amino acid sequence set forth in SEQ ID NO: 76.

In certain embodiments, the anti-L1CAM scFv comprises a V_{H} Comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof. SEQ ID NOs: 69-71 are provided in Table 39.

In certain embodiments, the anti-L1CAM scFv comprises a V_{L} comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof. SEQ ID NOs: 72-74 are provided in Table 39.

In certain embodiments, the anti-L1CAM scFv comprises a V_{H} Comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, a CDR3 comprising the amino acid sequence set forth in SEQ ID NO: 71 or a conservative modification thereof; and a V_{L} comprising a CDR1 comprising the amino acid sequence set forth in SEQ ID NO: 72 or a conservative modification thereof, a CDR2 comprising the amino acid sequence set forth in SEQ ID NO: 73 or a conservative modification, and a CDR3 comprising the amino acid sequence set forth in SEQ ID NO: 74 or a conservative modification thereof.

In certain embodiments, the anti-L1CAM scFv comprises a V_{H} Comprising a CDR1 comprising the amino acid sequence set forth in SEQ ID NO: 69, a CDR2 comprising the amino acid sequence set forth in SEQ ID NO: 70, a CDR3 comprising the amino acid sequence set forth in SEQ ID NO: 71; and a V_{L} comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the anti-L1CAM scFv comprises a V_{H} comprising the amino acid sequence set forth in SEQ ID NO: 75, and a V_{L} comprising the amino acid sequence set forth in SEQ ID NO: 76. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}. In certain embodiments, the anti-L1CAM scFv comprises the amino acid sequence set forth in SEQ ID NO: 77. In certain embodiments, the anti-L1CAM scFv comprises the amino acid sequence set forth in SEQ ID NO: 78.

TABLE 39

| CDRs | 1 | 2 | 3 |
|---|---|---|---|
| V_{H} | GYIFISYW | INPRNGRT | ARGGDYGGSFDY |
| | [SEQ ID NO: 69] | [SEQ ID NO: 70] | [SEQ ID NO: 71] |
| V_{L} | ESVDSYGNSF | RAS | QQSNEDPWT |
| | [SEQ ID NO: 72] | [SEQ ID NO: 73] | [SEQ ID NO: 74] |

| | |
|---|---|
| Full | QVQLQQPGAELVKPGASVKLSCKASGYIFISYWMHWVKQRPGQGLEWIGEINPRNG |
| V_{H} | RTNYNEKFKSKATLTVDKSSSTAYMQLTSLTSEDSAVYYCARGGDYGGSFDYWGQG |
| | TTLTVSS |
| | [SEQ ID NO: 75] |
| Full | DIVLTQSPASLAVSLGQRATISCRASESVDSYGNSFMHWYQQKPGQPPKLLIYRAS |
| V_{L} | NLESGIPARFSGSGSRTDFTLTVNPVEADDVATYYCQQSNEDPWTFGGGTKLEIK |
| | [SEQ ID NO: 76] |
| scFv | QVQLQQPGAELVKPGASVKLSCKASGYIFISYWMHWVKQRPGQGLEWIGEINPRNG |
| V_{H}-V_{L} | RTNYNEKFKSKATLTVDKSSSTAYMQLTSLTSEDSAVYYCARGGDYGGSFDYWGQG |
| | TTLTVSSGGGGSGGGGSGGGGSDIVLTQSPASLAVSLGQRATISCRASESVDSYGN |
| | SFMHWYQQKPGQPPKLLIYRASNLESGIPARFSGSGSRTDFTLTVNPVEADDVATY |
| | YCQQSNEDPWTFGGGTKLEIK [SEQ ID NO: 77] |
| scFv | DIVLTQSPASLAVSLGQRATISCRASESVDSYGNSFMHWYQQKPGQPPKLLIYRAS |
| V_{L}-V_{H} | NLESGIPARFSGSGSRTDFTLTVNPVEADDVATYYCQQSNEDPWTFGGGTKLEIKG |
| | GGGSGGGGSGGGGSQVQLQQPGAELVKPGASVKLSCKASGYIFISYWMHWVKQRPG |
| | QGLEWIGEINPRNGRTNYNEKFKSKATLTVDKSSSTAYMQLTSLTSEDSAVYYCAR |
| | GGDYGGSFDYWGQGTTLTVSS [SEQ ID NO: 78] |
| DNA | CAGGTACAGCTCCAGCAACCGGGGGCGGAGTTGGTAAAGCCGGGTGCGTCCGTAAA |
| scFv | ATTGTCATGCAAGGCCAGCGGCTATATCTTCATTAGCTACTGGATGCACTGGGTTA |
| V_{H}-V_{L} | AGCAACGGCCCGGCCAGGGTCTTGAGTGGATCGGGGAAATTAATCCGAGGAACGGC |
| | CGGACGAACTACAACGAAAAATTCAAATCTAAAGCTACTCTCACCGTGGATAAGTC |
| | TTCTTCTACAGCTTATATGCAGTTGACCAGTCTCACCAGTGAAGACAGTGCGGTAT |
| | ATTACTGTGCAAGGGGTGGAGACTATGGGGGCTCATTCGATTATTGGGGGCAAGGT |
| | ACTACACTGACTGTATCTTCCGGCGGTGGTGGCTCAGGCGGCGGAGGATCCGGGGG |
| | GGGGGGATCCGACATAGTACTCACTCAGAGCCCAGCTTCACTGGCTGTGTCTTTGG |
| | GACAACGCGCAACTATATCCTGCCGGGCGTCAGAATCTGTAGATAGCTATGGAAAC |
| | AGTTTTATGCATTGGTATCAGCAAAAACCCGGTCAGCCCCCTAAGTTGCTCATTTA |
| | TAGAGCCTCAAACCTCGAATCAGGGATTCCGGCACGCTTCAGTGGGTCTGGTAGTA |
| | GAACGGATTTCACACTTACAGTCAATCCAGTCGAGGCCGACGATGTGGCTACATAC |
| | TACTGCCAGCAATCCAATGAAGATCCTTGGACATTTGGTGGCGGGACAAAGCTGGA |
| | AATAAAG [SEQ ID NO: 79] |
| DNA | GACATAGTACTCACTCAGAGCCCAGCTTCACTGGCTGTGTCTTTGGGACAACGCGC |
| scFv | AACTATATCCTGCCGGGCGTCAGAATCTGTAGATAGCTATGGAAACAGTTTTATGC |
| V_{L}-V_{H} | ATTGGTATCAGCAAAAACCCGGTCAGCCCCCTAAGTTGCTCATTTATAGAGCCTCA |
| | AACCTCGAATCAGGGATTCCGGCACGCTTCAGTGGGTCTGGTAGTAGAACGGATTT |
| | CACACTTACAGTCAATCCAGTCGAGGCCGACGATGTGGCTACATACTACTGCCAGC |
| | AATCCAATGAAGATCCTTGGACATTTGGTGGCGGGACAAAGCTGGAAATAAAGGGC |
| | GGTGGTGGCTCAGGCGGCGGAGGATCCGGGGGGGGGGGATCCCAGGTACAGCTCCA |
| | GCAACCGGGGGCGGAGTTGGTAAAGCCGGGTGCGTCCGTAAAATTGTCATGCAAGG |
| | CCAGCGGCTATATCTTCATTAGCTACTGGATGCACTGGGTTAAGCAACGGCCCGGC |
| | CAGGGTCTTGAGTGGATCGGGGAAATTAATCCGAGGAACGGCCGGACGAACTACAA |
| | CGAAAAATTCAAATCTAAAGCTACTCTCACCGTGGATAAGTCTTCTTCTACAGCTT |
| | ATATGCAGTTGACCAGTCTCACCAGTGAAGACAGTGCGGTATATTACTGTGCAAGG |
| | GGTGGAGACTATGGGGGCTCATTCGATTATTGGGGGCAAGGTACTACACTGACTGT |
| | ATCTTCC |
| | [SEQ ID NO: 80] |

In certain embodiments, the anti-L1CAM antibody is an scFv, an scFv-Fc fusion protein or a full-length human IgG (or antibody fragment thereof) with V_{H} and V_{L} regions or CDRs selected from Table 40. In certain embodiments, the anti-L1CAM scFv comprises a V_{H} comprising the amino acid sequence set forth in SEQ ID NO: 87. In certain embodiments, the anti-L1CAM scFv comprises a V_{L} comprising the amino acid sequence set forth in SEQ ID NO: 88. SEQ ID NO: 87 and 88 are provided in Table 40. In certain embodiments, the scFv is designated as “14A10”.

In certain embodiments, the anti-L1CAM scFv comprises a V_{H} comprising the amino acid sequence set forth in SEQ ID NO: 87 and a V_{L} comprising the amino acid sequence set forth in SEQ ID NO: 88.

In certain embodiments, the anti-L1CAM scFv comprises a V_{H} Comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof. SEQ ID NOs: 81-83 are provided in Table 40.

In certain embodiments, the anti-L1CAM scFv comprises a V_{L} comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof. SEQ ID NOs: 84-86 are provided in Table 40.

In certain embodiments, the anti-L1CAM scFv comprises a V_{H} Comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, a CDR3 comprising the amino acid sequence set forth in SEQ ID NO: 83 or a conservative modification thereof; and a V_{L} comprising a CDR1 comprising the amino acid sequence set forth in SEQ ID NO: 84 or a conservative modification thereof, a CDR2 comprising the amino acid sequence set forth in SEQ ID NO: 85 or a conservative modification, and a CDR3 comprising the amino acid sequence set forth in SEQ ID NO: 86 or a conservative modification thereof.

In certain embodiments, the anti-L1CAM scFv comprises a V_{H} Comprising a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and a V_{L} comprising a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain embodiments, the anti-L1CAM scFv comprises a V_{H} comprising the amino acid sequence set forth in SEQ ID NO: 87, and a V_{L} comprising the amino acid sequence set forth in SEQ ID NO: 88. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}. In certain embodiments, the anti-L1CAM scFv comprises the amino acid sequence set forth in SEQ ID NO: 89. In certain embodiments, the anti-L1CAM scFv comprises the amino acid sequence set forth in SEQ ID NO: 90.

TABLE 40

| CDRs | 1 | 2 | 3 |
|---|---|---|---|
| V_{H} | GFIFSSYN | ISSSSSTT | ASLYSGYDYYYYYGMDV |
| | [SEQ ID NO: 81] | [SEQ ID NO: 82] | [SEQ ID NO: 83] |
| V_{L} | QSLVHSDGNTY | KIS | MQATQFPFT |
| | [SEQ ID NO: 84] | [SEQ ID NO: 85] | [SEQ ID NO: 86] |

| | |
|---|---|
| Full | EVQMVESGGGLVQPGGSLRLSCAAPGFIFSSYNMNWVRQAPGKGLEWVSYISSSSS |
| V_{H} | TTYYADSVKGRFTISRDNAKNSLYLQMNSLRDEDTAVYYCASLYSGYDYYYYYGMD |
| | VWGQGTTVTVSS |
| | [SEQ ID NO: 87] |
| Full | DIVMTQTPLSSPVSLGQPASISCRSSQSLVHSDGNTYLSWLQQRPGQPPRVLIYKI |
| V_{L} | SKRFSGVPDRFSGSGAGTDFTLKISRVEAEDVGVYYCMQATQFPFTFGPGTKVDIK |
| | [SEQ ID NO: 88] |
| scFv | EVQMVESGGGLVQPGGSLRLSCAAPGFIFSSYNMNWVRQAPGKGLEWVSYISSSSS |
| V_{H}-V_{L} | TTYYADSVKGRFTISRDNAKNSLYLQMNSLRDEDTAVYYCASLYSGYDYYYYYGMD |
| | VWGQGTTVTVSSGGGGSGGGGSGGGGSDIVMTQTPLSSPVSLGQPASISCRSSQSL |
| | VHSDGNTYLSWLQQRPGQPPRVLIYKISKRFSGVPDRFSGSGAGTDFTLKISRVEA |
| | EDVGVYYCMQATQFPFTFGPGTKVDIK |
| | [SEQ ID NO: 89] |
| scFv | DIVMTQTPLSSPVSLGQPASISCRSSQSLVHSDGNTYLSWLQQRPGQPPRVLIYKI |
| V_{L}-V_{H} | SKRFSGVPDRFSGSGAGTDFTLKISRVEAEDVGVYYCMQATQFPFTFGPGTKVDIK |
| | GGGGSGGGGSGGGGSEEVQMVESGGGLVQPGGSLRLSCAAPGFIFSSYNMNWVRQA |
| | PGKGLEWVSYISSSSSTTYYADSVKGRFTISRDNAKNSLYLQMNSLRDEDTAVYYC |
| | ASLYSGYDYYYYYGMDVWGQGTTVTVSS |
| | [SEQ ID NO: 90] |
| DNA | GAAGTCCAGATGGTGGAGTCAGGAGGGGGTCTGGTCCAACCGGGGGGTAGCCTGAG |
| scFv | GTTGTCCTGCGCGGCACCAGGATTTATCTTCTCAAGCTATAACATGAACTGGGTAC |
| V_{H}-V_{L} | GCCAAGCACCTGGCAAAGGACTGGAATGGGTAAGTTACATTTCTTCCAGTTCATCA |
| | ACAACTTACTATGCAGACAGTGTGAAAGGCCGGTTTACTATAAGCCGGGACAATGC |
| | TAAGAACTCACTCTATCTCCAAATGAACTCCCTCCGCGACGAGGACACAGCTGTCT |
| | ATTACTGTGCTAGTCTCTACTCTGGCTACGATTACTATTACTATTATGGAATGGAT |
| | GTGTGGGGGCAAGGCACTACTGTCACAGTCTCAAGCGGCGGTGGTGGCTCAGGCGG |
| | CGGAGGATCCGGGGGGGGGGGATCCGATATTGTTATGACGCAAACGCCCTTGTCAT |
| | CACCTGTATCATTGGGTCAGCCGGCTTCCATATCCTGCCGATCTAGTCAATCCTTG |
| | GTGCACTCAGACGGGAATACCTACCTTTCATGGCTCCAGCAGAGACCCGGGCAACC |
| | ACCGAGAGTGTTGATCTATAAAATTTCTAAACGGTTTAGCGGGGTCCCTGACAGAT |
| | TCTCCGGTTCCGGGGCTGGAACTGATTTTACACTTAAAATATCCCGAGTCGAAGCG |
| | GAAGACGTGGGGGTGTATTATTGCATGCAAGCAACTCAATTTCCCTTCACATTTGG |
| | GCCTGGTACAAAAGTGGACATCAAG |
| | [SEQ ID NO: 91] |
| DNA | GATATTGTTATGACGCAAACGCCCTTGTCATCACCTGTATCATTGGGTCAGCCGGC |
| scFv | TTCCATATCCTGCCGATCTAGTCAATCCTTGGTGCACTCAGACGGGAATACCTACC |
| V_{L}-V_{H} | TTTCATGGCTCCAGCAGAGACCCGGGCAACCACCGAGAGTGTTGATCTATAAAATT |
| | TCTAAACGGTTTAGCGGGGTCCCTGACAGATTCTCCGGTTCCGGGGCTGGAACTGA |
| | TTTTACACTTAAAATATCCCGAGTCGAAGCGGAAGACGTGGGGGTGTATTATTGCA |
| | TGCAAGCAACTCAATTTCCCTTCACATTTGGGCCTGGTACAAAAGTGGACATCAAG |
| | GGCGGTGGTGGCTCAGGCGGCGGAGGATCCGGGGGGGGGGGATCCGAAGTCCAGAT |
| | GGTGGAGTCAGGAGGGGGTCTGGTCCAACCGGGGGGTAGCCTGAGGTTGTCCTGCG |
| | CGGCACCAGGATTTATCTTCTCAAGCTATAACATGAACTGGGTACGCCAAGCACCT |
| | GGCAAAGGACTGGAATGGGTAAGTTACATTTCTTCCAGTTCATCAACAACTTACTA |
| | TGCAGACAGTGTGAAAGGCCGGTTTACTATAAGCCGGGACAATGCTAAGAACTCAC |
| | TCTATCTCCAAATGAACTCCCTCCGCGACGAGGACACAGCTGTCTATTACTGTGCT |
| | AGTCTCTACTCTGGCTACGATTACTATTACTATTATGGAATGGATGTGTGGGGGCA |
| | AGGCACTACTGTCACAGTCTCAAGC |
| | [SEQ ID NO: 92] |

In certain embodiments, the anti-L1CAM antibody is an scFv, an scFv-Fc fusion protein or a full-length human IgG (or antibody fragment thereof) with V_{H} and V_{L} regions or CDRs selected from Table 41. In certain embodiments, the anti-L1CAM scFv comprises a V_{H} comprising the amino acid sequence set forth in SEQ ID NO: 99. In certain embodiments, the anti-L1CAM scFv comprises a V_{L} comprising the amino acid sequence set forth in SEQ ID NO: 100. SEQ ID NO: 99 and 100 are provided in Table 41. In certain embodiments, the scFv is designated as “76H05”.

In certain embodiments, the anti-L1CAM scFv comprises a V_{H} comprising the amino acid sequence set forth in SEQ ID NO: 99 and a V_{L} comprising the amino acid sequence set forth in SEQ ID NO: 100.

In certain embodiments, the anti-L1CAM scFv comprises a V_{H} Comprising a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof. SEQ ID NOs: 93-95 are provided in Table 41.

In certain embodiments, the anti-L1CAM scFv comprises a V_{L} comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof. SEQ ID NOs: 96-98 are provided in Table 41.

In certain embodiments, the anti-L1CAM scFv comprises a V_{H} Comprising a CDR1 comprising the amino acid sequence set forth in SEQ ID NO: 93 or a conservative modification thereof, a CDR2 comprising the amino acid sequence set forth in SEQ ID NO: 94 or a conservative modification thereof, a CDR3 comprising the amino acid sequence set forth in SEQ ID NO: 95 or a conservative modification thereof; and a V_{L} comprising a CDR1 comprising the amino acid sequence set forth in SEQ ID NO: 96 or a conservative modification thereof, a CDR2 comprising the amino acid sequence set forth in SEQ ID NO: 97 or a conservative modification, and a CDR3 comprising the amino acid sequence set forth in SEQ ID NO: 98 or a conservative modification thereof.

In certain embodiments, the anti-L1CAM scFv comprises a V_{H} Comprising a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 95; and a V_{L} comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

In certain embodiments, the anti-L1CAM scFv comprises a V_{H} comprising the amino acid sequence set forth in SEQ ID NO: 99, and a V_{L} comprising the amino acid sequence set forth in SEQ ID NO: 100. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}. In certain embodiments, the anti-L1CAM scFv comprises the amino acid sequence set forth in SEQ ID NO: 101. In certain embodiments, the anti-L1CAM scFv comprises the amino acid sequence set forth in SEQ ID NO: 102.

TABLE 41

| CDRs | 1 | 2 | 3 |
|---|---|---|---|
| V_{H} | GFSLTSYG | IWGDGST | ATSTVGYAVDY |
| | [SEQ ID NO: 93] | [SEQ ID NO: 94] | [SEQ ID NO: 95] |
| V_{L} | ENIYSN | AAT | QHFWGSPYT |
| | [SEQ ID NO: 96] | [SEQ ID NO: 97] | [SEQ ID NO: 98] |

| | |
|---|---|
| Full | QVQLKESGPGLVAPSQSLSITCTISGFSLTSYGVHWVRQPPGKGLEWLVVIWGDGS |
| V_{H} | TSYNSALKSRLSISKDNSKSQVFLKMNSLQTDDTAMYFCATSTVGYAVDYWGQGTS |
| | VTVSS |
| | [SEQ ID NO: 99] |
| Full | DIQMTQSPASLSVSVGETVTITCRASENIYSNLAWYQQRQGKSPQLLVYAATNLAD |
| V_{L} | GVPSRFSGSGSGTQYSLKINSLQSEDFGSYYCQHFWGSPYTFGGGTKLEIK |
| | [SEQ ID NO: 100] |
| scFv | QVQLKESGPGLVAPSQSLSITCTISGFSLTSYGVHWVRQPPGKGLEWLVVIWGDGS |
| V_{H}-V_{L} | TSYNSALKSRLSISKDNSKSQVFLKMNSLQTDDTAMYFCATSTVGYAVDYWGQGTS |
| | VTVSSGGGGSGGGGSGGGGSDIQMTQSPASLSVSVGETVTITCRASENIYSNLAWY |
| | QQRQGKSPQLLVYAATNLADGVPSRFSGSGSGTQYSLKINSLQSEDFGSYYCQHFW |
| | GSPYTFGGGTKLEIK |
| | [SEQ ID NO: 101] |
| scFv | DIQMTQSPASLSVSVGETVTITCRASENIYSNLAWYQQRQGKSPQLLVYAATNLAD |
| V_{L}-V_{H} | GVPSRFSGSGSGTQYSLKINSLQSEDFGSYYCQHFWGSPYTFGGGTKLEIKGGGGS |
| | GGGGSGGGGSQVQLKESGPGLVAPSQSLSITCTISGFSLTSYGVHWVRQPPGKGLE |
| | WLVVIWGDGSTSYNSALKSRLSISKDNSKSQVFLKMNSLQTDDTAMYFCATSTVGY |
| | AVDYWGQGTSVTVSS |
| | [SEQ ID NO: 102] |
| DNA | CAAGTCCAGCTCAAGGAGAGCGGTCCAGGACTCGTAGCACCCTCTCAATCTTTGTC |
| scFv | TATCACTTGTACCATAAGCGGTTTTTCCTTGACAAGTTATGGCGTACATTGGGTGA |
| V_{H}-V_{L} | GACAACCGCCAGGTAAAGGACTCGAATGGTTGGTTGTTATCTGGGGGGATGGGAGT |
| | ACCAGCTACAACAGCGCACTCAAGTCTCGCCTTTCAATTTCTAAGGACAACAGTAA |
| | ATCACAGGTTTTTCTGAAGATGAATAGCCTGCAAACCGACGACACGGCGATGTATT |
| | TCTGTGCGACTTCTACGGTAGGTTATGCCGTCGACTATTGGGGTCAGGGTACGTCA |
| | GTTACCGTAAGTAGCGGCGGTGGTGGCTCAGGCGGCGGAGGATCCGGGGGGGGGGG |
| | ATCCGACATCCAGATGACCCAGTCACCGGCCAGTTTGTCCGTTTCCGTAGGCGAGA |
| | CCGTAACCATCACATGCCGAGCAAGCGAGAATATCTATTCCAACCTGGCCTGGTAT |
| | CAGCAGAGACAAGGCAAAAGTCCGCAACTTTTGGTTTATGCTGCCACTAATTTGGC |
| | CGATGGGGTCCCGAGTCGATTTTCTGGCTCTGGCTCCGGGACGCAGTATTCCCTGA |
| | AGATAAACTCTTTGCAGTCAGAAGATTTTGGTTCATATTATTGTCAGCATTTCTGG |
| | GGCAGCCCGTACACATTTGGAGGTGGGACCAAATTGGAAATAAAA |
| | [SEQ ID NO: 103] |
| DNA | GACATCCAGATGACCCAGTCACCGGCCAGTTTGTCCGTTTCCGTAGGCGAGACCGT |
| scFv | AACCATCACATGCCGAGCAAGCGAGAATATCTATTCCAACCTGGCCTGGTATCAGC |
| V_{L}-V_{H} | AGAGACAAGGCAAAAGTCCGCAACTTTTGGTTTATGCTGCCACTAATTTGGCCGAT |
| | GGGGTCCCGAGTCGATTTTCTGGCTCTGGCTCCGGGACGCAGTATTCCCTGAAGAT |
| | AAACTCTTTGCAGTCAGAAGATTTTGGTTCATATTATTGTCAGCATTTCTGGGGCA |
| | GCCCGTACACATTTGGAGGTGGGACCAAATTGGAAATAAAAGGCGGTGGTGGCTCA |
| | GGCGGCGGAGGATCCGGGGGGGGGGGATCCCAAGTCCAGCTCAAGGAGAGCGGTCC |
| | AGGACTCGTAGCACCCTCTCAATCTTTGTCTATCACTTGTACCATAAGCGGTTTTT |
| | CCTTGACAAGTTATGGCGTACATTGGGTGAGACAACCGCCAGGTAAAGGACTCGAA |
| | TGGTTGGTTGTTATCTGGGGGGATGGGAGTACCAGCTACAACAGCGCACTCAAGTC |
| | TCGCCTTTCAATTTCTAAGGACAACAGTAAATCACAGGTTTTTCTGAAGATGAATA |
| | GCCTGCAAACCGACGACACGGCGATGTATTTCTGTGCGACTTCTACGGTAGGTTAT |
| | GCCGTCGACTATTGGGGTCAGGGTACGTCAGTTACCGTAAGTAGC |
| | [SEQ ID NO: 104] |

In certain embodiments, the anti-L1CAM antibody is an scFv, an scFv-Fc fusion protein or a full-length human IgG (or antibody fragment thereof) with V_{H} and V_{L} regions or CDRs selected from Table 42. In certain embodiments, the anti-L1CAM scFv comprises a V_{H} comprising the amino acid sequence set forth in SEQ ID NO: 110. In certain embodiments, the anti-L1CAM scFv comprises a V_{L} comprising the amino acid sequence set forth in SEQ ID NO: 111. SEQ ID NO: 110 and 111 are provided in Table 42. In certain embodiments, the scFv is designated as “18H05”.

In certain embodiments, the anti-L1CAM scFv comprises a V_{H} comprising the amino acid sequence set forth in SEQ ID NO: 110 and a V_{L} comprising the amino acid sequence set forth in SEQ ID NO: 111.

In certain embodiments, the anti-L1CAM scFv comprises a V_{H} Comprising a CDR1 comprising the amino acid sequence set forth in SEQ ID NO: 105 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 105-107 are provided in Table 42.

In certain embodiments, the anti-L1CAM scFv comprises a V_{L} comprising a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof. SEQ ID NOs: 39, 108, and 109 are provided in Table 42.

In certain embodiments, the anti-L1CAM scFv comprises a V_{H} Comprising a CDR1 comprising the amino acid sequence set forth in SEQ ID NO: 105 or a conservative modification thereof, a CDR2 comprising the amino acid sequence set forth in SEQ ID NO: 106 or a conservative modification thereof, a CDR3 comprising the amino acid sequence set forth in SEQ ID NO: 107 or a conservative modification thereof; and a V_{L} Comprising a CDR1 comprising the amino acid sequence set forth in SEQ ID NO: 108 or a conservative modification thereof, a CDR2 comprising the amino acid sequence set forth in SEQ ID NO: 39 or a conservative modification, and a CDR3 comprising the amino acid sequence set forth in SEQ ID NO: 109 or a conservative modification thereof.

In certain embodiments, the anti-L1CAM scFv comprises a V_{H} Comprising a CDR1 comprising the amino acid sequence set forth in SEQ ID NO: 105, a CDR2 comprising the amino acid sequence set forth in SEQ ID NO: 106, a CDR3 comprising the amino acid sequence set forth in SEQ ID NO: 107; and a V_{L} comprising a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109.

In certain embodiments, the anti-L1CAM scFv comprises a V_{H} comprising the amino acid sequence set forth in SEQ ID NO: 110, and a V_{L} comprising the amino acid sequence set forth in SEQ ID NO: 111. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}. In certain embodiments, the anti-L1CAM scFv comprises the amino acid sequence set forth in SEQ ID NO: 112. In certain embodiments, the anti-L1CAM scFv comprises the amino acid sequence set forth in SEQ ID NO: 113.

TABLE 42

| CDRs | 1 | 2 | 3 |
|---|---|---|---|
| V_{H} | GDSISSYY | IYTSGTT | ASHSSGWYDAFDI |
| | [SEQ ID NO: 105] | [SEQ ID NO: 106] | [SEQ ID NO: 107] |
| V_{L} | QSVLYSSNNKNY | WAS | QQYYSTPPT |
| | [SEQ ID NO: 108] | [SEQ ID NO: 39] | [SEQ ID NO: 109] |

| | |
|---|---|
| Full | QVQLQESGPGLVKPSETLSLTCTVSGDSISSYYWTWIRQPAGKGLEWIGRIYTSGT |
| V_{H} | TNYNPSLKSRVTMSVDTSKNQFSLKLSSVTAADTAVYYCASHSSGWYDAFDIWGQG |
| | TMVTVSS |
| | [SEQ ID NO: 110] |
| Full | DIVMTQSPDSLAVSLGERATINCKSSQSVLYSSNNKNYLAWYQQKPGQPPKLFIYW |
| V_{L} | ASTRESGVPDRFSGSGSGTDFTLTISSLQAEDVAVYYCQQYYSTPPTFGQGTKVEI |
| | K |
| | [SEQ ID NO: 111] |
| scFv | QVQLQESGPGLVKPSETLSLTCTVSGDSISSYYWTWIRQPAGKGLEWIGRIYTSGT |
| V_{H}-V_{L} | TNYNPSLKSRVTMSVDTSKNQFSLKLSSVTAADTAVYYCASHSSGWYDAFDIWGQG |
| | TMVTVSSGGGGSGGGGSGGGGSDIVMTQSPDSLAVSLGERATINCKSSQSVLYSSN |
| | NKNYLAWYQQKPGQPPKLFIYWASTRESGVPDRFSGSGSGTDFTLTISSLQAEDVA |
| | VYYCQQYYSTPPTFGQGTKVEIK |
| | [SEQ ID NO: 112] |
| scFv | DIVMTQSPDSLAVSLGERATINCKSSQSVLYSSNNKNYLAWYQQKPGQPPKLFIYW |
| V_{L}-V_{H} | ASTRESGVPDRFSGSGSGTDFTLTISSLQAEDVAVYYCQQYYSTPPTFGQGTKVEI |
| | KGGGGSGGGGSGGGGSQVQLQESGPGLVKPSETLSLTCTVSGDSISSYYWTWIRQP |
| | AGKGLEWIGRIYTSGTTNYNPSLKSRVTMSVDTSKNQFSLKLSSVTAADTAVYYCA |
| | SHSSGWYDAFDIWGQGTMVTVSS |
| | [SEQ ID NO: 113] |
| DNA | CAGGTACAGCTTCAAGAAAGCGGTCCAGGACTTGTGAAGCCGAGCGAGACGCTTTC |
| scFv | ACTGACTTGCACAGTCAGTGGGGACAGTATTAGTTCCTATTACTGGACATGGATAC |
| V_{H}-V_{L} | GACAACCAGCAGGGAAAGGTCTGGAATGGATTGGGCGCATTTACACCAGTGGAACT |
| | ACAAATTACAACCCATCCCTCAAATCCAGGGTTACGATGAGCGTCGACACGAGTAA |
| | GAATCAGTTTTCCCTGAAGCTCTCCAGTGTGACGGCTGCAGATACTGCAGTGTATT |
| | ATTGCGCTTCCCACTCAAGTGGTTGGTACGATGCTTTCGACATCTGGGGGCAGGGT |
| | ACTATGGTGACCGTGTCCTCAGGCGGTGGTGGCTCAGGCGGCGGAGGATCCGGGGG |
| | GGGGGGATCCGACATCGTAATGACCCAATCACCTGATTCTCTGGCTGTGAGCCTTG |
| | GTGAGAGGGCAACAATAAACTGCAAGAGCTCTCAGTCCGTACTTTATAGTAGCAAC |
| | AACAAGAACTATTTGGCGTGGTACCAGCAGAAGCCGGGTCAGCCGCCAAAGCTGTT |
| | TATATATTGGGCCTCTACCCGCGAATCTGGAGTACCGGACAGGTTTAGTGGTAGTG |
| | GCTCAGGTACGGATTTTACGCTCACCATTAGCTCTCTGCAAGCAGAGGACGTAGCC |
| | GTATATTATTGCCAGCAATATTATTCTACGCCACCCACCTTTGGTCAGGGGACGAA |
| | AGTTGAGATCAAG |
| | [SEQ ID NO: 114] |
| DNA | GACATCGTAATGACCCAATCACCTGATTCTCTGGCTGTGAGCCTTGGTGAGAGGGC |
| scFv | AACAATAAACTGCAAGAGCTCTCAGTCCGTACTTTATAGTAGCAACAACAAGAACT |
| V_{L}-V_{H} | ATTTGGCGTGGTACCAGCAGAAGCCGGGTCAGCCGCCAAAGCTGTTTATATATTGG |
| | GCCTCTACCCGCGAATCTGGAGTACCGGACAGGTTTAGTGGTAGTGGCTCAGGTAC |
| | GGATTTTACGCTCACCATTAGCTCTCTGCAAGCAGAGGACGTAGCCGTATATTATT |
| | GCCAGCAATATTATTCTACGCCACCCACCTTTGGTCAGGGGACGAAAGTTGAGATC |
| | AAGGGCGGTGGTGGCTCAGGCGGCGGAGGATCCGGGGGGGGGGGATCCCAGGTACA |
| | GCTTCAAGAAAGCGGTCCAGGACTTGTGAAGCCGAGCGAGACGCTTTCACTGACTT |
| | GCACAGTCAGTGGGGACAGTATTAGTTCCTATTACTGGACATGGATACGACAACCA |
| | GCAGGGAAAGGTCTGGAATGGATTGGGCGCATTTACACCAGTGGAACTACAAATTA |
| | CAACCCATCCCTCAAATCCAGGGTTACGATGAGCGTCGACACGAGTAAGAATCAGT |
| | TTTCCCTGAAGCTCTCCAGTGTGACGGCTGCAGATACTGCAGTGTATTATTGCGCT |
| | TCCCACTCAAGTGGTTGGTACGATGCTTTCGACATCTGGGGGCAGGGTACTATGGT |
| | GACCGTGTCCTCA |
| | [SEQ ID NO: 115] |

In certain embodiments, the anti-L1CAM antibody is an scFv, an scFv-Fc fusion protein or a full-length human IgG (or antibody fragment thereof) with V_{H} and V_{L} regions or CDRs selected from Table 43. In certain embodiments, the anti-L1CAM scFv comprises a V_{H} comprising the amino acid sequence set forth in SEQ ID NO: 122. In certain embodiments, the anti-L1CAM scFv comprises a V_{L} comprising the amino acid sequence set forth in SEQ ID NO: 123. SEQ ID NO: 122 and 123 are provided in Table 43. In certain embodiments, the scFv is designated as “76H12”.

In certain embodiments, the anti-L1CAM scFv comprises a V_{H} comprising the amino acid sequence set forth in SEQ ID NO: 122 and a V_{L} comprising the amino acid sequence set forth in SEQ ID NO: 123.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 43.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 43.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 122, and a V_{L} comprising the amino acid sequence set forth in SEQ ID NO: 123. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}. In certain embodiments, the anti-scFv comprises the amino acid sequence set forth in SEQ ID NO: 124. In certain embodiments, the anti-scFv comprises the amino acid sequence set forth in SEQ ID NO: 125.

TABLE 43

| CDRs | 1 | 2 | 3 |
|---|---|---|---|
| V_{H} | GYTFTSYW | VYPGSGST | TRPGNYEGFAY |
| | [SEQ ID NO: 116] | [SEQ ID NO: 117] | [SEQ ID NO: 118] |
| V_{L} | ESVDSYGSSF | LAS | QQNNEDPWT |
| | [SEQ ID NO: 119] | [SEQ ID NO: 120] | [SEQ ID NO: 121] |

| | |
|---|---|
| Full | QVQLQQPGSDLVRPGTSVKLSCKASGYTFTSYWMHWVKQRPGQGLEWIGNVYPGSG |
| V_{H} | STKYDEKFKTKATLTVDTSSNTAYMELSSLTSEDSAVYYCTRPGNYEGFAYWGQGT |
| | LVTVSA |
| | [SEQ ID NO: 122] |
| Full | NIVLTQSPASLAVSLGQRATISCRASESVDSYGSSFMHWYQQKPGQPPKLLIYLAS |
| V_{L} | NLESGVPARFSGSGSRTDFTLTIDPVEADDAATYYCQQNNEDPWTFGGGTKLEIK |
| | [SEQ ID NO: 123] |
| scFv | QVQLQQPGSDLVRPGTSVKLSCKASGYTFTSYWMHWVKQRPGQGLEWIGNVYPGSG |
| V_{H}-V_{L} | STKYDEKFKTKATLTVDTSSNTAYMELSSLTSEDSAVYYCTRPGNYEGFAYWGQGT |
| | LVTVSAGGGGSGGGGSGGGGSNIVLTQSPASLAVSLGQRATISCRASESVDSYGSS |
| | FMHWYQQKPGQPPKLLIYLASNLESGVPARFSGSGSRTDFTLTIDPVEADDAATYY |
| | CQQNNEDPWTFGGGTKLEIK |
| | [SEQ ID NO: 124] |
| scFv | NIVLTQSPASLAVSLGQRATISCRASESVDSYGSSFMHWYQQKPGQPPKLLIYLAS |
| V_{L}-V_{H} | NLESGVPARFSGSGSRTDFTLTIDPVEADDAATYYCQQNNEDPWTFGGGTKLEIKG |
| | GGGSGGGGSGGGGSQVQLQQPGSDLVRPGTSVKLSCKASGYTFTSYWMHWVKQRPG |
| | QGLEWIGNVYPGSGSTKYDEKFKTKATLTVDTSSNTAYMELSSLTSEDSAVYYCTR |
| | PGNYEGFAYWGQGTLVTVSA |
| | [SEQ ID NO: 125] |
| DNA | CAGGTGCAACTTCAGCAGCCCGGCTCTGATCTTGTGCGCCCTGGTACTTCTGTAAA |
| scFv | GCTTAGCTGTAAAGCTTCTGGTTATACGTTCACGAGTTATTGGATGCACTGGGTAA |
| V_{H}-V_{L} | AGCAGCGGCCTGGACAGGGATTGGAGTGGATCGGAAACGTGTACCCTGGCAGTGGT |
| | TCTACAAAGTATGACGAGAAATTTAAGACAAAAGCAACACTCACTGTCGATACGTC |
| | TTCAAACACAGCGTACATGGAGTTGAGTTCATTGACCTCCGAGGACAGCGCTGTAT |
| | ATTACTGCACACGCCCTGGAAACTACGAAGGTTTCGCGTATTGGGGGCAGGGGACC |
| | TTGGTCACAGTGTCAGCCGGCGGTGGTGGCTCAGGCGGCGGAGGATCCGGGGGGGG |
| | GGGATCCAACATAGTCCTTACGCAAAGTCCAGCTTCACTGGCAGTATCTCTGGGTC |
| | AACGCGCTACCATTAGTTGTCGCGCAAGCGAATCCGTCGATAGCTATGGGTCTTCC |
| | TTTATGCACTGGTATCAGCAGAAGCCGGGACAGCCGCCCAAGCTGCTGATCTACCT |
| | CGCGTCCAACTTGGAGTCAGGCGTTCCCGCACGGTTTAGTGGAAGCGGAAGTAGAA |
| | CTGATTTTACTCTTACGATTGATCCTGTCGAGGCTGATGATGCTGCTACATATTAT |
| | TGTCAGCAGAATAATGAGGATCCTTGGACCTTCGGCGGGGGAACCAAATTGGAAAT |
| | TAAA |
| | [SEQ ID NO: 126] |
| DNA | AACATAGTCCTTACGCAAAGTCCAGCTTCACTGGCAGTATCTCTGGGTCAACGCGC |
| scFv | TACCATTAGTTGTCGCGCAAGCGAATCCGTCGATAGCTATGGGTCTTCCTTTATGC |
| V_{L}-V_{H} | ACTGGTATCAGCAGAAGCCGGGACAGCCGCCCAAGCTGCTGATCTACCTCGCGTCC |
| | AACTTGGAGTCAGGCGTTCCCGCACGGTTTAGTGGAAGCGGAAGTAGAACTGATTT |
| | TACTCTTACGATTGATCCTGTCGAGGCTGATGATGCTGCTACATATTATTGTCAGC |
| | AGAATAATGAGGATCCTTGGACCTTCGGCGGGGGAACCAAATTGGAAATTAAAGGC |
| | GGTGGTGGCTCAGGCGGCGGAGGATCCGGGGGGGGGGGATCCCAGGTGCAACTTCA |
| | GCAGCCCGGCTCTGATCTTGTGCGCCCTGGTACTTCTGTAAAGCTTAGCTGTAAAG |
| | CTTCTGGTTATACGTTCACGAGTTATTGGATGCACTGGGTAAAGCAGCGGCCTGGA |
| | CAGGGATTGGAGTGGATCGGAAACGTGTACCCTGGCAGTGGTTCTACAAAGTATGA |
| | CGAGAAATTTAAGACAAAAGCAACACTCACTGTCGATACGTCTTCAAACACAGCGT |
| | ACATGGAGTTGAGTTCATTGACCTCCGAGGACAGCGCTGTATATTACTGCACACGC |
| | CCTGGAAACTACGAAGGTTTCGCGTATTGGGGGCAGGGGACCTTGGTCACAGTGTC |
| | AGCC |
| | [SEQ ID NO: 127] |

In certain embodiments, the anti-L1CAM antibody is an scFv, an scFv-Fc fusion protein or a full-length human IgG (or antibody fragment thereof) with V_{H} and V_{L} regions or CDRs selected from Table 44. In certain embodiments, the anti-L1CAM scFv comprises a V_{H} comprising the amino acid sequence set forth in SEQ ID NO: 128. In certain embodiments, the anti-L1CAM scFv comprises a V_{L} comprising the amino acid sequence set forth in SEQ ID NO: 129. SEQ ID NO: 128 and 129 are provided in Table 44. In certain embodiments, the scFv is designated as “76H12v2”.

In certain embodiments, the anti-L1CAM scFv comprises a V_{H} comprising the amino acid sequence set forth in SEQ ID NO: 128 and a V_{L} comprising the amino acid sequence set forth in SEQ ID NO: 129.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 44.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 44.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 128, and a V_{L} comprising the amino acid sequence set forth in SEQ ID NO: 129. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 44

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWMGNVYPGSGSTKYAQKFQGRVTMTRDT |
| | STSTVYMELSSLRSEDTAVYYCARPGNYEGFAYWGQG |
| | TLVTVSS |
| | [SEQ ID NO: 128] |
| Full | NIQLTQSPSSLSASVGDRVTITCRASESVDSYGSSFM |
| V_{L} | HWYQQKPGKPPKLLIYLASNLESGVPSRFSGSGSGTD |
| | FTLTISSLQPEDFATYYCQQNNEDPWTFGQGTKLEIK |
| | [SEQ ID NO: 129] |

In certain embodiments, the anti-L1CAM antibody is an scFv, an scFv-Fc fusion protein or a full-length human IgG (or antibody fragment thereof) with V_{H} and V_{L} regions or CDRs selected from Table 45. In certain embodiments, the anti-L1CAM scFv comprises a V_{H} comprising the amino acid sequence set forth in SEQ ID NO: 130. In certain embodiments, the anti-L1CAM scFv comprises a V_{L} comprising the amino acid sequence set forth in SEQ ID NO: 131. SEQ ID NO: 130 and 131 are provided in Table 45. In certain embodiments, the scFv is designated as “76H12v3”.

In certain embodiments, the anti-L1CAM scFv comprises a V_{H} comprising the amino acid sequence set forth in SEQ ID NO: 130 and a V_{L} comprising the amino acid sequence set forth in SEQ ID NO: 131.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 45.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 44.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 130, and a V_{L} comprising the amino acid sequence set forth in SEQ ID NO: 131. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 45

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STSTVYMELSSLRSEDTAVYYCARPGNYEGFAYWGQG |
| | TLVTVSS |
| | [SEQ ID NO: 130] |
| Full | DIQLTQSPSSLSASVGDRVTITCRASESVDSYGSSFM |
| V_{L} | HWYQQKPGKPPKLLIYLASNLESGVPSRFSGSGSGTD |
| | FTLTISSLQPEDFATYYCQQNNEDPWTFGQGTKLEIK |
| | [SEQ ID NO: 131] |

In certain embodiments, the anti-L1CAM antibody is an scFv, an scFv-Fc fusion protein or a full-length human IgG (or antibody fragment thereof) with V_{H} and V_{L} regions or CDRs selected from Table 46. In certain embodiments, the anti-L1CAM scFv comprises a V_{H} comprising the amino acid sequence set forth in SEQ ID NO: 130. In certain embodiments, the anti-L1CAM scFv comprises a V_{L} comprising the amino acid sequence set forth in SEQ ID NO: 132. SEQ ID NO: 130 and 132 are provided in Table 46. In certain embodiments, the scFv is designated as “76H12v4”.

In certain embodiments, the anti-L1CAM scFv comprises a V_{H} comprising the amino acid sequence set forth in SEQ ID NO: 130 and a V_{L} comprising the amino acid sequence set forth in SEQ ID NO: 132.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 44.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 46.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 130, and a V_{L} comprising the amino acid sequence set forth in SEQ ID NO: 132. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 46

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STSTVYMELSSLRSEDTAVYYCARPGNYEGFAYWGQG |
| | TLVTVSS |
| | [SEQ ID NO: 130] |
| Full | DIQLTQSPSSLSASVGDRVTITCRASESVDSYGSSFM |
| V_{L} | HWYQQKPGKPPKLLIYLASNLESGVPSRFSGSGSRTD |
| | FTLTISSLQPEDFATYYCQQNNEDPWTFGQGTKLEIK |
| | [SEQ ID NO: 132] |

In certain embodiments, the anti-L1CAM antibody is an scFv, an scFv-Fc fusion protein or a full-length human IgG (or antibody fragment thereof) with V_{H} and V_{L} regions or CDRs selected from Table 47. In certain embodiments, the anti-L1CAM scFv comprises a V_{H} comprising the amino acid sequence set forth in SEQ ID NO: 130. In certain embodiments, the anti-L1CAM scFv comprises a V_{L} comprising the amino acid sequence set forth in SEQ ID NO: 133. SEQ ID NO: 130 and 133 are provided in Table 47. In certain embodiments, the scFv is designated as “76H12v5”.

In certain embodiments, the anti-L1CAM scFv comprises a V_{H} comprising the amino acid sequence set forth in SEQ ID NO: 130 and a V_{L} comprising the amino acid sequence set forth in SEQ ID NO: 133.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 47.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 47.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 130, and a V_{L} comprising the amino acid sequence set forth in SEQ ID NO: 133. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 47

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STSTVYMELSSLRSEDTAVYYCARPGNYEGFAYWGQG |
| | TLVTVSS |
| | [SEQ ID NO: 130] |
| Full | NIQLTQSPSSLSASVGDRVTITCRASESVDSYGSSFM |
| V_{L} | HWYQQKPGKPPKLLIYLASNLESGVPSRFSGSGSGTD |
| | FTLTISSLQPEDFATYYCQQNNEDPWTFGQGTKLEIK |
| | [SEQ ID NO: 133] |

In certain embodiments, the anti-L1CAM antibody is an scFv, an scFv-Fc fusion protein or a full-length human IgG (or antibody fragment thereof) with V_{H} and V_{L} regions or CDRs selected from Table 48. In certain embodiments, the anti-L1CAM scFv comprises a V_{H} comprising the amino acid sequence set forth in SEQ ID NO: 134. In certain embodiments, the anti-L1CAM scFv comprises a V_{L} comprising the amino acid sequence set forth in SEQ ID NO: 131. SEQ ID NO: 134 and 131 are provided in Table 48. In certain embodiments, the scFv is designated as “76H12v6”.

In certain embodiments, the anti-L1CAM scFv comprises a V_{H} comprising the amino acid sequence set forth in SEQ ID NO: 134 and a V_{L} comprising the amino acid sequence set forth in SEQ ID NO: 131.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 48.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 48.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 134, and a V_{L} comprising the amino acid sequence set forth in SEQ ID NO: 131. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 48

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STSTAYMELSSLRSEDTAVYYCTRPGNYEGFAYWGQG |
| | TLVTVSS |
| | [SEQ ID NO: 134] |
| Full | DIQLTQSPSSLSASVGDRVTITCRASESVDSYGSSFM |
| V_{L} | HWYQQKPGKPPKLLIYLASNLESGVPSRFSGSGSGTD |
| | FTLTISSLQPEDFATYYCQQNNEDPWTFGQGTKLEIK |
| | [SEQ ID NO: 131] |

In certain embodiments, the anti-L1CAM antibody is an scFv, an scFv-Fc fusion protein or a full-length human IgG (or antibody fragment thereof) with V_{H} and V_{L} regions or CDRs selected from Table 49. In certain embodiments, the anti-L1CAM scFv comprises a V_{H} comprising the amino acid sequence set forth in SEQ ID NO: 134. In certain embodiments, the anti-L1CAM scFv comprises a V_{L} comprising the amino acid sequence set forth in SEQ ID NO: 132. SEQ ID NO: 134 and 132 are provided in Table 49. In certain embodiments, the scFv is designated as “76H12v7”.

In certain embodiments, the anti-L1CAM scFv comprises a V_{H} comprising the amino acid sequence set forth in SEQ ID NO: 128 and a V_{L} comprising the amino acid sequence set forth in SEQ ID NO: 129.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 49.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 49.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 134, and a V_{L} comprising the amino acid sequence set forth in SEQ ID NO: 132. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 49

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STSTAYMELSSLRSEDTAVYYCTRPGNYEGFAYWGQG |
| | TLVTVSS |
| | [SEQ ID NO: 134] |
| Full | DIQLTQSPSSLSASVGDRVTITCRASESVDSYGSSFM |
| V_{L} | HWYQQKPGKPPKLLIYLASNLESGVPSRFSGSGSRTD |
| | FTLTISSLQPEDFATYYCQQNNEDPWTFGQGTKLEIK |
| | [SEQ ID NO: 132] |

In certain embodiments, the anti-L1CAM antibody is an scFv, an scFv-Fc fusion protein or a full-length human IgG (or antibody fragment thereof) with V_{H} and V_{L} regions or CDRs selected from Table 50. In certain embodiments, the anti-L1CAM scFv comprises a V_{H} comprising the amino acid sequence set forth in SEQ ID NO: 134. In certain embodiments, the anti-L1CAM scFv comprises a V_{L} comprising the amino acid sequence set forth in SEQ ID NO: 133. SEQ ID NO: 134 and 133 are provided in Table 50. In certain embodiments, the scFv is designated as “76H12v8”.

In certain embodiments, the anti-L1CAM scFv comprises a V_{H} comprising the amino acid sequence set forth in SEQ ID NO: 134 and a V_{L} comprising the amino acid sequence set forth in SEQ ID NO: 133.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 50.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 50.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 134, and a V_{L} comprising the amino acid sequence set forth in SEQ ID NO: 133. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 50

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STSTAYMELSSLRSEDTAVYYCTRPGNYEGFAYWGQG |
| | TLVTVSS |
| | [SEQ ID NO: 134] |
| Full | NIQLTQSPSSLSASVGDRVTITCRASESVDSYGSSFM |
| V_{L} | HWYQQKPGKPPKLLIYLASNLESGVPSRFSGSGSGTD |
| | FTLTISSLQPEDFATYYCQQNNEDPWTFGQGTKLEIK |
| | [SEQ ID NO: 133] |

In certain embodiments, the anti-L1CAM antibody is an scFv, an scFv-Fc fusion protein or a full-length human IgG (or antibody fragment thereof) with V_{H} and V_{L} regions or CDRs selected from Table 51. In certain embodiments, the anti-L1CAM scFv comprises a V_{H} comprising the amino acid sequence set forth in SEQ ID NO: 135. In certain embodiments, the anti-L1CAM scFv comprises a V_{L} comprising the amino acid sequence set forth in SEQ ID NO: 131. SEQ ID NO: 135 and 131 are provided in Table 51. In certain embodiments, the scFv is designated as “76H12v9”.

In certain embodiments, the anti-L1CAM scFv comprises a V_{H} comprising the amino acid sequence set forth in SEQ ID NO: 135 and a V_{L} comprising the amino acid sequence set forth in SEQ ID NO: 131.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 51.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 51.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 135, and a V_{L} comprising the amino acid sequence set forth in SEQ ID NO: 131. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 51

| CDRs | 1 | 2 | 3 |
|---|---|---|---|
| V_{H} | GYTFTSYW | VYPGSGST | TRPGNYEGFAY |
| | [SEQ ID | [SEQ ID | [SEQ ID |
| | NO: 116] | NO: 117] | NO: 118] |
| V_{L} | ESVDSYGSSF | LAS | QQNNEDPWT |
| | [SEQ ID | [SEQ ID | [SEQ ID |
| | NO: 119] | NO: 120] | NO: 121] |

| | |
|---|---|
| Full | QVQLVQSGAEVKKPGASVKVSCKASGYTFTSYWMHWV |
| V_{H} | RQAPGQGLEWIGNVYPGSGSTKYAQKFQGRATLTVDT |
| | STNTAYMELSSLRSEDTAVYYCTRPGNYEGFAYWGQG |
| | TLVTVSS |
| | [SEQ ID NO: 135] |
| Full | DIQLTQSPSSLSASVGDRVTITCRASESVDSYGSSFM |
| V_{L} | HWYQQKPGKPPKLLIYLASNLESGVPSRFSGSGSGTD |
| | FTLTISSLQPEDFATYYCQQNNEDPWTFGQGTKLEIK |
| | [SEQ ID NO: 131] |

In certain embodiments, the anti-L1CAM antibody is an scFv, an scFv-Fc fusion protein or a full-length human IgG (or antibody fragment thereof) with V_{H} and V_{L} regions or CDRs selected from Table 52. In certain embodiments, the anti-L1CAM scFv comprises a V_{H} comprising the amino acid sequence set forth in SEQ ID NO: 135. In certain embodiments, the anti-L1CAM scFv comprises a V_{L} comprising the amino acid sequence set forth in SEQ ID NO: 132. SEQ ID NO: 135 and 132 are provided in Table 52. In certain embodiments, the scFv is designated as “76H12v10”.

In certain embodiments, the anti-L1CAM scFv comprises a V_{H} comprising the amino acid sequence set forth in SEQ ID NO: 135 and a V_{L} comprising the amino acid sequence set forth in SEQ ID NO: 132.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 52.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 52.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 135, and a V_{L} comprising the amino acid sequence set forth in SEQ ID NO: 132. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 52

| CDRs | 1 | 2 | 3 |
|---|---|---|---|
| V_{H} | GYTFTSYW | VYPGSGST | TRPGNYEGFAY |
| | [SEQ ID NO: | [SEQ ID NO: | [SEQ ID NO: 118] |
| | 116] | 117] | |
| V_{L} | ESVDSYGSSF | LAS | QQNNEDPWT |
| | [SEQ ID NO: | [SEQ ID NO: | [ SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| V_{H} | STKYAQKFQGRATLTVDTSTNTAYMELSSLRSEDTAVYYCTRPGNYEGFAYWGQGT |
| | LVTVSS |
| | [SEQ ID NO: 135] |
| Full | DIQLTQSPSSLSASVGDRVTITCRASESVDSYGSSEMHWYQQKPGKPPKLLIYLAS |
| V_{L} | NLESGVPSRFSGSGSRTDFTLTISSLQPEDFATYYCQQNNEDPWTFGQGTKLEIK |
| | [SEQ ID NO: 132] |

In certain embodiments, the anti-L1CAM antibody is an scFv, an scFv-Fc fusion protein or a full-length human IgG (or antibody fragment thereof) with V_{H} and V_{L} regions or CDRs selected from Table 53. In certain embodiments, the anti-L1CAM scFv comprises a V_{H} comprising the amino acid sequence set forth in SEQ ID NO: 135. In certain embodiments, the anti-L1CAM scFv comprises a V_{L} comprising the amino acid sequence set forth in SEQ ID NO: 133. SEQ ID NO: 135 and 133 are provided in Table 53. In certain embodiments, the scFv is designated as “76H12v11”.

In certain embodiments, the anti-L1CAM scFv comprises a V_{H} comprising the amino acid sequence set forth in SEQ ID NO: 135 and a V_{L} comprising the amino acid sequence set forth in SEQ ID NO: 133.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 53.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 53.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 135, and a V_{L} comprising the amino acid sequence set forth in SEQ ID NO: 133. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 53

| CDRs | 1 | 2 | 3 |
|---|---|---|---|
| V_{H} | GYTFTSYW | VYPGSGST | TRPGNYEGFAY |
| | [SEQ ID NO: | [SEQ ID NO: | [SEQ ID NO: 118] |
| | 116] | 117] | |
| V_{L} | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full V_{H} | QVQLVQSGAEVKKPGASVKVSCKASGYTFTSYWMHWVRQAPGQG |
| | LEWIGNVYPGSGSTKYAQKFQGRATLTVDTSTNTAYMELSSLRSEDTAVYY |
| | CTRPGNYEGFAYWGQGTLVTVSS |
| | [SEQ ID NO: 135] |
| Full V_{L} | NIQLTQSPSSLSASVGDRVTITCRASESVDSYGSSFMHWYQQKPGKPP |
| | KLLIYLASNLESGVPSRFSGSGSGTDFTLTISSLQPEDFATYYCQQNNEDPWT |
| | FGQGTKLEIK |
| | [SEQ ID NO: 133] |

In certain embodiments, the anti-L1CAM antibody is an scFv, an scFv-Fc fusion protein or a full-length human IgG (or antibody fragment thereof) with V_{H} and V_{L} regions or CDRs selected from Table 54. In certain embodiments, the anti-L1CAM scFv comprises a V_{H} comprising the amino acid sequence set forth in SEQ ID NO: 135. In certain embodiments, the anti-L1CAM scFv comprises a V_{L} comprising the amino acid sequence set forth in SEQ ID NO: 136. SEQ ID NO: 135 and 136 are provided in Table 54. In certain embodiments, the scFv is designated as “76H12v12”.

In certain embodiments, the anti-L1CAM scFv comprises a V_{H} comprising the amino acid sequence set forth in SEQ ID NO: 135 and a V_{L} comprising the amino acid sequence set forth in SEQ ID NO: 136.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, and a CDR3 comprising the amino acid sequence set forth in SEQ ID NO: 118 or a conservative modification thereof. SEQ ID NOs: 116-118 are provided in Table 54.

In certain embodiments, the anti-L1CAM scFv comprises a V_{L} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOs: 119-121 are provided in Table 54.

In certain embodiments, the anti-L1CAM scFv comprises a V_{H} Comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a V_{L} Comprising a CDR1 comprising the amino acid sequence set forth in SEQ ID NO: 119 or a conservative modification thereof, a CDR2 comprising the amino acid sequence set forth in SEQ ID NO: 120 or a conservative modification, and a CDR3 comprising the amino acid sequence set forth in SEQ ID NO: 121 or a conservative modification thereof.

In certain embodiments, the anti-L1CAM scFv comprises a V_{H} Comprising a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

In certain embodiments, the anti-L1CAM scFv comprises a V_{H} comprising the amino acid sequence set forth in SEQ ID NO: 135, and a V_{L} comprising the amino acid sequence set forth in SEQ ID NO: 136. In certain embodiments, the V_{H} and V_{L} are linked via a linker. In certain embodiments, the linker comprises the amino acid sequence set forth in SEQ ID NO: 138, SEQ ID NO: 139, SEQ ID NO: 140, SEQ ID NO: 141, SEQ ID NO: 142, or SEQ ID NO: 143.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 54

| CDRs | 1 | 2 | 3 |
|---|---|---|---|
| V_{H} | GYTFTSYW | VYPGSGST | TRPGNYEGFAY |
| | [SEQ ID NO: | [SEQ ID NO: | [SEQ ID NO: 118] |
| | 116] | 117] | |
| V_{L} | ESVDSYGSSF | LAS | QQNNEDPWT |
| | [SEQ ID NO: | [SEQ ID NO: | [SEQ ID NO: 121] |
| | 119] | 120] | |

| | |
|---|---|
| Full | QVQLVQSGAEVKKPGASVKVSCKASGYTFTSYWMHWVRQAPGQGLEWIGNVYPGSG |
| V_{H} | STKYAQKFQGRATLTVDTSTNTAYMELSSLRSEDTAVYYCTRPGNYEGFAYWGQGT |
| | LVTVSS |
| | [SEQ ID NO: 135] |
| Full | DIQMTQSPSSLSASVGDRVTITCRASESVDSYGSSFMHWYQQKPGKAPKLLIYLAS |
| V_{L} | NLESGVPSRFSGSGSGTDFTLTISSLQPEDFATYYCQQNNEDPWTFGQGTKLEIK |
| | [SEQ ID NO: 136] |

### 3.2. Monoclonal Antibodies

The presently disclosed subject matter provides antibodies (e.g., human antibodies, e.g., human monoclonal antibodies) that specifically bind to L1CAM (e.g., human L1CAM). The V_{H} amino acid sequences of anti-L1CAM antibodies TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5- h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 8, 22, 24, 25, 27, 28, 29, 33, 41, 49, 60, 67, 75, 87, 99, 110, 122, 128, 130, 134, and 135. The V_{L} amino acid sequences of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 9, 19, 23, 30, 31, 32, 34, 42, 50, 53, 61, 68, 76, 88, 100, 111, 123, 129, 131, 132, 133, and 136.

Given that each of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4- h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies can bind to L1CAM, the V_{H} and V_{L} sequences can be “mixed and matched” to create other anti-L1CAM binding molecules. L1CAM binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) Comprising an amino acid sequence selected from SEQ ID NOs: 8, 22, 24, 25, 27, 28, 29, 33, 41, 49, 60, 67, 75, 87, 99, 110, 122, 128, 130, 134, and 135; and (b) a light chain variable region (V_{L}) comprising an amino acid sequence selected from SEQ ID NOs: 9, 19, 23, 30, 31, 32, 34, 42, 50, 53, 61, 68, 76, 88, 100, 111, 123, 129, 131, 132, 133, and 136; wherein the antibody or antigen-binding fragment specifically binds to L1CAM, e.g., human L1CAM. In certain embodiments, the V_{H} and V_{L} are selected from the group consisting of:
(a) a heavy chain variable region comprising the amino acid sequence set forth in SEQ ID NO: 8, and a light chain variable region comprising the amino acid sequence set forth in SEQ ID NO: 9;
(b) a heavy chain variable region comprising the amino acid sequence set forth in SEQ ID NO: 18, and a light chain variable region comprising the amino acid sequence set forth in SEQ ID NO: 19;
(c) a heavy chain variable region comprising the amino acid sequence set forth in SEQ ID NO: 22, and a light chain variable region comprising the amino acid sequence set forth in SEQ ID NO: 23;
(d) a heavy chain variable region comprising the amino acid sequence set forth in SEQ ID NO: 24, and a light chain variable region comprising the amino acid sequence set forth in SEQ ID NO: 23;
(e) a heavy chain variable region comprising the amino acid sequence set forth in SEQ ID NO: 25, and a light chain variable region comprising the amino acid sequence set forth in SEQ ID NO: 23;
(f) a heavy chain variable region comprising the amino acid sequence set forth in SEQ ID NO: 27, and a light chain variable region comprising the amino acid sequence set forth in SEQ ID NO: 23;
(g) a heavy chain variable region comprising the amino acid sequence set forth in SEQ ID NO: 28, and a light chain variable region comprising the amino acid sequence set forth in SEQ ID NO: 23;
(h) a heavy chain variable region comprising the amino acid sequence set forth in SEQ ID NO: 29, and a light chain variable region comprising the amino acid sequence set forth in SEQ ID NO: 23;
(i) a heavy chain variable region comprising the amino acid sequence set forth in SEQ ID NO: 22, and a light chain variable region comprising the amino acid sequence set forth in SEQ ID NO: 30;
(j) a heavy chain variable region comprising the amino acid sequence set forth in SEQ ID NO: 24, and a light chain variable region comprising the amino acid sequence set forth in SEQ ID NO: 30; and
(k) a heavy chain variable region comprising the amino acid sequence set forth in SEQ ID NO: 25, and a light chain variable region comprising the amino acid sequence set forth in SEQ ID NO: 30;
(l) a heavy chain variable region comprising the amino acid sequence set forth in SEQ ID NO: 27, and a light chain variable region comprising the amino acid sequence set forth in SEQ ID NO: 30;
(m) a heavy chain variable region comprising the amino acid sequence set forth in SEQ ID NO: 28, and a light chain variable region comprising the amino acid sequence set forth in SEQ ID NO: 30; and
(n) a heavy chain variable region comprising the amino acid sequence set forth in SEQ ID NO: 29, and a light chain variable region comprising the amino acid sequence set forth in SEQ ID NO: 30;
(o) a heavy chain variable region comprising the amino acid sequence set forth in SEQ ID NO: 22, and a light chain variable region comprising the amino acid sequence set forth in SEQ ID NO: 31;
(p) a heavy chain variable region comprising the amino acid sequence set forth in SEQ ID NO: 24, and a light chain variable region comprising the amino acid sequence set forth in SEQ ID NO: 31;
(q) a heavy chain variable region comprising the amino acid sequence set forth in SEQ ID NO: 25, and a light chain variable region comprising the amino acid sequence set forth in SEQ ID NO: 31;
(r) a heavy chain variable region comprising the amino acid sequence set forth in SEQ ID NO: 27, and a light chain variable region comprising the amino acid sequence set forth in SEQ ID NO: 31;
(s) a heavy chain variable region comprising the amino acid sequence set forth in SEQ ID NO: 28, and a light chain variable region comprising the amino acid sequence set forth in SEQ ID NO: 31;
(t) a heavy chain variable region comprising the amino acid sequence set forth in SEQ ID NO: 29, and a light chain variable region comprising the amino acid sequence set forth in SEQ ID NO: 31;
(u) a heavy chain variable region comprising the amino acid sequence set forth in SEQ ID NO: 22, and a light chain variable region comprising the amino acid sequence set forth in SEQ ID NO: 32;
(v) a heavy chain variable region comprising the amino acid sequence set forth in SEQ ID NO: 24, and a light chain variable region comprising the amino acid sequence set forth in SEQ ID NO: 32; and
(w) a heavy chain variable region comprising the amino acid sequence set forth in SEQ ID NO: 25, and a light chain variable region comprising the amino acid sequence set forth in SEQ ID NO: 32;
(x) a heavy chain variable region comprising the amino acid sequence set forth in SEQ ID NO: 27, and a light chain variable region comprising the amino acid sequence set forth in SEQ ID NO: 32;
(y) a heavy chain variable region comprising the amino acid sequence set forth in SEQ ID NO: 28, and a light chain variable region comprising the amino acid sequence set forth in SEQ ID NO: 32; and
(z) a heavy chain variable region comprising the amino acid sequence set forth in SEQ ID NO: 29, and a light chain variable region comprising the amino acid sequence set forth in SEQ ID NO: 32;
(aa) a heavy chain variable region comprising the amino acid sequence set forth in SEQ ID NO: 22, and a light chain variable region comprising the amino acid sequence set forth in SEQ ID NO: 19;
(ab) a heavy chain variable region comprising the amino acid sequence set forth in SEQ ID NO: 24, and a light chain variable region comprising the amino acid sequence set forth in SEQ ID NO: 19;
(ac) a heavy chain variable region comprising the amino acid sequence set forth in SEQ ID NO: 25, and a light chain variable region comprising the amino acid sequence set forth in SEQ ID NO: 23;
(ad) a heavy chain variable region comprising the amino acid sequence set forth in SEQ ID NO: 27, and a light chain variable region comprising the amino acid sequence set forth in SEQ ID NO: 19;
(ae) a heavy chain variable region comprising the amino acid sequence set forth in SEQ ID NO: 28, and a light chain variable region comprising the amino acid sequence set forth in SEQ ID NO: 19;
(af) a heavy chain variable region comprising the amino acid sequence set forth in SEQ ID NO: 29, and a light chain variable region comprising the amino acid sequence set forth in SEQ ID NO: 19;
(ag) a heavy chain variable region comprising the amino acid sequence set forth in SEQ ID NO: 33, and a light chain variable region comprising the amino acid sequence set forth in SEQ ID NO: 34;
(ah) a heavy chain variable region comprising the amino acid sequence set forth in SEQ ID NO: 41, and a light chain variable region comprising the amino acid sequence set forth in SEQ ID NO: 42;
(ai) a heavy chain variable region comprising the amino acid sequence set forth in SEQ ID NO: 49, and a light chain variable region comprising the amino acid sequence set forth in SEQ ID NO: 50;
(aj) a heavy chain variable region comprising the amino acid sequence set forth in SEQ ID NO: 49, and a light chain variable region comprising the amino acid sequence set forth in SEQ ID NO: 53;
(ak) a heavy chain variable region comprising the amino acid sequence set forth in SEQ ID NO: 60, and a light chain variable region comprising the amino acid sequence set forth in SEQ ID NO: 61;
(al) a heavy chain variable region comprising the amino acid sequence set forth in SEQ ID NO: 67, and a light chain variable region comprising the amino acid sequence set forth in SEQ ID NO: 68;
(am) a heavy chain variable region comprising the amino acid sequence set forth in SEQ ID NO: 75, and a light chain variable region comprising the amino acid sequence set forth in SEQ ID NO: 76;
(an) a heavy chain variable region comprising the amino acid sequence set forth in SEQ ID NO: 87, and a light chain variable region comprising the amino acid sequence set forth in SEQ ID NO: 88;
(ao) a heavy chain variable region comprising the amino acid sequence set forth in SEQ ID NO: 99, and a light chain variable region comprising the amino acid sequence set forth in SEQ ID NO: 100;
(ap) a heavy chain variable region comprising the amino acid sequence set forth in SEQ ID NO: 110, and a light chain variable region comprising the amino acid sequence set forth in SEQ ID NO: 111;
(aq) a heavy chain variable region comprising the amino acid sequence set forth in SEQ ID NO: 122, and a light chain variable region comprising the amino acid sequence set forth in SEQ ID NO: 123;
(ar) a heavy chain variable region comprising the amino acid sequence set forth in SEQ ID NO: 128, and a light chain variable region comprising the amino acid sequence set forth in SEQ ID NO: 129;
(as) a heavy chain variable region comprising the amino acid sequence set forth in SEQ ID NO: 130, and a light chain variable region comprising the amino acid sequence set forth in SEQ ID NO: 131;
(at) a heavy chain variable region comprising the amino acid sequence set forth in SEQ ID NO: 130, and a light chain variable region comprising the amino acid sequence set forth in SEQ ID NO: 132;
(au) a heavy chain variable region comprising the amino acid sequence set forth in SEQ ID NO: 130, and a light chain variable region comprising the amino acid sequence set forth in SEQ ID NO: 133;
(av) a heavy chain variable region comprising the amino acid sequence set forth in SEQ ID NO: 134, and a light chain variable region comprising the amino acid sequence set forth in SEQ ID NO: 131;
(aw) a heavy chain variable region comprising the amino acid sequence set forth in SEQ ID NO: 134, and a light chain variable region comprising the amino acid sequence set forth in SEQ ID NO: 132;
(ax) a heavy chain variable region comprising the amino acid sequence set forth in SEQ ID NO: 134, and a light chain variable region comprising the amino acid sequence set forth in SEQ ID NO: 133;
(ay) a heavy chain variable region comprising the amino acid sequence set forth in SEQ ID NO: 135, and a light chain variable region comprising the amino acid sequence set forth in SEQ ID NO: 131;
(az) a heavy chain variable region comprising the amino acid sequence set forth in SEQ ID NO: 135, and a light chain variable region comprising the amino acid sequence set forth in SEQ ID NO: 132;
(ba) a heavy chain variable region comprising the amino acid sequence set forth in SEQ ID NO: 135, and a light chain variable region comprising the amino acid sequence set forth in SEQ ID NO: 133; or
(bb) a heavy chain variable region comprising the amino acid sequence set forth in SEQ ID NO: 135, and a light chain variable region comprising the amino acid sequence set forth in SEQ ID NO: 136.

In certain embodiments, the presently disclosed subject matter provides antibodies or antigen-binding fragments thereof that comprise the heavy chain and light chain CDR1s, CDR2s and CDR3s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12.

The amino acid sequences of the VHCDR1s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3- h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 2, 12, 35, 43, 54, 62, 69, 81, 93, 105, 106, and 116. The amino acid sequences of the V_{H} CDR2s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5- h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies are set forth in SEQ ID NOs: 3, 13, 36, 44, 55, 63, 70, 82, 94, 106, and 117. The amino acid sequences of the V_{H} CDR3s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 4, 14, 26, 37, 45, 56, 64, 71, 83, 95, 108, and 118.

The amino acid sequences of the V_{L} CDR1s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3- h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 5, 15, 38, 46, 51, 57, 65, 72, 84, 96, 108, and 119. The amino acid sequences of the V_{L} CDR2s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5- h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 6, 16, 39, 47, 52, 58, 73, 85, 97, and 120. The amino acid sequences of the V_{L} CDR3s of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3- h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 are set forth in SEQ ID NOs: 7, 17, 40, 48, 59, 66, 74, 86, 98, 109, and 121. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to L1CAM and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-L1CAM binding molecules. L1CAM binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12.

In certain embodiments, the presently disclosed subject matter provides an anti-L1CAM antibody or an antigen-binding fragment thereof comprising:
(a) a heavy chain variable region CDR1 comprising the amino acid sequence set forth in SEQ ID NO: 2, SEQ ID NO: 12, SEQ ID NO: 35, SEQ ID NO: 43, SEQ ID NO: 54, SEQ ID NO: 62, SEQ ID NO: 69, SEQ ID NO: 81, SEQ ID NO: 93, SEQ ID NO: 105, or SEQ ID NO: 116;
(b) a heavy chain variable region CDR2 comprising the amino acid sequence set forth in SEQ ID NO: 3, SEQ ID NO: 13, SEQ ID NO: 36, SEQ ID NO: 44, SEQ ID NO: 55, SEQ ID NO: 63, SEQ ID NO: 70, SEQ ID NO: 82, SEQ ID NO: 94, SEQ ID NO: 106, or SEQ ID NO: 117;
(c) a heavy chain variable region CDR3 comprising the amino acid sequence set forth in SEQ ID NO: 4, SEQ ID NO: 14, SEQ ID NO: 26, SEQ ID NO: 37, SEQ ID NO: 45, SEQ ID NO: 56, SEQ ID NO: 64, SEQ ID NO: 71, SEQ ID NO: 83, SEQ ID NO: 95, SEQ ID NO: 107, or SEQ ID NO: 118;
(d) a light chain variable region CDR1 comprising the amino acid sequence set forth in SEQ ID NO: 5, SEQ ID NO: 15, SEQ ID NO: 38, SEQ ID NO: 46, SEQ ID NO: 51, SEQ ID NO: 57, SEQ ID NO: 65, SEQ ID NO: 72, SEQ ID NO: 84, SEQ ID NO: 96, SEQ ID NO: 108, or SEQ ID NO: 119;
(e) a light chain variable region CDR2 comprising the amino acid sequence set forth in SEQ ID NO: 6, SEQ ID NO: 16, SEQ ID NO: 39, SEQ ID NO: 47, SEQ ID NO: 52, SEQ ID NO: 58, SEQ ID NO: 73, SEQ ID NO: 85, SEQ ID NO: 97, or SEQ ID NO: 120; and
(f) a light chain variable region CDR3 comprising the amino acid sequence set forth in SEQ ID NO: 7, SEQ ID NO: 17, SEQ ID NO: 40, SEQ ID NO: 48, SEQ ID NO: 59, SEQ ID NO: 66, SEQ ID NO: 74, SEQ ID NO: 86, SEQ ID NO: 98, SEQ ID NO: 109, or SEQ ID NO: 121.

In certain embodiments, the anti-L1CAM antibody or antigen-binding fragment thereof comprises:
(a) a heavy chain variable region CDR1 comprising the amino acid sequence set forth in SEQ ID NO: 2;
(b) a heavy chain variable region CDR2 comprising the amino acid sequence set forth in SEQ ID NO: 3;
(c) a heavy chain variable region CDR3 comprising the amino acid sequence set forth in SEQ ID NO: 4;
(d) a light chain variable region CDR1 comprising the amino acid sequence set forth in SEQ ID NO: 5;
(e) a light chain variable region CDR2 comprising the amino acid sequence set forth in SEQ ID NO: 6; and
(f) a light chain variable region CDR3 comprising the amino acid sequence set forth in SEQ ID NO: 7.

In certain embodiments, the anti-L1CAM antibody or antigen-binding fragment thereof comprises:
(a) a heavy chain variable region CDR1 comprising the amino acid sequence set forth in SEQ ID NO: 12;
(b) a heavy chain variable region CDR2 comprising the amino acid sequence set forth in SEQ ID NO: 13;
(c) a heavy chain variable region CDR3 comprising the amino acid sequence set forth in SEQ ID NO: 14;
(d) a light chain variable region CDR1 comprising the amino acid sequence set forth in SEQ ID NO: 15;
(e) a light chain variable region CDR2 comprising the amino acid sequence set forth in SEQ ID NO: 16; and
(f) a light chain variable region CDR3 comprising the amino acid sequence set forth in SEQ ID NO: 17.

In certain embodiments, the anti-L1CAM antibody or antigen-binding fragment thereof comprises:
(a) a heavy chain variable region CDR1 comprising the amino acid sequence set forth in SEQ ID NO: 12;
(b) a heavy chain variable region CDR2 comprising the amino acid sequence set forth in SEQ ID NO: 13;
(c) a heavy chain variable region CDR3 comprising the amino acid sequence set forth in SEQ ID NO: 26;
(d) a light chain variable region CDR1 comprising the amino acid sequence set forth in SEQ ID NO: 15;
(e) a light chain variable region CDR2 comprising the amino acid sequence set forth in SEQ ID NO: 16; and
(f) a light chain variable region CDR3 comprising the amino acid sequence set forth in SEQ ID NO: 17.

In certain embodiments, the anti-L1CAM antibody or antigen-binding fragment thereof comprises:
(a) a heavy chain variable region CDR1 comprising the amino acid sequence set forth in SEQ ID NO: 35;
(b) a heavy chain variable region CDR2 comprising the amino acid sequence set forth in SEQ ID NO: 36;
(c) a heavy chain variable region CDR3 comprising the amino acid sequence set forth in SEQ ID NO: 37;
(d) a light chain variable region CDR1 comprising the amino acid sequence set forth in SEQ ID NO: 38;
(e) a light chain variable region CDR2 comprising the amino acid sequence set forth in SEQ ID NO: 39; and
(f) a light chain variable region CDR3 comprising the amino acid sequence set forth in SEQ ID NO: 40.

In certain embodiments, the anti-L1CAM antibody or antigen-binding fragment thereof comprises:
(a) a heavy chain variable region CDR1 comprising the amino acid sequence set forth in SEQ ID NO: 43;
(b) a heavy chain variable region CDR2 comprising the amino acid sequence set forth in SEQ ID NO: 44;
(c) a heavy chain variable region CDR3 comprising the amino acid sequence set forth in SEQ ID NO: 45;
(d) a light chain variable region CDR1 comprising the amino acid sequence set forth in SEQ ID NO: 46;
(e) a light chain variable region CDR2 comprising the amino acid sequence set forth in SEQ ID NO: 47; and
(f) a light chain variable region CDR3 comprising the amino acid sequence set forth in SEQ ID NO: 48.

In certain embodiments, the anti-L1CAM antibody or antigen-binding fragment thereof comprises:
(a) a heavy chain variable region CDR1 comprising the amino acid sequence set forth in SEQ ID NO: 43;
(b) a heavy chain variable region CDR2 comprising the amino acid sequence set forth in SEQ ID NO: 44;
(c) a heavy chain variable region CDR3 comprising the amino acid sequence set forth in SEQ ID NO: 45;
(d) a light chain variable region CDR1 comprising the amino acid sequence set forth in SEQ ID NO: 51;
(e) a light chain variable region CDR2 comprising the amino acid sequence set forth in SEQ ID NO: 52; and
(f) a light chain variable region CDR3 comprising the amino acid sequence set forth in SEQ ID NO: 40.

In certain embodiments, the anti-L1CAM antibody or antigen-binding fragment thereof comprises:
(a) a heavy chain variable region CDR1 comprising the amino acid sequence set forth in SEQ ID NO: 54;
(b) a heavy chain variable region CDR2 comprising the amino acid sequence set forth in SEQ ID NO: 55;
(c) a heavy chain variable region CDR3 comprising the amino acid sequence set forth in SEQ ID NO: 56;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 59.

In certain embodiments, the anti-L1CAM antibody or antigen-binding fragment thereof comprises:
(a) a heavy chain variable region CDR1 comprising the amino acid sequence set forth in SEQ ID NO: 62;
(b) a heavy chain variable region CDR2 comprising the amino acid sequence set forth in SEQ ID NO: 63;
(c) a heavy chain variable region CDR3 comprising the amino acid sequence set forth in SEQ ID NO: 64;
(d) a light chain variable region CDR1 comprising the amino acid sequence set forth in SEQ ID NO: 65;
(e) a light chain variable region CDR2 comprising the amino acid sequence set forth in SEQ ID NO: 58; and
(f) a light chain variable region CDR3 comprising the amino acid sequence set forth in SEQ ID NO: 66.

In certain embodiments, the anti-L1CAM antibody or antigen-binding fragment thereof comprises:
(a) a heavy chain variable region CDR1 comprising the amino acid sequence set forth in SEQ ID NO: 69;
(b) a heavy chain variable region CDR2 comprising the amino acid sequence set forth in SEQ ID NO: 70;
(c) a heavy chain variable region CDR3 comprising the amino acid sequence set forth in SEQ ID NO: 71;
(d) a light chain variable region CDR1 comprising the amino acid sequence set forth in SEQ ID NO: 72;
(e) a light chain variable region CDR2 comprising the amino acid sequence set forth in SEQ ID NO: 73; and
(f) a light chain variable region CDR3 comprising the amino acid sequence set forth in SEQ ID NO: 74.

In certain embodiments, the anti-L1CAM antibody or antigen-binding fragment thereof comprises:
(a) a heavy chain variable region CDR1 comprising the amino acid sequence set forth in SEQ ID NO: 81;
(b) a heavy chain variable region CDR2 comprising the amino acid sequence set forth in SEQ ID NO: 82;
(c) a heavy chain variable region CDR3 comprising the amino acid sequence set forth in SEQ ID NO: 83;
(d) a light chain variable region CDR1 comprising the amino acid sequence set forth in SEQ ID NO: 84;
(e) a light chain variable region CDR2 comprising the amino acid sequence set forth in SEQ ID NO: 85; and
(f) a light chain variable region CDR3 comprising the amino acid sequence set forth in SEQ ID NO: 86.

In certain embodiments, the anti-L1CAM antibody or antigen-binding fragment thereof comprises:
(a) a heavy chain variable region CDR1 comprising the amino acid sequence set forth in SEQ ID NO: 93;
(b) a heavy chain variable region CDR2 comprising the amino acid sequence set forth in SEQ ID NO: 94;
(c) a heavy chain variable region CDR3 comprising the amino acid sequence set forth in SEQ ID NO: 95;
(d) a light chain variable region CDR1 comprising the amino acid sequence set forth in SEQ ID NO: 96;
(e) a light chain variable region CDR2 comprising the amino acid sequence set forth in SEQ ID NO: 97; and
(f) a light chain variable region CDR3 comprising the amino acid sequence set forth in SEQ ID NO: 98.

In certain embodiments, the anti-L1CAM antibody or antigen-binding fragment thereof comprises:
(a) a heavy chain variable region CDR1 comprising the amino acid sequence set forth in SEQ ID NO: 105;
(b) a heavy chain variable region CDR2 comprising the amino acid sequence set forth in SEQ ID NO: 106;
(c) a heavy chain variable region CDR3 comprising the amino acid sequence set forth in SEQ ID NO: 107;
(d) a light chain variable region CDR1 comprising the amino acid sequence set forth in SEQ ID NO: 108;
(e) a light chain variable region CDR2 comprising the amino acid sequence set forth in SEQ ID NO: 39; and
(f) a light chain variable region CDR3 comprising the amino acid sequence set forth in SEQ ID NO: 109.

In certain embodiments, the anti-L1CAM antibody or antigen-binding fragment thereof comprises:
(a) a heavy chain variable region CDR1 comprising the amino acid sequence set forth in SEQ ID NO: 116;
(b) a heavy chain variable region CDR2 comprising the amino acid sequence set forth in SEQ ID NO: 117;
(c) a heavy chain variable region CDR3 comprising the amino acid sequence set forth in SEQ ID NO: 118;
(d) a light chain variable region CDR1 comprising the amino acid sequence set forth in SEQ ID NO: 119;
(e) a light chain variable region CDR2 comprising the amino acid sequence set forth in SEQ ID NO: 120; and
(f) a light chain variable region CDR3 comprising the amino acid sequence set forth in SEQ ID NO: 121.

In certain embodiments, the anti-L1CAM antibody or antigen-binding fragment thereof comprises a heavy chain constant region and/or a light chain constant region.

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 144. In certain embodiments, the heavy chain constant region comprises the amino acid sequence set forth in SEQ ID NO: 144. SEQ ID NO: 144 is provided below.

[SEQ ID NO: 144]
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHNHYT
QKSLSLSPGK

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 145. In certain embodiments, the heavy chain constant region comprises the amino acid sequence set forth in SEQ ID NO: 145. SEQ ID NO: 145 is provided below.

[SEQ ID NO: 145]
ASTKGPSVFPLAPCSRSTSESTAALGCLVKDYFPEPVTVSWNSGALTSGVHTFPAVLQSSGLYS
LSSVVTVPSSNFGTQTYTCNVDHKPSNTKVDKTVERKCCVECPPCPAPPVAGPSVFLFPPKPKD
TLMISRTPEVTCVVVDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQVSLTCLVKGFYPSDI
SVEWESNGQPENNYKTTPPMLDSDGSFFLYSKLTVDKSRWQQGNVFSCSVMHEALHNHYTQKSL
SLSPGK

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 146. In certain embodiments, the heavy chain constant region comprises the amino acid sequence set forth in SEQ ID NO: 146. SEQ ID NO: 146 is provided below.

[SEQ ID NO: 146]
ASTKGPSVFPLAPCSRSTSGGTAALGCLVKDYFPEPVTVSWNSGALTSGVHTFPAVLQSSGLYS
LSSVVTVPSSSLGTQTYTCNVNHKPSNTKVDKRVELKTPLGDTTHTCPRCPEPKSCDTPPPCPR
CPEPKSCDTPPPCPRCPEPKSCDTPPPCPRCPAPELLGGPSVFLFPPKPKDTLMISRTPEVTCV
VVDVSHEDPEVQFKWYVDGVEVHNAKTKPREEQYNSTFRVVSVLTVLHQDWLNGKEYKCKVSNK
ALPAPIEKTISKTKGQPREPQVYTLPPSREEMTKNQVSLTCLVKGFYPSDIAVEWESSGQPENN
YNTTPPMLDSDGSFFLYSKLTVDKSRWQQGNIFSCSVMHEALHNRFTQKSLSLSPGK

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 147. In certain embodiments, the heavy chain constant region comprises the amino acid sequence set forth in SEQ ID NO: 147. SEQ ID NO: 147 is provided below.

[SEQ ID NO: 147]
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPSCPAPEFLGGPSVFLFPPKPK
DTLMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKS
LSLSLGK

In certain embodiments, the light chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 148. In certain embodiments, the light chain constant region comprises the amino acid sequence set forth in SEQ ID NO: 148. SEQ ID NO: 148 is provided below.

[SEQ ID NO: 148]
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSENRGEC

In certain embodiments, the anti-L1CAM antibody or an antigen-binding fragment thereof comprises: (a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 144, SEQ ID NO: 145, SEQ ID NO: 146, or SEQ ID NO: 147; and (b) a light chain constant region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 148. In certain embodiments, the anti-L1CAM antibody or an antigen-binding fragment thereof comprises: (a) a heavy chain constant region comprising the amino acid sequence set forth in SEQ ID NO: 144, SEQ ID NO: 145, SEQ ID NO: 146, or SEQ ID NO: 147; and (b) a light chain constant region comprising the amino acid sequence set forth in SEQ ID NO: 148. In certain embodiments, the anti-L1CAM antibody or an antigen-binding fragment thereof comprises: (a) a heavy chain constant region comprising the amino acid sequence set forth in SEQ ID NO: 144; and (b) a light chain constant region comprising the amino acid sequence set forth in SEQ ID NO: 148.

The constant regions/framework regions of the anti-L1CAM antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-L1CAM antibody is a fully-human antibody, e.g., any one of TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4- h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12. Fully-human mAbs, when administered to humans, causing serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human L1CAM polypeptide (e.g., a polypeptide having the amino acid sequence set forth in SEQ ID NO: 1) for cancer therapy.

### 3.3. Homologous Antibodies

In certain embodiments, a presently disclosed anti-L1CAM antibody or antigen-binding fragment thereof comprises heavy and light chain variable regions comprising amino acid sequences that are homologous or identical to the amino acid sequences of the antibodies described herein (e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies), and wherein the antibodies or antigen-binding fragments thereof retain the desired functional properties of the anti-L1CAM antibodies or antigen-binding fragments thereof of the presently disclosed subject matter.

For example, the presently disclosed subject matter provides an anti-L1CAM antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 22, SEQ ID NO: 24, SEQ ID NO: 25, SEQ ID NO: 26, SEQ ID NO: 27, SEQ ID NO: 28, SEQ ID NO: 29, SEQ ID NO: 33, SEQ ID NO: 41, SEQ ID NO: 49, SEQ ID NO: 60, SEQ ID NO: 67, SEQ ID NO: 75, SEQ ID NO: 87, SEQ ID NO: 99, SEQ ID NO: 110, SEQ ID NO: 122, SEQ ID NO: 128, SEQ ID NO: 130, or SEQ ID NO: 135; and (b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 23, SEQ ID NO: 30, SEQ ID NO: 31, SEQ ID NO: 32, SEQ ID NO: 34, SEQ ID NO: 42, SEQ ID NO: 50, SEQ ID NO: 53, SEQ ID NO: 61, SEQ ID NO: 68, SEQ ID NO: 76, SEQ ID NO: 88, SEQ ID NO: 100, SEQ ID NO: 111, SEQ ID NO: 123, SEQ ID NO: 129, SEQ ID NO: 131, SEQ ID NO: 132, SEQ ID NO: 133, or SEQ ID NO: 136.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

In certain embodiments, a presently disclosed anti-L1CAM antibody or antigen-binding fragment thereof comprises heavy and light chain constant regions comprising amino acid sequences that are homologous or identical to the amino acid sequences of the antibodies described herein (e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies), and wherein the antibodies or antigen-binding fragments thereof retain the desired functional properties of the anti-L1CAM antibodies or antigen-binding fragments thereof of the presently disclosed subject matter.

For example, the presently disclosed subject matter provides an anti-L1CAM antibody or an antigen-binding fragment thereof, comprising a heavy chain constant region and a light chain constant region, wherein:
(a) the heavy chain constant region (C_{H}) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 144; and
(b) the light chain constant region (C_{L}) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 145.

In certain embodiments, the C_{H} and/or C_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having C_{H} and C_{L} regions having high (i.e., 80% or greater) homology or identity to the C_{H} and C_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

3.4. Antibodies with Conservative Modifications

In certain embodiments, a presently disclosed anti-L1CAM antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3- h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies), or a conservative modification thereof, and wherein the antibodies retain the desired functional properties of the anti-L1CAM antibodies or antigen-binding fragments thereof of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from SEQ ID NOs: 4, 14, 26, 37, 45, 56, 64, 71, 83, 95, 107, and 118, and conservative modifications thereof;
(b) the light chain variable region CDR3 sequence comprises an amino acid sequence selected from SEQ ID NOs: 7, 17, 40, 48, 59, 66, 74, 86, 98, 109, and 121, and conservative modifications thereof.

In certain embodiments, the heavy chain variable region CDR3 sequence comprises an amino acid sequence selected from SEQ ID NOs: 4, 14, 26, 37, 45, 56, 64, 71, 83, 95, 107, and 118, and conservative modifications thereof; and the light chain variable region CDR3 sequence comprises an amino acid sequence selected from SEQ ID NOs: 7, 17, 40, 48, 59, 66, 74, 86, 98, 109, and 121, and conservative modifications thereof.

In certain embodiments, the heavy chain variable region CDR2 sequence comprises an amino acid sequence selected from SEQ ID NOs: 3, 13, 36, 44, 55, 63, 70, 82, 94, 106, and 117, and conservative modifications thereof; and the light chain variable region CDR2 sequence comprises an amino acid sequence selected from SEQ ID NOs: 6, 16, 39, 47, 52, 58, 73, 85, 97, and 120, and conservative modifications thereof.

In certain embodiments, the heavy chain variable region CDR1 sequence comprises an amino acid sequence selected from SEQ ID NOs: 2, 12, 35, 43, 54, 62, 69, 81, 93, 105, and 116, and conservative modifications thereof; and the light chain variable region CDR1 sequence comprises an amino acid sequence selected from SEQ ID NOs: 5, 15, 38, 46, 51, 57, 65, 72, 84, 96, 108, and 119, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 55. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 55

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

3.5. Anti-L1CAM Antibodies that Cross-Compete for Binding to L1CAM with Anti-L1CAM Antibodies of the Present Disclosure

The presently disclosed subject matter provides antibodies or antigen-binding fragments thereof that cross-compete with any of the disclosed anti-L1CAM antibodies for binding to L1CAM (e.g., human L1CAM). For example, and not by way of limitation, the cross-competing antibodies can bind to the same epitope region, e.g., same epitope, adjacent epitope, or overlapping as any of the anti-L1CAM antibodies or antigen-binding fragments thereof of the presently disclosed subject matter. In certain embodiments, the reference antibody or reference antigen-binding fragments thereof for cross-competition studies can be any one of the anti-L1CAM antibodies or antigen-binding fragments thereof disclosed herein, e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5- h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies.

Such cross-competing antibodies can be identified based on their ability to cross-compete with any one of the presently disclosed anti-L1CAM antibodies or antigen-binding fragments thereof in standard L1CAM binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-L1CAM antibodies (e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3- h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies) to L1CAM (e.g., human L1CAM) demonstrates that the test antibody can compete with any one of the presently disclosed anti-L1CAM antibodies or antigen-binding fragments thereof for binding to L1CAM (e.g., human L1CAM) and thus binds to the same epitope region on L1CAM (e.g., human L1CAM) as any one of the presently disclosed anti-L1CAM antibodies or antigen-binding fragments thereof. In certain embodiments, the cross-competing antibody or antigen-binding fragment thereof binds to the same epitope on L1CAM (e.g., human L1CAM) as any one of the presently disclosed anti-L1CAM antibodies or antigen-binding fragments thereof.

### 3.6. Characterization of Antibody Binding to Antigen

Antibodies or antigen-binding fragments thereof of the presently disclosed subject can be tested for binding to L1CAM by, for example, standard ELISA. To determine if the selected anti-L1CAM antibodies bind to unique epitopes, each antibody can be biotinylated using commercially available reagents (Pierce, Rockford, IL). Competition studies using unlabeled monoclonal antibodies and biotinylated monoclonal antibodies can be performed using L1CAM coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-L1CAM human IgGs can be further tested for reactivity with L1CAM antigen by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ).

### 3.7. Multi-Specific Molecules

The presently disclosed subject matter provides multi-specific molecules comprising an anti-L1CAM antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed anti-L1CAM antibody or antigen-binding fragment thereof can in fact be derivatized or linked to more than one other functional molecules to generate multi-specific molecules that bind to more than two different binding sites and/or target molecules. To create a multi-specific molecule, a presently disclosed anti-L1CAM antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprise at least a first binding specificity for L1CAM and a second binding specificity for a second target epitope region. The second target epitope region can be a L1CAM epitope, or a non-L1CAM epitope, e.g., a different antigen. In certain embodiments, the multi-specific molecule comprises a first binding specificity for L1CAM, a second binding specificity for a second target, and a third binding specificity for a third target. In certain embodiments, the second target is an antigen expressed on the surface of an immune cell (e.g., a T cell, or a human immune effector cell). In certain embodiments, the multi-specific molecule is capable of recruiting the activity of that immune effector cell by specifically binding to the effector antigen on the human immune effector cell, thereby enhancing effector function.

In certain embodiments, the third target is an antigen expressed on a senescent cell. The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb×mAb, mAb×Fab, Fab×F(ab′)₂ or ligand×Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 3.8. Antibody Fragments

The presently disclosed subject matter provides antibody fragments of an anti-L1CAM antibody disclosed herein. In certain embodiments, the antibody fragment comprises an scFv as disclosed herein in Section 3.1. In certain embodiments, the antibody fragment is an scFv as disclosed herein in Section 3.1. In certain embodiments, the antibody fragment is a Fab fragment, a Fab′ fragment, a Fab′-SH fragment, or a F(ab′)₂ fragment.

In certain embodiments, the antibody fragment is a “Fab” fragments. “Fab” fragments can be produced by papain digestion of full length antibodies. Traditionally, Fab fragments contain the heavy-chain variable domain (V_{H}) and light-chain variable domain (V_{H}) and also the constant domain of the light chain (C_{L}) and the first constant domain of the heavy chain (CH1).

In certain embodiments, the antibody fragment is a “Fab′” fragments. Fab′ fragments can be distinguished from Fab fragments because including additional residues at the carboxy terminus of the CH1 domain. In certain embodiments, the Fab′ fragments include one or more cysteines from the antibody hinge region.

In certain embodiments, the antibody fragment is a “Fab′-SH” fragments. Fab′-SH are Fab′ fragments where at least one cysteine residue of the constant domains includes a free thiol group.

In certain embodiments, the antibody fragment is a “F(ab′)₂” fragment. F(ab′)₂ fragments can be obtained by pepsin digestion of full length antibodies. F(ab′)₂ fragments have two antigen-binding sites (e.g., two Fab fragments) and a portion of the Fc region.

In certain embodiments, the antibody fragment is a single-domain antibody. Single-domain antibody are antibody fragments including the heavy chain variable domain or a portion thereof of an antibody or the light chain variable domain or a portion thereof of an antibody.

### 3.9. Humanized or Chimeric Antibodies

The presently disclosed subject matter further provides chimeric and/or humanized versions of an anti-L1CAM antibody, or a fragment thereof, disclosed herein.

In certain embodiments, the anti-L1CAM antibody is a chimeric antibody. In certain embodiments, the chimeric antibody comprises a variable region derived from a non-human species (e.g., a variable region derived from a mouse, a rat, a hamster, a rabbit, or a non-human primate) and a human constant region. Alternatively or additionally, a chimeric antibody can be a “class-switched” antibody. In certain embodiments, the class-switched antibody is an antibody wherein the class or subclass has been modified from that of the parent antibody.

In certain embodiments, the anti-L1CAM antibody provided herein is a humanized antibody. In certain embodiments, the humanized antibody comprises at least one variable domain. In certain embodiments, the variable domain comprises CDRs derived from a non-human antibody, e.g., a mouse antibody.

In certain embodiments, the variable domain comprises framework regions (FR) derived from human antibody sequences. In certain embodiments, the FR include substitutions and/or modification. In certain embodiments, residues in a humanized antibody can be substituted with corresponding residues from a non-human antibody (e.g., the antibody from which the CDR residues are derived), to restore or improve antibody specificity or affinity.

In certain embodiments, the humanized antibody can also include a human constant region. In certain embodiments, humanization of a non-human antibody, e.g. a mouse antibody, reduces immunogenicity of the antibody in humans. In certain embodiments, humanization of a non-human antibody, e.g. a mouse antibody, does not impair the specificity and affinity of the parental non-human antibody.

### 4. Immunoconjugates

The presently disclosed subject provides an anti-L1CAM antibody or an antigen-binding fragment thereof, conjugated to a therapeutic moiety, such as a cytotoxin, a drug (e.g., an immunosuppressant), or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Non-limiting examples of cytotoxins include taxol (such as ricin, diphtheria and gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), hypomethylating agents (azacytidine and decitabine), and anti-mitotic agents (e.g., vincristine and vinblastine). Other non-limiting examples of therapeutic cytotoxins that can be conjugated to an anti-L1CAM antibody disclosed herein include duocarmycins, calicheamicins, maytansines, auristatins, calicheamicin, tubulysin, and pyrrolobenzodiazepines (PBD), and derivatives thereof.

In certain embodiments, the therapeutic agent is a compound of the class of dolastatins, maytansinoids, duocarmycins, anthracyclines, camptothecins, or amatoxins. In certain embodiments, the therapeutic agent is selected from the group consisting of monomethyl auristatin E, ABZ038, duocarmycin TM, PNU159682, SN38, and α-Amanitin. In certain embodiments, the therapeutic agent is PNU159682. PNU159682 is a highly potent metabolite of the anthracycline nemorubicin, a DNA topoisomerase II inhibitor. In certain embodiments, PNU159682 has formula:

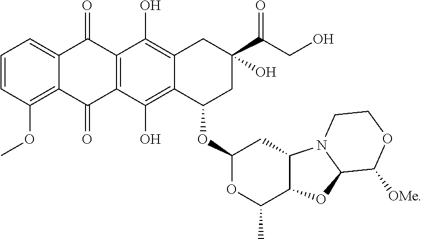 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1]

In certain embodiments, PNU159682 has formula:

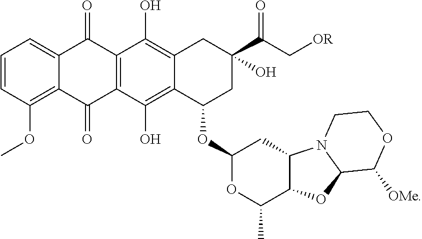

In certain embodiments, R is an anti-L1CAM antibody or an antigen-binding fragment thereof disclosed herein.

Cytotoxins can be conjugated to an anti-L1CAM antibody or an antigen-binding fragment thereof disclosed herein using linker technology available in the art. In certain embodiments, a linker is a molecule with two reactive termini, one for conjugation to an antibody and the other for conjugation to a cytotoxin (e.g., PNU159682). The antibody conjugation reactive terminus of the linker is typically a site that is capable of conjugation to the antibody through a cysteine thiol or lysine amine group on the antibody, and so is typically a thiol-reactive group such as a double bond (as in maleimide) or a leaving group such as a chloro, bromo, or iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). In certain embodiments, the linker can be conjugated to an anti-L1CAM antibody or an antigen-binding fragment thereof disclosed herein using a cysteine rebridging approach called ThioBridge®. Additional information on ThioBridge® can be found in Bird et al., Methods Mol Biol. 2020; 2078:113-129, the content of which is incorporated in its entirety.

In certain embodiments, the linker comprises Gly and Ser. In certain embodiments, the linker can, for example, and not by way of limitation, be between about 1 and about 25 or between about 5 and about 20 or between about 5 and about 15 amino acids in length. Non-limiting examples of linkers for use in the presently disclosure subject matter are disclosed in International Patent Publication WO 2017/165464 (e.g., SEQ ID NOs: 42, 44, 45, 75, 76, 77 and 78), the contents of which are incorporated by reference herein in its entirety.

For further discussion of types of cytotoxins, linkers, and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-L1CAM antibodies or antigen-binding fragments thereof of the presently disclosed subject matter also can be conjugated to a radioactive isotope to generate cytotoxic radiopharmaceuticals, also referred to as radioimmunoconjugates. Non-limiting examples of radioactive isotopes that can be conjugated to antibodies for use diagnostically or therapeutically include ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra, and ²²⁷Th. Methods for preparing radioimmunoconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the presently disclosed anti-L1CAM antibodies.

In certain embodiments, anti-L1CAM antibodies or antigen-binding fragments thereof of the presently disclosed subject matter can be conjugated to non-radioactive isotopes that can be irradiated, e.g., with neutrons, to undergo a decay reaction and generate alpha particles. For example, but not by way of limitation, an anti-L1CAM antibodies or antigen-binding fragments thereof of the presently disclosed subject matter also can be conjugated to a molecule comprising boron (e.g., ¹⁰B), a stable non-radioactive isotope of boron. The presently disclosed conjugates comprising boron can be used in boron neutron capture therapy (BNCT). As used herein, “boron neutron capture therapy” or “BNCT” refers to a targeted radiotherapy, wherein boron of the presently disclosed immunoconjugates comprising boron are irradiated with low energy thermal neutrons to yield biologically destructive alpha particles and lithium-7 nuclei. Additional information concerning immunoconjugates comprising boron and BNCT can be found in Guan et al., Proceedings of the National Academy of Sciences 95, no. 22 (1998): 13206-13210; Barth et al., Cancer Communications 38, no. 1 (2018): 1-15; Nedunchezhian et al., Journal of clinical and diagnostic research: JCDR 10, no. 12 (2016): ZE01; Malouff et al., Frontiers in oncology 11 (2021): 601820; and U.S. Pat. No. 4,624,846; the content of each of which is incorporated by reference in its entirety.

Anti-L1CAM antibodies or antigen-binding fragments thereof of the presently disclosed subject matter also can be conjugated to a self-assembly disassembly (SADA) polypeptide. A SADA domain is composed of multimerization domains which are each composed of helical bundles that associate in a parallel or anti-parallel orientation. Non-limiting examples of SADA domain containing human polypeptides include p53, p63, p73, heterogeneous nuclear Ribonucleoprotein C (hnRNPC), C or N-terminal domain of Synaptosomal-associated protein 23 (SNAP-23), Cyclin-D-related protein (CBFA2T1), variants thereof, or fragments thereof. In certain embodiments, these conjugates can multimerize to form a complex of a desired size under relevant conditions (e.g., in a solution in which the conjugate is present above a threshold concentration or pH and/or when present at a target site characterized by a relevant level or density of receptors for the payload), and disassemble to a smaller form under other conditions (e.g., absent the relevant environmental multimerization trigger). Additional information concerning the SADA polypeptides and immunoconjugates comprising the same can be found in International Patent Publication No. WO 2018/204873, the content of which is incorporated by reference in its entirety.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-7; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 4.1. Exemplary Immunoconjugates

In certain embodiments, the immunoconjugate comprises an anti-L1CAM antibody or an antigen-binding fragment thereof disclosed herein. In certain embodiments, the immunoconjugate comprises a compound from the class of anthracyclines. In certain embodiments, the immunoconjugate comprises PNU159682. In certain embodiments, the immunoconjugate comprises a linker. In certain embodiments, the linker is a non-cleavable linker. In certain embodiments, the immunoconjugate comprises a molecule of formula:

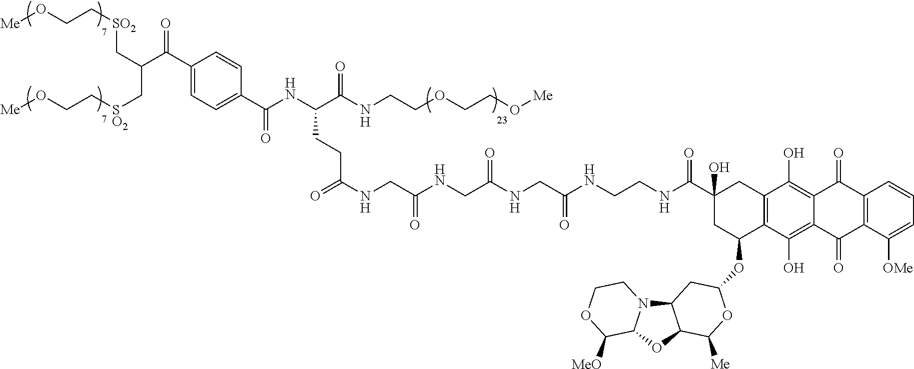

In certain embodiments, the immunoconjugate comprises an anti-L1CAM antibody or an antigen-binding fragment thereof disclosed herein. In certain embodiments, the immunoconjugate comprises a compound from the class of anthracyclines. In certain embodiments, the immunoconjugate comprises PNU159682. In certain embodiments, the immunoconjugate comprises a linker. In certain embodiments, the linker is a cleavable linker. In certain embodiments, the linker is a Cathepsin B cleavable linker. In certain embodiments, the immunoconjugate comprises a molecule of formula:

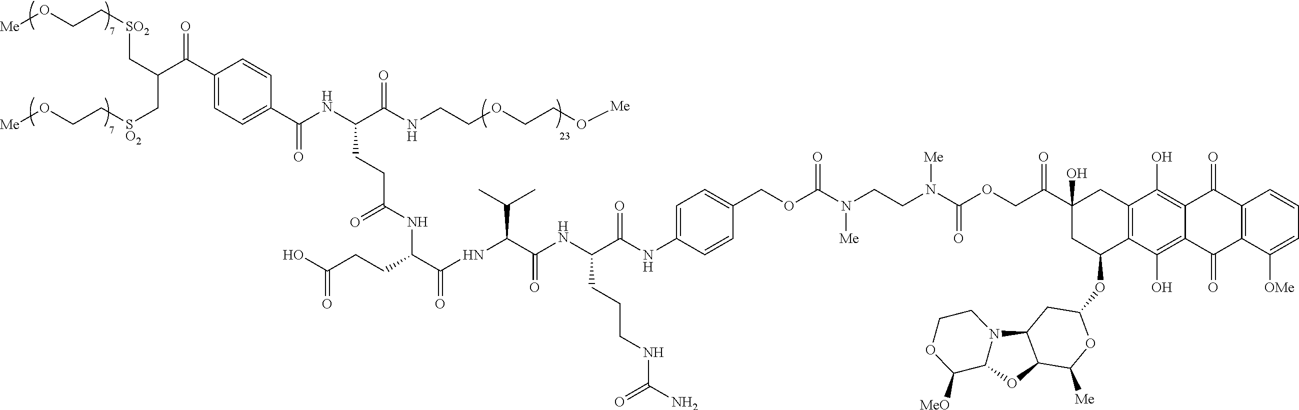

### 5. Nucleic Acids Encoding the Antibodies or Antigen-Binding Fragments and/or V_{H} and/or V_{L} Thereof

The presently disclosed subject matter provides nucleic acids encoding the anti-L1CAM antibodies or antigen-binding fragments thereof disclosed herein. The presently disclosed subject matter provides nucleic acids encoding the heavy chain variable region sequence of any one of the presently disclosed anti-L1CAM antibodies (e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3- h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies).

The presently disclosed subject matter provides nucleic acids encoding the light chain variable region sequence of any one of the presently disclosed anti-L1CAM antibodies (e.g., TDI-Y-005, TDI-Y-004, k1-h1, k1-h2, k1-h3, k1-h4, k1-h5, k1-h6, k2-h1, k2-h2, k2-h3, k2-h4, k2-h5, k2-h6, k3-h1, k3-h2, k3-h3, k3-h4, k3-h5, k3-h6, k4-h1, k4-h2, k4-h3, k4-h4, k4-h5, k4-h6, k5-h1, k5-h2, k5-h3, k5-h4, k5-h5, k5-h6, 143G03, 04B06, 12D10v1, 12D10v2, 13G04, 15G02, 13F04, 14A10, 76H05, 18H05, 76H12, 76H12v2, 76H12v3, 76H12v4, 76H12v5, 76H12v6, 76H12v7, 76H12v8, 76H12v9, 76H12v10, 76H12v11, and 76H12v12 antibodies).

Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

### 6. Pharmaceutical Compositions and Methods of Treatment

The presently disclosed subject matter provides compositions comprising a presently disclosed anti-L1CAM antibody or an antigen-binding fragment thereof, a presently disclosed immunoconjugate, a presently disclosed multi-specific molecule. In certain embodiments, the composition is a pharmaceutical composition further comprising a pharmaceutically acceptable carrier.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter provides various methods of using the anti-L1CAM antibodies or antigen-binding fragments thereof, the immunoconjugate, the multi-specific molecule, and the composition disclosed herein. For example, the presently disclosed subject matter provides methods for treating or ameliorating a disease or disorder in a subject. In certain embodiments, the method comprises administering one or more of the anti-L1CAM antibodies or antigen-binding fragments thereof, the immunoconjugate, the multi-specific molecule, or the composition disclosed herein to the subject. In certain embodiments, the disease or disorder is associated with L1CAM. In certain embodiments, the disease or disorder is associated with overexpression of L1CAM. In certain embodiments, the disease or disorder is tumor.

The presently disclosed subject matter provides methods of reducing tumor burden in a subject. In certain embodiments, the method comprises administering one or more of the anti-L1CAM antibodies or antigen-binding fragments thereof, the immunoconjugate, the multi-specific molecule, or the composition disclosed herein to the subject. The presently disclosed anti-L1CAM antibodies or antigen-binding fragments thereof can reduce the number of tumor cells, reduce tumor size, and/or eradicate the tumor in the subject.

The presently disclosed subject matter also provides methods of increasing or lengthening survival of a subject having a tumor. In certain embodiments, the method comprises administering one or more of the anti-L1CAM antibodies or antigen-binding fragments thereof, the immunoconjugate, the multi-specific molecule, or the composition disclosed herein to the subject. The method can reduce or eradicate tumor burden in the subject.

The presently disclosed subject matter further provides methods for treating and/or preventing a tumor in a subject. In certain embodiments, the method comprises administering one or more of the anti-L1CAM antibodies or antigen-binding fragments thereof, the immunoconjugate, the multi-specific molecule, or the composition disclosed herein to the subject.

Such methods comprise administering the presently disclosed anti-L1CAM antibodies or antigen-binding fragments thereof in an amount effective, a presently disclosed composition (e.g., a pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the tumor is a cancer. In certain embodiments, the tumor is a metastatic cancer. In certain embodiments, the metastatic cancer is an advanced metastatic cancer. Metastatic cancer is a cancer that has spread from the part of the body where it started (e.g., the primary site) to other parts of the body. When cancer cells break away from a tumor, they can travel to other parts of the body through the bloodstream or the lymph system. In certain non-limiting embodiments, the metastatic cancer localizes in at least one tissue selected from the group consisting of bone, liver, lung, brain, peritoneum, adrenal gland, skin, lymph nodes, pleura, and meninges.

In certain embodiments, the presently disclosed subject matter also provides methods of reducing the risk of metastatic spread in a subject in need thereof. As used herein, “reducing the risk of metastatic spread” is relative to the risk of metastatic spread in a comparable control subject not treated with the presently disclosed antibodies.

In certain embodiments, the presently disclosed subject matter also provides methods of inhibiting metastatic spread of a primary cancer in a subject in need thereof. As used herein, the term “inhibiting metastatic spread of a primary cancer” means one or more of reducing the number, location(s), and/or size, of metastasis/es, and/or increasing the period of time to occurrence of metastasis/es, and/or prolonging survival, relative to a comparable control subject not treated with the presently disclosed antibodies.

In certain embodiments, the presently disclosed subject matter also provides methods of inhibiting progression of metastatic disease in a subject in need thereof. As used herein, “inhibiting progression of metastatic disease” means one or more of the following: decreasing the size of existing metastasis/es, reducing the rate of growth of existing metastasis/es, reducing the incidence of newly detectable metastasis/es, improving quality of life, and/or increasing time to recurrence, and/or prolonging survival, relative to a comparable control subject not treated with the presently disclosed antibodies.

Any suitable method or route can be used to administer a presently disclosed anti-L1CAM antibody, and optionally, to co-administer antineoplastic agents. Routes of administration include, but are not limited to, oral, intravenous, intraperitoneal, subcutaneous, intramuscular, intranodal, intratumoral, intraosseous, intrathecal, pleural, intrapleural, topical, and direct administration. It should be emphasized, however, that the presently disclosed subject matter is not limited to any particular method or route of administration.

The presently disclosed anti-L1CAM antibodies or antigen-binding fragments thereof can be administered as a conjugate, which binds specifically to the receptor and delivers a toxic, lethal payload following ligand-toxin internalization.

In certain embodiments, the presently disclosed methods include administering the anti-L1CAM antibodies or antigen-binding fragments thereof, the immunoconjugate, the multi-specific molecule, or the composition disclosed herein in combination with a second therapeutic agent. As used here, the term “in combination with” means that the anti-L1CAM antibodies or antigen-binding fragments thereof, the immunoconjugate, the multi-specific molecule, or the composition disclosed herein, and one or more agents, e.g., an second therapeutic agent, are administered to a subject as part of a treatment regimen or plan.

In certain embodiments, the second therapeutic agent targets components of the immune system to fight cancer can be used with the presently disclosed methods. Non-limiting examples of second therapeutic agents include immune checkpoint inhibitors, dendritic cells, therapeutic antibodies (e.g., anti-CD33 antibodies, anti-CD11b antibodies), cancer vaccines, cytokines (e.g., IL-12, GM-CSF, IL-2, IFNβ, IFNγ, MIP-1, MCP-1, IL-8), Bacillus Calmette-Gudrin (BCG), and any combinations thereof. In certain embodiments, the second therapeutic agent is an immune checkpoint inhibitor. In certain embodiments, the immune checkpoint inhibitor is selected from anti-PD1 antibodies, anti-PD-L1 antibodies, anti-CTLA-4 antibodies, anti-BTLA antibodies, anti-TIM3 antibodies, anti-LAG-3 antibodies, and any combinations thereof. For example, but without any limitation, the immune checkpoint inhibitor can be pembrolizumab (KEYTRUDA®), nivolumab (OPDIVO®), cemiplimab (LIBTAYO®), atezolizumab (TECENTRIQ®), avelumab (BAVENCIO®), durvalumab (IMFINZI®), or ipilimumab (YERVOY®).

In certain embodiments, the second therapeutic agent is a chemotherapy. For example, but without any limitation, chemotherapy includes CHOP (cyclophosphamide, doxorubicin, vincristie, prednisone), EPOCH (etoposide, vincristine, doxorubicin, cyclophosphamide, prednisone), or any other multidrug regimens.

In certain embodiments, the anti-L1CAM antibodies or antigen-binding fragments thereof, the immunoconjugate, the multi-specific molecule, or the composition disclosed herein and the second therapeutic agent can be administered to the subject as part of a treatment regimen. In certain embodiments, the anti-L1CAM antibodies or antigen-binding fragments thereof, the immunoconjugate, the multi-specific molecule, or the composition disclosed herein and the second therapeutic agent can be administered concurrently to the subject. In certain embodiments, the anti-L1CAM antibodies or antigen-binding fragments thereof, the immunoconjugate, the multi-specific molecule, or the composition disclosed herein and the second therapeutic agent can be administered at the same time. In certain embodiments, the anti-L1CAM antibodies or antigen-binding fragments thereof, the immunoconjugate, the multi-specific molecule, or the composition disclosed herein and the second therapeutic agent can be administered sequentially in any order (e.g., the anti-L1CAM antibodies or antigen-binding fragments thereof, the immunoconjugate, the multi-specific molecule, or the composition disclosed herein are administered to the subject after the second therapeutic agent is administered) or at different points in time (e.g., the anti-L1CAM antibodies or antigen-binding fragments thereof, the immunoconjugate, the multi-specific molecule, or the composition disclosed herein and the second therapeutic agent are administered to the subject on the same day but different hours; or the anti-L1CAM antibodies or antigen-binding fragments thereof, the immunoconjugate, the multi-specific molecule, or the composition disclosed herein and the second therapeutic agent are administered to the subject in the same week but on different days).

### 7. Diagnostic and Prognostic Methods

The presently disclosed anti-L1CAM antibodies, antigen-binding fragments thereof, multi-specific molecules, and nucleic acids encode thereof can be used for diagnostic and prognostic applications as well as use as research tools for detection of L1CAM in a biological sample, in a cell, a tissue, or a blood sample. The presently disclosed subject matter provides methods for detecting L1CAM in a cell, a tissue, or a blood sample. In certain embodiments, the method comprises: contacting a cell, a tissue, or a blood sample with the antibody, antigen-binding fragment thereof, or multi-specific molecule disclosed herein, wherein the antibody, antigen-binding fragment thereof or multi-specific molecule comprises a detectable label; and determining the amount of the labeled antibody, antigen-binding fragment thereof, or multi-specific molecule bound to the cell, tissue, or blood sample by measuring the amount of detectable label associated with the cell or tissue, wherein the amount of bound antibody, antigen-binding fragment thereof, or multi-specific molecule indicates the amount of L1CAM in the cell, tissue, or a blood sample. The cell or tissue can be any cell or tissue, including any normal, healthy, or cancerous cells and tissues. In certain embodiments, the blood sample is a peripheral blood sample.

The presently disclosed anti-L1CAM antibodies or antigen-binding fragments thereof can be used in methods known in the art relating to the localization and/or quantitation of L1CAM polypeptides (e.g., for use in measuring levels of the L1CAM protein within appropriate physiological samples, for use in diagnostic methods, for use in imaging the polypeptide, and the like). The presently disclosed anti-L1CAM antibodies or antigen-binding fragments thereof can be used to isolate a L1CAM polypeptide by standard techniques, such as affinity chromatography or immunoprecipitation. The presently disclosed anti-L1CAM antibodies or antigen-binding fragments thereof can facilitate the purification of natural immunoreactive

L1CAM proteins from biological samples, e.g., mammalian sera or cells as well as recombinantly-produced immunoreactive L1CAM proteins expressed in a host system. Moreover, anti-L1CAM antibodies of the present technology can be used to detect an immunoreactive L1CAM protein (e.g., in plasma, a cellular lysate or cell supernatant) in order to evaluate the abundance and pattern of expression of the immunoreactive polypeptide. The presently disclosed anti-L1CAM antibodies or antigen-binding fragments thereof can be used diagnostically to monitor immunoreactive L1CAM protein levels in tissue as part of a clinical testing procedure, e.g., to determine the efficacy of a given treatment regimen. As noted above, the detection can be facilitated by coupling (i.e., physically linking) the presently disclosed anti-L1CAM antibodies or antigen-binding fragments thereof to a detectable substance.

An exemplary method for detecting the presence or absence of an immunoreactive L1CAM protein in a biological sample comprises contacting a biological sample from a subject with a presently disclosed anti-L1CAM antibody or an antigen-binding fragment thereof, wherein the presence of an immunoreactive L1CAM protein is detected in the biological sample. Detection may be accomplished by means of a detectable label attached to the antibody.

The term “labeled” with regard to the anti-L1CAM antibody or antigen-binding fragment thereof is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-L1CAM antibodies or antigen-binding fragments thereof are conjugated to one or more detectable labels. For such uses, the presently disclosed anti-L1CAM antibodies or antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive L1CAM protein in a biological sample in vitro as well as in vivo. Non-limiting examples of in vitro techniques for detection of an immunoreactive L1CAM protein include enzyme linked immunosorbent assays (ELISAs), Western blots, immunoprecipitations, radioimmunoassay, and immunofluorescence. Furthermore, in vivo techniques for detection of an immunoreactive L1CAM protein include introducing into a subject a labeled anti-L1CAM antibody or an antigen-binding fragment thereof. For example, the anti-L1CAM antibody or antigen-binding fragment thereof can be labeled with a radioactive marker whose presence and location in a subject can be detected by standard imaging techniques. In certain embodiments, the biological sample comprises L1CAM protein molecules from the test subject.

The presently disclosed anti-L1CAM antibodies or antigen-binding fragments thereof can be used to assay immunoreactive L1CAM protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive L1CAM protein levels in a biological sample, the presently disclosed anti-L1CAM antibodies or antigen-binding fragments thereof may be used for in vivo imaging of L1CAM. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-L1CAM antibodies by labeling of nutrients for the relevant scFv clone.

The presently disclosed anti-L1CAM antibodies or antigen-binding fragments thereof, which are labeled with an appropriate detectable imaging moiety (such as a radioisotope (e.g., ¹³¹I, ¹¹¹IN, ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled anti-L1CAM antibody or antigen-binding fragment thereof then accumulates at the location of cells which contain the specific target polypeptide. For example, the labeled anti-L1CAM antibodies or antigen-binding fragments thereof accumulate within the subject in cells and tissues in which the L1CAM protein has localized.

Thus, the presently disclosed subject matter provides diagnostic methods of a medical condition. In certain embodiments, the method comprises: (a) assaying the expression of immunoreactive L1CAM protein by measuring binding of a presently disclosed anti-L1CAM antibody or an antigen-binding fragment thereof in cells or body fluid of an individual; and (b) comparing the amount of immunoreactive L1CAM protein present in the sample with a standard reference, wherein an increase or decrease in immunoreactive L1CAM protein levels compared to the standard is indicative of a medical condition.

Furthermore, the presently disclosed anti-L1CAM antibodies or antigen-binding fragments thereof may be used to purify immunoreactive L1CAM protein from a sample. In certain embodiments, the antibodies are immobilized on a solid support. Non-limiting examples of such solid supports include plastics such as polycarbonate, complex carbohydrates such as agarose and sepharose, acrylic resins and such as polyacrylamide and latex beads. Techniques for coupling antibodies to such solid supports are well known in the art.

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed anti-L1CAM antibodies or antigen-binding fragments thereof are useful in diagnostic methods. As such, the presently disclosed subject matter provides methods using the presently disclosed anti-L1CAM antibodies or antigen-binding fragments thereof in diagnosis of L1CAM activity in a subject. The presently disclosed anti-L1CAM antibodies or antigen-binding fragments thereof may be selected such that they have any level of epitope binding specificity and high binding affinity to a L1CAM polypeptide.

The presently disclosed anti-L1CAM antibodies or antigen-binding fragments thereof can be used to detect an immunoreactive L1CAM protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of L1CAM protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. Such assays use one antibody, e.g., the anti-L1CAM antibody or a population of anti-L1CAM antibodies immobilized to a solid phase, and another anti-L1CAM antibody or a population of anti-L1CAM antibodies in solution. Typically, the solution anti-L1CAM antibody or population of anti-L1CAM antibodies is labeled. If an antibody population is used, the population can contain antibodies binding to different epitope specificities within the target polypeptide. Accordingly, the same population can be used for both solid phase and solution antibody. If anti-L1CAM monoclonal antibodies are used, first and second L1CAM monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the L1CAM protein with the anti-L1CAM antibody, a sample is incubated for a period that usually varies from about 10 min to about 24 hr and is usually about 1 hr. A wash step is then performed to remove components of the sample not specifically bound to the anti-L1CAM antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive L1CAM protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the L1CAM protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-L1CAM antibodies can be joined to a linker molecule, such as biotin for attachment to a surface bound linker, such as avidin.

In certain embodiments, the presently disclosed anti-L1CAM antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed anti-L1CAM antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 8. Kits

The presently disclosed subject matter provides kits for treatment or ameliorating a disease or disorder associated with L1CAM (e.g., a metastatic cancer cell), and/or detecting L1CAM. In certain embodiments, the kit comprises the anti-L1CAM antibodies or antigen-binding fragments thereof, the immunoconjugate, the multi-specific molecule, or the composition disclosed herein.

In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-L1CAM antibodies or antigen-binding fragments thereof, the immunoconjugate, the multi-specific molecule, or the composition disclosed herein to a subject in need the treatment. The instructions can generally include information about the use of the anti-L1CAM antibodies or antigen-binding fragments thereof, the immunoconjugate, the multi-specific molecule, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 9. Exemplary Embodiments

Embodiment 1. An anti-L1CAM antibody or an antigen-binding fragment thereof, comprising: (a) a heavy chain variable region comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, a CDR3 comprising the amino acid sequence set forth in SEQ ID NO: 4 or a conservative modification thereof; and a light chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof; (b) a heavy chain variable region comprising CDR1 comprising the amino acid sequence set forth in SEQ ID NO: 12 or a conservative modification thereof, a CDR2 comprising an amino acid sequence set forth in SEQ ID NO: 13 or a conservative modification thereof, a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof; (c) a heavy chain variable region comprising CDR1 comprising the amino acid sequence set forth in SEQ ID NO: 12 or a conservative modification thereof, a CDR2 comprising an amino acid sequence set forth in SEQ ID NO: 13 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof; (d) a heavy chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, a CDR3 comprising the amino acid sequence set forth in SEQ ID NO: 37 or a conservative modification thereof; and a light chain variable region comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof; (e) a heavy chain variable region comprising CDR1 comprising the amino acid sequence set forth in SEQ ID NO: 43 or a conservative modification thereof, a CDR2 comprising an amino acid sequence set forth in SEQ ID NO: 44 or a conservative modification thereof, a CDR3 comprising the amino acid sequence set forth in SEQ ID NO: 45 or a conservative modification thereof; and a light chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof; (f) a heavy chain variable region comprising CDR1 comprising the amino acid sequence set forth in SEQ ID NO: 43 or a conservative modification thereof, a CDR2 comprising an amino acid sequence set forth in SEQ ID NO: 44 or a conservative modification thereof, a CDR3 comprising the amino acid sequence set forth in SEQ ID NO: 45 or a conservative modification thereof; and a light chain variable region comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof; (g) a heavy chain variable region comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, a CDR3 comprising the amino acid sequence set forth in SEQ ID NO: 56 or a conservative modification thereof; and a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof; (h) a heavy chain variable region comprising CDR1 comprising the amino acid sequence set forth in SEQ ID NO: 62 or a conservative modification thereof, a CDR2 comprising an amino acid sequence set forth in SEQ ID NO: 63 or a conservative modification thereof, a CDR3 comprising the amino acid sequence set forth in SEQ ID NO: 64 or a conservative modification thereof; and a light chain variable region comprising a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof; (i) a heavy chain variable region comprising CDR1 comprising the amino acid sequence set forth in SEQ ID NO: 69 or a conservative modification thereof, a CDR2 comprising an amino acid sequence set forth in SEQ ID NO: 70 or a conservative modification thereof, a CDR3 comprising the amino acid sequence set forth in SEQ ID NO: 71 or a conservative modification thereof; and a light chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof; (j) a heavy chain variable region comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, a CDR3 comprising the amino acid sequence set forth in SEQ ID NO: 83 or a conservative modification thereof; and a light chain variable region comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof; (k) a heavy chain variable region comprising CDR1 comprising the amino acid sequence set forth in SEQ ID NO: 93 or a conservative modification thereof, a CDR2 comprising an amino acid sequence set forth in SEQ ID NO: 94 or a conservative modification thereof, a CDR3 comprising the amino acid sequence set forth in SEQ ID NO: 95 or a conservative modification thereof; and a light chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof; (1) a heavy chain variable region comprising CDR1 comprising the amino acid sequence set forth in SEQ ID NO: 105 or a conservative modification thereof, a CDR2 comprising an amino acid sequence set forth in SEQ ID NO: 106 or a conservative modification thereof, a CDR3 comprising the amino acid sequence set forth in SEQ ID NO: 107 or a conservative modification thereof; and a light chain variable region comprising a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof; or (m) a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a light chain variable region comprising a CDR1 comprising the amino acid sequence set forth in SEQ ID NO: 119 or a conservative modification thereof, a CDR2 comprising the amino acid sequence set forth in SEQ ID NO: 120, and a CDR3 comprising the amino acid sequence set forth in SEQ ID NO: 121 or a conservative modification thereof.

Embodiment 2. The anti-L1CAM antibody or an antigen-binding fragment thereof of embodiment 1, wherein: (a) the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; (b) the heavy chain variable region comprises CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; (c) the heavy chain variable region comprises CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 26; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; (d) the heavy chain variable region comprises a CDR1 comprising the amino acid sequence set forth in SEQ ID NO: 35, a CDR2 comprising the amino acid sequence set forth in SEQ ID NO: 36, a CDR3 comprising the amino acid sequence set forth in SEQ ID NO: 37; and the light chain variable region comprises a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40v; (e) the heavy chain variable region comprises CDR1 comprising the amino acid sequence set forth in SEQ ID NO: 43, a CDR2 comprising an amino acid sequence set forth in SEQ ID NO: 44, a CDR3 comprising the amino acid sequence set forth in SEQ ID NO: 45; and the light chain variable region comprises a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; (f) the heavy chain variable region comprises CDR1 comprising the amino acid sequence set forth in SEQ ID NO: 43, a CDR2 comprising an amino acid sequence set forth in SEQ ID NO: 44, a CDR3 comprising the amino acid sequence set forth in SEQ ID NO: 45; and the light chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40; (g) the heavy chain variable region comprises a CDR1 comprising the amino acid sequence set forth in SEQ ID NO: 54, a CDR2 comprising the amino acid sequence set forth in SEQ ID NO: 55, a CDR3 comprising the amino acid sequence set forth in SEQ ID NO: 56; and the light chain variable region comprises a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59; (h) the heavy chain variable region comprises CDR1 comprising the amino acid sequence set forth in SEQ ID NO: 62, a CDR2 comprising an amino acid sequence set forth in SEQ ID NO: 63, a CDR3 comprising the amino acid sequence set forth in SEQ ID NO: 64; and the light chain variable region comprises a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66; (i) the heavy chain variable region comprises CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; (j) the heavy chain variable region comprises a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86; (k) the heavy chain variable region comprises CDR1 comprising the amino acid sequence set forth in SEQ ID NO: 93, a CDR2 comprising an amino acid sequence set forth in SEQ ID NO: 94, a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; (1) the heavy chain variable region comprises CDR1 comprising the amino acid sequence set forth in SEQ ID NO: 105, a CDR2 comprising an amino acid sequence set forth in SEQ ID NO: 106, a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109; or (m) the heavy chain variable region comprises a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and the light chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

Embodiment 3. The anti-L1CAM antibody or antigen-binding fragment thereof of embodiment 1 or 2, comprising: (a) the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprising a CDR1 comprises the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; (b) the heavy chain variable region comprises a CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or (c) the heavy chain variable region comprises a CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

Embodiment 4. The anti-L1CAM antibody or antigen-binding fragment thereof of any one of embodiments 1-3, wherein the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprising a CDR1 comprises the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

Embodiment 5. The anti-L1CAM antibody or antigen-binding fragment thereof of any one of embodiments 1-3, wherein the heavy chain variable region comprises a CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

Embodiment 6. The anti-L1CAM antibody or antigen-binding fragment thereof of any one of embodiments 1-3, wherein the heavy chain variable region comprises a CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

Embodiment 7. The anti-L1CAM antibody or an antigen-binding fragment thereof of any one of embodiments 1-6, comprising (a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 22, SEQ ID NO: 24, SEQ ID NO: 25, SEQ ID NO: 27, SEQ ID NO: 28, SEQ ID NO: 29, SEQ ID NO: 33, SEQ ID NO: 41, SEQ ID NO: 49, SEQ ID NO: 60, SEQ ID NO: 67, SEQ ID NO: 75, SEQ ID NO: 87, SEQ ID NO: 99, SEQ ID NO: 110, SEQ ID NO: 122, SEQ ID NO: 128, SEQ ID NO: 130, SEQ ID NO: 134, or SEQ ID NO: 135; or (b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 9, SEQ ID NO: 19, SEQ ID NO: 23, SEQ ID NO: 30, SEQ ID NO: 31, SEQ ID NO: 32, SEQ ID NO: 34, SEQ ID NO: 42, SEQ ID NO: 50, SEQ ID NO: 53, SEQ ID NO: 61, SEQ ID NO: 68, SEQ ID NO: 76, SEQ ID NO: 88, SEQ ID NO: 100, SEQ ID NO: 111, SEQ ID NO: 123, SEQ ID NO: 129, SEQ ID NO: 131, SEQ ID NO: 132, or SEQ ID NO: 133.

Embodiment 8. The anti-L1CAM antibody or an antigen-binding fragment thereof of any one of embodiments 1-7, comprising: (a) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 9; (b) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 18, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (c) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23; (d) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23; (e) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23; (f) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23; (g) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23; (h) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23; (i) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30; (j) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30; (k) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30; (1) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30; (m) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30; (n) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30; (o) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31; (p) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31; (q) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31; (r) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31; (s) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31; (t) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31; (u) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (v) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (w) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (x) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (y) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (z) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32; (aa) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (ab) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (ac) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (ad) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (ae) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (af) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19; (ag) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 33, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 34; (ah) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 41, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 42; (ai) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 50; (aj) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 53; (ak) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 60, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 61; (al) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 67, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 68; (am) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 75, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 76; (an) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 87, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 88; (ao) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 99, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 100; (ap) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 110, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 111; (aq) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 122, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 123; (ar) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 128, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 129; (as) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131; (at) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132; (au) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133; (av) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131; (aw) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132; (ax) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133; (ay) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131; (az) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132; (ba) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133; and (bb) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 136.

Embodiment 9. The antibody or antigen-binding fragment thereof of any one of embodiments 1-8, wherein (a) the heavy chain variable region comprises the amino acid sequence set forth in SEQ ID NO: 8, and the light chain variable region comprises the amino acid sequence set forth in SEQ ID NO: 9; (b) the heavy chain variable region comprises the amino acid sequence set forth in SEQ ID NO: 18, and the light chain variable region comprises the amino acid sequence set forth in SEQ ID NO: 19; (c) the heavy chain variable region comprises the amino acid sequence set forth in SEQ ID NO: 22, and the light chain variable region comprises the amino acid sequence set forth in SEQ ID NO: 23; (d) the heavy chain variable region comprises the amino acid sequence set forth in SEQ ID NO: 24, and the light chain variable region comprises the amino acid sequence set forth in SEQ ID NO: 23; (e) the heavy chain variable region comprises the amino acid sequence set forth in SEQ ID NO: 25, and the light chain variable region comprises the amino acid sequence set forth in SEQ ID NO: 23; (f) the heavy chain variable region comprises the amino acid sequence set forth in SEQ ID NO: 27, and the light chain variable region comprises the amino acid sequence set forth in SEQ ID NO: 23; (g) the heavy chain variable region comprises the amino acid sequence set forth in SEQ ID NO: 28, and the light chain variable region comprises the amino acid sequence set forth in SEQ ID NO: 23; (h) the heavy chain variable region comprises the amino acid sequence set forth in SEQ ID NO: 29, and the light chain variable region comprises the amino acid sequence set forth in SEQ ID NO: 23; (i) the heavy chain variable region comprises the amino acid sequence set forth in SEQ ID NO: 22, and the light chain variable region comprises the amino acid sequence set forth in SEQ ID NO: 30; (j) the heavy chain variable region comprises the amino acid sequence set forth in SEQ ID NO: 24, and the light chain variable region comprises the amino acid sequence set forth in SEQ ID NO: 30; (k) the heavy chain variable region comprises the amino acid sequence set forth in SEQ ID NO: 25, and the light chain variable region comprises the amino acid sequence set forth in SEQ ID NO: 30; (1) the heavy chain variable region comprises the amino acid sequence set forth in SEQ ID NO: 27, and the light chain variable region comprises the amino acid sequence set forth in SEQ ID NO: 30; (m) the heavy chain variable region comprises the amino acid sequence set forth in SEQ ID NO: 28, and the light chain variable region comprises the amino acid sequence set forth in SEQ ID NO: 30; (n) the heavy chain variable region comprises the amino acid sequence set forth in SEQ ID NO: 29, and the light chain variable region comprises the amino acid sequence set forth in SEQ ID NO: 30; (o) the heavy chain variable region comprises the amino acid sequence set forth in SEQ ID NO: 22, and the light chain variable region comprises the amino acid sequence set forth in SEQ ID NO: 31; (p) the heavy chain variable region comprises the amino acid sequence set forth in SEQ ID NO: 24, and the light chain variable region comprises the amino acid sequence set forth in SEQ ID NO: 31; (q) the heavy chain variable region comprises the amino acid sequence set forth in SEQ ID NO: 25, and the light chain variable region comprises the amino acid sequence set forth in SEQ ID NO: 31; (r) the heavy chain variable region comprises the amino acid sequence set forth in SEQ ID NO: 27, and the light chain variable region comprises the amino acid sequence set forth in SEQ ID NO: 31; (s) the heavy chain variable region comprises the amino acid sequence set forth in SEQ ID NO: 28, and the light chain variable region comprises the amino acid sequence set forth in SEQ ID NO: 31; (t) the heavy chain variable region comprises the amino acid sequence set forth in SEQ ID NO: 29, and the light chain variable region comprises the amino acid sequence set forth in SEQ ID NO: 31; (u) the heavy chain variable region comprises the amino acid sequence set forth in SEQ ID NO: 22, and the light chain variable region comprises the amino acid sequence set forth in SEQ ID NO: 32; (v) the heavy chain variable region comprises the amino acid sequence set forth in SEQ ID NO: 24, and the light chain variable region comprises the amino acid sequence set forth in SEQ ID NO: 32; (w) the heavy chain variable region comprises the amino acid sequence set forth in SEQ ID NO: 25, and the light chain variable region comprises the amino acid sequence set forth in SEQ ID NO: 32; (x) the heavy chain variable region comprises the amino acid sequence set forth in SEQ ID NO: 27, and the light chain variable region comprises the amino acid sequence set forth in SEQ ID NO: 32; (y) the heavy chain variable region comprises the amino acid sequence set forth in SEQ ID NO: 28, and the light chain variable region comprises the amino acid sequence set forth in SEQ ID NO: 32; (z) the heavy chain variable region comprises the amino acid sequence set forth in SEQ ID NO: 29, and the light chain variable region comprises the amino acid sequence set forth in SEQ ID NO: 32; (aa) the heavy chain variable region comprises the amino acid sequence set forth in SEQ ID NO: 22, and the light chain variable region comprises the amino acid sequence set forth in SEQ ID NO: 19; (ab) the heavy chain variable region comprises the amino acid sequence set forth in SEQ ID NO: 24, and the light chain variable region comprises the amino acid sequence set forth in SEQ ID NO: 19; (ac) the heavy chain variable region comprises the amino acid sequence set forth in SEQ ID NO: 25, and the light chain variable region comprises the amino acid sequence set forth in SEQ ID NO: 19; (ad) the heavy chain variable region comprises the amino acid sequence set forth in SEQ ID NO: 27, and the light chain variable region comprises the amino acid sequence set forth in SEQ ID NO: 19; (ae) the heavy chain variable region comprises the amino acid sequence set forth in SEQ ID NO: 28, and the light chain variable region comprises the amino acid sequence set forth in SEQ ID NO: 19; (af) the heavy chain variable region comprises the amino acid sequence set forth in SEQ ID NO: 29, and the light chain variable region comprises the amino acid sequence set forth in SEQ ID NO: 19; (ag) the heavy chain variable region comprising the amino acid sequence set forth in SEQ ID NO: 33, and the light chain variable region comprising the amino acid sequence set forth in SEQ ID NO: 34; (ah) the heavy chain variable region comprising the amino acid sequence set forth in SEQ ID NO: 41, and the light chain variable region comprising the amino acid sequence set forth in SEQ ID NO: 42; (ai) the heavy chain variable region comprising the amino acid sequence set forth in SEQ ID NO: 49, and the light chain variable region comprising the amino acid sequence set forth in SEQ ID NO: 50; (aj) the heavy chain variable region comprising the amino acid sequence set forth in SEQ ID NO: 49, and the light chain variable region comprising the amino acid sequence set forth in SEQ ID NO: 53; (ak) the heavy chain variable region comprising the amino acid sequence set forth in SEQ ID NO: 60, and the light chain variable region comprising the amino acid sequence set forth in SEQ ID NO: 61; (al) the heavy chain variable region comprising the amino acid sequence set forth in SEQ ID NO: 67, and the light chain variable region comprising the amino acid sequence set forth in SEQ ID NO: 68; (am) the heavy chain variable region comprising the amino acid sequence set forth in SEQ ID NO: 75, and the light chain variable region comprising the amino acid sequence set forth in SEQ ID NO: 76; (an) the heavy chain variable region comprising the amino acid sequence set forth in SEQ ID NO: 87, and the light chain variable region comprising the amino acid sequence set forth in SEQ ID NO: 88; (ao) the heavy chain variable region comprising the amino acid sequence set forth in SEQ ID NO: 99, and the light chain variable region comprising the amino acid sequence set forth in SEQ ID NO: 100; (ap) the heavy chain variable region comprising the amino acid sequence set forth in SEQ ID NO: 110, and the light chain variable region comprising the amino acid sequence set forth in SEQ ID NO: 111; (aq) the heavy chain variable region comprising the amino acid sequence set forth in SEQ ID NO: 122, and the light chain variable region comprising the amino acid sequence set forth in SEQ ID NO: 123; (ar) the heavy chain variable region comprising the amino acid sequence set forth in SEQ ID NO: 128, and the light chain variable region comprising the amino acid sequence set forth in SEQ ID NO: 129; (as) the heavy chain variable region comprising the amino acid sequence set forth in SEQ ID NO: 130, and the light chain variable region comprising the amino acid sequence set forth in SEQ ID NO: 131; (at) the heavy chain variable region comprising the amino acid sequence set forth in SEQ ID NO: 130, and the light chain variable region comprising the amino acid sequence set forth in SEQ ID NO: 132; (au) the heavy chain variable region comprising the amino acid sequence set forth in SEQ ID NO: 130, and the light chain variable region comprising the amino acid sequence set forth in SEQ ID NO: 133; (av) the heavy chain variable region comprising the amino acid sequence set forth in SEQ ID NO: 134, and the light chain variable region comprising the amino acid sequence set forth in SEQ ID NO: 131; (aw) the heavy chain variable region comprising the amino acid sequence set forth in SEQ ID NO: 134, and the light chain variable region comprising the amino acid sequence set forth in SEQ ID NO: 132; (ax) the heavy chain variable region comprising the amino acid sequence set forth in SEQ ID NO: 134, and the light chain variable region comprising the amino acid sequence set forth in SEQ ID NO: 133; (ay) the heavy chain variable region comprising the amino acid sequence set forth in SEQ ID NO: 135, and the light chain variable region comprising the amino acid sequence set forth in SEQ ID NO: 131; (az) the heavy chain variable region comprising the amino acid sequence set forth in SEQ ID NO: 135, and the light chain variable region comprising the amino acid sequence set forth in SEQ ID NO: 132; (ba) the heavy chain variable region comprising the amino acid sequence set forth in SEQ ID NO: 135, and the light chain variable region comprising the amino acid sequence set forth in SEQ ID NO: 133; or (bb) the heavy chain variable region comprising the amino acid sequence set forth in SEQ ID NO: 135, and the light chain variable region comprising the amino acid sequence set forth in SEQ ID NO: 136.

Embodiment 10. The anti-L1CAM antibody or antigen-binding fragment thereof of any one of embodiments 1-9, wherein the antibody comprises a comprises a heavy chain constant region and/or a light chain constant region.

Embodiment 11. The anti-L1CAM antibody or antigen-binding fragment thereof of embodiment 10, wherein: (a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence set forth in SEQ ID NO: 144, SEQ ID NO: 145, SEQ ID NO: 146, or SEQ ID NO: 147; and/or (b) the light chain constant region comprises an amino acid sequence that is at least about 80%, at least about 85%, at least about 90%, at least about 95%, at least about 96%, at least about 97%, at least about 98%, at least about 99% at least about 100% identical to the amino acid sequence set forth in SEQ ID NO: 148.

Embodiment 12. The anti-L1CAM antibody or antigen-binding fragment thereof of embodiment 10 or 11, wherein: (a) the heavy chain constant region comprises the amino acid sequence set forth in SEQ ID NO: 144, SEQ ID NO: 145, SEQ ID NO: 146, or SEQ ID NO: 147; and/or (b) the light chain constant region comprises the amino acid sequence set forth in SEQ ID NO: 148.

Embodiment 13. The anti-L1CAM antibody or antigen-binding fragment thereof of any one of embodiments 1-12, wherein the antibody comprises a human variable region framework region.

Embodiment 14. The anti-L1CAM antibody or antigen-binding fragment thereof of any one of embodiments 1-13, which is a fully human or an antigen-binding fragment thereof.

Embodiment 15. The anti-L1CAM antibody or antigen-binding fragment thereof of any one of embodiments 1-12, which is a chimeric antibody or an antigen-binding fragment thereof.

Embodiment 16. The anti-L1CAM antibody or antigen-binding fragment thereof of any one of embodiments 1-12, which is a humanized antibody or an antigen-binding fragment thereof.

Embodiment 17. The anti-L1CAM antibody or antigen-binding fragment thereof of any one of embodiments 1-16, wherein the antigen-binding fragment is a Fab, Fab′, F(ab′)2, variable fragment (Fv), or single chain variable region (scFv).

Embodiment 18. The anti-L1CAM antibody or antigen-binding fragment thereof of embodiment 17, wherein the antigen-binding fragment is an scFv.

Embodiment 19. An anti-L1CAM antibody or an antigen-binding fragment thereof comprising (a) a heavy chain variable region comprising a CDR1, a CDR2, and a CDR3 of the heavy chain variable region comprising the amino acid sequence set forth in SEQ ID NO: 8, and a light chain variable region comprising a CDR1, a CDR2, and a CDR3 of the light chain variable region comprising the amino acid sequence set forth in SEQ ID NO: 9; (b) a heavy chain variable region comprising a CDR1, a CDR2, and a CDR3 of the heavy chain variable region comprising the amino acid sequence set forth in SEQ ID NO: 18, and a light chain variable region comprising a CDR1, a CDR2, and a CDR3 of the light chain variable region comprising the amino acid sequence set forth in SEQ ID NO: 19; (c) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 23; (d) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 23; (e) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 23; (f) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 23; (g) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 23; (h) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 23; (i) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 30; (j) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 30; (k) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 30; (1) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 30; (m) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 30; (n) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 30; (o) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 31; (p) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 31; (q) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 31; (r) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 31; (s) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 31; (t) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 31; (u) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 32; (v) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 32; (w) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 32; (x) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 32; (y) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 32; (z) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 32; (aa) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 19; (ab) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 19; (ac) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 19; (ad) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 19; (ae) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 19; (af) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 19; (ag) the heavy chain variable region comprising the amino acid sequence set forth in SEQ ID NO: 33, and the light chain variable region comprising the amino acid sequence set forth in SEQ ID NO: 34; (ah) the heavy chain variable region comprising the amino acid sequence set forth in SEQ ID NO: 41, and the light chain variable region comprising the amino acid sequence set forth in SEQ ID NO: 42; (ai) the heavy chain variable region comprising the amino acid sequence set forth in SEQ ID NO: 49, and the light chain variable region comprising the amino acid sequence set forth in SEQ ID NO: 50; (aj) the heavy chain variable region comprising the amino acid sequence set forth in SEQ ID NO: 49, and the light chain variable region comprising the amino acid sequence set forth in SEQ ID NO: 53; (ak) the heavy chain variable region comprising the amino acid sequence set forth in SEQ ID NO: 60, and the light chain variable region comprising the amino acid sequence set forth in SEQ ID NO: 61; (al) the heavy chain variable region comprising the amino acid sequence set forth in SEQ ID NO: 67, and the light chain variable region comprising the amino acid sequence set forth in SEQ ID NO: 68; (am) the heavy chain variable region comprising the amino acid sequence set forth in SEQ ID NO: 75, and the light chain variable region comprising the amino acid sequence set forth in SEQ ID NO: 76; (an) the heavy chain variable region comprising the amino acid sequence set forth in SEQ ID NO: 87, and the light chain variable region comprising the amino acid sequence set forth in SEQ ID NO: 88; (ao) the heavy chain variable region comprising the amino acid sequence set forth in SEQ ID NO: 99, and the light chain variable region comprising the amino acid sequence set forth in SEQ ID NO: 100; (ap) the heavy chain variable region comprising the amino acid sequence set forth in SEQ ID NO: 110, and the light chain variable region comprising the amino acid sequence set forth in SEQ ID NO: 111; (aq) the heavy chain variable region comprising the amino acid sequence set forth in SEQ ID NO: 122, and the light chain variable region comprising the amino acid sequence set forth in SEQ ID NO: 123; (ar) the heavy chain variable region comprising the amino acid sequence set forth in SEQ ID NO: 128, and the light chain variable region comprising the amino acid sequence set forth in SEQ ID NO: 129; (as) the heavy chain variable region comprising the amino acid sequence set forth in SEQ ID NO: 130, and the light chain variable region comprising the amino acid sequence set forth in SEQ ID NO: 131; (at) the heavy chain variable region comprising the amino acid sequence set forth in SEQ ID NO: 130, and the light chain variable region comprising the amino acid sequence set forth in SEQ ID NO: 132; (au) the heavy chain variable region comprising the amino acid sequence set forth in SEQ ID NO: 130, and the light chain variable region comprising the amino acid sequence set forth in SEQ ID NO: 133; (av) the heavy chain variable region comprising the amino acid sequence set forth in SEQ ID NO: 134, and the light chain variable region comprising the amino acid sequence set forth in SEQ ID NO: 131; (aw) the heavy chain variable region comprising the amino acid sequence set forth in SEQ ID NO: 134, and the light chain variable region comprising the amino acid sequence set forth in SEQ ID NO: 132; (ax) the heavy chain variable region comprising the amino acid sequence set forth in SEQ ID NO: 134, and the light chain variable region comprising the amino acid sequence set forth in SEQ ID NO: 133; (ay) the heavy chain variable region comprising the amino acid sequence set forth in SEQ ID NO: 135, and the light chain variable region comprising the amino acid sequence set forth in SEQ ID NO: 131; (az) the heavy chain variable region comprising the amino acid sequence set forth in SEQ ID NO: 135, and the light chain variable region comprising the amino acid sequence set forth in SEQ ID NO: 132; (ba) the heavy chain variable region comprising the amino acid sequence set forth in SEQ ID NO: 135, and the light chain variable region comprising the amino acid sequence set forth in SEQ ID NO: 133; or (bb) the heavy chain variable region comprising the amino acid sequence set forth in SEQ ID NO: 135, and the light chain variable region comprising the amino acid sequence set forth in SEQ ID NO: 136.

Embodiment 20. An anti-L1CAM antibody or an antigen-binding fragment thereof comprising: (a) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 77 or SEQ ID NO: 78; (b) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 89 or SEQ ID NO: 90; (c) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 101 or SEQ ID NO: 102; (d) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 112 or SEQ ID NO: 113; or (e) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 124 or SEQ ID NO: 125.

Embodiment 21. The anti-L1CAM antibody or an antigen-binding fragment thereof of embodiment 20, wherein: (a) the scFv comprises the amino acid sequence set forth in SEQ ID NO: 77 or SEQ ID NO: 78; (b) the scFv comprises the amino acid sequence set forth in SEQ ID NO: 89 or SEQ ID NO: 90; (c) the scFv comprises the amino acid sequence set forth in SEQ ID NO: 101 or SEQ ID NO: 102; (d) the scFv comprises the amino acid sequence set forth in SEQ ID NO: 112 or SEQ ID NO: 113; or (e) the scFv comprises the amino acid sequence set forth in SEQ ID NO: 124 or SEQ ID NO: 125.

Embodiment 22. An anti-L1CAM antibody or an antigen-binding fragment thereof comprising a heavy chain variable region comprising a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

Embodiment 23. An anti-L1CAM antibody or an antigen-binding fragment thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

Embodiment 24. An anti-L1CAM antibody or an antigen-binding fragment thereof comprising a heavy chain variable region comprising a CDR1 comprising the amino acid sequence set forth in SEQ ID NO: 69, a CDR2 comprising the amino acid sequence set forth in SEQ ID NO: 70, a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

Embodiment 25. An anti-L1CAM antibody or an antigen-binding fragment thereof comprising a heavy chain variable region comprising a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

Embodiment 26. An anti-L1CAM antibody or an antigen-binding fragment thereof comprising a heavy chain variable region comprising a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 95; and a light chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

Embodiment 27. An anti-L1CAM antibody or an antigen-binding fragment thereof comprising a heavy chain variable region comprising a heavy chain variable region comprising the amino acid sequence set forth in SEQ ID NO: 8, and a light chain variable region comprising the amino acid sequence set forth in SEQ ID NO: 9.

Embodiment 28. An anti-L1CAM antibody or an antigen-binding fragment thereof comprising a heavy chain variable region comprising a heavy chain variable region comprising the amino acid sequence set forth in SEQ ID NO: 18, and a light chain variable region comprising the amino acid sequence set forth in SEQ ID NO: 19.

Embodiment 29. An anti-L1CAM antibody or an antigen-binding fragment thereof comprising a heavy chain variable region comprising a heavy chain variable region comprising the amino acid sequence set forth in SEQ ID NO: 75, and a light chain variable region comprising the amino acid sequence set forth in SEQ ID NO: 76.

Embodiment 30. An anti-L1CAM antibody or an antigen-binding fragment thereof comprising a heavy chain variable region comprising a heavy chain variable region comprising the amino acid sequence set forth in SEQ ID NO: 87, and a light chain variable region comprising the amino acid sequence set forth in SEQ ID NO: 88.

Embodiment 31. An anti-L1CAM antibody or an antigen-binding fragment thereof comprising a heavy chain variable region comprising a heavy chain variable region comprising the amino acid sequence set forth in SEQ ID NO: 99, and a light chain variable region comprising the amino acid sequence set forth in SEQ ID NO: 100.

Embodiment 32. An anti-L1CAM antibody or an antigen-binding fragment thereof comprising the scFv comprises the amino acid sequence set forth in SEQ ID NO: 77 or SEQ ID NO: 78.

Embodiment 33. An anti-L1CAM antibody or an antigen-binding fragment thereof comprising the scFv comprises the amino acid sequence set forth in SEQ ID NO: 89 or SEQ ID NO: 90.

Embodiment 34. An anti-L1CAM antibody or an antigen-binding fragment thereof comprising the scFv comprises the amino acid sequence set forth in SEQ ID NO: 101 or SEQ ID NO: 102.

Embodiment 35. An antibody or an antigen-binding fragment thereof, which cross-competes for binding to L1CAM with an anti-L1CAM antibody or an antigen-binding fragment thereof of any one of embodiments 1-34.

Embodiment 36. An antibody or an antigen-binding fragment thereof, which binds to the same epitope region on L1CAM with an anti-L1CAM antibody or an antigen-binding fragment thereof of any one of embodiments 1-34.

Embodiment 37. A composition comprising the anti-L1CAM antibody or antigen-binding fragment thereof of any one of embodiments 1-36.

Embodiment 38. The composition of embodiment 37, which is a pharmaceutical composition that further comprises a pharmaceutically acceptable carrier.

Embodiment 39. An immunoconjugate comprising the anti-L1CAM antibody or antigen-binding fragment thereof of any one of embodiments 1-36, linked to a therapeutic agent. Embodiment 40. The immunoconjugate of embodiment 39, wherein the therapeutic agent is a drug, a cytotoxin, or a radioactive isotope.

Embodiment 41. The immunoconjugate of embodiment 39 or 40, wherein the therapeutic agent is a compound selected from the group consisting of dolastatins, maytansinoids, duocarmycins, anthracyclines, camptothecins, and amatoxins.

Embodiment 42. The immunoconjugate of embodiment 41, wherein the therapeutic agent is selected from the group consisting of monomethyl auristatin E, ABZ038, duocarmycin TM, PNU159682, SN38, and α-Amanitin.

Embodiment 43. The immunoconjugate of embodiment 41 or 42, wherein the therapeutic agent is PNU159682.

Embodiment 44. The immunoconjugate of any one of embodiments 39-43 comprising a linker.

Embodiment 45. The immunoconjugate of embodiment 44, wherein the linker is a cleavable linker or a non-cleavable linker.

Embodiment 46. The immunoconjugate of any one of embodiments 39-45, comprising a molecule of formula:

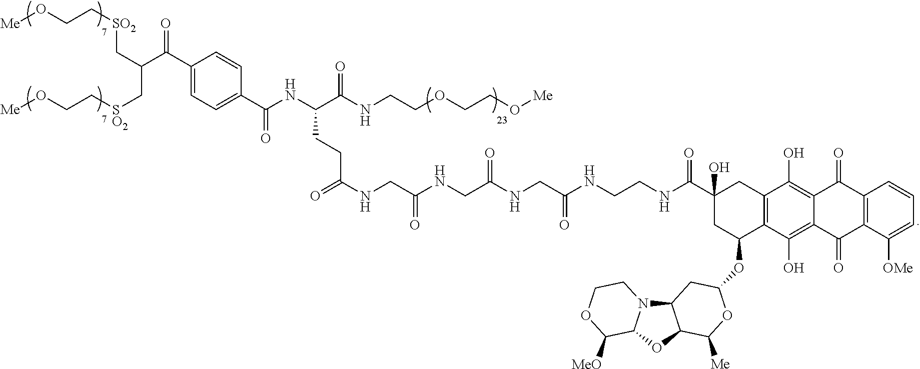

Embodiment 47. The immunoconjugate of any one of embodiments 39-46, comprising a molecule of formula:

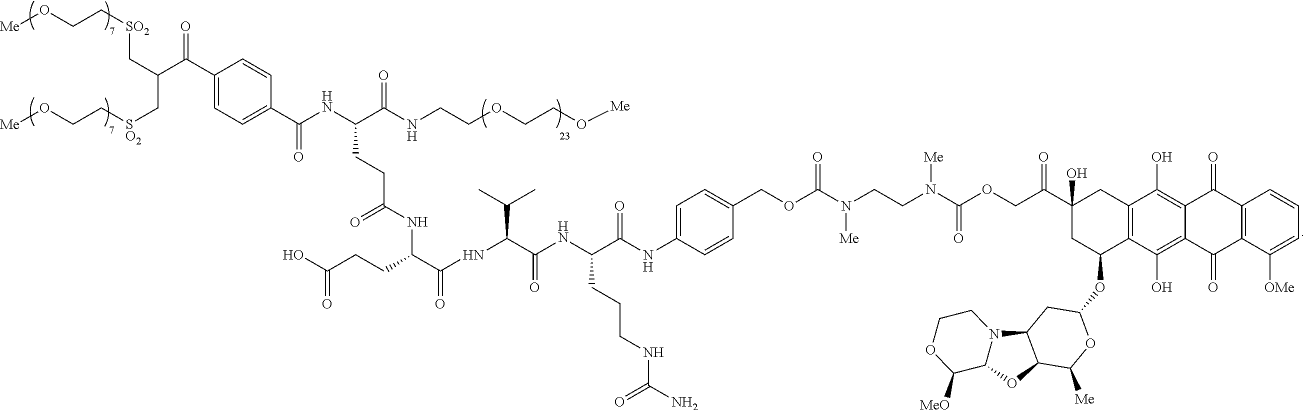

Embodiment 48. A composition comprising the immunoconjugate of any one of embodiments 39-47.

Embodiment 49. The composition of embodiment 48, which is a pharmaceutical composition that further comprises a pharmaceutically acceptable carrier.

Embodiment 50. A multi-specific molecule comprising the antibody or antigen-binding fragment thereof of any one of embodiments 1-36, linked to one or more functional moieties. Embodiment 51. The multi-specific molecule of embodiment 50, wherein the one or more functional moieties have a different binding specificity than the antibody or antigen binding fragment thereof.

Embodiment 52. A composition comprising the multi-specific molecule of embodiment 50 or 51.

Embodiment 53. The composition of embodiment 52, which is a pharmaceutical composition that further comprises a pharmaceutically acceptable carrier.

Embodiment 54. A nucleic acid that encodes an antibody or antigen-binding fragment thereof of any one of embodiments 1-36.

Embodiment 55. A nucleic acid that encodes a heavy chain variable region of an antibody or antigen-binding fragment thereof of any one of embodiments 1-36.

Embodiment 56. A nucleic acid that encodes a light chain variable region of an antibody or antigen-binding fragment thereof of any one of embodiments 1-36.

Embodiment 57. A vector comprising the nucleic acid of any one of embodiments 54-56.

Embodiment 58. A host cell comprising the vector of embodiment 57.

Embodiment 59. A method for detecting L1CAM in a whole cell, a tissue, or a blood sample, comprising: (a) contacting a cell, tissue or blood sample with the antibody or antigen-binding fragment thereof of any one of embodiments 1-36, wherein the antibody or antigen-binding fragment thereof comprises a detectable label; and (b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of L1CAM in the cell, tissue or blood sample.

Embodiment 60. A method of treating or ameliorating a disease or disorder associated with L1CAM in a subject, comprising administering to the subject the antibody or antigen-binding fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53.

Embodiment 61. The method of embodiment 60, wherein the disease or disorder is a tumor.

Embodiment 62. A method of reducing tumor burden in a subject, comprising administering to the subject an antibody or antigen-binding fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53.

Embodiment 63. The method of embodiment 62, wherein the method reduces the number of the tumor cells, reduces the tumor size, and/or eradicates the tumor in the subject.

Embodiment 64. A method of treating and/or preventing a tumor in a subject, comprising administering to the subject an antibody or antigen-binding fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53.

Embodiment 65. A method of increasing or lengthening survival of a subject having a tumor, comprising administering to the subject an antibody or antigen-binding fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53.

Embodiment 66. The method of embodiment 65, wherein the method reduces or eradicates tumor burden in the subject.

Embodiment 67. The method of any one of embodiments 60-66, wherein the tumor is cancer.

Embodiment 68. The method of any one of embodiments 60-67, wherein the tumor is a metastatic cancer.

Embodiment 69. The method of embodiment 68, wherein metastatic cancer is an advanced metastatic cancer.

Embodiment 70. The method of any one of embodiments 60-69, wherein the subject is a human.

Embodiment 71. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53.

Embodiment 72. The kit of embodiment 71, wherein the kit further comprises written instructions for using the antibody or antigen-binding fragment thereof, immunoconjugate, multi-specific molecule, or composition for treating or ameliorating a disease or disorder in a subject, reducing tumor burden in a subject, treating and/or preventing a tumor in a subject, and/or increasing or lengthening survival of a subject having a tumor.

Embodiment 73. The antibody or antigen fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53 for use in treating or ameliorating a disease or disorder in a subject, reducing tumor burden in a subject, treating and/or preventing a tumor in a subject, and/or increasing or lengthening survival of a subject having a tumor.

Embodiment 74. The antibody or antigen fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53 for treating or ameliorating a disease or disorder in a subject, reducing tumor burden in a subject, treating and/or preventing a tumor in a subject, and/or increasing or lengthening survival of a subject having a tumor.

Embodiment 75. Use of the antibody or antigen fragment thereof of any one of embodiments 1-36, the immunoconjugate of any one of embodiments 39-47, the multi-specific molecule of embodiment 50 or 51, or the composition of any one of embodiments 37, 38, 48, 49, 52, or 53 for the manufacturing of a medicament for treating or ameliorating a disease or disorder in a subject, reducing tumor burden in a subject, treating and/or preventing a tumor in a subject, and/or increasing or lengthening survival of a subject having a tumor.

### Examples

The following examples are put forth so as to provide those of ordinary skill in the art with a complete disclosure and description of how to make and use the antibodies, make and use immunoconjugates of such antibodies, compositions comprising thereof, screening, and therapeutic methods of the presently disclosed subject matter, and are not intended to limit the scope of what the inventors regard as their presently disclosed subject matter. It is understood that various other embodiments can be practiced, given the general description provided above.

### Example 1—LJ CAM is Associated with Metastasis and Poor Prognosis in Cancers

Residual metastatic disease after surgery or systemic therapy persists as single cancer cells and slow-cycling small cell clusters that are inherently resistant to anti-mitotic chemotherapy or to drugs targeting oncogenic drivers such as EGFR. Such metastasis stem cells (MetSCs) ultimately reinitiate tumor regrowth and drive lethal relapse, highlighting the clinical necessity to develop novel therapies targeting this unique population of cancer cells (Ganesh et al., Nat Med 27, 34-44 (2021); Massague & Ganesh, Cancer Discov 11, 971-994 (2021); Oskarsson et al., Cell Stem Cell 14, 306-321 (2014)).

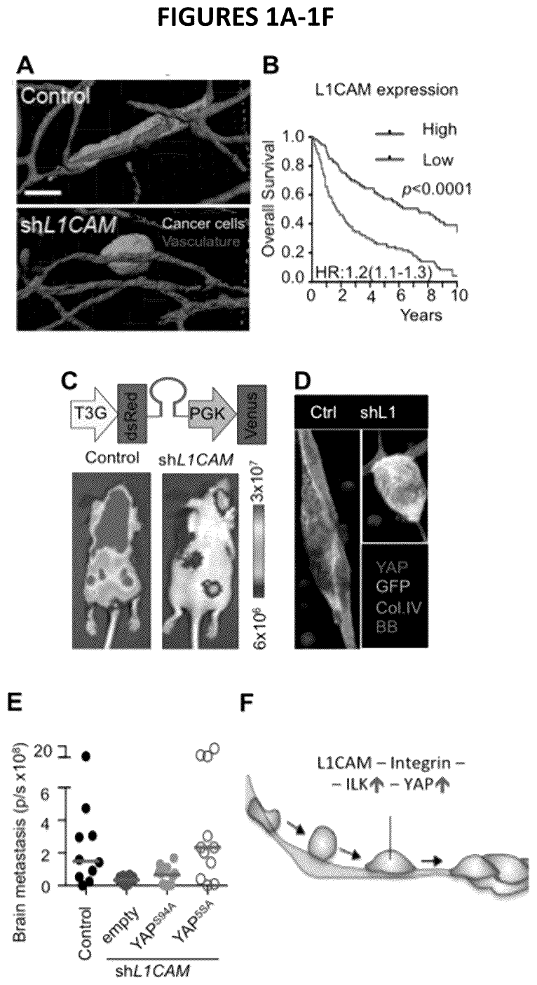

L1 cell adhesion molecule (L1CAM) was identified as an attractive target for the treatment of advanced solid tumors (Er et al., Nat Cell Biol 20, 966-978 (2018); Ganesh et al., Nat Cancer 1, 28-45 (2020); Valiente et al., Cell 156, 1002-1016 (2014)). The significance of L1CAM in cancer is three-fold: first, L1CAM is critical for the metastatic outgrowth of multiple solid tumors in three of the most prominent causes of cancer death, including lung, breast and colorectal cancer. Next, L1CAM is critical for metastatic outgrowth in multiple organ sites such as the bone, lung, liver and brain. Last, L1CAM is critical for the regeneration of aggressive tumors in multiple organs from small numbers of therapy-resistant, stem-like cells. In the brain and other organs, isolated MetSCs remain closely associated with the vasculature (Valiente et al., Cell 156, 1002-1016 (2014); Kienast et al., Nat Med 16, 116-122 (2010)). To initiate proliferation, MetSCs spread over capillaries, a process that requires L1CAM (FIG. 1A). L1CAM spreads and outgrowths in perivascular cell of colorectal, lung and breast carcinoma cells in multiple organ sites via the growth-promoting Hippo pathway effector YAP4 (FIGS. 1B-1F).

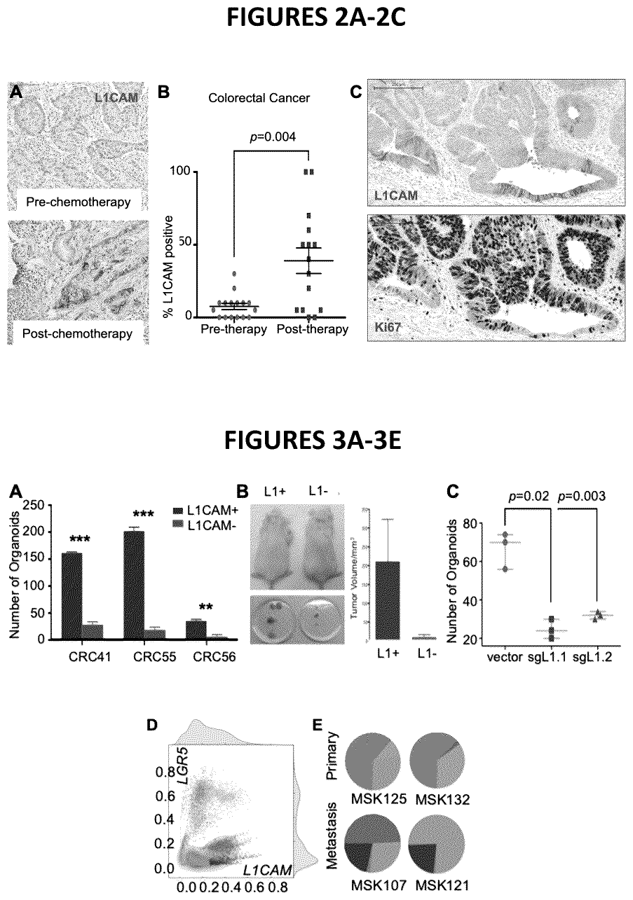

Originally identified as a neuronal adhesion molecule, L1CAM is associated with metastasis and poor prognosis in several cancers, including colorectal, lung and breast (Altevogt et al., Int J Cancer 147, 3292-3296 (2020); Altevogt et al., Int J Cancer 138, 1565-1576 (2016); Boo et al., Ann Surg Oncol 14, 1703-1711 (2007); Schroder et al., Oncol Rep 22, 1109-1117 (2009); Tischler et al., Mol Cancer 10, 127 (2011); Yu et al., Int J Clin Exp Pathol 12, 2665-2671 (2019)). In lung adenocarcinoma and colorectal cancer (CRC) patient samples, L1CAM⁺ cells were strongly enriched in the residual disease that survived chemotherapy in primary tumors, and in metastases (FIGS. 2A and 2B). Within individual tumors, only a subpopulation of cancer cells expresses L1CAM and unexpectedly the L1CAM⁺ cells in tumors are Ki67-low, suggesting that L1CAM+ cancer cells have the capacity to enter a slow-cycling or quiescent state (FIG. 2C).

Notably, quiescent L1CAM^{high} cells isolated by fluorescence-activated cell sorting (FACS) from surgically resected human CRC metastases preferentially regenerate heterogeneous organoids containing both L1CAM^{high} and L1CAM^{low} progeny. Organoid forming activity is widely regarded as the best in vitro correlate of cancer stem cell function (FIGS. 3A-3C). Using patient-derived metastatic CRC organoids and mouse models, it was demonstrated that disseminating CRCs undergo a dynamic phenotypic switch from an LGR5⁺ tumor-initiating cancer stem cell (CSC) state to an L1CAM⁺ state required for metastasis (FIGS. 3D and 3E).

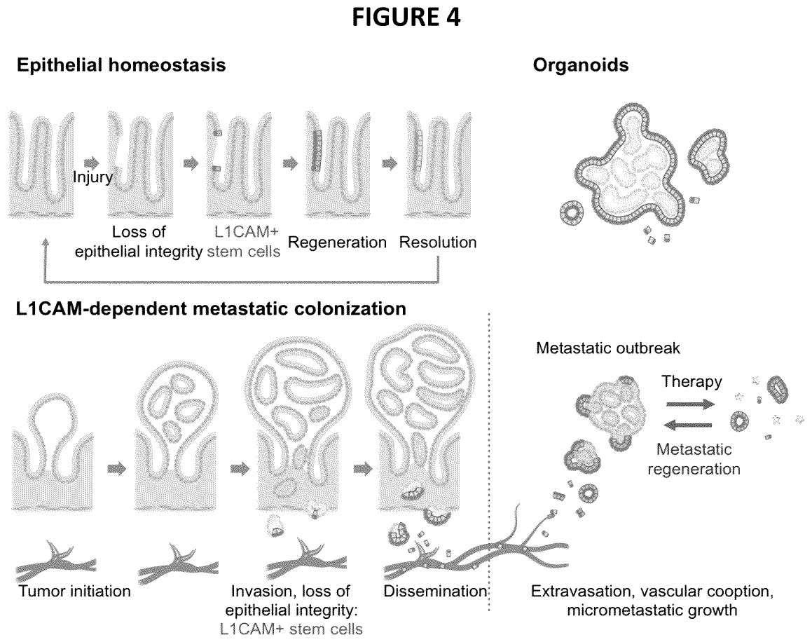

L1CAM⁺ MetSCs are functionally distinct from tumor-initiating LGR5⁺ CSCs: L1CAM is required for organoid formation, the regeneration of intestinal epithelium after colitis, and formation of tumors after metastatic dissemination or after therapy. But unlike LGR5, it is dispensable for epithelial homeostasis or intestinal tumor initiation. Similar to regeneration after tissue injury, disruption of epithelial integrity is an obligatory step in the metastatic cascade: cancer cells detaching from intact epithelia to invade the basal membrane, disseminating as single cells or small clusters, seeding distant organs or surviving as residual disease following therapy must evolve mechanisms to escape the loss of survival signaling provided by epithelial contact in intact tissues (FIG. 4). Thus, entry into an L1CAM^{high} regenerative stem-like state is a crucial requirement for epithelial regeneration during metastasis and tumor regeneration after therapy.

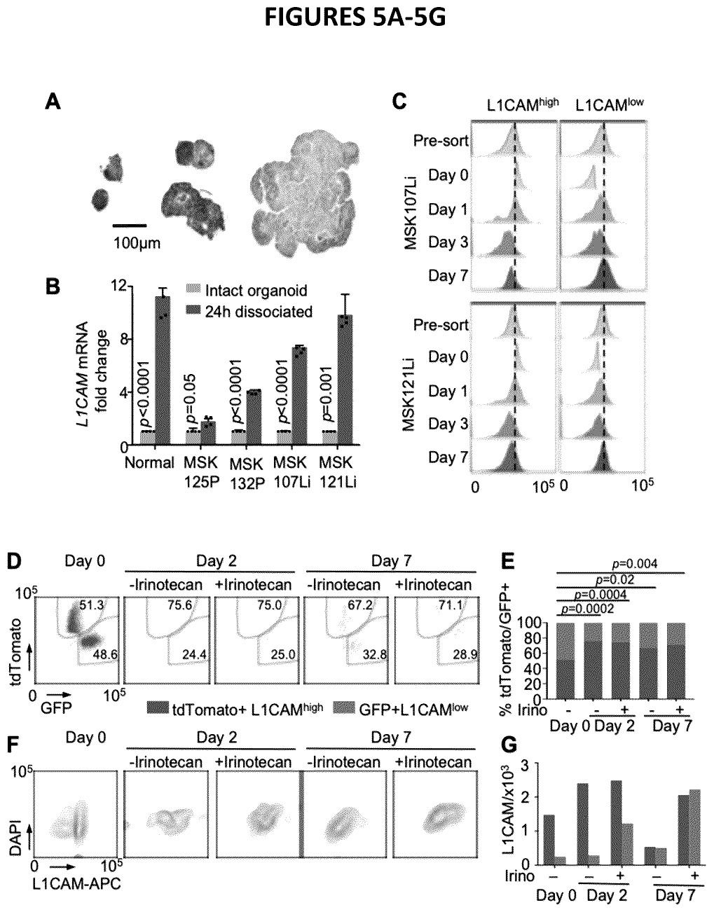

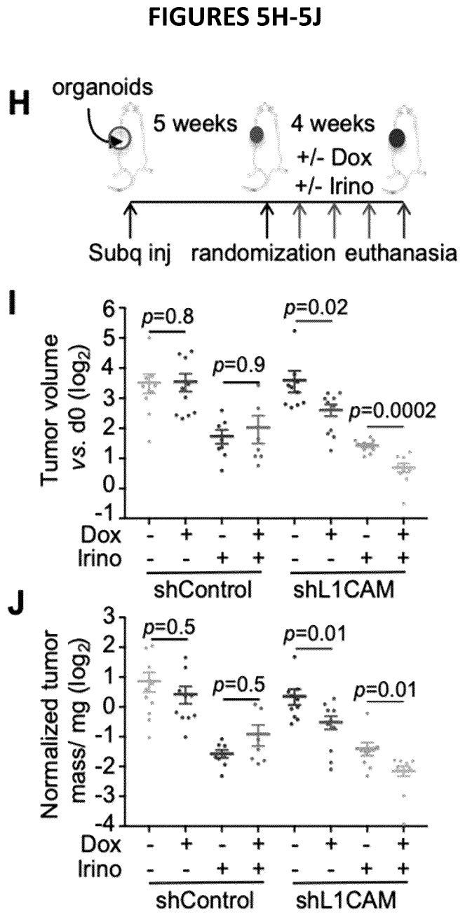

The identification of conserved molecular mediators of regenerative cell states required for metastasis and therapy resistance offers a powerful new approach to treat highly plastic advanced solid tumors more effectively. Plasticity poses a great challenge for cancer treatment since tumors can evade therapy by dynamically altering their phenotype. However, entry into a regenerative L1CAM⁺ MetSC state represents a conserved bottleneck of advanced cancers, which could be targeted therapeutically (Ganesh & Massague, Nat Med 27, 34-44 (2021); Massague & Ganesh, Cancer Discov 11, 971-994 (2021); Ganesh et al., Nat Cancer 1, 28-45 (2020)) (FIGS. 5A-5G). L1CAM⁺ cells isolated by fluorescence-activating cell sorting (FACS) from a wide variety of human tumors selectively regenerate organoids and xenograft tumors. By lineage tracing using the organoid regenerative capacity of L1CAM⁺/L1CAM⁻ cells with different fluorescent markers, it was demonstrated that a minority of L1CAM⁻ cells dynamically enter an L1CAM⁺ state and then regenerate organoids to organoid formation. Conventional therapies are effective at debulking rapidly proliferating L1CAM⁻ cancer cells but are unable to clear tumors due to the persistence of L1CAM⁺ MetSCs. These data demonstrated that combining chemotherapy with L1CAM inhibition was more effective at tumor inhibition than chemotherapy alone (FIGS. 5H-5J).

### Therapeutic Concept

L1CAM is present on cancer cells and clearly associated with aggressive disease, therapy resistance and metastatic outgrowth, whereas the protein is not expressed in the vast majority of normal tissues other than during neuronal development. These features make L1CAM an ideal target for approaches that seek to kill L1CAM-expressing cancer cells. As a cell adhesion molecule, L1CAM has a high intrinsic turnover rate with continuous internalization of the molecule. Leveraging this property, an ADC approach was selected as the therapeutic modality.

To maximize efficacy while maintaining safety, key design goals for the antibodies included high affinity (single-digit nM or sub-nM), selectivity over close homologs and the absence of non-specific binding, which could result in dose-limiting toxicities. In addition, the toxic payload and linker were carefully selected and modified to maximize the therapeutic window for the ADCs.

### Clinical Differentiation

A phase I clinical trial using L1CAM-targeted CAR-T cells in patients with neuroblastoma reported safety in six patients (Park et al., Mol Ther 15, 825-833 (2007)). These early results suggest that L1CAM, which is required during neuronal development (but has no clear role in normal adult physiology) can be safely targeted in metastatic cancer. Another study showed a human anti-L1CAM antibody that “significantly inhibited tumor growth in a human cholangiocarcinoma xenograft nude mouse model” (Cho et al., MAbs 8, 414-425 (2016)).

Although L1CAM-targeted antibodies have previously been described and showed efficacy in xenograft models, none has advanced to a clinical trial. This may be due in part to the fact that L1CAM has a large, multi-domain extracellular region. Antibodies binding in the extracellular domain may not necessarily disrupt the specific interactions that allow L1CAM to promote metastatic outgrowth. The ADC approach overcomes these limitations by delivering a cytotoxic drug payload to regenerating MetSCs that express L1CAM.

### Example 2—Antibody Generation

This example describes the generation of antibodies that bind to L1CAM. Antibody generation was carried out by immunizing mice with recombinant L1CAM proteins that were sourced commercially. Hybridoma supernatants were screened for antibodies that bind L1CAM in solution and on cells. Antibodies purified from the hybridoma supernatants were tested for internalization, followed by subcloning and sequencing of the top hits.

Antibodies 143G03 and 14A10 were selected as the front-runner lead candidates for formatting and testing as ADCs based on the following properties:
Binding to human L1CAM in solution at high affinity
Binding to human L1CAM on cells at high potency
Cross-reactivity to soluble cynomolgus monkey L1CAM
Cross-reactivity to soluble mouse L1CAM (14A10 only)
Efficient internalization in L1CAM-expressing cells

### Antibody Generation

Immunogens. Recombinant L1CAM proteins were sourced commercially for immunizations and hybridoma screening. The extracellular domain of human L1CAM (isoform 1, residues 1-1120) fused to a poly-histidine tag via its carboxy terminus was obtained from Creative Biomart [L1CAM-His]. In addition, the extracellular portions of human L1CAM (isoform 1, residues 20-1120 with a heterologous signal sequence at the amino terminus) or mouse L1CAM (residues 1-1123), each with a human Fc fused to the carboxy terminus, were procured from R&D Systems [L1CAM-Fc]. All recombinant proteins were produced in mammalian cells.

Immunizations. Two cohorts of 6 mice each (Balb/c and A/J strains) and 8 AlivaMab® mice with a human immunoglobulin repertoire were primed with a mixture of human and mouse L1CAM-Fc, followed by 3 booster injections with human L1CAM-His over the course of 8-10 weeks. To further stimulate antigen presentation, 3 mice in each group were given anti-mouse CD40 (FGK5 clone, BioXcell) the day of immunogen administration and 3 days after. TiterMax® Gold (Sigma-Aldrich) was used as adjuvant for all doses. Immunogens were administered subcutaneously. Serum was collected periodically to assess the immune response to L1CAM. After the third dose (prime+2 boosts), the serum from most mice had mid-point titers of 1:10,000 or greater when analyzed by enzyme-linked immunosorbent assay (ELISA) using recombinant human L1CAM-His. In addition, most sera demonstrated strong binding to stably transfected HEK293T cells overexpressing human L1CAM.

Mice with high serum mid-point titers from each strain were selected for an acute boost with human L1CAM-His. Three days later, splenocytes were collected and fused with mouse myeloma cells using polyethylene glycol to generate hybridomas, which were plated for screening.

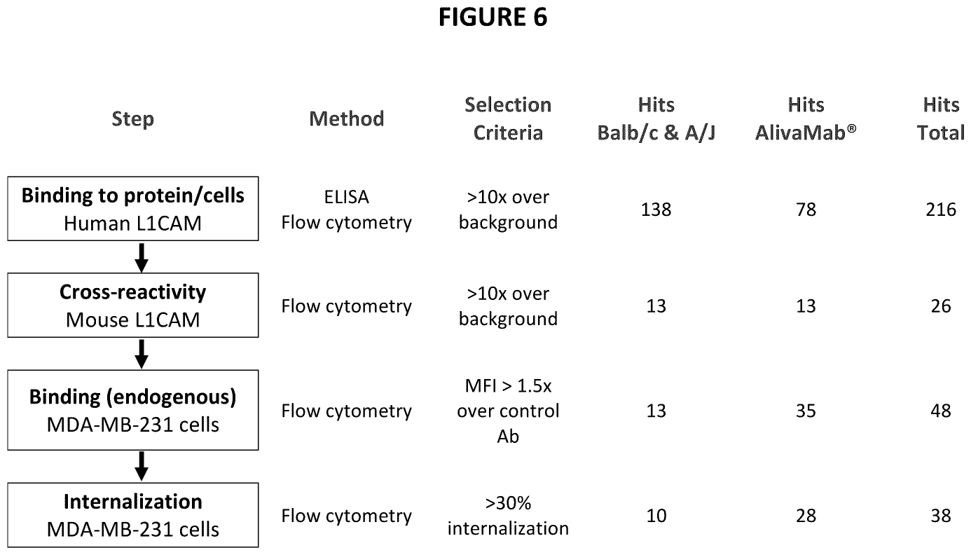

Hybridoma Screening. Hybridoma supernatants were analyzed using a screening funnel shown in FIG. 6. Binding to human L1CAM protein was tested by ELISA and to a HEK293T cell line overexpressing human L1CAM by flow cytometry. Supernatants that showed binding to human L1CAM on cells were further tested for their ability to bind to HEK293FT cells transiently transfected with mouse L1CAM. Cross-reactivity to cynomolgus L1CAM was not assessed at this point, as it is >99% homologous to human L1CAM. The top hits, including subsets of antibodies that did not cross-react with mouse L1CAM, were purified from hybridoma supernatants. The antibodies were tested for binding to MDA-MB-231 cells, a breast cancer cell line that endogenously expresses L1CAM, followed by analysis of their internalization efficacy in MDA-MB-231 cells.

The primary screen by ELISA using soluble, His-tagged human L1CAM and by flow cytometry using HEK293T cells overexpressing human L1CAM resulted in 216 confirmed hits. Of these, 26 were cross-reactive with mouse L1CAM expressed by transiently transfected HEK293FT cells. Furthermore, 76 antibodies with a strong signal over background in the binding assays (ELISA, flow cytometry) were purified from hybridoma supernatant to test for binding to MDA-MB-231 cells. The top 48 binders were tested for internalization in MDA-MB-231 cells, leading to the final hit list with 38 antibodies that fulfilled the criteria defined by the screening funnel.

Subcloning and Sequencing. A total of 32 hybridomas were subcloned by limiting dilution. Clonal supernatants were tested for binding to human L1CAM by ELISA and flow cytometry. Three mAb subclones were selected for each parent, and hybridoma cells were expanded and frozen. The variable regions of the antibodies were sequenced, resulting in 26 unique sequences (10 wild-type mice, 16 AlivaMab®) with good diversity in both the heavy and light chains.

Binding Analysis. To identify lead candidate antibodies, the binding profile of seven mAbs that showed strong internalization was characterized in detail using material purified from the supernatant of hybridoma clones. Dissociation constants (K_{D}) for binding to soluble human and cynomolgus monkey L1CAM-His were determined using biolayer interferometry (BLI). In addition, EC50 values for binding to cell-bound L1CAM were established by flow cytometry using HEK293T/FT cells overexpressing human or mouse L1CAM, MCF-7 cells, and MDA-MB-231 cells. In addition, the epitope regions of the mAbs were determined by BLI interaction analysis with L1CAM subdomains (Ig and FN) expressed individually, and by a BLI competition experiment to identify epitope bins. The results are summarized in Table 56.

TABLE 56
Binding profile of L1CAM mAbs.

| | | | | | | | | MCF-7 | MDA-231 |
|---|---|---|---|---|---|---|---|---|---|
| | | | | huL1CAM | cyL1CAM | huL1CAM | moL1CAM | Cells | Cells |
| | | Epitope | Epitope | Protein | Protein | 293T cells | 293FT cells | EC₅₀ | EC₅₀ |
| mAb | Mouse | Bin | Domain | K_{D} [nM] | K_{D} [nM] | EC₅₀ [nM] | EC₅₀ [nM] | [nM] | [nM] |
| 13F04 | WT | A | Ig | 1.13 | 19.8 | 4 | NB | 1.2 | 1.7 |
| 143G03 | WT | C | FN | 0.12 | 0.34 | 1.0 | NB | 0.35 | 0.9 |
| 04B06 | AMM | D | FN | 0.07 | 0.6 | 2.3 | NB | 1.1 | 9.6 |
| 12D10 | AMM | C | FN | 0.15 | 1.9 | 1.2 | NB | 0.77 | 2 |
| 13G04 | AMM | D | FN | <0.02* | 3.2 | 4.8 | NB | 4.8 | 6.7 |
| 14A10 | AMM | B | Ig | 1.2 | 3.2 | 3.5 | 5.6 | 5.3 | 8.9 |
| 15G02 | AMM | E | FN | 0.58 | 0.93 | 2.6 | NB | 1.0 | 0.98 |

WT: wild-type mice;
AMM: AlivaMab ® Mice
Ig: immunoglobulin domain;
FN: fibronectin domain
NB: no binding
huL1CAM: human L1CAM;
cyL1CAM: cynomolgus L1CAM;
moL1CAM: mouse L1CAM
*off-rate below detection limit

The panel of lead candidate mAbs, identified in both wild-type and AlivaMab® mice, showed good epitope diversity, with five distinct bins (A to E) spread across the two subdomains in the extracellular portion of L1CAM (Ig and FN). All mAbs bound human L1CAM protein at high affinity, with dissociation constants (KD) ranging from low single-digit nM to sub-nM. Cross-reactivity with cynomolgus monkey L1CAM protein was observed for all mAbs. However, only 3 mAbs had dissociation constants within 3-fold of the human protein. The EC50 values for binding to HEK293T cells overexpressing human L1CAM or cells endogenously expressing L1CAM (MCF-7 and MDA-MB-231) were in the single-digit nM or sub-nM range for all mAbs. Notably, 14A10, which is in a separate epitope bin (B), was the only mAb that cross-reacted with mouse L1CAM, as shown with transiently transfected HEK293FT cells.

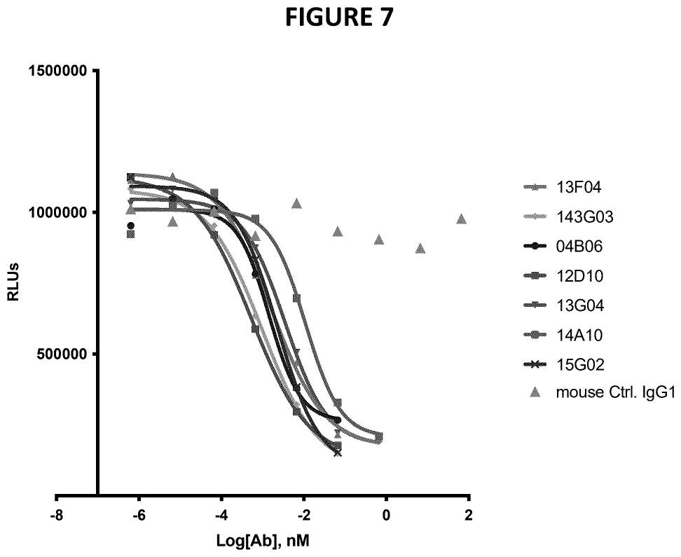

Internalization. Dose-response curves were established for the internalization potency of the lead candidate mAbs using a Fab-ZAP assay. Saporin (a plant ribosome inhibitor) conjugated to a Fab fragment that binds to the Fc portion of mouse IgG was pre-incubated with the L1CAM mAbs, followed by addition of the complex to HEK293T cells overexpressing human L1CAM. Five days later, the viability of the cells was analyzed using a colorimetric assay that detects the cellular metabolic activities (FIG. 7).

A Fab-ZAP assay was used to determine the potency of internalization for the L1CAM lead candidate mAbs in HEK293T cells overexpressing human L1CAM. The L1CAM mAbs and a mouse IgG1 isotype control were pre-incubated at various concentrations with a saporin-conjugated Fab fragment that binds mouse Fc. Five days later, cell viability was measured and plotted against mAb concentrations.

All L1CAM lead candidate mAbs were internalized potently, confirming the results from the screening with antibodies purified from the hybridoma supernatants. Their EC50 values for killing were in the sub-nM range for all mAbs within ˜10× from each other.

Full dose-response curves could not be established for MDA-MB-231 cells, which express substantially lower amounts of L1CAM on the surface than HEK293T cells overexpressing exogenous L1CAM. The one-step mix-and-read format of the Fab-ZAP assay resulted in a “hook effect” at higher L1CAM antibody concentrations prior to reaching the lower plateau.

Based on the binding and internalization data for the seven lead candidate mAbs, 143G03 and 14A10 were selected for antibody-drug conjugate formatting and testing. 143G03 was selected based on its high-affinity binding profile to proteins and cells, and its potent internalization in L1CAM-expressing cells. 14A10 was advanced due its ability to cross-react with mouse L1CAM, enabling the assessment of on-target toxicities in mice, and testing in syngeneic tumor models. 15G02 was identified as the primary backup candidate but was not explored beyond this point.

### Example 3—Modification of Lead Antibodies

Summary. Humanization of the murine 143G03 mAb variable region was carried out by homology modeling followed by assessment of the productivity and antigen binding properties of a panel of candidate molecules. The selected humanized candidate 143G03 mAb and the fully human 14A10 mAb variable region sequences were analyzed in silico for potential post-translational modification sites. An N61Q mutation was subsequently introduced into humanized 143G03 mAb to remove a potential deamidation ‘hot-spot’ site. Production, product quality and binding attributes of hz143G03 (N61Q) were evaluated and shown to match the parent molecule. The hz143G03 (N61Q) mAb and the fully human 14A10 mAb were formatted as IgG1 and evaluated in developability studies to identify any development liabilities. Based on these results, which indicated no requirement for further modification, the humanized 143G03 (N61Q) mAb and the fully human 14A10 mAb were selected as final lead candidates and designated “TDI-Y-004” and “TDI-Y-005,” respectively.

Humanization of Murine mAb 143G03. To humanize the parental murine 143G03 mAb, an approach utilizing a 3D structure generated by homology modeling and complementarity-determining regions (CDR) grafting was employed. A panel of 30 humanized antibodies were generated from candidate sequences designed to maximize the amount of human sequence while retaining the parental mAb specificity and affinity. The 30 humanized antibodies were generated from a parental antibody comprising V_{H} and V_{L} regions, and CDRs disclosed in Table 57.

TABLE 57

| CDRs | 1 | 2 | 3 |
|---|---|---|---|
| V_{H} | GYTFTNYY | IGPGSGSP | ARWLLPFDY |
| | [SEQ ID NO: 12] | [SEQ ID NO: 13] | [SEQ ID NO: 14] |
| V_{L} | SSVNY [SEQ ID | ATS [SEQ ID NO: | QQFSSDPLT |
| | NO: 15] | 16] | [SEQ ID NO: 17] |

| | |
|---|---|
| Full | QVQLRQSGAELVRPGTSVKMSCKASGYTFTNYYIHWVKQRPGQGLEWIGKIGPGSG |
| V_{H} | SPYYNGMFKDKATLTVDTSSSTAYMQLSSLTSEDSAVYFCARWLLPFDYWGQGTTL |
| | TVSS [SEQ ID NO: 33] |
| Full | QIVLSQSPGILSASPGEKVTMTCRASSSVNYMHWYQQKSGSSPKPWIFATSILASG |
| V_{L} | VPPRFSGSGSGTSYSLTISSVEAEDTATYYCQQFSSDPLTFGAGTKLELK [SEQ |
| | ID NO: 34] |

Selection of the candidate humanized mAb from the panel was determined based on evaluation of productivity and binding to the target antigen. The final humanized candidate was formatted as a human IgG.

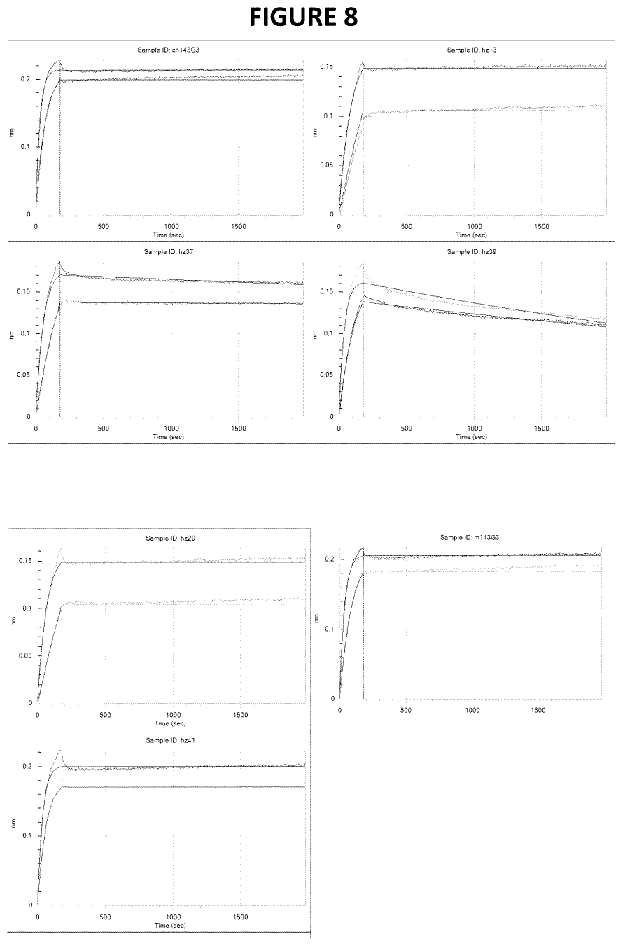

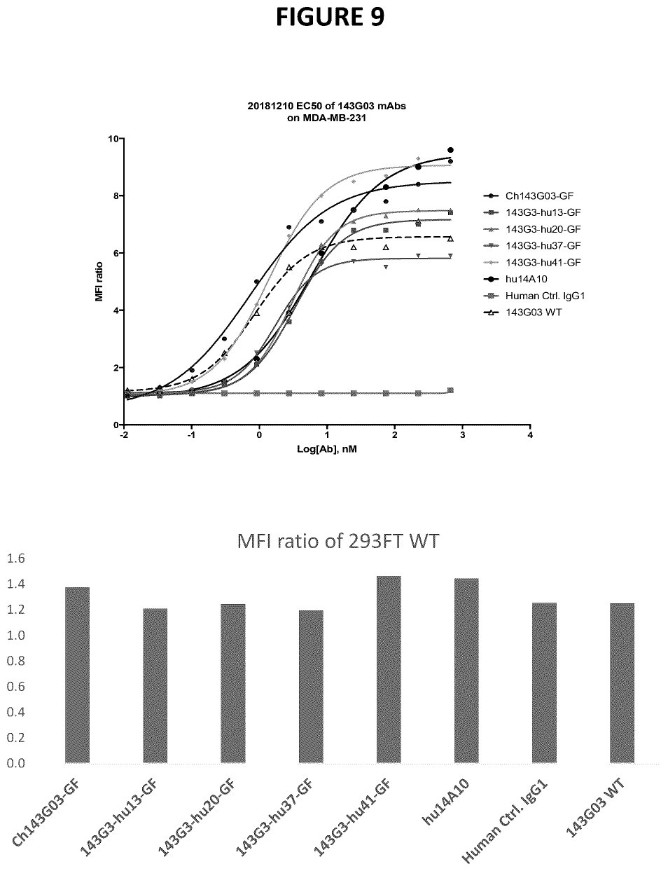

Briefly, CDR-grafted heavy and light chains were expressed in various combinations and the antigen-binding properties were evaluated. Dissociation constant (K_{D}) of various humanized antibodies was determined by biolayer interferometry (BLI) (FIG. 8). Further, binding to 293 FT cells and MDA-MB-231 cells expressing L1CAM was detected by FACS analysis (FIG. 9). EC50 values are reported in Table 58.

TABLE 58
EC50 values.

| | Ch143G03- | 143G3- | 143G3- | 143G3- | 143G3- | | Human Ctrl. | |
|---|---|---|---|---|---|---|---|---|
| Name | GF | hu13-GF | hu20-GF | hu37-GF | hu41-GF | hu14A10 | IgG1 | 143G03 WT |
| EC50 | 0.7273 nM | 3.375 nM | 3.058 nM | 1.723 nM | 1.362 nM | 5.93 nM | NA | 0.8496 nM |

The data showed that the binding of the tested antibodies was specific and that 143G3hu41 had the lowest EC50 among the tested antibodies.

Variable Region Modification of Humanized mAb 143G03. Analysis of the humanized mAb 143G03 variable region sequence identified a potential deamidation ‘hot-spot’ (an asparagine followed by a glycine, NG) adjacent to CDR2 in the heavy-chain (this position is within some non-IMGT definitions of CDR2, e.g., Kabat). To evaluate the impact of deamidation on the binding properties of the humanized 143G03 antibody, a charge mimic mutation (N61D) was introduced. The charge mimic variant, mAb hz143G03 (N61D), retained the specificity and affinity of the parent antibody.

To further de-risk the molecule, an N61Q mutation was introduced to ablate the potential deamidation site, generating mAb hz143G03 (N61Q). The introduction of the conservative N to Q change had no impact on the affinity of mAb as measured by BLI relative to the parent mAb.

Developability of Humanized 143G03 (N61Q) mAb (“TDI-Y-004”) and Human 14A10 mAb (“TDI-Y-005”). A comprehensive developability study was run to establish baseline biophysical parameters and evaluate the chemical and physical stability of the lead molecules to identify potential development challenges. No critical liabilities were identified in the biophysical attributes exhibited by the lead molecules or by the forced stress studies run to evaluate chemical and physical stability. Additionally, both molecules exhibited good solubility at 20 mg/ml and minimal non-specific binding in heparin and baculovirus particle binding assays. The results of the developability study are summarized in Table 59-62.

TABLE 59
Summary of TDI-Y-004 Characteristics.

| Method | Criteria / Goal | Results |
|---|---|---|
| SEC-HPLC | >95% monomer | SEC analysis indicated that the mAb was > 98% |
| | | monomeric |
| HIC | Establish baseline | HIC analysis indicated that the mAb was hydrophobic and |
| | | there was a 14% modification. MS analysis can confirm the |
| | | nature of the species. |
| cIEF | Establish baseline | cIEF analysis indicated that the pI range was between 7.2- |
| | | 7.8 |
| CE-SDS | >90% purity | The molecule had high purity > 98% under denaturing |
| | | (reduction and non-reducing) conditions |

TABLE 60
Summary of Degradation Studies for TDI-Y-004.

| Forced Degradation | Results |
|---|---|
| Low and High pH | The molecule was stable under deferent pH conditions |
| stress | |
| H₂O₂ and tBMP | Slight acidic shift was observed as a result of H₂O₂. Peptide mapping and BLI |
| Oxidation | analysis can identify the location and the impact of the modification on the |
| | molecule. |
| Thermal Stress | cIEF acidic shift was observed as a result of thermal stress. Peptide mapping and |
| | BLI analysis can identify the location and the impact of the modification on the |
| | molecule. |

TABLE 61
Summary of TDI-Y-005 Characteristics.

| Method | Criteria / Goal | Results |
|---|---|---|
| SEC-HPLC | >95% monomer | SEC analysis indicated that the mAb was >97% |
| | | monomeric |
| HIC | Establish baseline | HIC analysis indicated that the mAb was hydrophobic |
| cIEF | Establish baseline | cIEF analysis indicated that the pI range was between 8.5- |
| | | 8.9 |
| CE-SDS | >90% purity | The molecule had high purity >98% under denaturing |
| | | (reduction and non-reducing) conditions |

TABLE 62
Summary of Degradation Studies for TDI-Y-005.

| Forced Degradation | Results |
|---|---|
| Low and High pH | The molecule was stable under deferent pH conditions |
| stress | |
| H₂O₂ and tBMP | Slight acidic shift was observed as a result of H₂O₂. Peptide mapping and BLI |
| Oxidation | analysis can identify the location and the impact of the modification on the |
| | molecule. |
| Thermal Stress | cIEF acidic shift was observed as a result of thermal stress. However, TDI-Y-005 |
| | appear to be thermally more stable than TDI-Y-005. Peptide mapping and BLI |
| | analysis can identify the location and the impact of the modification on the |
| | molecule. |

Epitope Mapping. Before starting the epitope mapping, a high-mass MALDI analysis was performed on the candidate antibodies in order to verify their integrity and aggregation level. Notably, no aggregate were detected for each one of TDI-Y-004, TDI-Y-005, and multiples of human L1CAM. Next, the molecular interfaces of human L1CAM/TDI-Y-004 complex and human L1CAM/TDI-Y-005 complex were determined. Briefly, each protein complex was incubated with deuterated cross-linkers and subjected to multi-enzymatic cleavage. After enrichment of the cross-linked peptides, the samples were analyzed by high-resolution mass spectrometry.

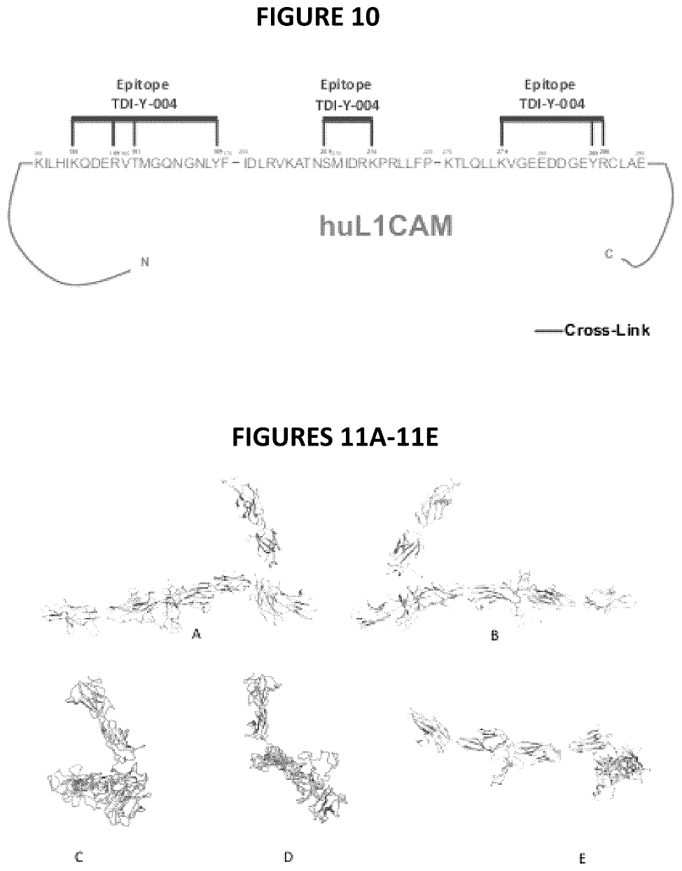

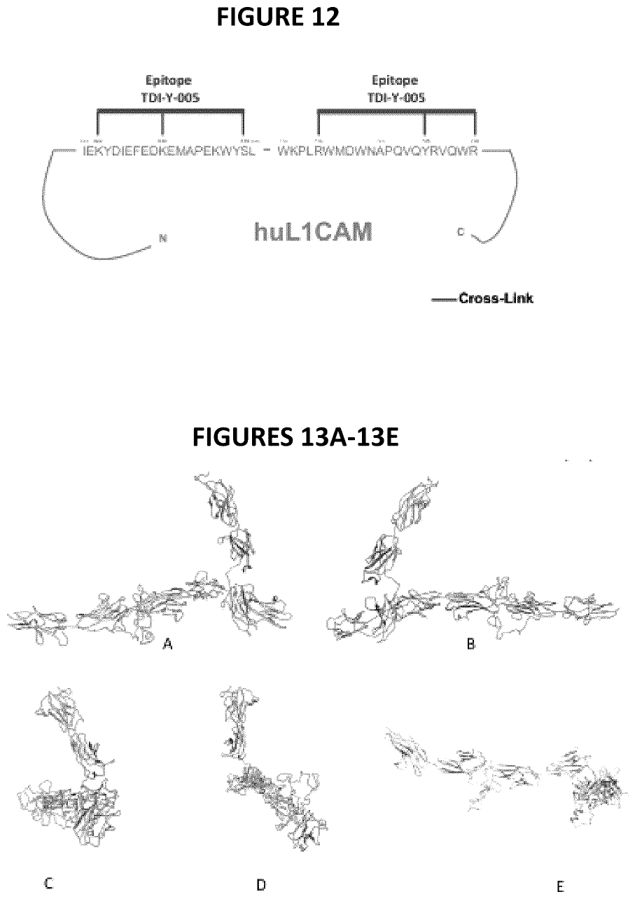

Using chemical cross-linking, high-mass MALDI mass spectrometry, and nLC-Orbitrap mass spectrometry, it was determined that TDI-Y-004 interacted with the amino acid residues 155, 159, 161, 169, 209, 214, 279, 285, and 286 of the human L1CAM protein. These interactions are summarized in FIGS. 10 and 11A-11E. Moreover, it was determined that TDI-Y-005 interacted with the amino acid residues 622, 630, 639, 714, 725, 730 of the human L1CAM protein. These interactions are summarized in FIGS. 12 and 13A-13E.

In Silico Immunogenicity Prediction. Sequences were analyzed by artificial neural networks for their affinity to MHC class II alleles. Peptides were identified as strong binders when their predicted affinity to the MHC class II molecules is 50 nM or greater. As expected, a number of potential T cell epitopes were predicted to occur in both chains of each of the candidate antibodies including CDRs. The predicted epitopes may include highly conserved T regulatory epitopes known to bind multiple HLA subtypes (Tables 63-66).

TABLE 63
TDI-Y-004 Heavy Chain immunogenicity analysis.

| Allele | Peptide | Identity | Pos | Core | Core_Rel | Affinity(nM) |
|---|---|---|---|---|---|---|
| DRB1_0101 | NYYIHWVRQAPGQGL | TDI-Y- | 5 | WVRQAPGQG | 0.29 | 47.23 |
| | | 004-HC | | | | |
| DRB1_0101 | YYIHWVRQAPGQGLE | TDI-Y- | 5 | VRQAPGQGL | 0.395 | 37.71 |
| | | 004-HC | | | | |
| DRB1_0101 | YIHWVRQAPGQGLEW | TDI-Y- | 4 | VRQAPGQGL | 0.555 | 34.53 |
| | | 004-HC | | | | |
| DRB1_0101 | IHWVRQAPGQGLEWM | TDI-Y- | 3 | VRQAPGQGL | 0.695 | 33.91 |
| | | 004-HC | | | | |
| DRB1_0101 | ESTSTAYMELSSLRS | TDI-Y- | 6 | YMELSSLRS | 0.89 | 13.53 |
| | | 004-HC | | | | |
| DRB1_0101 | STSTAYMELSSLRSE | TDI-Y- | 5 | YMELSSLRS | 0.91 | 8.43 |
| | | 004-HC | | | | |
| DRB1_0101 | TSTAYMELSSLRSED | TDI-Y- | 4 | YMELSSLRS | 0.9 | 8.59 |
| | | 004-HC | | | | |
| DRB1_0101 | STAYMELSSLRSEDT | TDI-Y- | 3 | YMELSSLRS | 0.915 | 8.22 |
| | | 004-HC | | | | |
| DRB1_0101 | TAYMELSSLRSEDTA | TDI-Y- | 2 | YMELSSLRS | 0.895 | 8.99 |
| | | 004-HC | | | | |
| DRB1_0101 | AYMELSSLRSEDTAV | TDI-Y- | 1 | YMELSSLRS | 0.825 | 12.05 |
| | | 004-HC | | | | |
| DRB1_0101 | YMELSSLRSEDTAVY | TDI-Y- | 0 | YMELSSLRS | 0.49 | 29.58 |
| | | 004-HC | | | | |
| DRB1_0101 | WGQGTLVTVSSASTK | TDI-Y- | 5 | LVTVSSAST | 0.89 | 27.98 |
| | | 004-HC | | | | |
| DRB1_0101 | GQGTLVTVSSASTKG | TDI-Y- | 4 | LVTVSSAST | 0.885 | 22.69 |
| | | 004-HC | | | | |
| DRB1_0101 | QGTLVTVSSASTKGP | TDI-Y- | 3 | LVTVSSAST | 0.905 | 25.23 |
| | | 004-HC | | | | |
| DRB1_0101 | GTLVTVSSASTKGPS | TDI-Y- | 2 | LVTVSSAST | 0.875 | 31.95 |
| | | 004-HC | | | | |
| DRB1_0101 | LTSGVHTFPAVLQSS | TDI-Y- | 4 | VHTFPAVLQ | 0.845 | 42.84 |
| | | 004-HC | | | | |
| DRB1_0101 | TSGVHTFPAVLQSSG | TDI-Y- | 3 | VHTFPAVLQ | 0.845 | 45.93 |
| | | 004-HC | | | | |
| DRB1_0101 | HTFPAVLQSSGLYSL | TDI-Y- | 5 | VLQSSGLYS | 0.33 | 40.42 |
| | | 004-HC | | | | |
| DRB1_0101 | TFPAVLQSSGLYSLS | TDI-Y- | 4 | VLQSSGLYS | 0.45 | 37.13 |
| | | 004-HC | | | | |
| DRB1_0101 | FPAVLQSSGLYSLSS | TDI-Y- | 3 | VLQSSGLYS | 0.5 | 39.14 |
| | | 004-HC | | | | |
| DRB1_0101 | VLQSSGLYSLSSVVT | TDI-Y- | 6 | LYSLSSVVT | 0.715 | 36.96 |
| | | 004-HC | | | | |
| DRB1_0101 | LQSSGLYSLSSVVTV | TDI-Y- | 5 | LYSLSSVVT | 0.79 | 16.93 |
| | | 004-HC | | | | |
| DRB1_0101 | QSSGLYSLSSVVTVP | TDI-Y- | 4 | LYSLSSVVT | 0.765 | 15.63 |
| | | 004-HC | | | | |
| DRB1_0101 | SSGLYSLSSVVTVPS | TDI-Y- | 3 | LYSLSSVVT | 0.755 | 14.73 |
| | | 004-HC | | | | |
| DRB1_0101 | SGLYSLSSVVTVPSS | TDI-Y- | 2 | LYSLSSVVT | 0.6 | 16.28 |
| | | 004-HC | | | | |
| DRB1_0101 | GLYSLSSVVTVPSSS | TDI-Y- | 4 | LSSVVTVPS | 0.39 | 22 |
| | | 004-HC | | | | |
| DRB1_0101 | LYSLSSVVTVPSSSL | TDI-Y- | 3 | LSSVVTVPS | 0.65 | 27.84 |
| | | 004-HC | | | | |
| DRB1_0101 | YSLSSVVTVPSSSLG | TDI-Y- | 5 | VVTVPSSSL | 0.49 | 29.84 |
| | | 004-HC | | | | |
| DRB1_0101 | SLSSVVTVPSSSLGT | TDI-Y- | 4 | VVTVPSSSL | 0.75 | 27.29 |
| | | 004-HC | | | | |
| DRB1_0101 | LSSVVTVPSSSLGTQ | TDI-Y- | 3 | VVTVPSSSL | 0.855 | 27.85 |
| | | 004-HC | | | | |
| DRB1_0101 | SSVVTVPSSSLGTQT | TDI-Y- | 2 | VVTVPSSSL | 0.835 | 44.27 |
| | | 004-HC | | | | |
| DRB1_0101 | CPAPELLGGPSVFLF | TDI-Y- | 5 | LLGGPSVFL | 0.675 | 39.98 |
| | | 004-HC | | | | |
| DRB1_0101 | PAPELLGGPSVFLFP | TDI-Y- | 4 | LLGGPSVFL | 0.685 | 40.14 |
| | | 004-HC | | | | |
| DRB1_0101 | APELLGGPSVFLFPP | TDI-Y- | 3 | LLGGPSVFL | 0.715 | 45.32 |
| | | 004-HC | | | | |
| DRB1_0101 | EQYNSTYRVVSVLTV | TDI-Y- | 6 | YRVVSVLTV | 0.64 | 42.5 |
| | | 004-HC | | | | |
| DRB1_0101 | QYNSTYRVVSVLTVL | TDI-Y- | 5 | YRVVSVLTV | 0.775 | 23.54 |
| | | 004-HC | | | | |
| DRB1_0101 | YNSTYRVVSVLTVLH | TDI-Y- | 4 | YRVVSVLTV | 0.765 | 19.11 |
| | | 004-HC | | | | |
| DRB1_0101 | NSTYRVVSVLTVLHQ | TDI-Y- | 3 | YRVVSVLTV | 0.735 | 18.1 |
| | | 004-HC | | | | |
| DRB1_0101 | STYRVVSVLTVLHQD | TDI-Y- | 2 | YRVVSVLTV | 0.72 | 21.09 |
| | | 004-HC | | | | |
| DRB1_0101 | TYRVVSVLTVLHQDW | TDI-Y- | 1 | YRVVSVLTV | 0.645 | 30.93 |
| | | 004-HC | | | | |
| DRB1_0101 | DGSFFLYSKLTVDKS | TDI-Y- | 4 | FLYSKLTVD | 0.29 | 43.3 |
| | | 004-HC | | | | |
| DRB1_0101 | GSFFLYSKLTVDKSR | TDI-Y- | 5 | YSKLTVDKS | 0.455 | 27.5 |
| | | 004-HC | | | | |
| DRB1_0101 | SFFLYSKLTVDKSRW | TDI-Y- | 4 | YSKLTVDKS | 0.605 | 24.93 |
| | | 004-HC | | | | |
| DRB1_0101 | FFLYSKLTVDKSRWQ | TDI-Y- | 3 | YSKLTVDKS | 0.77 | 28.12 |
| | | 004-HC | | | | |
| DRB1_0101 | FLYSKLTVDKSRWQQ | TDI-Y- | 2 | YSKLTVDKS | 0.845 | 43.24 |
| | | 004-HC | | | | |
| DRB1_0101 | EALHNHYTQKSLSLS | TDI-Y- | 5 | HYTQKSLSL | 0.595 | 42.79 |
| | | 004-HC | | | | |
| DRB1_0101 | ALHNHYTQKSLSLSP | TDI-Y- | 4 | HYTQKSLSL | 0.555 | 36.93 |
| | | 004-HC | | | | |
| DRB1_0101 | LHNHYTQKSLSLSPG | TDI-Y- | 3 | HYTQKSLSL | 0.54 | 36.24 |
| | | 004-HC | | | | |
| DRB1_0101 | HNHYTQKSLSLSPGK | TDI-Y- | 2 | HYTQKSLSL | 0.435 | 42.13 |
| | | 004-HC | | | | |
| DRB1_0101 | SPGKNHYTQKSLSLS | TDI-Y- | 5 | HYTQKSLSL | 0.595 | 48.92 |
| | | 004-HC | | | | |
| DRB1_0101 | PGKNHYTQKSLSLSP | TDI-Y- | 4 | HYTQKSLSL | 0.54 | 40.76 |
| | | 004-HC | | | | |
| DRB1_0101 | GKNHYTQKSLSLSPG | TDI-Y- | 3 | HYTQKSLSL | 0.505 | 38.46 |
| | | 004-HC | | | | |
| DRB1_0101 | KNHYTQKSLSLSPGK | TDI-Y- | 2 | HYTQKSLSL | 0.435 | 37.93 |
| | | 004-HC | | | | |
| DRB1_0401 | STAYMELSSLRSEDT | TDI-Y- | 3 | YMELSSLRS | 0.86 | 48.8 |
| | | 004-HC | | | | |
| DRB1_0701 | QYNSTYRVVSVLTVL | TDI-Y- | 5 | YRVVSVLTV | 0.65 | 48.08 |
| | | 004-HC | | | | |
| DRB1_0701 | YNSTYRVVSVLTVLH | TDI-Y- | 4 | YRVVSVLTV | 0.71 | 44.03 |
| | | 004-HC | | | | |
| DRB1_0701 | NSTYRVVSVLTVLHQ | TDI-Y- | 3 | YRVVSVLTV | 0.68 | 42.67 |
| | | 004-HC | | | | |

TABLE 64
TDI-Y-004 Light Chain immunogenicity analysis.

| Allele | Peptide | Identity | Pos | Core | Core_Rel | Affinity(nM) |
|---|---|---|---|---|---|---|
| DRB1_0101 | EIVLTQSPGTLSLSP | TDI-Y- | 3 | LTQSPGTLS | 0.53 | 16.29 |
| | | 004-LC | | | | |
| DRB1_0101 | IVLTQSPGTLSLSPG | TDI-Y- | 2 | LTQSPGTLS | 0.695 | 24.07 |
| | | 004-LC | | | | |
| DRB1_0101 | GQAPRLLIYATSILA | TDI-Y- | 6 | LIYATSILA | 0.61 | 18.45 |
| | | 004-LC | | | | |
| DRB1_0101 | QAPRLLIYATSILAS | TDI-Y- | 5 | LIYATSILA | 0.705 | 11.77 |
| | | 004-LC | | | | |
| DRB1_0101 | APRLLIYATSILASG | TDI-Y- | 4 | LIYATSILA | 0.695 | 11.21 |
| | | 004-LC | | | | |
| DRB1_0101 | PRLLIYATSILASGI | TDI-Y- | 3 | LIYATSILA | 0.67 | 10.62 |
| | | 004-LC | | | | |
| DRB1_0101 | RLLIYATSILASGIP | TDI-Y- | 2 | LIYATSILA | 0.63 | 12.32 |
| | | 004-LC | | | | |
| DRB1_0101 | LLIYATSILASGIPD | TDI-Y- | 1 | LIYATSILA | 0.59 | 20.69 |
| | | 004-LC | | | | |
| DRB1_0101 | LIYATSILASGIPDR | TDI-Y- | 0 | LIYATSILA | 0.4 | 45.11 |
| | | 004-LC | | | | |
| DRB1_0101 | GTKVEIKRTVAAPSV | TDI-Y- | 5 | IKRTVAAPS | 0.885 | 22.04 |
| | | 004-LC | | | | |
| DRB1_0101 | TKVEIKRTVAAPSVF | TDI-Y- | 4 | IKRTVAAPS | 0.905 | 13.19 |
| | | 004-LC | | | | |
| DRB1_0101 | KVEIKRTVAAPSVFI | TDI-Y- | 3 | IKRTVAAPS | 0.86 | 10.34 |
| | | 004-LC | | | | |
| DRB1_0101 | VEIKRTVAAPSVFIF | TDI-Y- | 2 | IKRTVAAPS | 0.67 | 11.73 |
| | | 004-LC | | | | |
| DRB1_0101 | EIKRTVAAPSVFIFP | TDI-Y- | 1 | IKRTVAAPS | 0.54 | 17.09 |
| | | 004-LC | | | | |
| DRB1_0101 | IKRTVAAPSVFIFPP | TDI-Y- | 3 | TVAAPSVFI | 0.505 | 33.56 |
| | | 004-LC | | | | |
| DRB1_0101 | DEQLKSGTASVVCLL | TDI-Y- | 3 | LKSGTASVV | 0.93 | 39.3 |
| | | 004-LC | | | | |
| DRB1_0101 | EQLKSGTASVVCLLN | TDI-Y- | 2 | LKSGTASVV | 0.94 | 36.88 |
| | | 004-LC | | | | |
| DRB1_0101 | DSKDSTYSLSSTLTL | TDI-Y- | 6 | YSLSSTLTL | 0.535 | 30.17 |
| | | 004-LC | | | | |
| DRB1_0101 | SKDSTYSLSSTLTLS | TDI-Y- | 5 | YSLSSTLTL | 0.645 | 17.43 |
| | | 004-LC | | | | |
| DRB1_0101 | KDSTYSLSSTLTLSK | TDI-Y- | 4 | YSLSSTLTL | 0.695 | 14.17 |
| | | 004-LC | | | | |
| DRB1_0101 | DSTYSLSSTLTLSKA | TDI-Y- | 3 | YSLSSTLTL | 0.745 | 14.8 |
| | | 004-LC | | | | |
| DRB1_0101 | STYSLSSTLTLSKAD | TDI-Y- | 2 | YSLSSTLTL | 0.8 | 15.76 |
| | | 004-LC | | | | |
| DRB1_0101 | TYSLSSTLTLSKADY | TDI-Y- | 1 | YSLSSTLTL | 0.785 | 29.3 |
| | | 004-LC | | | | |

TABLE 65
TDI-Y-005 Heavy Chain immunogenicity analysis.

| Allele | Peptide | Identity | Pos | Core | Core_Rel | Affinity(nM) |
|---|---|---|---|---|---|---|
| DRB1_0101 | SGGGLVQPGGSLRLS | TDI-Y- | 5 | VQPGGSLRL | 0.785 | 33.61 |
| | | 005-HC | | | | |
| DRB1_0101 | GGGLVQPGGSLRLSC | TDI-Y- | 4 | VQPGGSLRL | 0.805 | 28.48 |
| | | 005-HC | | | | |
| DRB1_0101 | GGLVQPGGSLRLSCA | TDI-Y- | 3 | VQPGGSLRL | 0.83 | 24.79 |
| | | 005-HC | | | | |
| DRB1_0101 | GLVQPGGSLRLSCAA | TDI-Y- | 2 | VQPGGSLRL | 0.84 | 32.31 |
| | | 005-HC | | | | |
| DRB1_0101 | QPGGSLRLSCAAPGF | TDI-Y- | 5 | LRLSCAAPG | 0.94 | 24.32 |
| | | 005-HC | | | | |
| DRB1_0101 | PGGSLRLSCAAPGFI | TDI-Y- | 4 | LRLSCAAPG | 0.93 | 13.9 |
| | | 005-HC | | | | |
| DRB1_0101 | GGSLRLSCAAPGFIF | TDI-Y- | 3 | LRLSCAAPG | 0.855 | 10.88 |
| | | 005-HC | | | | |
| DRB1_0101 | GSLRLSCAAPGFIFS | TDI-Y- | 2 | LRLSCAAPG | 0.825 | 11.59 |
| | | 005-HC | | | | |
| DRB1_0101 | SLRLSCAAPGFIFSS | TDI-Y- | 1 | LRLSCAAPG | 0.75 | 16.78 |
| | | 005-HC | | | | |
| DRB1_0101 | NMNWVRQAPGKGLEW | TDI-Y- | 4 | VRQAPGKGL | 0.62 | 40.95 |
| | | 005-HC | | | | |
| DRB1_0101 | MNWVRQAPGKGLEWV | TDI-Y- | 3 | VRQAPGKGL | 0.695 | 34.74 |
| | | 005-HC | | | | |
| DRB1_0101 | KGLEWVSYISSSSST | TDI-Y- | 4 | WVSYISSSS | 0.57 | 39.31 |
| | | 005-HC | | | | |
| DRB1_0101 | GLEWVSYISSSSSTT | TDI-Y- | 3 | WVSYISSSS | 0.365 | 23.38 |
| | | 005-HC | | | | |
| DRB1_0101 | LEWVSYISSSSSTTY | TDI-Y- | 5 | YISSSSSTT | 0.6 | 13.09 |
| | | 005-HC | | | | |
| DRB1_0101 | EWVSYISSSSSTTYY | TDI-Y- | 4 | YISSSSSTT | 0.71 | 10.54 |
| | | 005-HC | | | | |
| DRB1_0101 | WVSYISSSSSTTYYA | TDI-Y- | 3 | YISSSSSTT | 0.78 | 9.71 |
| | | 005-HC | | | | |
| DRB1_0101 | VSYISSSSSTTYYAD | TDI-Y- | 2 | YISSSSSTT | 0.86 | 12.92 |
| | | 005-HC | | | | |
| DRB1_0101 | SYISSSSSTTYYADS | TDI-Y- | 1 | YISSSSSTT | 0.82 | 23.21 |
| | | 005-HC | | | | |
| DRB1_0101 | DNAKNSLYLQMNSLR | TDI-Y- | 5 | SLYLQMNSL | 0.365 | 34.91 |
| | | 005-HC | | | | |
| DRB1_0101 | NAKNSLYLQMNSLRD | TDI-Y- | 6 | YLQMNSLRD | 0.66 | 14.65 |
| | | 005-HC | | | | |
| DRB1_0101 | AKNSLYLQMNSLRDE | TDI-Y- | 5 | YLQMNSLRD | 0.78 | 10.11 |
| | | 005-HC | | | | |
| DRB1_0101 | KNSLYLQMNSLRDED | TDI-Y- | 4 | YLQMNSLRD | 0.81 | 10.79 |
| | | 005-HC | | | | |
| DRB1_0101 | NSLYLQMNSLRDEDT | TDI-Y- | 3 | YLQMNSLRD | 0.84 | 10.83 |
| | | 005-HC | | | | |
| DRB1_0101 | SLYLQMNSLRDEDTA | TDI-Y- | 2 | YLQMNSLRD | 0.86 | 12.37 |
| | | 005-HC | | | | |
| DRB1_0101 | LYLQMNSLRDEDTAV | TDI-Y- | 1 | YLQMNSLRD | 0.885 | 19.69 |
| | | 005-HC | | | | |
| DRB1_0101 | LTSGVHTFPAVLQSS | TDI-Y- | 4 | VHTFPAVLQ | 0.845 | 42.84 |
| | | 005-HC | | | | |
| DRB1_0101 | TSGVHTFPAVLQSSG | TDI-Y- | 3 | VHTFPAVLQ | 0.845 | 45.93 |
| | | 005-HC | | | | |
| DRB1_0101 | HTFPAVLQSSGLYSL | TDI-Y- | 5 | VLQSSGLYS | 0.33 | 40.42 |
| | | 005-HC | | | | |
| DRB1_0101 | TFPAVLQSSGLYSLS | TDI-Y- | 4 | VLQSSGLYS | 0.45 | 37.13 |
| | | 005-HC | | | | |
| DRB1_0101 | FPAVLQSSGLYSLSS | TDI-Y- | 3 | VLQSSGLYS | 0.5 | 39.14 |
| | | 005-HC | | | | |
| DRB1_0101 | VLQSSGLYSLSSVVT | TDI-Y- | 6 | LYSLSSVVT | 0.715 | 36.96 |
| | | 005-HC | | | | |
| DRB1_0101 | LQSSGLYSLSSVVTV | TDI-Y- | 5 | LYSLSSVVT | 0.79 | 16.93 |
| | | 005-HC | | | | |
| DRB1_0101 | QSSGLYSLSSVVTVP | TDI-Y- | 4 | LYSLSSVVT | 0.765 | 15.63 |
| | | 005-HC | | | | |
| DRB1_0101 | SSGLYSLSSVVTVPS | TDI-Y- | 3 | LYSLSSVVT | 0.755 | 14.73 |
| | | 005-HC | | | | |
| DRB1_0101 | SGLYSLSSVVTVPSS | TDI-Y- | 2 | LYSLSSVVT | 0.6 | 16.28 |
| | | 005-HC | | | | |
| DRB1_0101 | GLYSLSSVVTVPSSS | TDI-Y- | 4 | LSSVVTVPS | 0.39 | 22 |
| | | 005-HC | | | | |
| DRB1_0101 | LYSLSSVVTVPSSSL | TDI-Y- | 3 | LSSVVTVPS | 0.65 | 27.84 |
| | | 005-HC | | | | |
| DRB1_0101 | YSLSSVVTVPSSSLG | TDI-Y- | 5 | VVTVPSSSL | 0.49 | 29.84 |
| | | 005-HC | | | | |
| DRB1_0101 | SLSSVVTVPSSSLGT | TDI-Y- | 4 | VVTVPSSSL | 0.75 | 27.29 |
| | | 005-HC | | | | |
| DRB1_0101 | LSSVVTVPSSSLGTQ | TDI-Y- | 3 | VVTVPSSSL | 0.855 | 27.85 |
| | | 005-HC | | | | |
| DRB1_0101 | SSVVTVPSSSLGTQT | TDI-Y- | 2 | VVTVPSSSL | 0.835 | 44.27 |
| | | 005-HC | | | | |
| DRB1_0101 | CPAPELLGGPSVFLF | TDI-Y- | 5 | LLGGPSVFL | 0.675 | 39.98 |
| | | 005-HC | | | | |
| DRB1_0101 | PAPELLGGPSVFLFP | TDI-Y- | 4 | LLGGPSVFL | 0.685 | 40.14 |
| | | 005-HC | | | | |
| DRB1_0101 | APELLGGPSVFLFPP | TDI-Y- | 3 | LLGGPSVFL | 0.715 | 45.32 |
| | | 005-HC | | | | |
| DRB1_0101 | EQYNSTYRVVSVLTV | TDI-Y- | 6 | YRVVSVLTV | 0.64 | 42.5 |
| | | 005-HC | | | | |
| DRB1_0101 | QYNSTYRVVSVLTVL | TDI-Y- | 5 | YRVVSVLTV | 0.775 | 23.54 |
| | | 005-HC | | | | |
| DRB1_0101 | YNSTYRVVSVLTVLH | TDI-Y- | 4 | YRVVSVLTV | 0.765 | 19.11 |
| | | 005-HC | | | | |
| DRB1_0101 | NSTYRVVSVLTVLHQ | TDI-Y- | 3 | YRVVSVLTV | 0.735 | 18.1 |
| | | 005-HC | | | | |
| DRB1_0101 | STYRVVSVLTVLHQD | TDI-Y- | 2 | YRVVSVLTV | 0.72 | 21.09 |
| | | 005-HC | | | | |
| DRB1_0101 | TYRVVSVLTVLHQDW | TDI-Y- | 1 | YRVVSVLTV | 0.645 | 30.93 |
| | | 005-HC | | | | |
| DRB1_0101 | DGSFFLYSKLTVDKS | TDI-Y- | 4 | FLYSKLTVD | 0.29 | 43.3 |
| | | 005-HC | | | | |
| DRB1_0101 | GSFFLYSKLTVDKSR | TDI-Y- | 5 | YSKLTVDKS | 0.455 | 27.5 |
| | | 005-HC | | | | |
| DRB1_0101 | SFFLYSKLTVDKSRW | TDI-Y- | 4 | YSKLTVDKS | 0.605 | 24.93 |
| | | 005-HC | | | | |
| DRB1_0101 | FFLYSKLTVDKSRWQ | TDI-Y- | 3 | YSKLTVDKS | 0.77 | 28.12 |
| | | 005-HC | | | | |
| DRB1_0101 | FLYSKLTVDKSRWQQ | TDI-Y- | 2 | YSKLTVDKS | 0.845 | 43.24 |
| | | 005-HC | | | | |
| DRB1_0101 | EALHNHYTQKSLSLS | TDI-Y- | 5 | HYTQKSLSL | 0.595 | 42.79 |
| | | 005-HC | | | | |
| DRB1_0101 | ALHNHYTQKSLSLSP | TDI-Y- | 4 | HYTQKSLSL | 0.555 | 36.93 |
| | | 005-HC | | | | |
| DRB1_0101 | LHNHYTQKSLSLSPG | TDI-Y- | 3 | HYTQKSLSL | 0.54 | 36.24 |
| | | 005-HC | | | | |
| DRB1_0101 | HNHYTQKSLSLSPGK | TDI-Y- | 2 | HYTQKSLSL | 0.435 | 42.13 |
| | | 005-HC | | | | |
| DRB1_0101 | SPGKNHYTQKSLSLS | TDI-Y- | 5 | HYTQKSLSL | 0.595 | 48.92 |
| | | 005-HC | | | | |
| DRB1_0101 | PGKNHYTQKSLSLSP | TDI-Y- | 4 | HYTQKSLSL | 0.54 | 40.76 |
| | | 005-HC | | | | |
| DRB1_0101 | GKNHYTQKSLSLSPG | TDI-Y- | 3 | HYTQKSLSL | 0.505 | 38.46 |
| | | 005-HC | | | | |
| DRB1_0101 | KNHYTQKSLSLSPGK | TDI-Y- | 2 | HYTQKSLSL | 0.435 | 37.93 |
| | | 005-HC | | | | |
| DRB1_0401 | GGSLRLSCAAPGFIF | TDI-Y- | 3 | LRLSCAAPG | 0.935 | 49.57 |
| | | 005-HC | | | | |
| DRB1_0401 | GLEWVSYISSSSSTT | TDI-Y- | 3 | WVSYISSSS | 0.34 | 43.84 |
| | | 005-HC | | | | |
| DRB1_0401 | LEWVSYISSSSSTTY | TDI-Y- | 5 | YISSSSSTT | 0.545 | 29.01 |
| | | 005-HC | | | | |
| DRB1_0401 | EWVSYISSSSSTTYY | TDI-Y- | 4 | YISSSSSTT | 0.68 | 25.49 |
| | | 005-HC | | | | |
| DRB1_0401 | WVSYISSSSSTTYYA | TDI-Y- | 3 | YISSSSSTT | 0.785 | 24.99 |
| | | 005-HC | | | | |
| DRB1_0401 | VSYISSSSSTTYYAD | TDI-Y- | 2 | YISSSSSTT | 0.83 | 33.18 |
| | | 005-HC | | | | |
| DRB1_0401 | AKNSLYLQMNSLRDE | TDI-Y- | 5 | YLQMNSLRD | 0.77 | 46.58 |
| | | 005-HC | | | | |
| DRB1_0401 | KNSLYLQMNSLRDED | TDI-Y- | 4 | YLQMNSLRD | 0.79 | 49.59 |
| | | 005-HC | | | | |
| DRB1_0405 | LEWVSYISSSSSTTY | TDI-Y- | 5 | YISSSSSTT | 0.41 | 46.11 |
| | | 005-HC | | | | |
| DRB1_0405 | EWVSYISSSSSTTYY | TDI-Y- | 4 | YISSSSSTT | 0.565 | 45.11 |
| | | 005-HC | | | | |
| DRB1_0405 | WVSYISSSSSTTYYA | TDI-Y- | 3 | YISSSSSTT | 0.72 | 49.43 |
| | | 005-HC | | | | |
| DRB1_0405 | NAKNSLYLQMNSLRD | TDI-Y- | 6 | YLQMNSLRD | 0.725 | 35.93 |
| | | 005-HC | | | | |
| DRB1_0405 | AKNSLYLQMNSLRDE | TDI-Y- | 5 | YLQMNSLRD | 0.8 | 25.15 |
| | | 005-HC | | | | |
| DRB1_0405 | KNSLYLQMNSLRDED | TDI-Y- | 4 | YLQMNSLRD | 0.79 | 26.43 |
| | | 005-HC | | | | |
| DRB1_0405 | NSLYLQMNSLRDEDT | TDI-Y- | 3 | YLQMNSLRD | 0.795 | 27.3 |
| | | 005-HC | | | | |
| DRB1_0405 | SLYLQMNSLRDEDTA | TDI-Y- | 2 | YLQMNSLRD | 0.815 | 30.92 |
| | | 005-HC | | | | |
| DRB1_0405 | LYLQMNSLRDEDTAV | TDI-Y- | 1 | YLQMNSLRD | 0.83 | 49.27 |
| | | 005-HC | | | | |
| DRB1_0701 | LEWVSYISSSSSTTY | TDI-Y- | 5 | YISSSSSTT | 0.585 | 37.8 |
| | | 005-HC | | | | |
| DRB1_0701 | EWVSYISSSSSTTYY | TDI-Y- | 4 | YISSSSSTT | 0.68 | 31.52 |
| | | 005-HC | | | | |
| DRB1_0701 | WVSYISSSSSTTYYA | TDI-Y- | 3 | YISSSSSTT | 0.725 | 29.06 |
| | | 005-HC | | | | |
| DRB1_0701 | VSYISSSSSTTYYAD | TDI-Y- | 2 | YISSSSSTT | 0.77 | 40.61 |
| | | 005-HC | | | | |
| DRB1_0701 | QYNSTYRVVSVLTVL | TDI-Y- | 5 | YRVVSVLTV | 0.65 | 48.08 |
| | | 005-HC | | | | |
| DRB1_0701 | YNSTYRVVSVLTVLH | TDI-Y- | 4 | YRVVSVLTV | 0.71 | 44.03 |
| | | 005-HC | | | | |
| DRB1_0701 | NSTYRVVSVLTVLHQ | TDI-Y- | 3 | YRVVSVLTV | 0.68 | 42.67 |
| | | 005-HC | | | | |

TABLE 66
TDI-Y-005 Light Chain immunogenicity analysis.

| Allele | Peptide | Identity | Pos | Core | Core_Rel | Affinity(nM) |
|---|---|---|---|---|---|---|
| DRB1_0101 | DIVMTQTPLSSPVSL | TDI-Y- | 1 | IVMTQTPLS | 0.62 | 27.56 |
| | | 005-LC | | | | |
| DRB1_0101 | AEDVGVYYCMQATQF | TDI-Y- | 6 | YYCMQATQF | 0.76 | 39.67 |
| | | 005-LC | | | | |
| DRB1_0101 | EDVGVYYCMQATQFP | TDI-Y- | 5 | YYCMQATQF | 0.695 | 21.67 |
| | | 005-LC | | | | |
| DRB1_0101 | DVGVYYCMQATQFPF | TDI-Y- | 4 | YYCMQATQF | 0.645 | 14.04 |
| | | 005-LC | | | | |
| DRB1_0101 | VGVYYCMQATQFPFT | TDI-Y- | 3 | YYCMQATQF | 0.6 | 12.24 |
| | | 005-LC | | | | |
| DRB1_0101 | GVYYCMQATQFPFTF | TDI-Y- | 2 | YYCMQATQF | 0.545 | 12.87 |
| | | 005-LC | | | | |
| DRB1_0101 | VYYCMQATQFPFTFG | TDI-Y- | 1 | YYCMQATQF | 0.495 | 16.32 |
| | | 005-LC | | | | |
| DRB1_0101 | YYCMQATQFPFTFGP | TDI-Y- | 1 | YCMQATQFP | 0.48 | 35.78 |
| | | 005-LC | | | | |
| DRB1_0101 | ATQFPFTFGPGTKVD | TDI-Y- | 5 | FTFGPGTKV | 0.815 | 46.59 |
| | | 005-LC | | | | |
| DRB1_0101 | TQFPFTFGPGTKVDI | TDI-Y- | 4 | FTFGPGTKV | 0.845 | 25.13 |
| | | 005-LC | | | | |
| DRB1_0101 | QFPFTFGPGTKVDIK | TDI-Y- | 3 | FTFGPGTKV | 0.845 | 22.39 |
| | | 005-LC | | | | |
| DRB1_0101 | FPFTFGPGTKVDIKR | TDI-Y- | 2 | FTFGPGTKV | 0.84 | 27.68 |
| | | 005-LC | | | | |
| DRB1_0101 | GTKVDIKRTVAAPSV | TDI-Y- | 5 | IKRTVAAPS | 0.86 | 29.89 |
| | | 005-LC | | | | |
| DRB1_0101 | TKVDIKRTVAAPSVF | TDI-Y- | 4 | IKRTVAAPS | 0.88 | 16.38 |
| | | 005-LC | | | | |
| DRB1_0101 | KVDIKRTVAAPSVFI | TDI-Y- | 3 | IKRTVAAPS | 0.825 | 12.28 |
| | | 005-LC | | | | |
| DRB1_0101 | VDIKRTVAAPSVFIF | TDI-Y- | 2 | IKRTVAAPS | 0.62 | 13.48 |
| | | 005-LC | | | | |
| DRB1_0101 | DIKRTVAAPSVFIFP | TDI-Y- | 1 | IKRTVAAPS | 0.495 | 19.63 |
| | | 005-LC | | | | |
| DRB1_0101 | IKRTVAAPSVFIFPP | TDI-Y- | 3 | TVAAPSVFI | 0.505 | 33.56 |
| | | 005-LC | | | | |
| DRB1_0101 | DEQLKSGTASVVCLL | TDI-Y- | 3 | LKSGTASVV | 0.93 | 39.3 |
| | | 005-LC | | | | |
| DRB1_0101 | EQLKSGTASVVCLLN | TDI-Y- | 2 | LKSGTASVV | 0.94 | 36.88 |
| | | 005-LC | | | | |
| DRB1_0101 | DSKDSTYSLSSTLTL | TDI-Y- | 6 | YSLSSTLTL | 0.535 | 30.17 |
| | | 005-LC | | | | |
| DRB1_0101 | SKDSTYSLSSTLTLS | TDI-Y- | 5 | YSLSSTLTL | 0.645 | 17.43 |
| | | 005-LC | | | | |
| DRB1_0101 | KDSTYSLSSTLTLSK | TDI-Y- | 4 | YSLSSTLTL | 0.695 | 14.17 |
| | | 005-LC | | | | |
| DRB1_0101 | DSTYSLSSTLTLSKA | TDI-Y- | 3 | YSLSSTLTL | 0.745 | 14.8 |
| | | 005-LC | | | | |
| DRB1_0101 | STYSLSSTLTLSKAD | TDI-Y- | 2 | YSLSSTLTL | 0.8 | 15.76 |
| | | 005-LC | | | | |
| DRB1_0101 | TYSLSSTLTLSKADY | TDI-Y- | 1 | YSLSSTLTL | 0.785 | 29.3 |
| | | 005-LC | | | | |
| DRB1_0701 | PRVLIYKISKRFSGV | TDI-Y- | 3 | LIYKISKRF | 0.525 | 49.75 |
| | | 005-LC | | | | |
| DRB1_0701 | KVDIKRTVAAPSVFI | TDI-Y- | 3 | IKRTVAAPS | 0.75 | 48.96 |
| | | 005-LC | | | | |
| DRB1_0701 | DSKDSTYSLSSTLTL | TDI-Y- | 6 | YSLSSTLTL | 0.82 | 42.43 |
| | | 005-LC | | | | |
| DRB1_0701 | SKDSTYSLSSTLTLS | TDI-Y- | 5 | YSLSSTLTL | 0.875 | 26.93 |
| | | 005-LC | | | | |
| DRB1_0701 | KDSTYSLSSTLTLSK | TDI-Y- | 4 | YSLSSTLTL | 0.85 | 22.69 |
| | | 005-LC | | | | |
| DRB1_0701 | DSTYSLSSTLTLSKA | TDI-Y- | 3 | YSLSSTLTL | 0.81 | 22.23 |
| | | 005-LC | | | | |
| DRB1_0701 | STYSLSSTLTLSKAD | TDI-Y- | 2 | YSLSSTLTL | 0.825 | 24.34 |
| | | 005-LC | | | | |
| DRB1_0701 | TYSLSSTLTLSKADY | TDI-Y- | 1 | YSLSSTLTL | 0.755 | 37.45 |
| | | 005-LC | | | | |
| DRB1_1101 | PGQPPRVLIYKISKR | TDI-Y- | 6 | VLIYKISKR | 0.455 | 47.91 |
| | | 005-LC | | | | |
| DRB1_1101 | GQPPRVLIYKISKRF | TDI-Y- | 6 | LIYKISKRF | 0.385 | 23.99 |
| | | 005-LC | | | | |
| DRB1_1101 | QPPRVLIYKISKRFS | TDI-Y- | 6 | IYKISKRFS | 0.375 | 13.23 |
| | | 005-LC | | | | |
| DRB1_1101 | PPRVLIYKISKRFSG | TDI-Y- | 5 | IYKISKRFS | 0.45 | 11.29 |
| | | 005-LC | | | | |
| DRB1_1101 | PRVLIYKISKRFSGV | TDI-Y- | 4 | IYKISKRFS | 0.42 | 10.07 |
| | | 005-LC | | | | |
| DRB1_1101 | RVLIYKISKRFSGVP | TDI-Y- | 3 | IYKISKRFS | 0.405 | 10.48 |
| | | 005-LC | | | | |
| DRB1_1101 | VLIYKISKRFSGVPD | TDI-Y- | 2 | IYKISKRFS | 0.43 | 13.98 |
| | | 005-LC | | | | |
| DRB1_1101 | LIYKISKRFSGVPDR | TDI-Y- | 1 | IYKISKRFS | 0.485 | 21.25 |
| | | 005-LC | | | | |
| DRB1_1101 | IYKISKRFSGVPDRF | TDI-Y- | 1 | YKISKRFSG | 0.545 | 48.71 |
| | | 005-LC | | | | |
| DRB1_1501 | GQPPRVLIYKISKRF | TDI-Y- | 5 | VLIYKISKR | 0.44 | 49.85 |
| | | 005-LC | | | | |
| DRB1_1501 | QPPRVLIYKISKRFS | TDI-Y- | 5 | LIYKISKRF | 0.45 | 32.08 |
| | | 005-LC | | | | |
| DRB1_1501 | PPRVLIYKISKRFSG | TDI-Y- | 4 | LIYKISKRF | 0.43 | 32.14 |
| | | 005-LC | | | | |
| DRB1_1501 | PRVLIYKISKRFSGV | TDI-Y- | 3 | LIYKISKRF | 0.46 | 27.98 |
| | | 005-LC | | | | |
| DRB1_1501 | RVLIYKISKRFSGVP | TDI-Y- | 2 | LIYKISKRF | 0.46 | 31 |
| | | 005-LC | | | | |

Allele: MHC molecule name
Peptide: Amino acid sequence
Identity: Name of the input sequence
Pos: Starting position of the optimal binding core
Core: Binding core register
Core_Rel: Reliability of the binding core
Affinity (nM): Predicted binding affinity in nanomolar IC50

Candidate Antibody Protein Sequences. The full-length protein sequences of the TDI-Y-004 and TDI-Y-005 antibodies evaluated in candidate manufacturability studies are shown in Tables 3 and 4, Section 3.

### Example 4—Lead Characterization

Summary of Lead Characterization. To establish the target candidate profile (TCP), the development candidate antibodies TDI-Y-004 and TDI-Y-005 produced in stable CHO pools were tested for binding, cross-reactivity and selectivity in a series of in vitro assays using recombinant L1CAM protein and cells expressing L1CAM. ADCs generated with TDI-Y-004 and TDI-Y-005, a cleavable or non-cleavable linker and the PNU-159682 payload were tested in cytotoxicity experiments, followed by in vivo safety and efficacy studies. TDI-Y-004 and TDI-Y-005 bound to L1CAM in solution and on cells with high affinity. Both mAbs were cross-reactive with cynomolgus L1CAM. In addition, TDI-Y-005 was cross-reactive with mouse L1CAM. Both mAbs were highly selective over close homologs of L1CAM (e.g., CHL-1, Neurofascin and NrCAM).

Binding to L1CAM in Solution. Affinity to human L1CAM was measured by BLI using an Octet Red96e instrument. His-tagged, monomeric human L1CAM protein was captured on Ni-NTA biosensors, and an 8-point, 2-fold dilution series of TDI-Y-004 or TDI-Y-005 starting at 50 nM was used as analyte. The data was prepared by reference well subtraction, and response curves were globally fit to a 1:1 Langmuir binding model. The results are shown in Table 67. TDI-Y-004 and TDI-Y-005 bound to L1CAM in with dissociation constants (K_{D}) of 0.35 nM and 4.3 nM, respectively. The about 10-fold difference in K_{D} can be largely attributed to the faster on-rate of TDI-Y-004 compared to TDI-Y-005, whereas the off-rates between the two mAbs were similar.

TABLE 67
Binding kinetics of TDI-Y-004 and TDI-Y-005 to human LICAM.

| Antibody | Kₒₙ (1/Ms) | K_{off} (1/s) | K_{D} (nM) |
|---|---|---|---|
| TDI-Y-004 | 2.7 × 10⁵ | 9.5 × 10⁻⁵ | 0.35 nM |
| TDI-Y-005 | 3.3 × 10⁴ | 1.4 × 10⁻⁴ | 4.3 nM |

Cross-reactivity to Cynomolgus Monkey L1CAM. Affinity to cynomolgus L1CAM was measured by BLI using an Octet Red96e instrument. His-tagged, monomeric cynomolgus L1CAM protein was captured on Ni-NTA biosensors, and an 8-point, 2-fold dilution series of TDI-Y-004 or TDI-Y-005 starting at 50 nM was used as analyte. The data was prepared by reference well subtraction, and response curves were globally fit to a 1:1 Langmuir binding model. The results are shown in Table 68. TDI-Y-004 and TDI-Y-005 bound to cynomolgus monkey L1CAM in with dissociation constants (K_{D}) of 0.3 nM and 1.3 nM, respectively. Compared to human L1CAM, these values were within 1.2-fold for TDI-Y-004 and 3.3-fold for TDI-Y-005.

TABLE 68
Binding kinetics of TDI-Y-004 and TDI-
Y-005 to cynomolgus monkey L1CAM.

| Antibody | Kₒₙ (1/Ms) | K_{off} (1/s) | K_{D} (nM) |
|---|---|---|---|
| TDI-Y-004 | 1.6 × 10⁵ | 4.7 × 10⁻⁵ | 0.30 nM |
| TDI-Y-005 | 9.3 × 10⁴ | 1.2 × 10⁻⁴ | 1.3 nM |

Cross-reactivity to Mouse L1CAM. Affinity to mouse L1CAM was measured by BLI using an Octet Red96e instrument. His-tagged, monomeric cynomolgus L1CAM protein was captured on Ni-NTA biosensors, and an 8-point, 2-fold dilution series of TDI-Y-004 or TDI-Y-005 starting at 50 nM was used as analyte. The data was prepared by reference well subtraction, and response curves were globally fit to a 1:1 Langmuir binding model. The results are shown in Table 69. No binding to mouse L1CAM was observed for TDI-Y-004, whereas TDI-Y-005 bound with a dissociation constant (K_{D}) of 34 nM, which is within 8-fold of the KD for human L1CAM.

TABLE 69
Binding kinetics of TDI-Y-004 and TDI-Y-005 to mouse LICAM.

| | | | |
|---|---|---|---|
| Antibody | Kₒₙ (1/Ms) | K_{off} (1/s) | K_{D} (nM) |

| | |
|---|---|
| TDI-Y-004 | No binding |

| | | | |
|---|---|---|---|
| TDI-Y-005 | 3.1 × 10⁴ | 1.1 × 10⁻³ | 34 nM |

Selectivity. CHL-1 (L1CAM-2), Neurofascin and NrCAM were used to assess the selectivity of TDI-Y-004 and TDI-Y-005 for L1CAM. These proteins are other members of the L1 family of cell adhesion proteins and have between 49% and 55% sequence similarity with L1CAM in the extracellular portion. ELISA plates were coated with human CHL-1 (residues 25-1096 with a C-terminal 10×His tag, R&D Systems), human Neurofascin (residues 25-1039 with a C-terminal 10×His tag, R&D Systems) and human NrCAM (residues 30-600 with a C-terminal human IgG1 Fc tag, R&D Systems), followed by incubation of TDI-Y-004 or TDI-Y-005 at various concentrations. Positive control antibodies were included for each homolog. No binding of TDI-Y-004 or TDI-Y-005 was observed to CHL-1, Neurofascin or NrCAM at 1 μM. Epitope. The extracellular portion of L1CAM contains six immunoglobulin-like (Ig) domains and five fibronectin type III (FN) domains. To narrow down the binding region of the development candidate mAbs, truncated versions of human L1CAM protein (Ig domain, residues 21-605; FN domain, residues 606-1116; each with a C-terminal 6×His tag) were recombinantly produced, followed by a BLI experiment to test binding at 50 and 100 nM. TDI-Y-004 binds to the membrane-proximal FN domain, whereas TDI-Y-005 binds to the membrane-distal Ig domain. 6×His tag is set forth in SEQ ID NO: 316.

Binding to L1CAM on Cells. To assess the binding of TDI-Y-004 and TDI-Y-005 to cell surface-bound L1CAM, HEK293T cells overexpressing human L1CAM as well as the MCF-7 and MDA-MB-231 human breast cancer cell lines, which express endogenous L1CAM at various levels, were used. TDI-Y-004 and TDI-Y-005 were added to cells starting at 100 μg/ml, with 10 serial 3-fold dilutions, followed by washing. Cells were then incubated with an AlexaFluor 647 goat anti-human Fc secondary antibody. After washing, the stained cells were analyzed by flow cytometry.

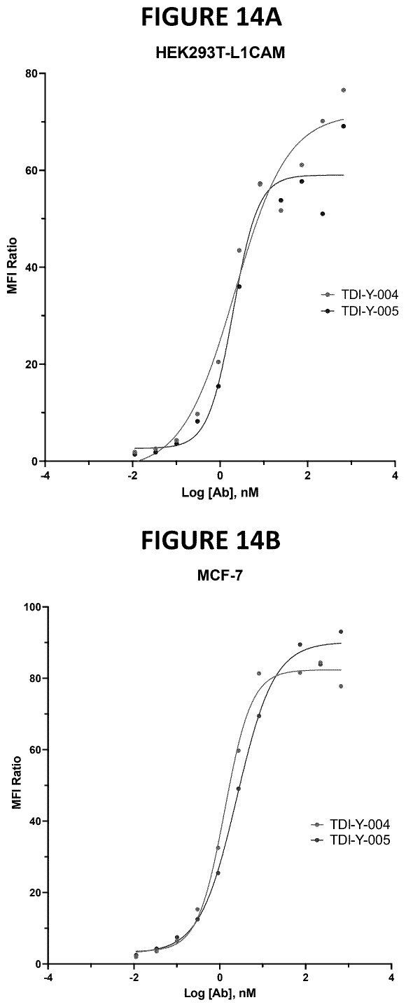

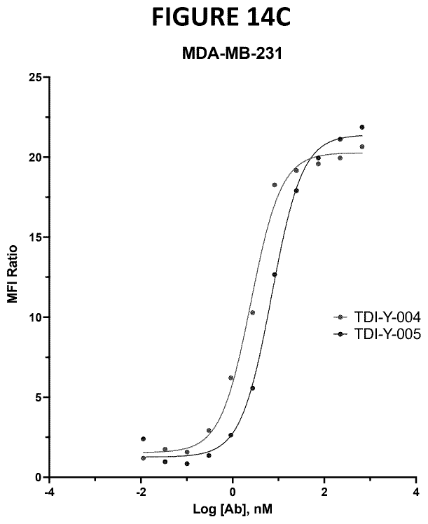

The binding curves for each antibody and cell line normalized to secondary antibody only staining (MFI ratio) are shown in FIGS. 14A-14C. EC50 values were determined by non-linear regression (Table 70). TDI-Y-004 and TDI-Y-005 bound L1CAM in the single-digit nM range on all three cell types. TDI-Y-004 bound cancer cell lines that endogenously express L1CAM with 2- to 3-fold higher affinity than TDI-Y-005, whereas binding to HEK293T cells overexpressing L1CAM was similar between the two mAbs. It is possible that differences in the conformation and oligomerization of L1CAM, depending on the surface expression level, contributed to the binding pattern. TDI-Y-005 bound to HEK293FT cells transiently transfected with mouse L1CAM with high affinity (EC50=0.9 nM; data not shown). An avidity effect due to high levels of L1CAM surface expression may have contributed to the strong binding observed in this experiment.

TABLE 70
EC50 Binding Values of TDI-Y-004 and
TDI-Y-005 on Different Cell Lines.

| Cell Line | Antibody | EC₅₀ (nM) |
|---|---|---|
| HEK293T-hL1CAM | TDI-Y-004 | 2.2 |
| | TDI-Y-005 | 2.0 |
| MCF-7 | TDI-Y-004 | 1.3 |
| | TDI-Y-005 | 2.5 |
| MDA-MB-231 | TDI-Y-004 | 2.5 |
| | TDI-Y-005 | 7.1 |

### Example 5—Generation and Analysis of Antibody-Drug Conjugates

Generation of ADCs with Various Payloads. The 143G03 and 14A10 mAbs were produced recombinantly in Expi293F cells and scaled up for ADC scouting. The original formats for 143G03 (mouse IgG1) and 14A10 (AlivaMab® with a mouse IgG1 Fc) were maintained. A one-step purification using a HiTrap Protein G HP column resulted in material that passed the TDI quality control criteria (<5% aggregation, monodisperse peak by size exclusion chromatography and endotoxin <0.1 EU/mg).

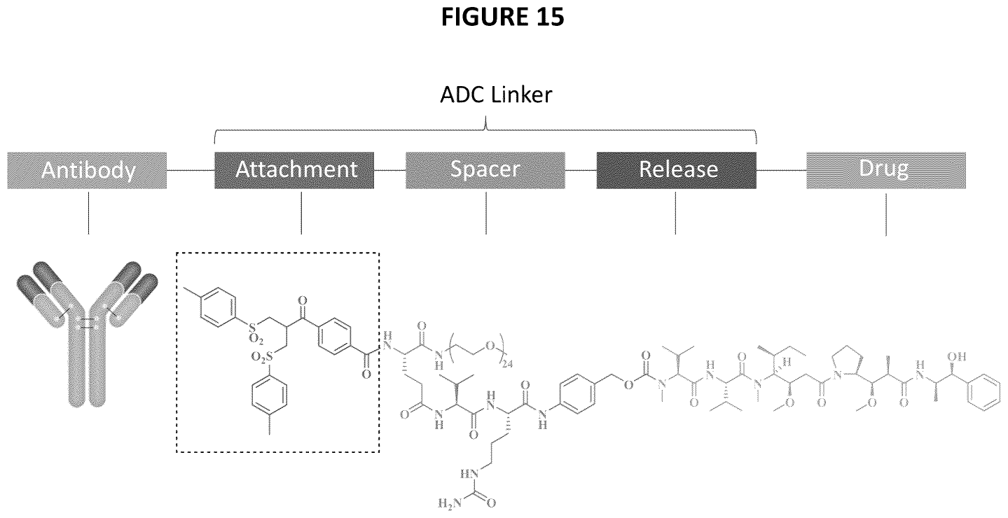

To produce ADCs, the ThioBridge® platform for linking toxic payloads to the antibodies was used (FIG. 15). The technology employs the selective reduction of disulfide bonds between the heavy and light chains as well as the hinge region of the antibodies. The conjugation of a bis-sulfone cysteine-bridging linker under mild aqueous conditions results in the production of stable ADCs with highly homogeneous drug-to-antibody ratios (DAR) (Anami et al., Nat Commun 9, 2512 (2018); Bird et al., Methods Mol Biol 2078, 113-129 (2020)) with improved preclinical profiles (Yang et al., Mol Cancer Ther (2021)). Interchain disulfide bonds between the heavy and light chains and the hinge regions of IgGs are reduced to produce pairs of cysteine residues with free thiols, which serve as acceptor sites for a bis-sulfone bis-alkylating attachment unit to covalently rebridge the disulfides. A spacer comprising a polymer chain is included to increase the hydrophilicity of the ADC. The toxic drug is conjugated via a self-immolative release linker.

To screen for ADCs that potently kill L1CAM expressing cells, a series of derivatives were produced with a matrix of ThioBridge® linker-payloads that contained different cytotoxins, polymers and release mechanisms. These ADCs were then compared in a series of developability assessments, including in vitro, ex vivo and in vivo efficacy and safety evaluations. Six toxic payloads with various mechanisms of action were selected for conjugation to 143G03 and 14A10 (Table 71). A linker was utilized for each payload. The linkers included a valine-citrulline (Val-Cit) cathepsin B cleavage site for release of the payload in the lysosome, and a cyclic or linear PEG spacer unit to reduce the hydrophobicity of the ADCs. In addition, an isotype control mouse IgG1 antibody was included in all reactions.

TABLE 71
ADC Screening: toxic payloads and their mechanisms of action.

| Payload | Class | Mechanism of Action |
|---|---|---|
| Monomethyl Auristatin E | Dolastatin | Microtubule polymerization inhibitor |
| ABZ038 | Maytansinoid | Microtubule polymerization inhibitor |
| Duocarmycin TM | Duocarmycin | DNA alkylator |
| PNU-159682 | Anthracyclin | DNA intercalator/alkylator |
| SN38 | Camptothecin | DNA Topoisomerase I inhibitor |
| a-Amanitin | Amatoxin | RNA Polymerase II inhibitor |

After reduction of the interchain disulfides, the mAbs were treated with a ThioBridge® linker-payload reagent. The mixture was buffer-exchanged and concentrated by ultrafiltration and diafiltration, and further processed by incubation with activated charcoal before final sterile filtration. ADCs were quantified by UV/VIS and characterized for DAR by UV and LC-MS, for purity by SEC and SDS-PAGE, for free reagent related species by SEC, and endotoxin levels.

Cleavable Linkers. The cleavable linkers used for the ADC screening included a valine-citrulline (Val-Cit) dipeptide and a para-aminobenzyl (PAB)-dimethylethylenediamine (DMAE) spacer connected to PNU-159682. The amide bond at the Val-Cit and PAB junction is prone to cleavage by Ces1c, an extracellular carboxyl esterase present in mouse serum. This can lead to ADC instability and premature release of the payload from the antibody prior to internalization by target cells, causing potential systemic toxicities and a poor PK profile in mouse studies (Dorywalska et al., Mol Cancer Ther 15, 958-970 (2016)). Even though Ces1c is not present in the serum of humans and non-human primates, it can severely impair the preclinical development and testing of ADC drugs with cleavable linkers.

The maximum tolerated dose for free PNU-159682 in mice is 15 μg/kg (Quintieri et al., Clin Cancer Res 11, 1608-1617 (2005)). This amount of free drug can be reached even if only a small portion of drug is released from the ADCs in serum by Ces1c after administration of typical doses for mouse efficacy experiments. To mitigate this risk, the serum stability of the ADCs was assessed in vitro prior to testing in animals. The ADCs were incubated in 75% IgG-depleted mouse serum for 96 hours, followed by analysis of the average DAR by HIC. A 15-20% loss in average DAR was observed for 143G03, 14A10 and the mouse IgG control ADCs with PNU-159682.

A pilot safety experiment in mice with the L1CAM ADCs revealed a maximum tolerated dose of 0.1-0.2 mg/kg when administered intraperitoneally once a week. A subsequent pilot efficacy xenograft study showed that the therapeutic window was too small to achieve sufficient tumor killing without serious toxicity in the animals, indicating the need for linker modification to achieve the desired efficacy.

Addition of a glutamic acid at the N-terminus of the Val-Cit dipeptide substantially reduces cleavage in mouse serum while maintaining cathepsin B-mediated drug release in the endosome (Anami et al., Nat Commun 9, 2512 (2018)). This modified Glu-Val-Cit tripeptide linker was incorporated into the L1CAM ADCs. In parallel, additional ADC variants with a non-cleavable linker, containing a tri-glycine spacer linked to ethylene diamine (EDA), were generated. The antibodies with the final humanized or human sequences (e.g., TDI-Y-004 and TDI-Y-005) plus a human IgG1 isotype control mAb were utilized to produce these ADCs.

Analysis of the ADCs in in vitro mouse serum confirmed that the cleavable linker resulted in increased stability, with a reduction in average DAR of 3% or less, with one degradation product identified by LC-MS. Incubation of the ADCs with the cleavable linker in human serum did not result in any payload release. The ADCs with the non-cleavable linker were fully stable in mouse and human serum after 96 hours, with no payload release observed.

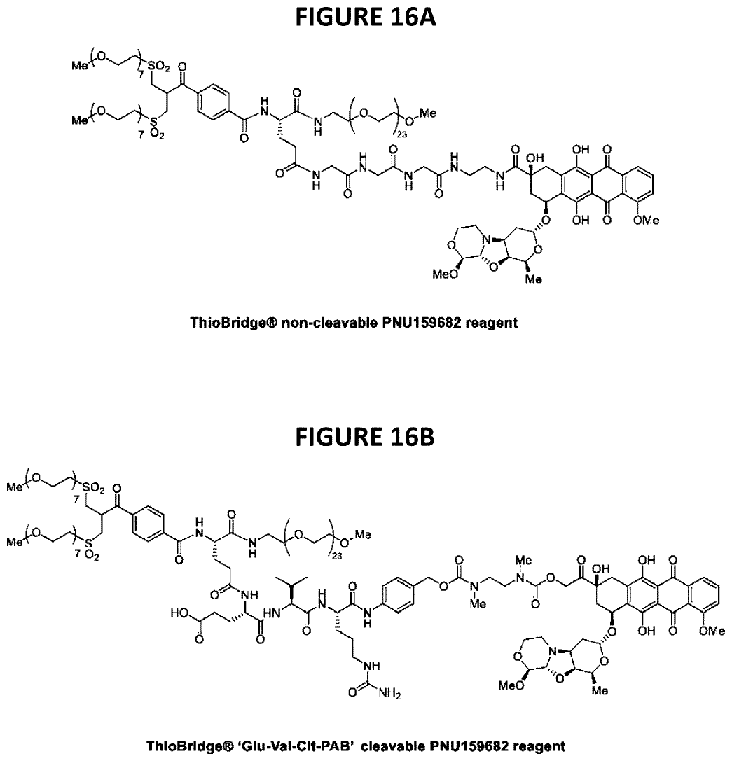

The structures of the final cleavable and the non-cleavable linkers, which were used for in vitro potency and in vivo efficacy studies with antibody drug conjugates of TDI-Y-004 and TDI-Y-005, are depicted in FIGS. 16A and 16B.

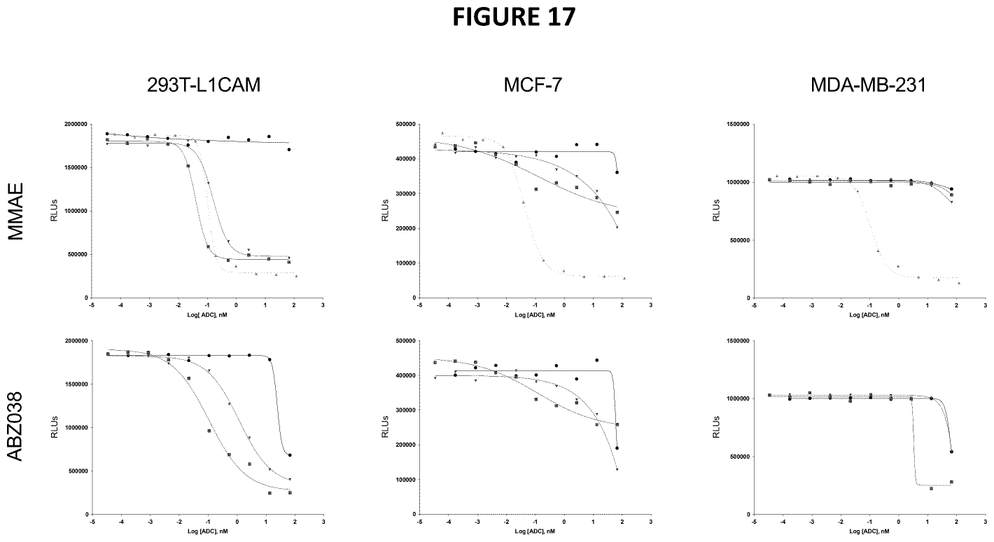

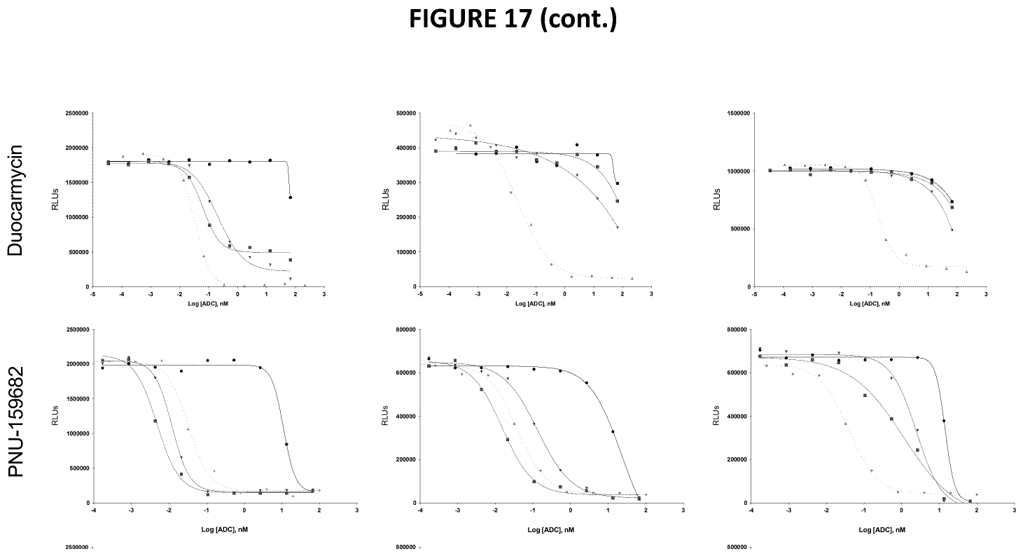

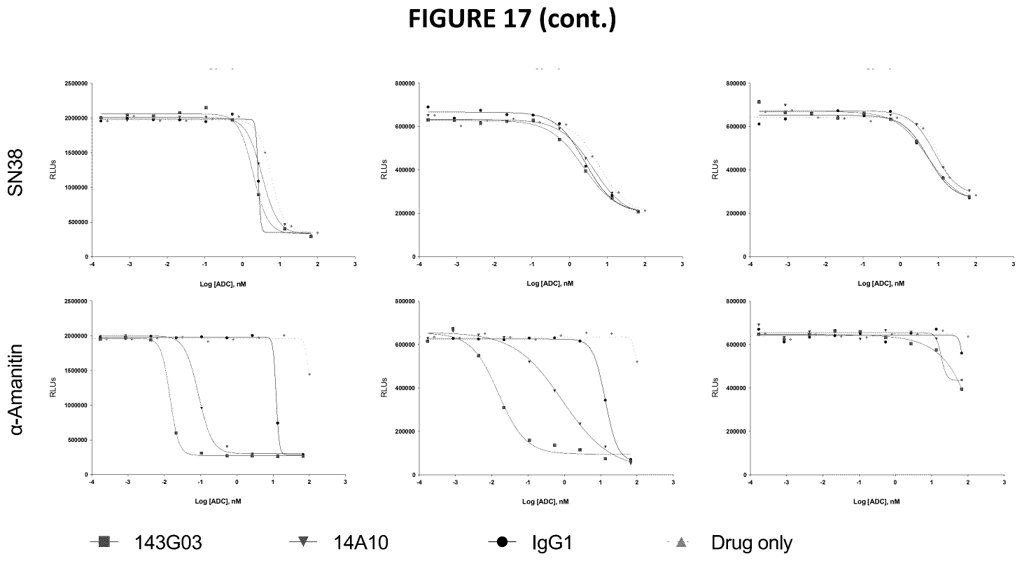

ADC Cytotoxicity Analysis. The potency of the ADCs was tested in cytotoxicity assays using various cell types that express human L1CAM at different levels, including stably transfected HEK293T cells (overexpression), the MCF-7 breast cancer cell line (medium expression) and the MDA-MB-231 breast cancer cell line (low-medium expression). The ADCs were added to the cells at various concentrations ranging from 0.1 pM to 100 nM, and cell viability was assessed 96 hours later. Killing curves for the two lead candidate mAbs plus isotype control, the payloads and cells tested are shown in FIG. 17. All payloads except for SN38 showed strong cytotoxicity for HEK293T cells overexpressing L1CAM, with EC50 values in the sub-nM range. In MCF-7 cells, which endogenously express L1CAM, strong cytotoxicity was observed for ADCs with either PNU-159682 or α-Amanitin. PNU-159682 was the only payload that showed killing of MDA-MB-231 cells, which express lower levels of L1CAM than MCF-7.

In general, the killing potency was 0.5 to 1 log higher for 143G03 than for 14A10. This correlates with the binding affinities of the two mAbs for L1CAM. Non-specific killing was observed for the IgG1 isotype control at higher concentrations for most payloads, suggesting additional uptake mechanisms including Fc receptor-mediated endocytosis or pinocytosis.

ADCs with six different toxic payloads conjugated to 143G03, 14A10 and an isotype control mouse IgG1 were screened for their killing potency on various cell types as indicated. Free drug was included where available.

In addition to the six toxic payloads tested using Abzena's ThioBridge® platform, two payloads were analyzed in a pilot experiment using non-directed conjugation to surface-exposed lysines on 143G03 with a target DAR of 4 (data not shown). ADCs with a pyrrolobenzodiazepine (PBD) payload showed strong killing potency on HEK293T cells overexpressing L1CAM, whereas limited cytotoxicity was observed for ADCs with monomethyl auristatin F (MMAF).

Based on these results, PNU-159682, a metabolite of the anthracycline nemorubicin, was used as payload for further modification and testing in animal models to establish in vivo proof-of-concept.

### Example 6—Cytotoxicity of Antibody-Drug Conjugates

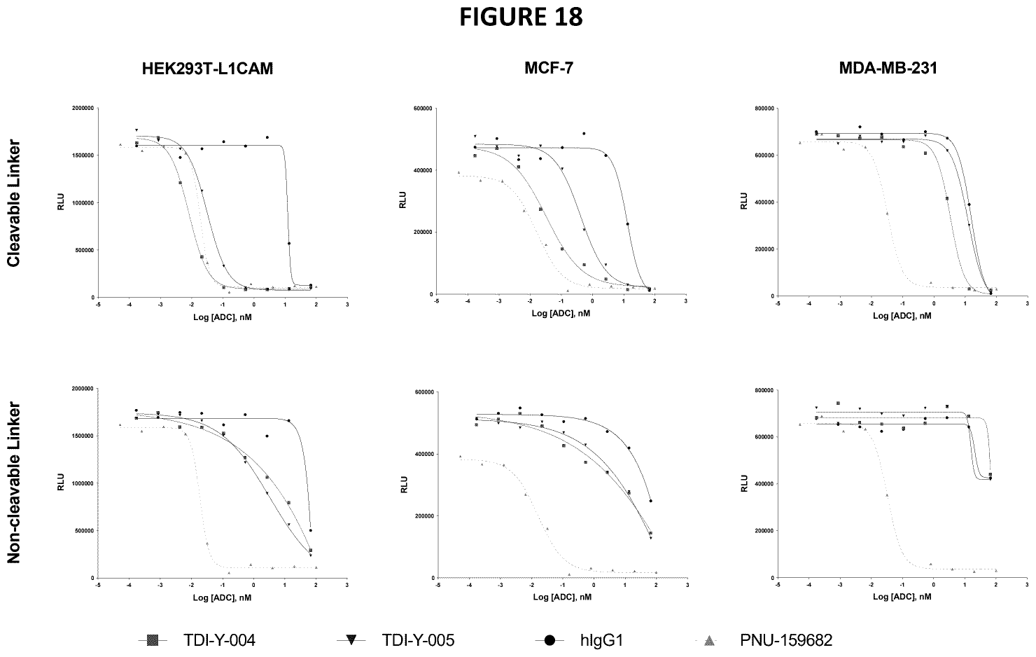

ADCs generated using TDI-Y-004 and TDI-Y-005 plus a human IgG1 isotype control with the PNU-159682 payload, and either a cleavable Glu-Val-Cit linker or a non-cleavable Gly3 linker, were tested for cytotoxicity using HEK293T cells overexpressing human L1CAM, and the MCF-7 and MDA-MB-231 human breast cancer cell lines. The ADCs were added to the cells at various concentrations, with 10 serial 5-fold dilutions starting at 10 μg/ml. Cell viability was assessed 96 hours later using CellTiterGlo reagent, and cell viability was plotted against antibody concentration (FIG. 18).

The EC50 values for cytotoxicity of the TDI-Y-004 and TDI-Y-005 ADCs with a cleavable linker for HEK293T cells overexpressing human L1CAM and for MCF-7 cells was in the sub-nM range (Table 72). On MDA-MB-231 cells, cytotoxicity potency was reduced, especially for TDI-Y-005 with an EC50 of 12 nM. TDI-Y-004 consistently showed higher potency than TDI-Y-005, which is likely due to the higher affinity of TDI-Y-004 compared to TDI-Y-005 for L1CAM.

TABLE 72
Cytotoxicity Values of TDI-Y-004 and TDI-
Y-005 ADCs with a cleavable linker

| Cell Line | ADC | EC₅₀ (nM) |
|---|---|---|
| HEK293T-hL1CAM | TDI-Y-004 | 0.008 nM |
| | TDI-Y-005 | 0.03 nM |
| MCF-7 | TDI-Y-004 | 0.03 nM |
| | TDI-Y-005 | 0.4 nM |
| MDA-MB-231 | TDI-Y-004 | 3.3 nM |
| | TDI-Y-005 | 12.4 nM |

Killing for the TDI-Y-004 and TDI-Y-005 ADCs with a non-cleavable linker was modest in L1CAM-overexpressing HEK293T cells, further reduced in MCF-7 cells and largely absent in MDA-MB-231 cells. The non-cleavable Gly3 linker does not include an enzyme-driven release mechanism and requires degradation of the antibody in the lysosome, which likely contributes to the lower potency in the tissue culture experiments. Since L1CAM expression and turnover may be different in a tumor setting, a decision was made to include the ADCs with the non-cleavable linker in the in vivo experiments.

Safety of Antibody-Drug Conjugates. The ADCs generated with TDI-Y-004 and TDI-Y-005, PNU-159682 and a cleavable or non-cleavable linker were tested in dose range-finding studies in female treatment-naïve NSG mice. The ADCs were administered intraperitoneally once a week, with 4 doses total. The mice were monitored for weight and clinical signs. At the time of sacrifice, blood was collected for complete blood count (CBC) and clinical chemistry analysis.

The ADCs with the cleavable linker, which was modified for reduced payload release in mouse serum, were tolerated at concentrations up to 0.5 mg/kg in up to four doses. Since minor weight loss and other clinical signs occurred after the third and fourth doses, the final dose for the in vivo efficacy experiments was set at 0.3 mg/kg.

Mice injected with the ADCs with the non-cleavable linker at 1 mg/kg tolerated the drug well, with no significant weight loss or other clinical signs after four doses. Therefore, the dose for in vivo efficacy studies was set at 1 mg/kg.

Notably, there was no difference in toxicity observed between TDI-Y-004 and TDI-Y-005 for the ADCs with either linker. Since only TDI-Y-005 cross-reacts with mouse L1CAM, the toxicities observed after multiple administration of the ADCs with the cleavable linker can be attributed to a payload-related effect rather than toxicities caused by binding to L1CAM in other organs including neurons or kidney tubules.

### Example 7—Production of ThioBridge® h14A10, Hz143G03 (N61Q), and hIgG1 Antibody-Drug Conjugates with PNU159682 Payloads

The cytotoxic payload PNU159682 was employed for all ADCs. ADCs were prepared with ThioBridge®, a disulfide rebridging conjugation technology. For each antibody, two different ThioBridge® reagent formats were conjugated, a ThioBridge® ‘Glu-Val-Cit-PAB’ cleavable PNU159682 reagent and a ThioBridge® non-cleavable PNU159682 reagent (see FIGS. 16A and 16B). Both reagent formats were designed for high mouse serum stability. An average DAR of 4 was targeted for all conjugates. To allow subsequent biological testing, ADCs were prepared in 5 mg target amounts.

### Methods

LC-MS analysis. LC-MS analysis was carried out using a Waters XEVO G2S TOF mass spectrometer and a POROSHELL 300SB C3 column (2.1×12.5 mm, 5 μm) connected to a Waters Acquity H Class UPLC system. The mobile phase was buffer A (0.1% formic acid in water). A gradient (2.5 min 10% B, 10-80% B gradient in 3.5 min) was applied using Buffer B (acetonitrile, 0.1% formic acid) at a flow rate of 0.4 mL/min. The column was maintained at 60° C. throughout the analysis. ADCs were analyzed after dilution to 0.1 mg/mL. 10 μL of ADC solution were injected for analysis. Average DAR was calculated as the weighted average of the observed DAR species based on main glycoforms signal intensity (SI) on the deconvoluted m/z spectra. Average MW was calculated as the weighted average of the observed DAR species main glycoforms MWs on the deconvoluted m/z spectra, as follows:

$$Average DAR = \frac{{\sum }_{i = 0}^{n} {SI}_{i} \times i}{{\sum }_{i = 0}^{n} {SI}_{i}} \\ Average MW = \frac{{\sum }_{i = 0}^{n} {SI}_{i} \times {MW}_{i}}{{\sum }_{i = 0}^{n} {SI}_{i}}$$

SEC analysis. Analytical SEC (size exclusion chromatography) was carried out using an ACQUITY UPLC BEH SEC column (4.6 mm×15 cm, 200 A, 1.7 μm) and guard column (4.6 mm×3 cm), connected to a Dionex Ultimate 3000 UPLC system. The mobile phase was 0.2 M potassium phosphate buffer, pH 6.8, 0.2 M potassium chloride, 15% (v/v) isopropanol. The flow rate was kept constant at 0.35 mL/min. The column was maintained at 30° C. throughout the analysis. The analysis was carried out in a 10 min isocratic elution with UV detection at 280 nm and 495 nm. 10 μg of ADC were injected for analysis. The percentage of high molecular weight (HMW) species was calculated by comparing the peak area corresponding to HMW species at 280 nm to total peak area corresponding to HMW and monomeric species at 280 nm. The percentage of free reagent related species was calculated by comparing the peak area corresponding to free reagent related species at 495 nm to total peak area corresponding to HMW and monomeric species at 495 nm.

HIC analysis. Analytical HIC was carried out using a TOSOH Bioscience TSKgel Butyl-NPR column (4.6 mm×3.5 cm, 2.5 μm), connected to a Dionex Ultimate 3000 UPLC system. The mobile phase was buffer A: 1.5 M ammonium sulfate, 50 mM sodium phosphate, pH 7.0. A linear gradient (0-100% B in 10.5 min) was applied using Buffer B (20% isopropanol, 50 mM sodium phosphate, pH 7.0) at a flow rate of 1.35 mL/min to elute bound species. The column was maintained at 30° C. throughout the analysis. The analysis was carried with UV detection at 280 nm. 10 μg of ADC were injected per analysis.

SDS-PAGE analysis. SDS-PAGE analysis was carried out using NuPAGE® 4-12% Bis-Tris gels (Invitrogen) under non-reducing conditions with MES buffer. Prior to analysis, each sample was incubated for 1 h at 40° C. in 10% SDS solution. For analysis, 1 μg of ADC sample (based on protein) was loaded onto the gel per lane. Electrophoresis was carried out at 200 V for 35 min. The gels were stained with InstantBlue™ (Expedeon) for protein detection and analyzed using a ChemiDoc™ imaging system (Bio-Rad).

Concentration and average DAR determination by UV absorbance. The concentration and average DAR of the conjugates were determined by UV absorbance at 280 nm (A280) and at a wavelength λ corresponding to a maximum of absorbance for the linker-payload (Aλ) using a Nanodrop 2000 spectrophotometer. Measurements were taken in triplicate and the average values used for calculations. The average DAR was calculated using the following formula:

$$DAR = \frac{{ε}_{mAB}^{λ} - \frac{{A}_{λ}}{{A}_{280}} \times {ε}_{mAB}^{280}}{\frac{{A}_{λ}}{{A}_{280}} \times {ε}_{LP}^{280} - {ε}_{LP}^{λ}}$$

Where:

$$ε \frac{λ}{mAb}$$

molar extinction coefficient of antibody at wavelength λ (M⁻¹·cm⁻¹)
EmAb=molar extinction coefficient of antibody at 280 nm (M⁻¹·cm⁻¹)
ELAp=molar extinction coefficient of linker-payload at wavelength, (M⁻¹·cm⁻¹)
EU°=molar extinction coefficient of linker-payload at 280 nm (M⁻¹·cm⁻¹)
AA=absorbance at wavelength A
A280=absorbance at 280 nm

Molar extinction coefficients used for antibodies and linker-payloads were taken from the manufacturer's CoA or determined experimentally (Tables 73 and 74).

TABLE 73
Molar extinction coefficients for mAbs h14A10, hz143G03
(N61Q) and hIgG1 at different wavelengths.

| | λ | ε^{λmAb} | |
|---|---|---|---|
| Antibody | (nm) | (M⁻¹ · cm⁻¹) | Source |
| mAb h14A10 | 280 | 217196 | Calculated from manufacturer CoA |
| | 495 | N/A | for lot # U675ZEG190-8/P7EH001 |
| mAb | 280 | 219355 | Calculated from manufacturer CoA |
| hz143G03 | | | |
| (N61Q) | 495 | N/A | for lot # U675ZEG190-3/P7EH001 |
| mAb hIgG1 | 280 | 197708 | Calculated from manufacturer CoA |
| | 495 | N/A | for lot # 659519M2 |

TABLE 74
Molar extinction coefficients for linker-
payloads at different wavelengths

| Linker-Payload | λ (nm) | ε^{λ}LP(M⁻¹ · cm⁻¹) | Source |
|---|---|---|---|
| ThioBridge ®-Glu[- | 280 | 17118 | Experimental |
| Glu(OH)-Val-Cit-PAB- | | | |
| DMEA-PNU159682]- | 495 | 10007 | |
| PEG(24) | | | |
| ThioBridge ® Glu(-Gly3- | 280 | 12191 | Experimental |
| EDA-PNU159682)- | | | |
| PEG(24 u) | 495 | 8268 | |

The following formula was used to calculate the conjugates' concentration:

$$c = \frac{{A}_{280}}{( {ε}_{mAB}^{280} + DAR \times {ε}_{LP}^{280} ) \times 1} \times ( {MW}_{mAB} + DAR \times {MW}_{LP} ) \\ Where : c = concentration ( {( mg / mL )}^{- 1} \cdot {cm}^{- 1} ) \\ {A}_{280} = absorbance at 280 nm \\ ε \frac{280}{mAb} = molar extinction coefficient of antibody at 280 nm ( {M}^{- 1} \cdot {cm}^{- 1} ) \\ ε \frac{280}{LP} = molar extinction coefficient of linker ‐ payload at 280 nm ( {M}^{- 1} \cdot {cm}^{- 1} ) \\ DAR = average DAR \\ ι = optical path ( cm ) \\ {MW}_{mAb} = antibody MW ( g \cdot {mo}^{- 1} ) \\ {MW}_{LP} = linker ‐ payload MW ( g \cdot {mol}^{- 1} )$$

Endotoxin level determination method. The EndoSafe®-PTST™ platform (Charles River) was used to determine the level of endotoxin. EndoSafe®-PTST™ is a chromogenic kinetic test system aligned with USP <85> and Pharm Eur 2.6.14 that provides quantitative Limulus Amebocyte Lysate (LAL) results. The EndoSafe®-PTST™ utilizes LAL reagents in FDA-licensed disposable test cartridges which are pre-loaded with all the reagents required to perform a LAL test. The EndoSafe®-PTST™ mimics licensed LAL kinetic chromogenic methodology by measuring color intensity directly related to the endotoxin concentration in a sample; the concentrations are calculated against an internal standard curve (0.01-1.00 EU/mL) associated with the lot number of the cartridges.

Conjugation of mAb h14A10 with ThioBridge®-Glu[-Glu(OH)-Val-Cit-PAB-DMEA-PNU159682]-PEG(24). mAb h14A10 at 4.64 mg/mL in PBS, pH 7.4 (1.509 mL; 7.0 mg; 46.6 nmol; 1.0 eq.) was diluted with Dulbecco's PBS, pH 7.4, (26.4 μL). A 5 mM solution of TCEP in endotoxin-free water (55.9 μL; 280 nmol; 6 eq.) was added to the dilute mAb h14A10 solution. The reduction was allowed to proceed at 40° C. for 1 h with a final antibody concentration of 4.4 mg/mL. After 1 h at 40° C., the reduction mixture was diluted with Dulbecco's PBS, pH 7.4 (54.1 μL) and 500 mM EDTA solution in water (17.5 μL) and allowed to cool down to 22° C. A 11.15 mg/mL (3.24 mM) solution of B4-Glu[-Glu(OH)-Val-Cit-PAB-DMEA-PNU159682]-PEG(24) in DMF was prepared by dissolving 3.00 mg (873 nmol) of B4-Glu[-Glu(OH)-Val-Cit-PAB-DMEA-PNU159682]-PEG(24) (MW=3437.0 g/mol) into 269 μL of DMF. The B4-Glu[-Glu(OH)-Val-Cit-PAB-DMEA-PNU159682]-PEG(24) solution in DMF (69.0 μL; 0.769 mg; 224 nmol; 4.8 eq.) was added to the reduced mAb h14A10 solution resulting in a final concentration of 5% DMF and a final antibody concentration of 4.0 mg/mL. The conjugation reaction was allowed to proceed at 22° C. for 20 h. After 20 h at 22° C., the reaction mixture was buffer-exchanged and concentrated to 4.02 mg/mL by ultrafiltration/diafiltration using a Vivaspin 20 centrifugal concentrator (PES membrane, 30 kDa MWCO) equilibrated with Dulbecco's PBS, pH 7.1. Concentrated conjugate sample (7.5 mg; 1.86 mL) was sterile filtered through a 0.22 μm pore size, PVDF membrane filter. h14A10 ThioBridge®-Glu[-Glu(OH)-Val-Cit-PAB-DMEA-PNU159682]-PEG(24) conjugate was characterized by HIC, SEC, LC-MS, and SDS-PAGE and quantified by UV and endotoxin levels were determined.

Conjugation of mAb hz143G03 (N61Q) with ThioBridge®-Glu[-Glu(OH)-Val-Cit-PAB-DMEA-PNU159682]-PEG(24). mAb hz143G03 (N61Q) at 4.88 mg/mL in PBS, pH 7.4 (1.434 mL; 7.0 mg; 47.6 nmol; 1.0 eq.) was diluted with Dulbecco's PBS, pH 7.4, (99.3 μL). A 5 mM solution of TCEP in endotoxin-free water (57.2 μL; 286 nmol; 6 eq.) was added to the dilute mAb hz143G03 (N61Q) solution. The reduction was allowed to proceed at 40° C. for 1 h with a final antibody concentration of 4.4 mg/mL. After 1 h at 40° C., the reduction mixture was diluted with Dulbecco's PBS, pH 7.4 (54.1 μL) and 500 mM EDTA solution in water (17.5 μL) and allowed to cool down to 22° C. A 11.15 mg/mL (3.24 mM) solution of B4-Glu[-Glu(OH)-Val-Cit-PAB-DMEA-PNU159682]-PEG(24) in DMF was prepared by dissolving 3.00 mg (873 nmol) of B4-Glu[-Glu(OH)-Val-Cit-PAB-DMEA-PNU159682]-PEG(24) (MW=3437.0 g/mol) into 269 μL of DMF. The B4-Glu[-Glu(OH)-Val-Cit-PAB-DMEA-PNU159682]-PEG(24) solution in DMF (70.5 μL; 0.786 mg; 229 nmol; 4.8 eq.) was added to the reduced mAb hz143G03 (N61Q) solution resulting in a final concentration of 5% DMF and a final antibody concentration of 4.0 mg/mL. The conjugation reaction was allowed to proceed at 22° C. for 20 h. After 20 h at 22° C., the reaction mixture was buffer-exchanged and concentrated to 5.03 mg/mL by ultrafiltration/diafiltration using a Vivaspin 20 centrifugal concentrator (PES membrane, 30 kDa MWCO) equilibrated with Dulbecco's PBS, pH 7.1. Concentrated conjugate sample (7.3 mg; 1.45 mL) was sterile filtered through a 0.22 μm pore size, PVDF membrane filter. h143G03 ThioBridge®-Glu[-Glu(OH)-Val-Cit-PAB-DMEA-PNU159682]-PEG(24) conjugate was characterized by HIC, SEC, LC-MS, SDS-PAGE and quantified by UV and endotoxin levels were determined.

Conjugation of mAb hIgG1 with ThioBridge®-Glu[-Glu(OH)-Val-Cit-PAB-DMEA-PNU159682]-PEG(24). mAb hIgG1 at 6.68 mg/mL in PBS, pH 7.0 (1.048 mL; 7.0 mg; 47.1 nmol; 1.0 eq.) was diluted with Dulbecco's PBS, pH 7.4, (486 μL). A 5 mM solution of TCEP in endotoxin-free water (56.5 μL; 283 nmol; 6 eq.) was added to the dilute mAb hIgG1 solution. The reduction was allowed to proceed at 40° C. for 1 h with a final antibody concentration of 4.4 mg/mL. After 1 h at 40° C., the reduction mixture was diluted with Dulbecco's PBS, pH 7.4 (54.1 μL) and 500 mM EDTA solution in water (17.5 μL) and allowed to cool down to 22° C. A 11.15 mg/mL (3.24 mM) solution of B4-Glu[-Glu(OH)-Val-Cit-PAB-DMEA-PNU159682]-PEG(24) in DMF was prepared by dissolving 3.00 mg (873 nmol) of B4-Glu[-Glu(OH)-Val-Cit-PAB-DMEA-PNU159682]-PEG(24) (MW=3437.0 g/mol) into 269 μL of DMF. The B4-Glu[-Glu(OH)-Val-Cit-PAB-DMEA-PNU159682]-PEG(24) solution in DMF (62.4 μL; 0.696 mg; 202 nmol; 4.3 eq.) was added to the reduced mAb hIgG1 solution resulting in a final concentration of 5% DMF and a final antibody concentration of 4.0 mg/mL. The conjugation reaction was allowed to proceed at 22° C. for 20 h. After 20 h at 22° C., the reaction mixture was buffer-exchanged and concentrated to 5.36 mg/mL by ultrafiltration/diafiltration using a Vivaspin 20 centrifugal concentrator (PES membrane, 30 kDa MWCO) equilibrated with Dulbecco's PBS, pH 7.1. Concentrated conjugate sample (7.3 mg; 1.36 mL) was sterile filtered through a 0.22 μm pore size, PVDF membrane filter. hIgG1 ThioBridge®-Glu[-Glu(OH)-Val-Cit-PAB-DMEA-PNU159682]-PEG(24) conjugate was characterized by HIC, SEC, LC-MS, SDS-PAGE and quantified by UV and endotoxin levels were determined.

Conjugation of mAb h14A10 with ThioBridge®-Glu(-Gly3-EDA-PNU159682)-PEG(24u). mAb h14A10 at 4.64 mg/mL in PBS, pH 7.4 (1.509 mL; 7.0 mg; 46.6 nmol; 1.0 eq.) was diluted with Dulbecco's PBS, pH 7.4, (26.4 μL). A 5 mM solution of TCEP in endotoxin-free water (55.9 μL; 280 nmol; 6 eq.) was added to the dilute mAb h14A10 solution. The reduction was allowed to proceed at 40° C. for 1 h with a final antibody concentration of 4.4 mg/mL. After 1 h at 40° C., the reduction mixture was diluted with Dulbecco's PBS, pH 7.4 (54.1 μL) and 500 mM EDTA solution in water (17.5 p L) and allowed to cool down to 22° C. A 9.68 mg/mL (3.24 mM) solution of B4-Glu(-Gly3-EDA-PNU159682)-PEG(24u) in DMF was prepared by dissolving 3.50 mg (1172 nmol) of B4-Glu(-Gly3-EDA-PNU159682)-PEG(24u) (MW=2985.0 g/mol) into 361 p L of DMF. The B4-Glu(-Gly3-EDA-PNU159682)-PEG(24u) solution in DMF (86.2 μL; 0.834 mg; 280 nmol; 6.0 eq.) was added to the reduced mAb h14A10 solution resulting in a final concentration of 5% DMF and a final antibody concentration of 4.0 mg/mL. The conjugation reaction was allowed to proceed at 22° C. for 20 h. After 20 h at 22° C., the reaction mixture was buffer-exchanged and concentrated to 3.95 mg/mL by ultrafiltration/diafiltration using a Vivaspin 20 centrifugal concentrator (PES membrane, 30 kDa MWCO) equilibrated with Dulbecco's PBS, pH 7.1. Concentrated conjugate sample (7.1 mg; 1.79 mL) was sterile filtered through a 0.22 μm pore size, PVDF membrane filter. h14A10 ThioBridge®-Glu(-Gly3-EDA-PNU159682)-PEG(24u)conjugate was characterized by HIC, SEC, LC-MS, SDS-PAGE and quantified by UV and endotoxin levels were determined.

Conjugation of mAb hz143G03 (N61Q) with ThioBridge®-Glu(-Gly3-EDA-PNU159682)-PEG(24u). mAb hz143G03 (N61Q) at 4.88 mg/mL in PBS, pH 7.4 (1.434 mL; 7.0 mg; 47.6 nmol; 1.0 eq.) was diluted with Dulbecco's PBS, pH 7.4, (99.3 μL). A 5 mM solution of TCEP in endotoxin-free water (57.2 μL; 286 nmol; 6 eq.) was added to the dilute mAb hz143G03 (N61Q) solution. The reduction was allowed to proceed at 40° C. for 1 h with a final antibody concentration of 4.4 mg/mL. After 1 h at 40° C., the reduction mixture was diluted with Dulbecco's PBS, pH 7.4 (54.1 μL) and 500 mM EDTA solution in water (17.5 μL) and allowed to cool down to 22° C. A 9.68 mg/mL (3.24 mM) solution of B4-Glu(-Gly3-EDA-PNU159682)-PEG(24u) in DMF was prepared by dissolving 3.50 mg (1172 nmol) of B4-Glu(-Gly3-EDA-PNU159682)-PEG(24u) (MW=2985.0 g/mol) into 361 μL of DMF. The B4-Glu(-Gly3-EDA-PNU159682)-PEG(24u) solution in DMF (88.1 μL; 0.853 mg; 286 nmol; 6.0 eq.) was added to the reduced mAb hz143G03 (N61Q) solution resulting in a final concentration of 5% DMF and a final antibody concentration of 4.0 mg/mL. The conjugation reaction was allowed to proceed at 22° C. for 20 h. After 20 h at 22° C., the reaction mixture was buffer-exchanged and concentrated to 4.97 mg/mL by ultrafiltration/diafiltration using a Vivaspin 20 centrifugal concentrator (PES membrane, 30 kDa MWCO) equilibrated with Dulbecco's PBS, pH 7.1. Concentrated conjugate sample (7.4 mg; 1.48 mL) was sterile filtered through a 0.22 μm pore size, PVDF membrane filter. hz143AG03 (N61Q) ThioBridge®-Glu(-Gly3-EDA-PNU159682)-PEG(24u)conjugate was characterized by HIC, SEC, LC-MS, SDS-PAGE and quantified by UV and endotoxin levels were determined.

Conjugation of mAb hIgG1 with ThioBridge®-Glu(-Gly3-EDA-PNU159682)-PEG(24u). mAb hIgG1 at 6.68 mg/mL in PBS, pH 7.0 (1.198 mL; 8.0 mg; 53.8 nmol; 1.0 eq.) was diluted with Dulbecco's PBS, pH 7.4, (556 μL). A 5 mM solution of TCEP in endotoxin-free water (64.6 μL; 323 nmol; 6 eq.) was added to the dilute mAb hIgG1 solution. The reduction was allowed to proceed at 40° C. for 1 h with a final antibody concentration of 4.4 mg/mL. After 1 h at 40° C., the reduction mixture was diluted with Dulbecco's PBS, pH 7.4 (61.8 μL) and 500 mM EDTA solution in water (20.0 p L) and allowed to cool down to 22° C. A 9.68 mg/mL (3.24 mM) solution of B4-Glu(-Gly3-EDA-PNU159682)-PEG(24u) in DMF was prepared by dissolving 3.50 mg (1172 nmol) of B4-Glu(-Gly3-EDA-PNU159682)-PEG(24u) (MW=2985.0 g/mol) into 361 p L of DMF. The B4-Glu-(-Gly3-EDA-PNU159682)-PEG(24u) solution in DMF (99.6 μL; 0.964 mg; 323 nmol; 6.0 eq.) was added to the reduced mAb hIgG1 solution resulting in a final concentration of 5% DMF and a final antibody concentration of 4.0 mg/mL. The conjugation reaction was allowed to proceed at 22° C. for 20 h. After 20 h at 22° C., the reaction mixture was buffer-exchanged and concentrated to 4.53 mg/mL by ultrafiltration/diafiltration using a Vivaspin 20 centrifugal concentrator (PES membrane, 30 kDa MWCO) equilibrated with Dulbecco's PBS, pH 7.1. Concentrated conjugate sample (7.2 mg; 1.60 mL) was sterile filtered through a 0.22 μm pore size, PVDF membrane filter. hIgG1 ThioBridge®-Glu(-Gly3-EDA-PNU159682)-PEG(24u) conjugate was characterized by HIC, SEC, LC-MS, SDS-PAGE and quantified by UV and endotoxin levels were determined.

### Results

Analytical Results for the ADCs are summarized in Tables 75-80.

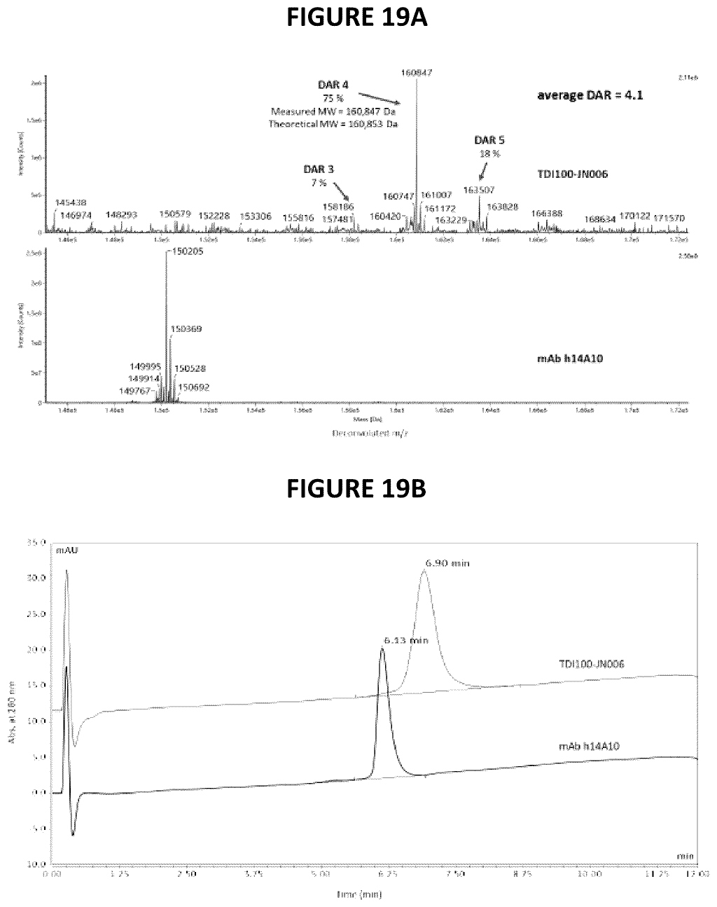

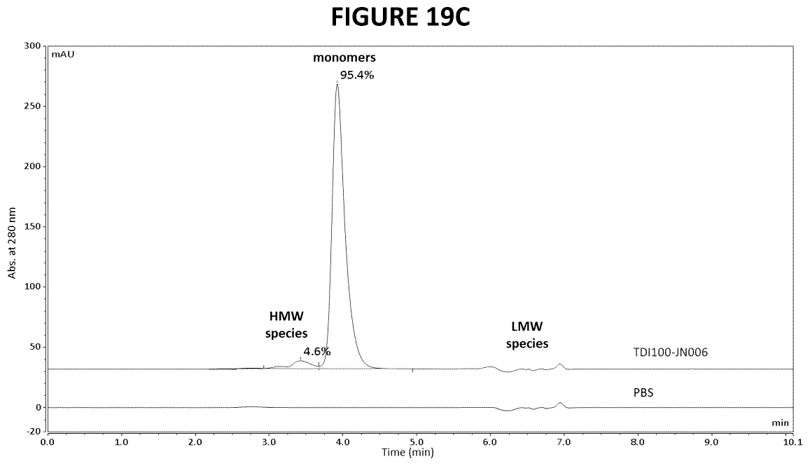

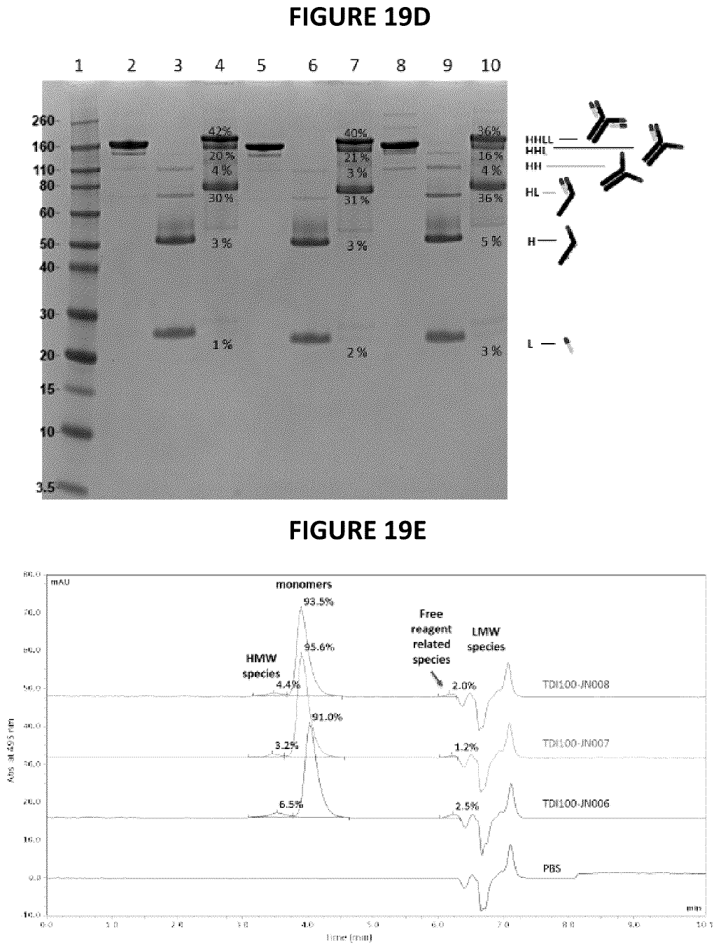

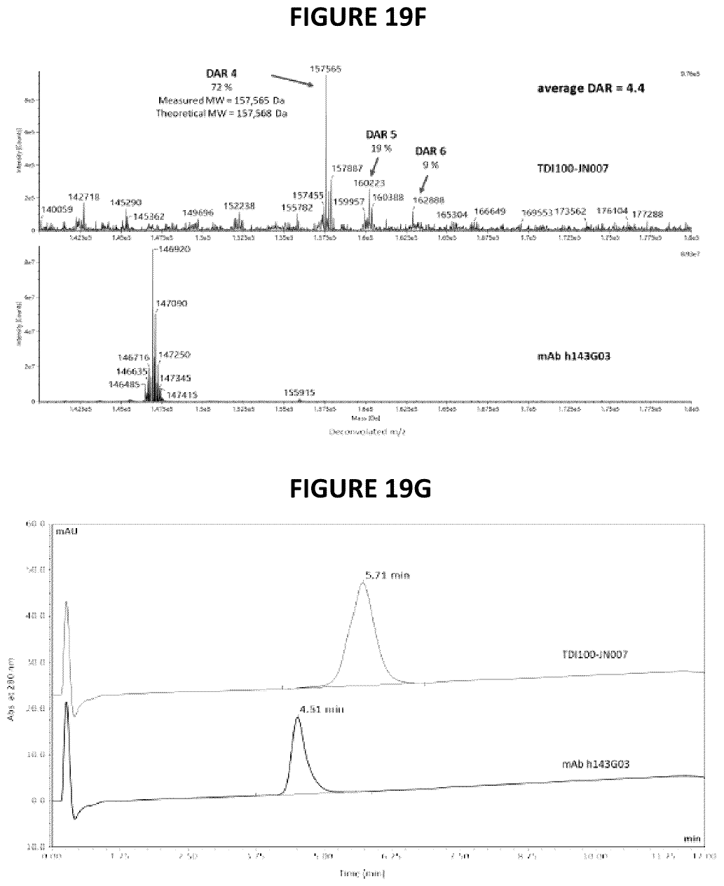

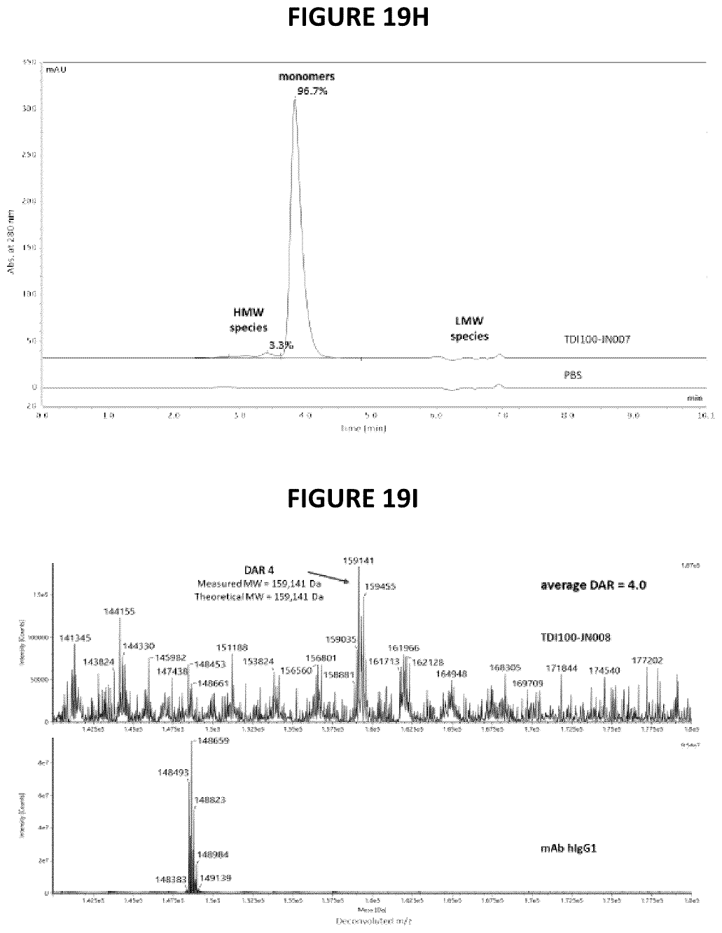

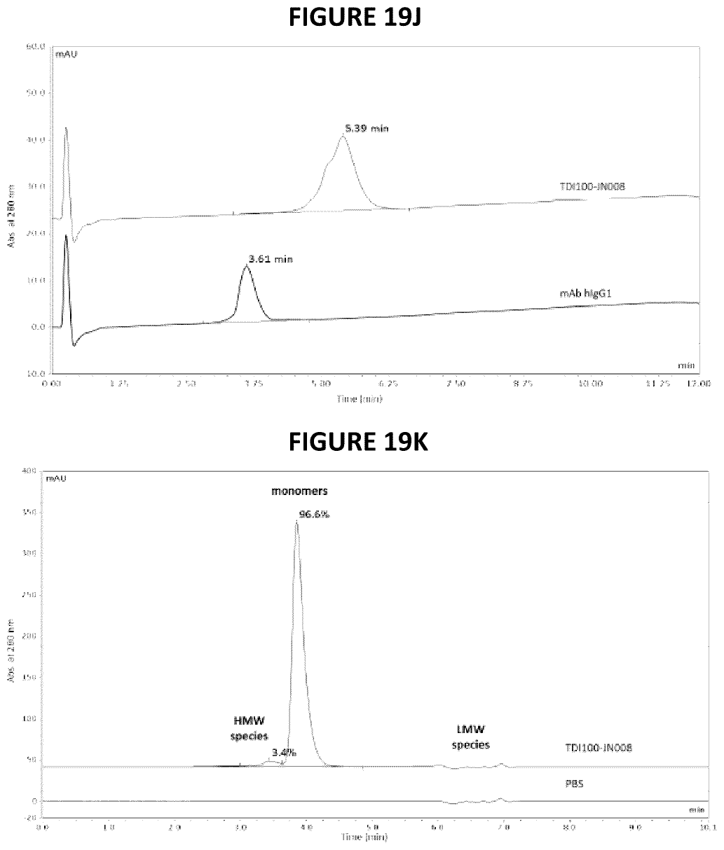

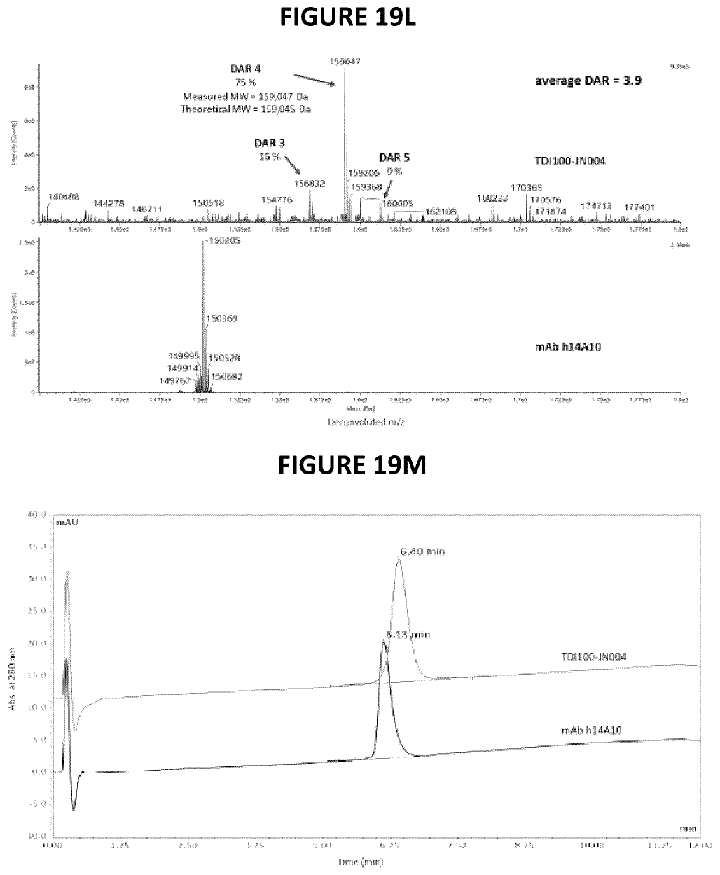

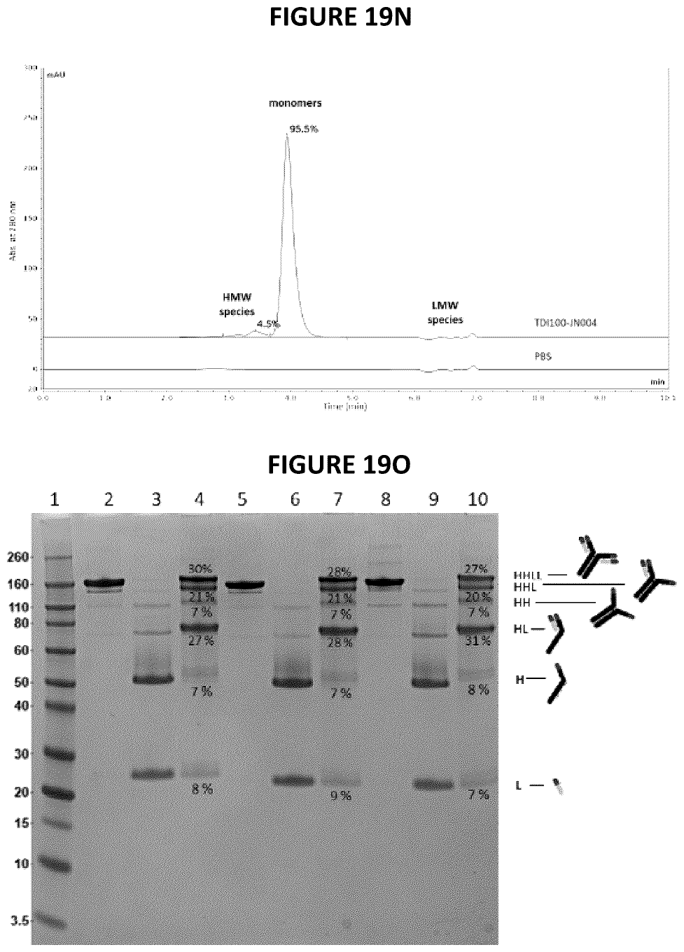

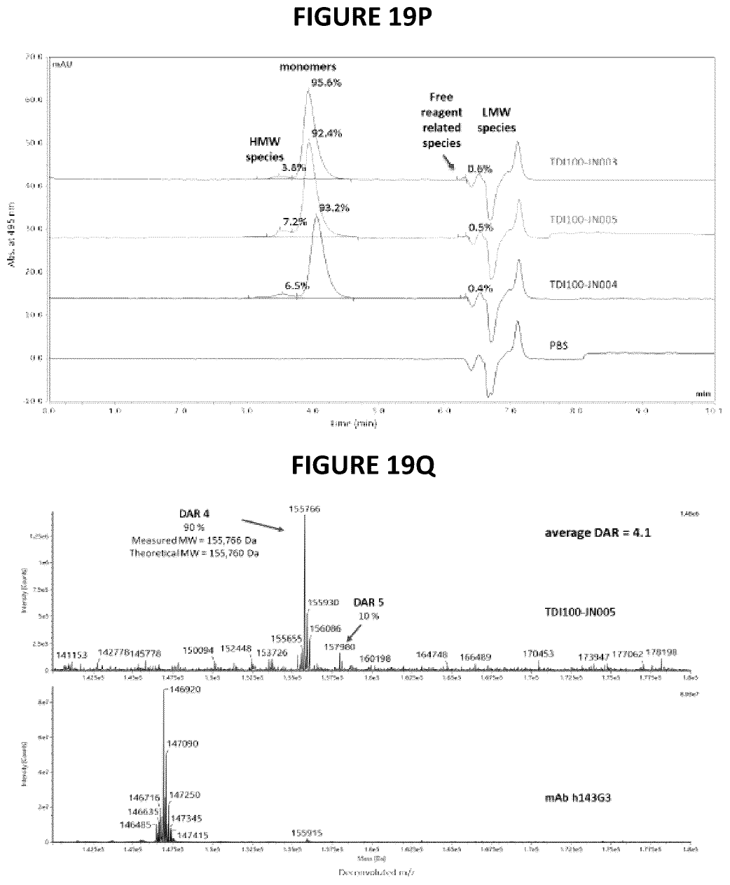

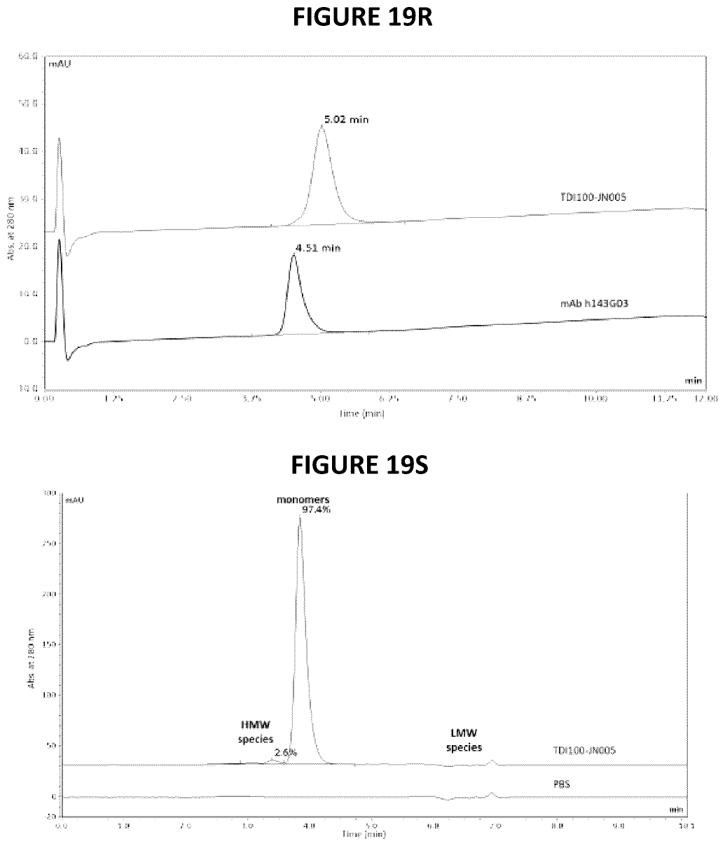

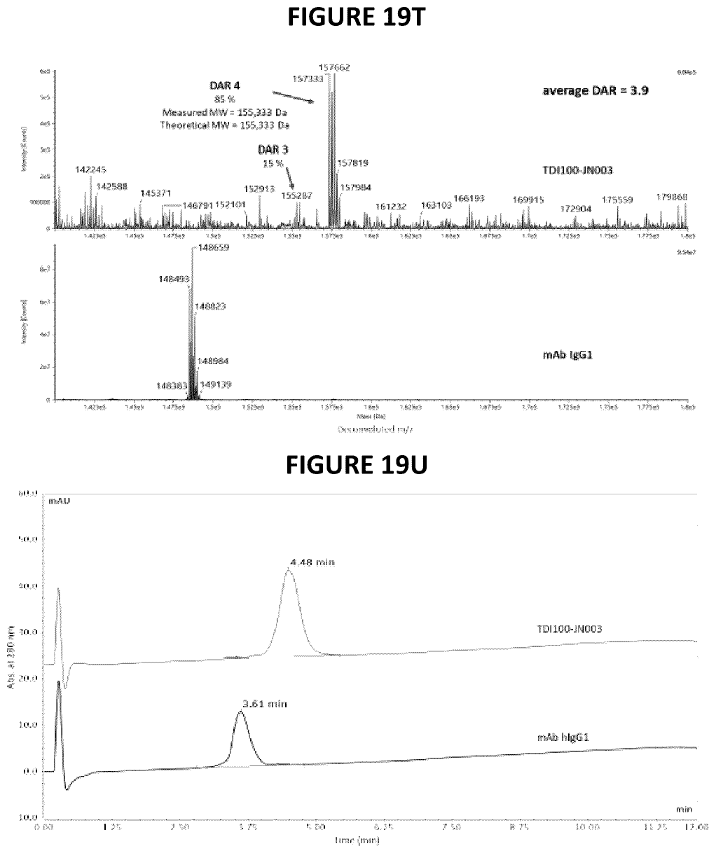

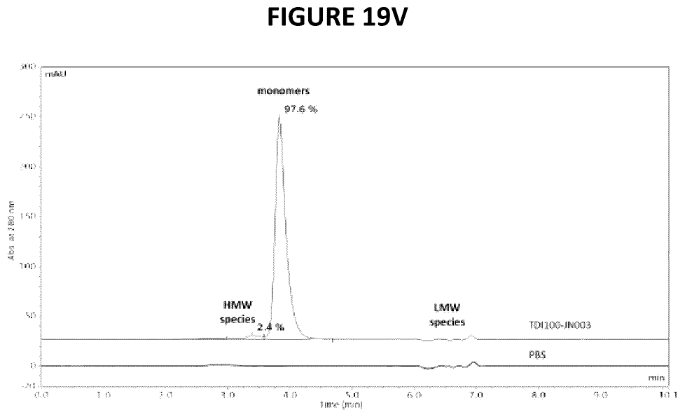

TABLE 75
Analytical summary of h14A10 ThioBridge ®-Glu[-Glu(OH)-
Val-Cit-PAB-DMEA-PNU159682]-PEG(24).

| Analysis | Results | |
|---|---|---|
| Sample Name | h14A10 ThioBridge ®-Glu[-Glu(OH)-Val-Cit-PAB- | |
| | DMEA-PNU159682]-PEG(24) | |
| Batch Code | TDI100-JN006 | |
| Appearance | Clear red solution | |
| Identity (LC-MS) | MW confirmed | |
| | See FIG. 19A for spectra | |
| DAR variants (LC-MS) | DAR 3: 7% | |
| | DAR 4: 75% | |
| | DAR 5: 18% | |
| | Average DAR: 4.1 | |
| | FIG. 19A for spectra | |
| Average DAR (UV) | Average DAR: 4.1 | |
| SDS-PAGE | See FIG. 19D | |
| Retention time (HIC) | for gel 6.90 min | |
| | See FIG. 19B for chromatogram | |
| % Purity (SEC) | 95.4% monomeric | |
| | See FIG. 19C for | |
| % free reagent related species | chromatogram 2.5 | |
| | See FIG. 19E for | |
| Endotoxin (EU/mg) | chromatogram 1.1 | |
| Concentration (UV) | 4.02 | mg/mL |
| Amount (by UV Analysis) | 7.5 | mg |
| Average MW | 161,119 | Da |

TABLE 76
Analytical summary of hz143G03 (N61Q) ThioBridge ®-Glu[-Glu(OH)-
Val-Cit-PAB-DMEA-PNU159682]-PEG(24).

| Analysis | Results | |
|---|---|---|
| Sample Name | hz143G03 (N61Q) ThioBridge ®-Glu [-Glu(OH)-Val-Cit- | |
| | PAB-DMEA-PNU159682]-PEG(24) | |
| Batch Code | TDI100-JN007 | |
| Appearance | Clear red solution | |
| Identity (LC-MS) | MW confirmed | |
| | See FIG. 19F for spectra | |
| DAR variants (LC-MS) | DAR 4: 72% | |
| | DAR 5: 19% | |
| | DAR 6: 9% | |
| | Average DAR: 4.4 | |
| | See FIG. 19F for spectra | |
| Average DAR (UV) | Average DAR: 3.9 | |
| SDS-PAGE | See FIG. 19D for gel | |
| Retention time (HIC) | 5.71 min See FIG. 19G | |
| | for chromatogram | |
| % Purity (SEC) | 96.7% monomeric See FIG. 19H | |
| | for chromatogram | |
| % free reagent related species | 1.2 See FIG. 19E | |
| | for chromatogram | |
| Endotoxin (EU/mg) | 1.5 | |
| Concentration (UV) | 5.03 | mg/mL |
| Amount (by UV Analysis) | 7.3 | mg |
| Average MW | 158,633 | Da |

TABLE 77
Analytical summary of hIgG1 ThioBridge ®-Glu[-Glu(OH)-
Val-Cit-PAB-DMEA-PNU159682]-PEG(24).

| Analysis | Results | |
|---|---|---|
| Sample Name | hIgG1 ThioBridge ®-Glu[-Glu(OH)-Val-Cit-PAB-DMEA- | |
| | PNU159682]-PEG(24) | |
| Batch Code | TDI100-JN008 | |
| Appearance | Clear red solution | |
| Identity (LC-MS) | MW confirmed | |
| | See FIG. 14I for spectra | |
| DAR variants (LC-MS) | DAR 4: 100% | |
| | Average DAR: 4.0 | |
| | See 19I for spectra | |
| Average DAR (UV) | Average DAR: 3.5 | |
| SDS-PAGE | See FIG. 19D for gel | |
| Retention time (HIC) | 5.39 min See FIG. 19J | |
| | for chromatogram | |
| % Purity (SEC) | 96.6% monomeric | |
| | See FIG. 19K for chromatogram | |
| % free reagent related species | 2.0 See FIG. 19E | |
| | for chromatogram | |
| Endotoxin (EU/mg) | 0.9 | |
| Concentration (UV) | 5.36 | mg/mL |
| Amount (by UV Analysis) | 7.3 | mg |
| Average MW | 159,307 | Da |

TABLE 78
Analytical summary of h14A10 ThioBridge ®-Glu(-
Gly3-EDA-PNU159682)-PEG(24 u).

| Analysis | Results | |
|---|---|---|
| Sample Name | h14A10 ThioBridge ®-Glu(-Gly3-EDA-PNU159682)- | |
| | PEG(24 u) | |
| Batch Code | TDI100-JN004 | |
| Appearance | Clear red solution | |
| Identity (LC-MS) | MW confirmed | |
| | See FIG. 19L for spectra | |
| DAR variants (LC-MS) | DAR 3: 16% | |
| | DAR 4: 75% | |
| | DAR 5: 9% | |
| | Average DAR: 3.9 | |
| | See FIG. 19L for spectra | |
| Average DAR (UV) | Average DAR: 3.9 | |
| SDS-PAGE | See FIG. 190 for gel | |
| Retention time (HIC) | 6.40 min See FIG. 19M | |
| | for chromatogram | |
| % Purity (SEC) | 95.5% monomeric | |
| | See FIG. 19N for chromatogram | |
| % free reagent related species | 0.4 See FIG. 19P | |
| | for chromatogram | |
| Endotoxin (EU/mg) | 0.35 | |
| Concentration (UV) | 3.95 | mg/mL |
| Amount (by UV Analysis) | 7.1 | mg |
| Average MW | 158,824 | Da |

TABLE 79
Analytical summary of hz143G03 (N61Q) ThioBridge ®-Glu(-
Gly3-EDA-PNU159682)-PEG(24 u).

| Analysis | Results | |
|---|---|---|
| Sample Name | hz143G03 (N61Q) ThioBridge ®-Glu(-Gly3-EDA-PN | |
| | U159682)-PEG(24 u) | |
| Batch Code | TDI100-JN005 | |
| Appearance | Clear red solution | |
| Identity (LC-MS) | MW confirmed | |
| | See FIG. 19Q for spectra | |
| DAR variants (LC-MS) | DAR 4: 90% | |
| | DAR 5: 10% | |
| | Average DAR: 4.1 | |
| | See FIG. 19Q for spectra | |
| Average DAR (UV) | Average DAR: 4.0 | |
| SDS-PAGE | See FIG. 190 for gel | |
| Retention time (HIC) | 5.02 min See FIG. 19R | |
| | for chromatogram | |
| % Purity (SEC) | 97.4% monomeric | |
| | See FIG. 19S for chromatogram | |
| % free reagent related species | 0.5 See FIG. 19P | |
| | for chromatogram | |
| Endotoxin (EU/mg) | 0.75 | |
| Concentration (UV) | 4.97 | mg/mL |
| Amount (by UV Analysis) | 7.4 | mg |
| Average MW | 155,981 | Da |

TABLE 80
Analytical summary of hIgG1 ThioBridge ®-Glu(-
Gly3-EDA-PNU159682)-PEG(24 u).

| Analysis | Results | |
|---|---|---|
| Sample Name | hIgG1 ThioBridge ®-Glu(-Gly3-EDA-PN U159682)- | |
| | PEG(24 u) | |
| Batch Code | TDI100-JN003 | |
| Appearance | Clear red solution | |
| Identity (LC-MS) | MW confirmed | |
| | See FIG. 19T for spectra | |
| DAR variants (LC-MS) | DAR 3: 15% | |
| | DAR 4:85% | |
| | Average DAR: 3.9 | |
| | See FIG. 19T for spectra | |
| Average DAR (UV) | Average DAR: 3.8 | |
| SDS-PAGE | See FIG. 190 for gel | |
| Retention time (HIC) | 4.48 min See FIG. 19U | |
| | for chromatogram | |
| % Purity (SEC) | 97.6% monomeric See FIG. 19V | |
| | for chromatogram | |
| % free reagent related species | 0.6 See FIG. 19P | |
| | for chromatogram | |
| Endotoxin (EU/mg) | 0.41 | |
| Concentration (UV) | 4.53 | mg/mL |
| Amount (by UV Analysis) | 7.2 | mg |
| Average MW | 157,278 | Da |

### Discussion

Six ADCs were successfully synthesized by conjugation of cleavable ThioBridge®-Glu[-Glu(OH)-Val-Cit-PAB-DMEA-PNU159682]-PEG(24) and non-cleavable ThioBridge®-Glu(-Gly3-EDA-PNU159682)-PEG(24u) linker-payloads to two anti-L1CAM human IgG1 antibodies (h14A10 and hz143G03 (N61Q)) and one isotype control antibody (hIgG1). All ADCs were isolated with an average DAR of 3.9 to 4.4 as determined by LC-MS. The average DAR of all three ADCs with non-cleavable ThioBridge®-Glu(-Gly3-EDA-PNU159682)-PEG(24u) linker-payload and ADCs h14A10 ThioBridge®-Glu[-Glu(OH)-Val-Cit-PAB-DMEA-PNU159682]-PEG(24) and hIgG1 ThioBridge®-Glu[-Glu(OH)-Val-Cit-PAB-DMEA-PNU159682]-PEG(24) with cleavable linker-payload were in the narrower 3.9-4.1 average DAR range. Average DAR values determined by UV-vis were in good agreement with the values determined by LC-MS, with a difference of <0.5 units of DAR observed between the different analytical methods. Low resolution could be achieved by analytical HIC between the different DAR species for the ThioBridge® ADCs. A hydrophobic shift could be observed for all conjugates as compared to the unconjugated parent antibody though, which is indicative of antibody conjugation.

All ADCs had high monomeric purity within the 95.4% to 97.7% range as determined by analytical SEC. Monomeric purity was ca. 95% for the two ADCs with mAb h14A10 and ca. 96% to 97% for the remaining four ADCs with either mAb hz143G03 (N61Q) or hIgG1. Much higher homogeneity for the monomer peak was observed with the ThioBridge® ADCs prepared from the humanized anti-L1CAM antibodies hz143G03 (N61Q) and h14A10 than for the ThioBridge® ADCs prepared with the murine 143G03 and chimeric (murine constant domains and humanized variable domains) 14A10 antibodies used previously.

Low levels of free payload related species were observed by analytical SEC with detection at A=495 nm: 1.2% to 2.5% levels for the ADCs bearing the cleavable ThioBridge®-Glu[-Glu(OH)-Val-Cit-PAB-DMEA-PNU159682]-PEG(24) linker-payload and 0.4% to 0.6% levels of free payload related species for ADCs bearing the non-cleavable ThioBridge®-Glu(-Gly3-EDA-PNU159682)-PEG(24u) linker-payload. These levels of free payload related species were confirmed to be suitable for the planned in vitro and in vivo evaluation of the conjugates.

LAL assay also confirmed the low levels of endotoxins in all ADC samples (<1.5 EU/mg). LC-MS intact mass analysis performed for all six ADCs confirmed the identity of the conjugated species.

Profiles for conjugation with ThioBridge® reagents were observed for the ADCs by SDS-PAGE, with presence of interchain disulfide bridged conjugated species including intact antibody conjugate LHHL, fragment species HHL and HH and of ‘half-antibody’ fragment species HL. 56-66% inter heavy-chains bridging was observed for ADCs bearing the cleavable ThioBridge®-Glu[-Glu(OH)-Val-Cit-PAB-DMEA-PNU159682]-PEG(24) linker-payload and 56-58% inter heavy-chains bridging for ADCs bearing the non-cleavable ThioBridge®-Glu(-Gly3-EDA-PNU159682)-PEG(24u) linker-payload. Presence of free L and free H species was observed at 4% to 8% levels for ADCs bearing the cleavable ThioBridge®-Glu[-Glu(OH)-Val-Cit-PAB-DMEA-PNU159682]-PEG(24) linker-payload and at 15% to 16% levels for ADCs bearing the non-cleavable ThioBridge®-Glu(-Gly3-EDA-PNU159682)-PEG(24u) linker-payload. All six ADCs were isolated in very high yields (86-99% recovery).

### Conclusions

Six ADCs were successfully synthesized by conjugation of cleavable ThioBridge®-Glu[-Glu(OH)-Val-Cit-PAB-DMEA-PNU159682]-PEG(24) and non-cleavable ThioBridge®-Glu(-Gly3-EDA-PNU159682)-PEG(24u) linker-payloads to two anti-L1CAM human IgG1 antibodies (h14A10 and h143G03) and one human isotype control antibody (hIgG1), in 7.1 to 7.5 mg amounts, allowing subsequent analytical characterization and biological evaluation to be performed. All ADCs were isolated with an average DAR between 3.9-4.4, with low levels of high molecular weight species (<5%) and low endotoxin levels (<1.5 EU/mg).

### Example 8—ADC Stability in Mouse and Human Serum

The aim of the present example was to monitor the stability of six ADCs formed by two anti-L1CAM human IgG1 antibodies (hz143G03 (N61Q) [TDI-Y-004] and h14A10 [TDI-Y-005]) and one isotype control antibody (hIgG1) conjugated to ‘Glu-Val-Cit-PAB’ cleavable and non-cleavable ThioBridge® PNU159682 linker-payloads in IgG depleted mouse and human sera and to identify reagent related species in sera for the six ADCs. Phosphate buffer saline (PBS) was tested alongside as negative comparator. ADCs were spiked into mouse and human sera at 1 mg/mL (75% sera) and incubated at 37° C. over a 96-hour period. To evaluate the changes in DAR profile throughout serum incubation, ADCs were analyzed by HIC-UV (280 nm) and reagent related species in sera for the six ADC were identified using LC-MS/MS.

### Methods

HIC method. HIC-UV analysis was used to monitor ADCs drug loading profile during serum stability. The chromatographic separation was performed using a TOSOH TSKgel Butyl-NPR 4.6 mm×35 mm HIC column (TOSOH Bioscience, 14947) connected to a Dionex Ultimate 3000RS HPLC system (ThermoFisher Scientific, Hemel Hempstead, UK). The method consisted of a linear gradient from 100% buffer A (buffer A is 50 mM Sodium Phosphate pH 7.0, 1.5 M ammonium sulphate) to 100% buffer B (buffer B is 50 mM Sodium Phosphate pH 7.0, 20% isopropanol) over 10.5 min. The flow rate was 1.35 mL/min and the temperature was maintained at 30° C. throughout the analysis. Detection was carried out by UV absorption at 214 nm.

Intact mass measurement. Intact mass measurements by LC-MS analysis were conducted on a Waters Xevo G2-XS TOF mass spectrometer interfaced with a Waters Acquity H-class UPLC. Chromatography was carried out on an Agilent POROSHELL 300SB-C3 column (2.1×12.5 mm, 5 am) at a flow rate of 0.4 mL/min. Mobile phase A comprised LC-MS grade water containing 0.1% (v/v) formic acid and mobile B comprised acetonitrile containing 0.1% (v/v) formic acid. The analysis was conducted using a 10-80% buffer B gradient as follows: 2.5 min 10% B, 10-80% B gradient in 5 min, 80% B isocratic 1 min, re-equilibration 10% B 4 min. The temperature of the column oven was maintained at 60° C. MS analysis was conducted using electrospray ionization (ESI) in positive ion mode (ES+). For each analysis, 2 g of sample was injected.

ADCs incubation in serum—samples preparation. ADCs were added to mouse serum at 1.0 mg/mL, in 75% serum. The ADC containing serum samples were aliquoted for t=0, 24 and 96 hours, incubated at 37° C. and stored at −80° C. after collection, for further analysis. After thawing, samples were analyzed by HIC-UV. As negative controls, 75% solutions of mouse and human sera in PBS were analyzed.

Immuno-capture method. The immuno-capture procedure for ADCs uses CaptureSelect™ human IgG-Fc PK biotin conjugate immobilized on streptavidin beads. The ratio used for affinity capture was: 75 L beads:30 g CaptureSelect™ antibody:50 g ADC. For affinity capture, 100 L ADC in PBS/serum were added to the CaptureSelect™ antibody pre-coated beads and incubated for 1 hour at 22° C. at 1350 rpm on a Thermomixer. For elution, the beads containing affinity captured ADC were incubated with 100 L 2 mM HCl for 5 minutes at 22° C. and the eluate was neutralized using 5 L of 0.5M Ammonium Bicarbonate buffer, pH7.8. After elution, samples were analyzed by LC-MS, after reduction. As negative controls, 75% solutions of serum in PBS were affinity captured and analyzed.

Sample preparation for reagent related species investigation in serum incubated ADCs. Aliquots of human and mouse serum incubated ADCs (100 L) were precipitated with 4× volumes (400 L) of ice-cold acetonitrile. The mixture was centrifuged, and the supernatant was carefully removed into a clean tube and dried out using vacuum. Dried supernatant was resuspended in 5 L acetonitrile followed by addition of 15 L water and it was analyzed by LC-MS/MS for low molecular mass reagent related species identification.

### Results

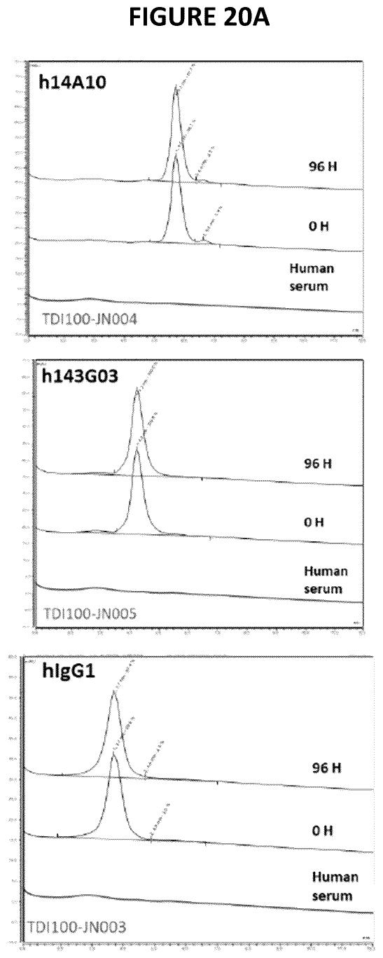

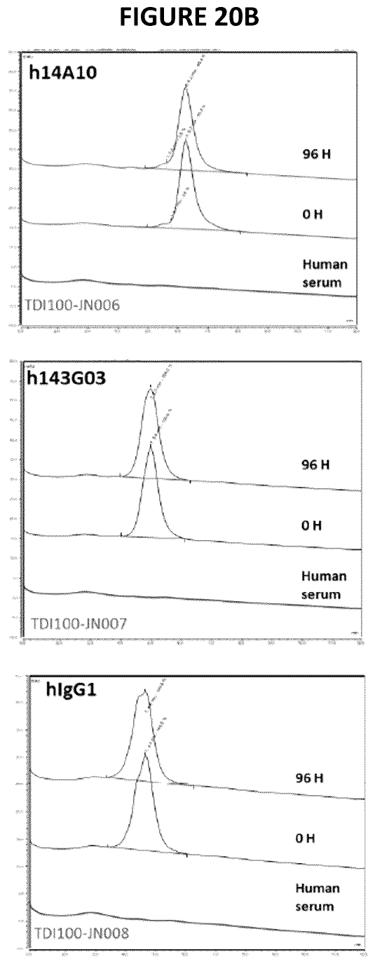

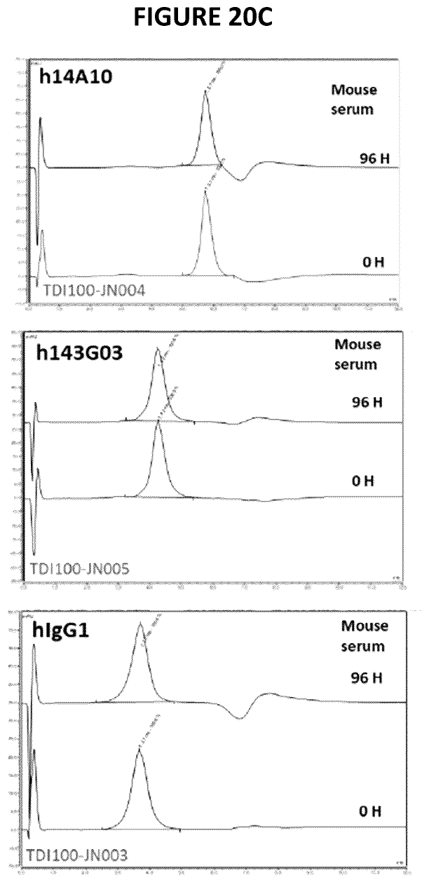

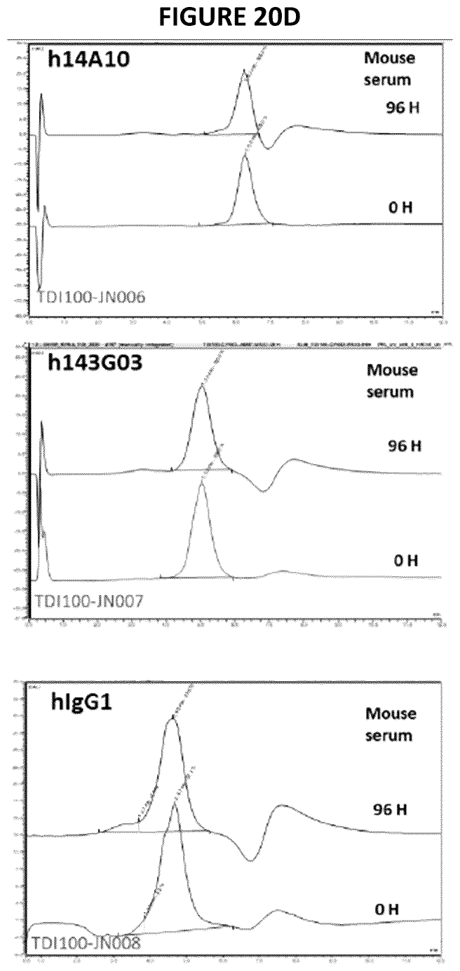

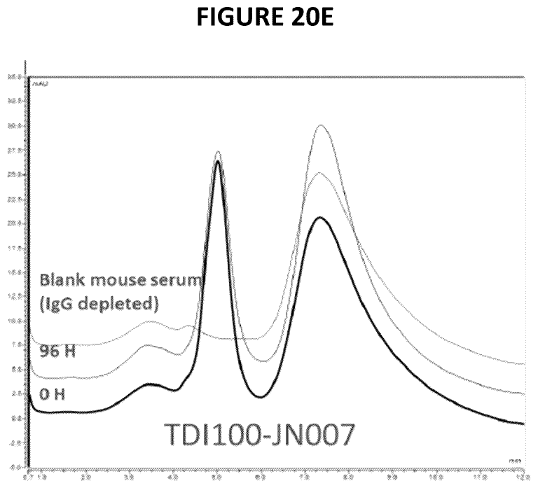

Change in drug loading following incubation in serum (HIC-UV). The six ADCs incubated in human serum were analyzed using HIC-UV (direct injection) to assess the changes in HIC profile induced upon incubation in serum at 37° C. No changes in HIC profile of ADCs could be observed after incubation in IgG depleted human serum for 96 h at 37° C. (FIGS. 20A and 20B). Mouse serum incubated ADC samples were analyzed by HIC (direct injection). No changes in HIC profile could be observed for all tested ADCs after incubation in IgG depleted mouse serum for 96 h at 37° C. (FIGS. 20C and 20D). Matrix serum chromatogram subtraction from ADCs chromatogram was required for DAR profile evaluation. The IgG depleted mouse serum does not contain IgG related peaks; however, it contains a broader peak, with a later r.t., most probably generated by residual IgM (FIG. 20E) and this is why subtraction of matrix chromatogram was necessary for DAR profile evaluation of ADCs in mouse serum.

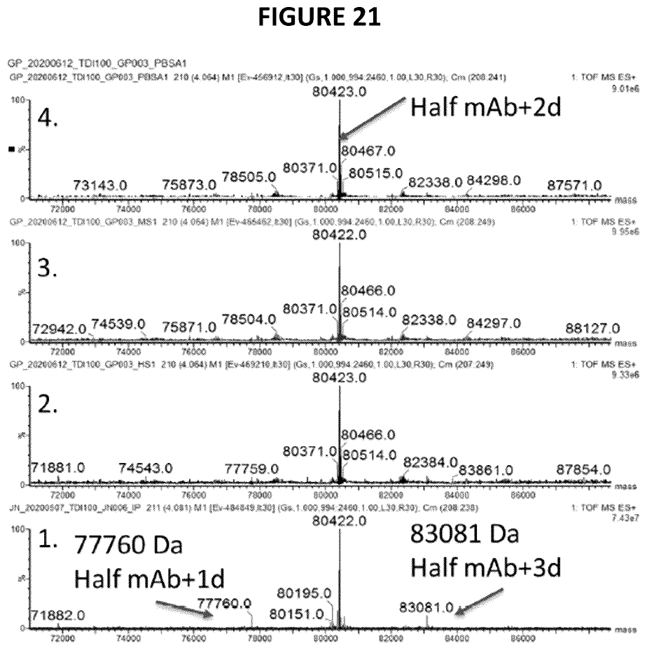

Change in drug loading for h14A10 ThioBridge® cleavable PNU159682 ADC following incubation in serum (LC-MS analysis). A method for the affinity capture of h14A10 ThioBridge® ‘Glu-Val-Cit-PAB’ cleavable PNU159682 (TDI100-JN006) from PBS, human and mouse IgG depleted sera was developed. Complete capture of the ADC from PBS was obtained. HIC analysis of the supernatant after ADC incubation in PBS with pre-coated beads showed no trace of remaining ADC. No changes in DAR profile were induced by the affinity capture or elution, as determined using LC-MS; however, a lower signal to noise ratio was observed after affinity capture from all matrices (FIG. 21). h14A10 ThioBridge® ‘Glu-Val-Cit-PAB’ cleavable PNU159682 (TDI100-JN006) was affinity captured from human and mouse serum incubated samples, after 0 and 96 hours incubation, and analyzed by LC-MS to evaluate potential changes in DAR.

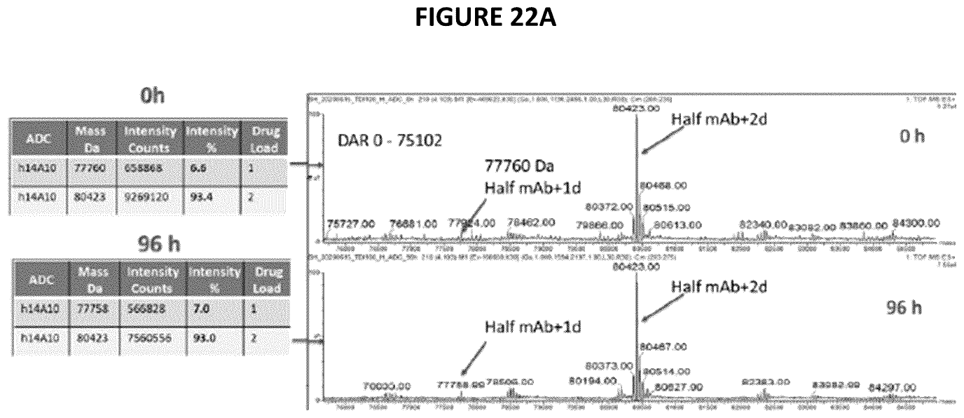

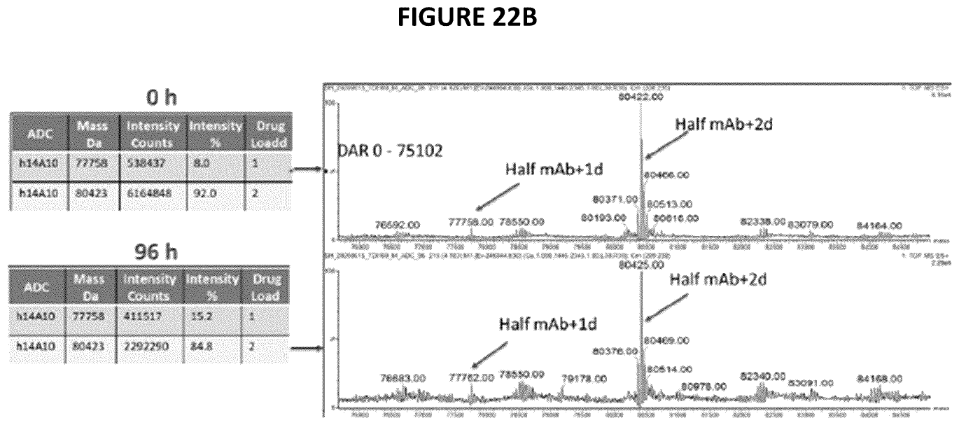

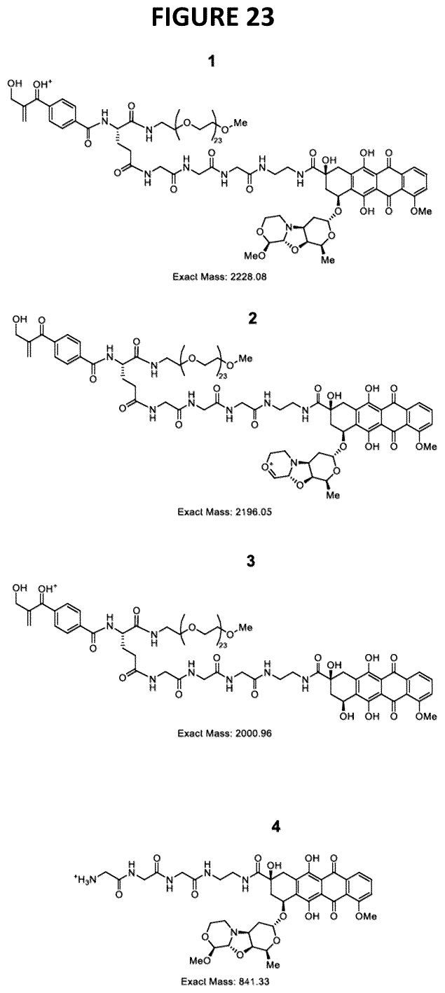

Analysis of human serum incubated ADC showed no changes in DAR profile, after 0 hours and 96 hours incubation in serum and affinity capture (FIG. 22A), based on the analysis of deconvoluted LC-MS spectra of half ADC. Analysis of mouse serum incubated ADC showed no significant change in DAR profile after 0 hours incubation in serum and affinity capture and slight increase in the single loaded half ADC after 96 h of incubation in mouse serum (FIG. 22B).

Reagent related species investigated in mouse and human incubated ADC samples.

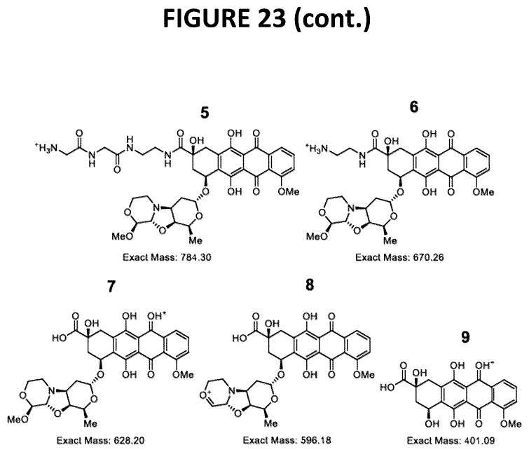

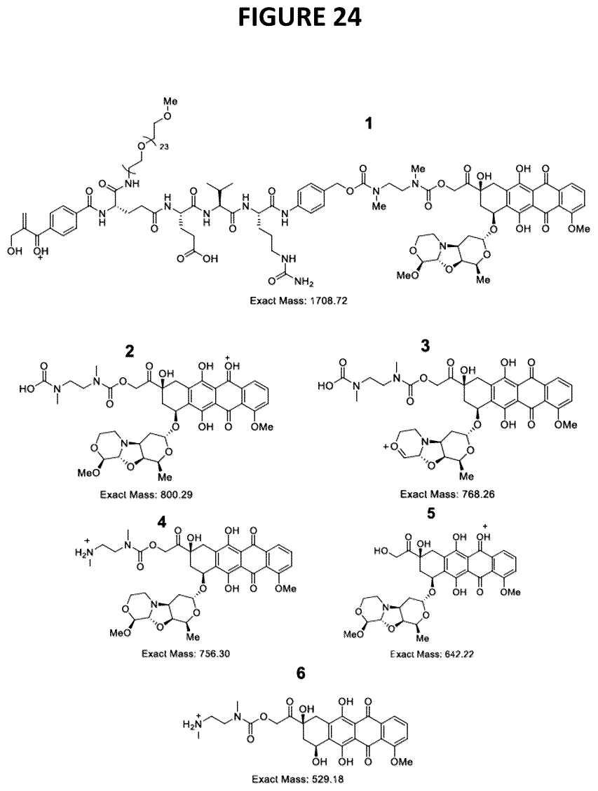

FIG. 16A shows the structure of ThioBridge® non-cleavable PNU159682 reagent, while FIG. 23 shows the structures of reagent related species (structures 1 to 9) investigated by LC-MS/MS. No reagent related species were detected for the 3 cleavable ThioBridge® ADCs incubated in human or mouse serum. FIG. 16B shows the structure of ThioBridge® ‘Glu-Val-Cit-PAB’ cleavable PNU159682 reagent, while FIG. 24 shows the structures of reagent related species (structures 1 to 6) investigated by LC-MS/MS. A reagent related fragment with the exact mass 800.29 was detected by LC-MS/MS analysis for the 3 cleavable ThioBridge® ADCs incubated in mouse serum (only traces at 0 h and significantly more after 96 hours).

### Conclusions

Stability of 6 ADCs with 2 anti-L1CAM human IgG1 antibodies (h14A10 & hz143G03 (N61Q)) and 1 isotype control antibody (hIgG1) functionalized with cleavable and non-cleavable ThioBridge® PNU159682 linker-payloads was successfully investigated in mouse and human sera. HIC analysis of ADCs incubated in serum showed no changes in DAR profile. Stability of h14A10 ThioBridge® cleavable PNU159682 ADC in human and mouse sera (IgG depleted) was also investigated based on affinity capture and LC-MS analysis of half ADC (reduced). No process-related changes in DAR were observed by LC-MS analysis, albeit with lower signal to noise ratio for affinity captured ADCs. LC-MS analysis of hz143G03 (N61Q) ThioBridge® ‘Glu-Val-Cit-PAB’ cleavable PNU159682 (TDI100-JN007) affinity captured from mouse serum, after 96 hours incubation at 37° C., showed a slight increase in mono-loaded half ADC, compared to the sample at 0 hours. No changes in drug loading were observed for hz143G03 (N61Q) ThioBridge® ‘Glu-Val-Cit-PAB’ cleavable PNU159682 captured from human serum, after 0- or 96-hours incubation at 37° C.

A reagent related fragment with the exact mass 800.29 was detected by LC-MS/MS analysis for the 3 cleavable ThioBridge® ADCs incubated in mouse serum (only traces at 0 h and significantly more after 96 hours), while no reagent related species were detected by LC-MS analysis for the 3 cleavable ThioBridge® ADCs incubated in human serum. No reagent related species were detected by LC-MS analysis for the 3 non-cleavable ThioBridge® ADCs incubated in mouse and human serum.

### Example 9—Antitumor Effect of Antibody-Drug Conjugates on Organoid Models

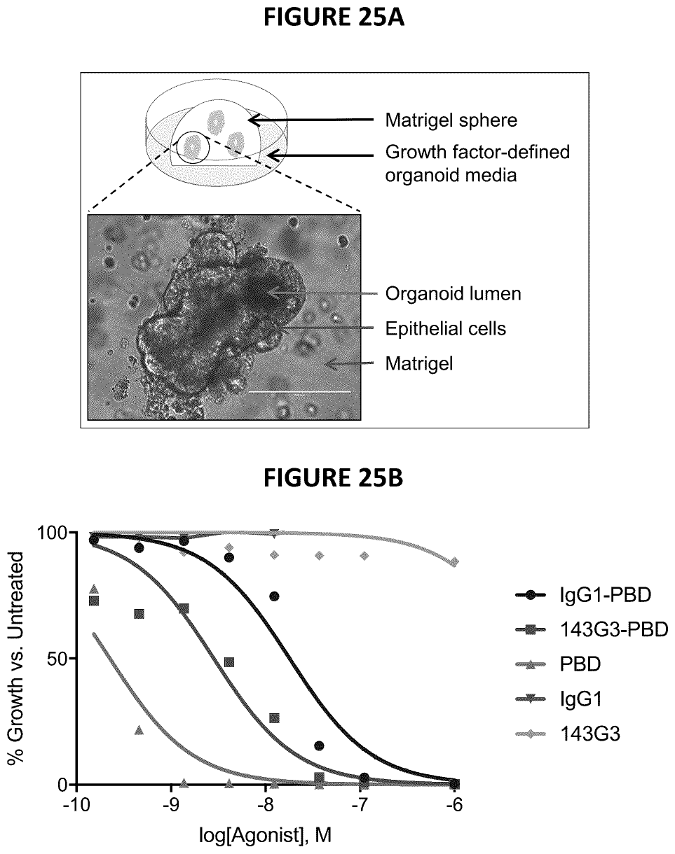

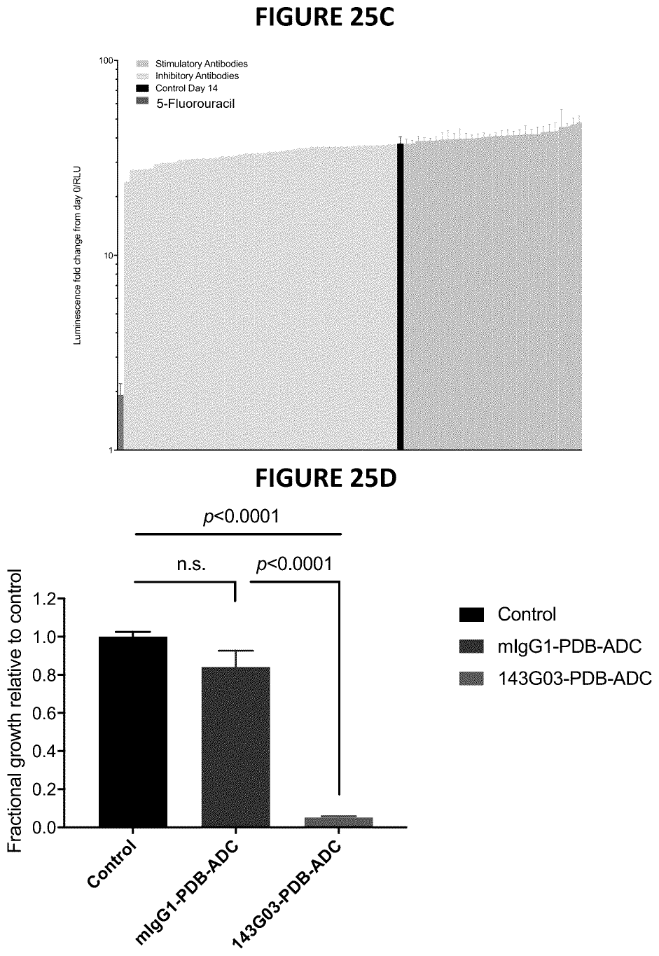

Liver metastases from colorectal cancers can be grown as stem-cell enriched organoids (FIG. 25A) (Ganesh et al., Nat Cancer 1, 28-45 (2020)). Since, L1CAM shRNA knockdown results in reduced and sustained organoid growth, the present example verified the hypothesis that the presently disclosed antibodies could be used for targeted killing of tumor cells expressing L1CAM. Initially, it was determined whether immunoconjugates disclosed herein could inhibit the growth of the organoid model. Organoid models were incubated with 143G03 mAb alone or conjugated with pyrrolobenzodiazepine (PBD) as payload. As shown in FIG. 25B, 143G03-PBD immunoconjugates were able of significantly reduce the organoid growth when compared to the antibody alone (143G3).

Next, it was confirmed whether the 143G03-PDB monoclonal antibody (mAb) had an L1CAM-specific inhibitor effect. As seen in FIGS. 25C and 25D, the 143G03-PDB mAb had an inhibitory effect on organoid models as compare to the correspondent control (mIgG1-PDB-ADC). Thus, it was concluded that the 143G03 conjugated antibody had an L1CAM-specific inhibitor of colorectal cancer organoid regeneration.

### Example 10—Antitumor Effect of Antibody-Drug Conjugates in Murine Xenograft Models

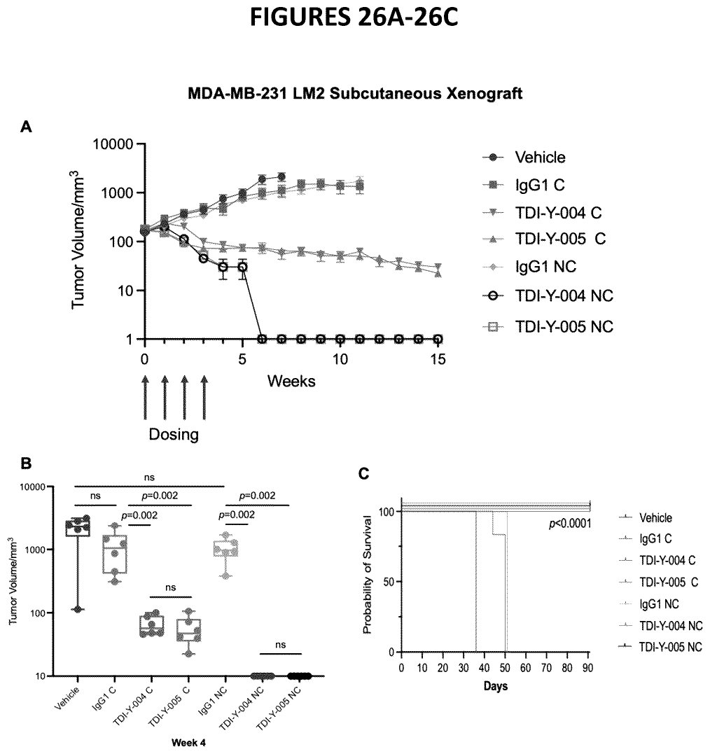

MDA-MB-231-LM2 Subcutaneous Xenografts. Doses of 5×10⁶ cells of the aggressive lung metastatic derivative (MDA231-LM2) of the MDA-MB-231 triple negative breast cancer cell line were injected into the flank of athymic mice, and tumors were grown until they reached 100 mm³ in size. Four weekly doses of PNU-159682-conjugated TDI-Y-004 or PNU-159682-conjugated TDI-Y-005 with both cleavable and non-cleavable linkers, significantly inhibited the growth of xenograft tumors in comparison to PNU-159682 conjugated IgG or vehicle controls (p=0.002) (FIGS. 26A and 26B). All TDI-Y-004 or TDI-Y-005 ADCs demonstrated tumor control even after treatment was withdrawn, surviving over 90 days from the initiation of treatment, in contrast to vehicle (survival=36 days) or IgG ADC controls (survival=50 days) (FIG. 26C).

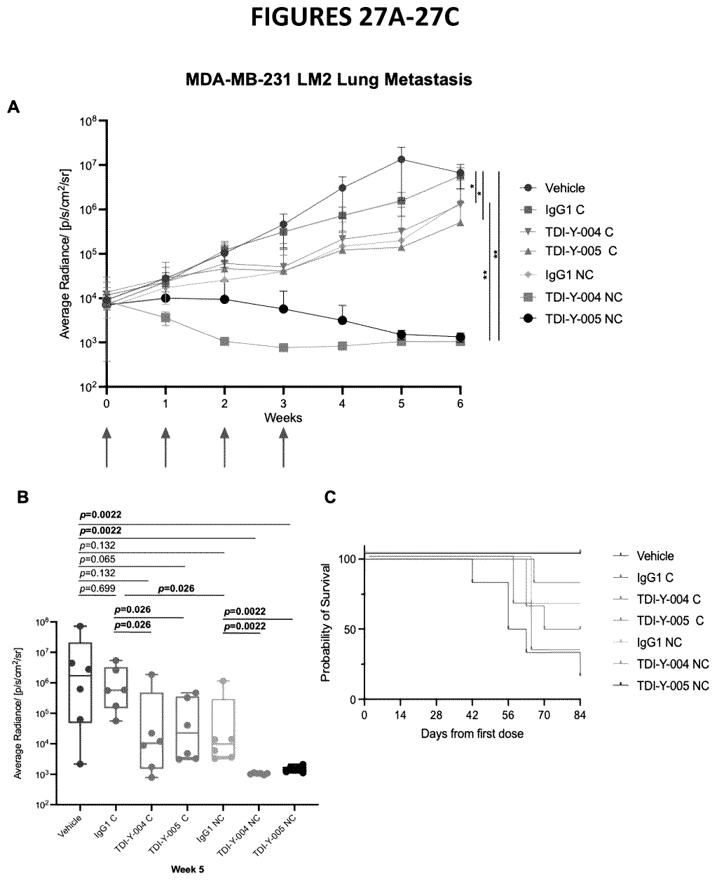

MDA-MB-231-LM2 Lung Metastasis. To assess the efficacy of the L1CAM ADCs in the context of an intact tumor microenvironment, 5×10⁴ MDA-MB-231-LM2 cells were introduced via tail vein injection into athymic mice to generate orthotopic lung metastases. Four weekly doses of PNU-159682-conjugated TDI-Y-004 or PNU-159682-conjugated TDI-Y-005 with cleavable and non-cleavable linkers significantly inhibited lung metastasis in comparison to PNU-159682-conjugated IgG or vehicle controls (FIGS. 27A and 27B). Animals treated with TDI-Y-004 or TDI-Y-005 ADCs with a non-cleavable linker showed complete tumor regression by week 6, with all mice in these groups surviving at least 84 days (FIG. 27C).

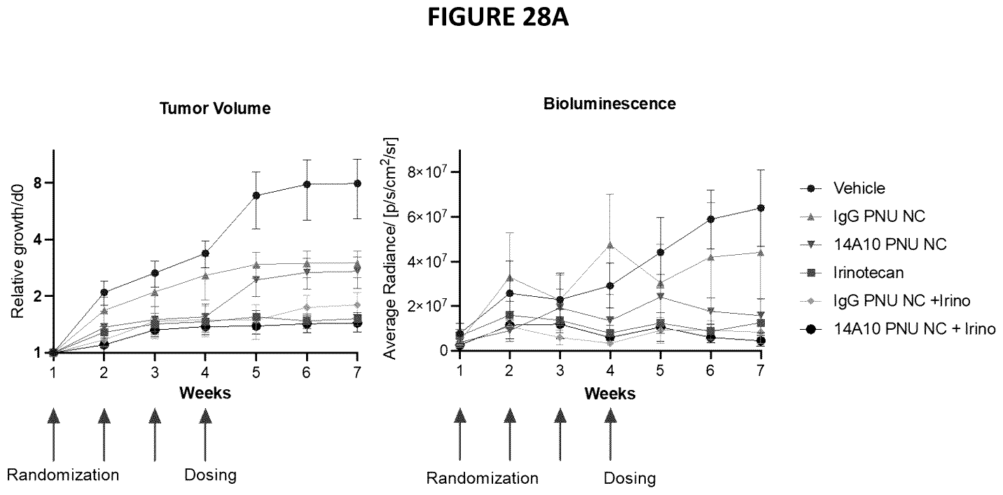

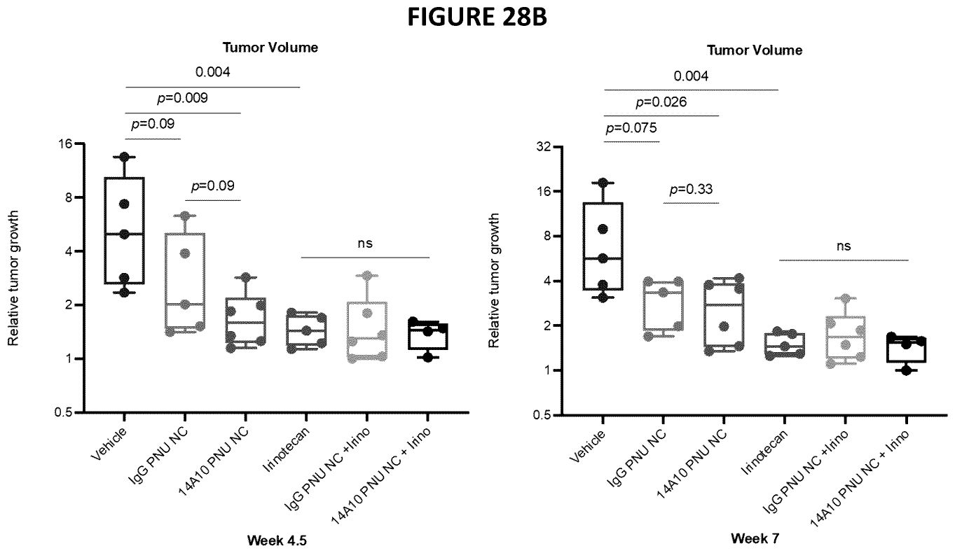

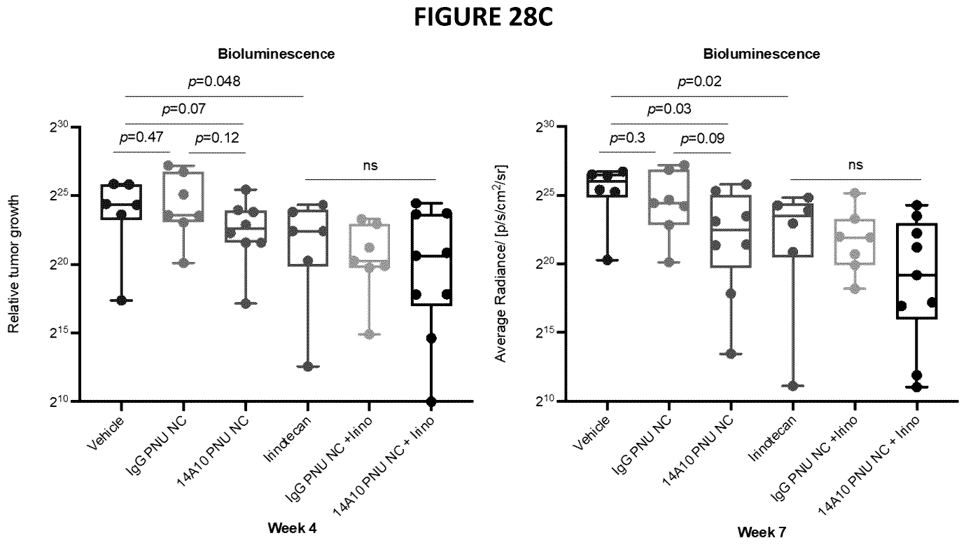

CRC107 Subcutaneous Xenografts. Colorectal cancer metastatic stem cells (CRC107) were injected into the flank of athymic mice, and tumors were grown. Four weekly doses of PNU-159682-conjugated TDI-Y-005 with both cleavable and non-cleavable linkers, significantly inhibited the growth of xenograft tumors in comparison to PNU-159682 conjugated IgG or vehicle controls. Notably, the combination of the ADC with the chemotherapeutic irinotecan did not have any additive effect. Thus, the presently disclosed ADCs demonstrated tumor control even after treatment was withdrawn, surviving over 7 weeks from the initiation of treatment (FIGS. 28A-28C).

In vivo Studies Summary. When formatted as ADCs with PNU-159682, TDI-Y-004 and TDI-Y-005 potently killed L1CAM-expressing cells. Mouse experiments demonstrated strong efficacy in both subcutaneous tumor and lung colonization xenograft models. Although the ADCs were toxic in mice at higher concentrations, the therapeutic window was large enough to firmly establish in vivo proof-of-concept.

The TDI-Y-004 and TDI-Y-005 ADCs with the PNU-159682 payload conjugated via cleavable and non-cleavable linkers demonstrated significant anti-tumor activity both against established subcutaneous xenografts and against lung metastasis. However, differences were noted between the performance of the cleavable and non-cleavable linkers, particularly in the lung metastasis model. This may be due to the higher dose administered (1 mg/kg vs. 0.3 mg/kg) of the ADCs with the non-cleavable vs. cleavable linker, due to the differences in safety and tolerability noted. In addition, organ-specific effects on linker cleavability may be contributing to the relatively reduced efficacy of the cleavable linker compounds in the lung tumor microenvironment.

### Example 11—Manufacturability and Productivity Attributes

Summary of Manufacturability Assessment. The purpose of the manufacturability studies was to gain a preliminary understanding of the development properties of the candidate molecule, including productivity, biophysical characteristics, solubility, stability under stress conditions and degradation pathways. The results of these studies are described in Table 81. To assess product quality attributes of TDI-Y-004 and TDI-Y-005, bulk stable pools of Chinese Hamster Ovary (CHO) cells were generated and scaled up for 2 L production runs. Final purified product was used to support manufacturability studies.

TABLE 81
Summary of Manufacturability Assessment

| Method | Criteria/Goal | Results |
|---|---|---|
| SEC-HPLC | >95% monomer | SEC analysis indicated that both TDI-Y-004 and TDI-Y-005 were |
| | | >98% monomeric at 10, 50, and 100 mg/ml. Both TDI-Y-004 and |
| | | TDI-Y-005 were soluble at 100 mg/ml |
| DLS | <15% | DSL analysis indicates a monodal distribution for both TDI-Y-004 |
| | polydispersity | and TDI-Y-005 with a high homogeneity and a polydispersity index |
| | (Pd %) | <15% at 20, 50, and 100 mg/ml |
| Protein | A260/A280 <0.55 | Both TDI-Y-004 and TDI-Y-005 had a protein purity ratio |
| purity ratio | | A260/A280 at 0.49 at 50 and 100 mg/ml. Pure protein prep with no |
| | | nucleic acid contamination |

### Manufacturability of TDI-Y-004 & TDI-Y-005

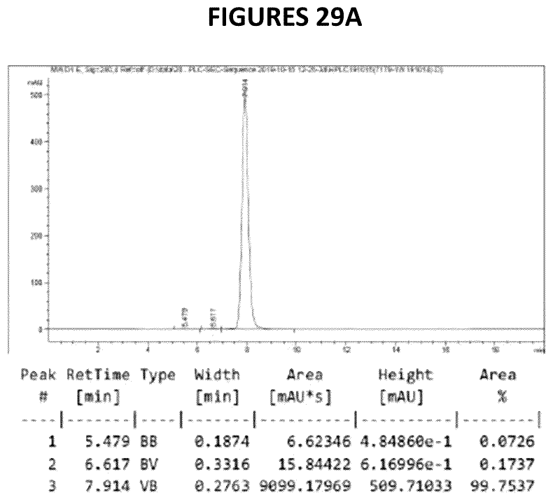

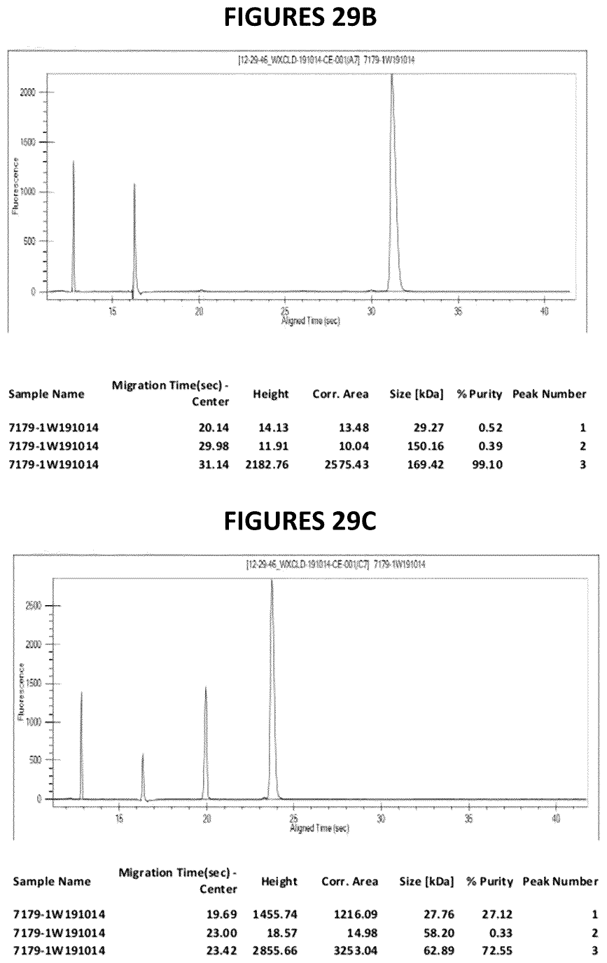

TDI-Y-004 Production. To evaluate upstream and downstream process behavior, a stable pool of CHO cells expressing TDI-Y-004 was generated. The cell culture volume was scaled up to 2 L for a 25-day, fed-batch production run. Culture supernatant was harvested and clarified by depth-filtration. The titer was 1.71 g/L. Material was purified by protein A (Pro-A) capture and evaluated for purity. The Pro-A eluate was then taken through a SEC polishing process. Drug substance release tests were performed. The final product was formulated in a platform buffer at 21.3 mg/mL (20 mM Histidine-HCl, 150 mM NaCl, pH 6.0). The final purified product was 99.8% monomer as determined by size exclusion high-performance liquid chromatography (SE-HPLC). The endotoxin level in the purified product was well within acceptable limits with residual Pro-A measured at <1.0 EU/mg. The antibody demonstrated acceptable upstream and downstream process performance to warrant continued development. The purified material from the production run was used in the manufacturability studies (FIGS. 29A-29C).

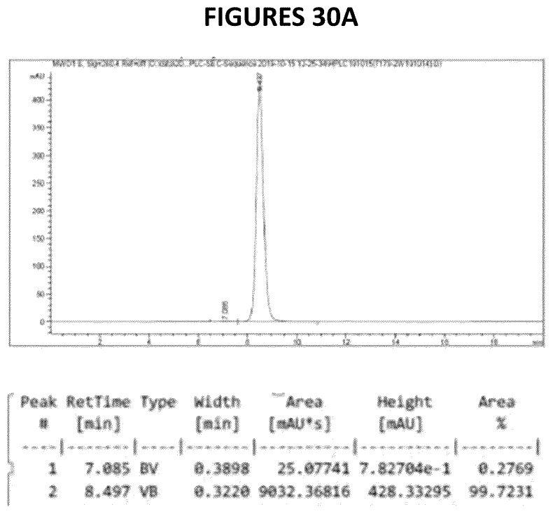

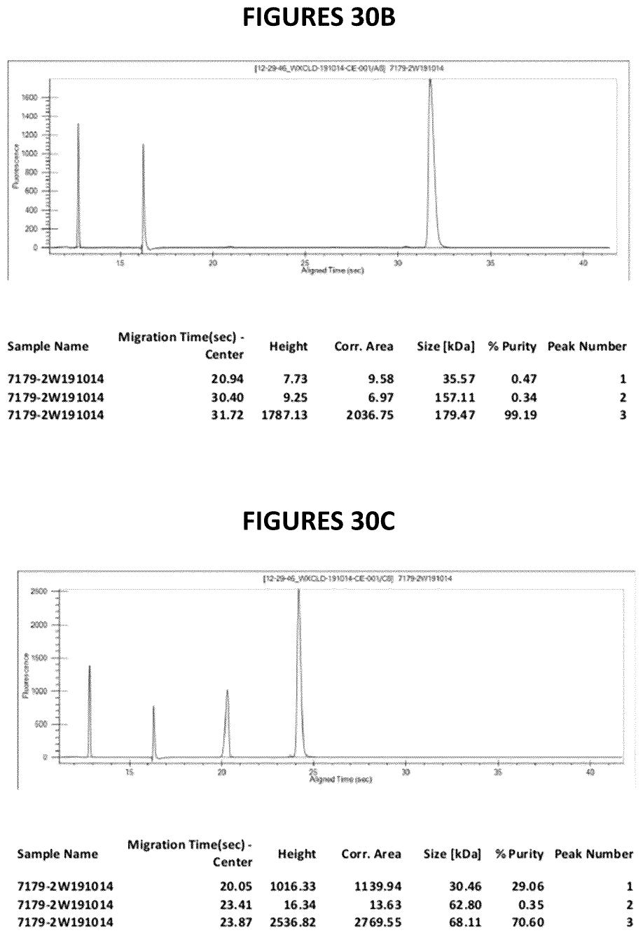

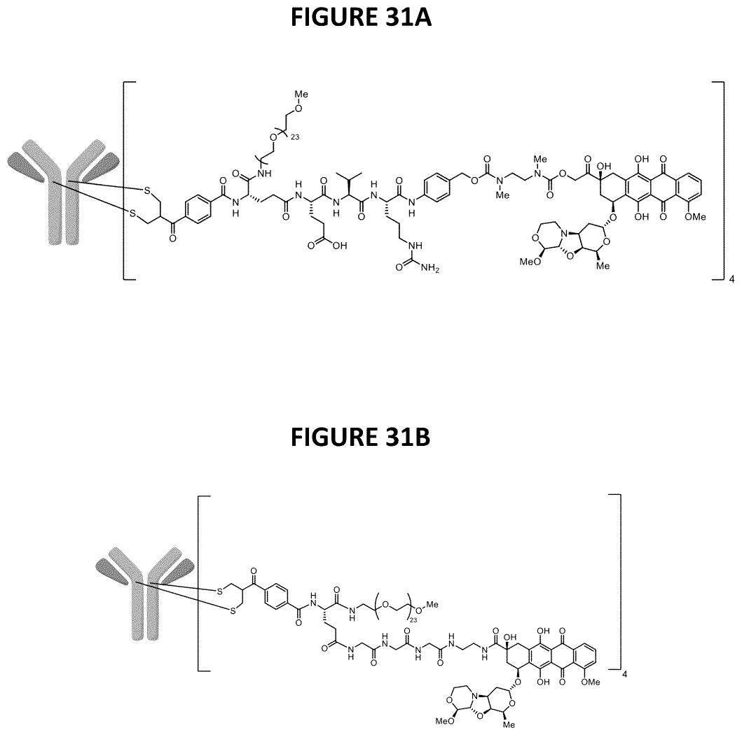

TDI-Y-005 Production. To evaluate upstream and downstream process behavior, a stable pool of CHO cells expressing TDI-Y-005 was generated. The cell culture volume was scaled up to 2 L for a 25-day, fed-batch production run. Culture supernatant was harvested and clarified by depth-filtration. The titer was 1.81 g/L. Material was purified by Pro-A capture and evaluated for purity. The Pro-A eluate was then taken through a SEC polishing process. The final product was formulated in a platform buffer at 20.8 mg/mL (20 mM Histidine-HCl, 150 mM NaCl, pH 6.0). The final purified product was 99.7% monomer as determined by SE-HPLC. The endotoxin level in the purified product was well within acceptable limits with residual Pro-A measured at <1.0 EU/mg. The antibody demonstrated acceptable upstream and downstream process performance to warrant continued development. The purified material from the production run was used in the manufacturability studies (FIGS. 30A-30C).

Biophysical Characterization. Biophysical characterization of the CHO stable pool-derived material was performed before initiation of the solubility and stress studies to establish baseline concentrations, visual appearance, % monomer (SE-HPLC), thermal unfolding (DSF), hydrodynamic radius (DLS), polydispersity (DLS), intact mass (LC-MS) and post-translational modifications within the CDRs (LC-MS).

Solubility of TDI-Y-004 and TDI-Y-005. TDI-Y-004 and TDI-Y-005 exhibited good solubility in formulation buffers tested, 20 mM His, 150 mM NaCl, pH 6.0 and 1×PBS. The antibody is soluble at concentrations up to 100 mg/ml with no material loss or aggregation observed.

Forced Stress Studies. The stability of TDI-Y-004 and TDI-Y-005 was evaluated in a number of forced stress conditions including incubation at 37° C., oxidation, extreme pH and freeze/thaw. The starting material for all tests was formulated in 1×PBS at 20 mg/ml.

Freeze/Thaw of TDI-Y-004 and TDI-Y-005. Samples were evaluated after 5 slow freeze/thaw cycles. No significant changes were observed in any of the parameters measured relative to baseline including visual appearance, concentration, Rh, % HMW species and hydrophobicity.

Oxidative Stress of TDI-Y-004 and TDI-Y-005. Samples were exposed to 0.1% (w/v) H₂02 and tert-Butyl Hydroperoxide (tBHP) for 24 hours, visually inspected and then buffer exchanged back to 1×PBS. No significant changes were observed in the majority of the parameters measured relative to baseline including visual appearance, concentration, Rh, % HMW species and hydrophobicity. Hydrogen peroxide treatment resulted in an altered analytical HIC elution profile. Mass spectrometry analysis of the H₂O₂ treated sample indicated a 98% increase in oxidation of M4, M259 and M435 for TDI-Y-004, and a 96% increase oxidation of M63 and M251 for TDI-Y-005. Given that these residues are known to be surface-exposed, the results were expected.

pH Stress of TDI-Y-004 and TDI-Y-005. Samples were buffer exchanged to 50 mM acetate buffer pH 3.5 or 50 mM Tris-HCl buffer pH 9.0, and incubated overnight at room temperature. Samples were then buffer exchanged to 1×PBS solution. No significant changes were observed in any of the parameters measured relative to baseline including visual appearance, concentration, Rh, % high molecular weight (HMW) species and hydrophobicity.

Thermal Stress of TDI-Y-004 and TDI-Y-005. Antibody formulated in 1×PBS or 20 mM His, 150 mM NaCl, pH6.0 at 20 mg/ml was subjected to extended thermal stress. Samples were incubated at 37° C. for 1 or 2 weeks. No significant changes were observed in visual appearance, concentration, Rh or % HMW species.

Manufacturability Assessment. In summary, both TDI-Y-004 and TDI-Y-005 mAbs exhibit good production, solubility, stability and product quality.

### Example 12—Profiles of TDI-Y-004 and TDI-Y-005 Antibodies and ADCs of Such Antibodies

The present example summarizes in Tables 82 and 83 the properties of the TDI-Y-004 and TDI-Y-005 antibodies disclosed herein and ADCs of such antibodies.

TABLE 82
Molecular Pharmacology Properties

| | | |
|---|---|---|
| PARAMETER | TDI-Y-004 | TDI-Y-005 |

Binding to Target

| | | |
|---|---|---|
| Binding to soluble human | KD: 0.35 nM | KD: 4.3 nM |
| L1CAM (monomer) | kon: 2.7 × 105 M-1s-1 | kon: 3.3 × 104 M-1s-1 |
| | koff: 9.5 × 10-5 s-1 | koff: 1.4 × 10-4 s-1 |
| Binding to cells overexpressing | EC50: 2.2 nM | EC50: 2.0 nM |
| human L1CAM (HEK293T) | | |
| Binding to cells with | EC50: 1.3 nM | EC50: 2.5 nM |
| endogenous L1CAM (MCF-7) | | |
| Binding to cells with | EC50: 2.5 nM | EC50: 7.1 nM |
| endogenous L1CAM (MDA- | | |
| MB-231) | | |
| Epitope | Fibronectin domain | Immunoglobulin domain |
| Cross-reactivity to soluble | KD: 0.3 nM | KD: 1.3 nM |
| cynomolgus monkey L1CAM | kon: 1.6 × 105M−1 s−1 | kon: 9.3 × 104M−1 s−1 |
| (monomer) | koff: 4.7 × 10−5 s−1 | koff: 1.2 × 10−4 s−1 |
| PARAMETER | TDI-Y-004 | TDI-Y-005 |
| Cross-reactivity to soluble | No binding | KD: 34 nM |
| mouse CD40 (monomer) | | kon: 3.1 × 104M−1 s−1 |
| | | koff: 1.1 × 10−3 s−1 |
| Cross-reactivity to mouse | No binding | EC50: 0.9 nM |
| LICAM on cells (HEK293T) | | |

Molecular Selectivity

| | | |
|---|---|---|
| Binding to human CHL-1 | No binding at 1 μM | No binding at 1 μM |
| (L1CAM2) | | |
| Binding to human NrCAM | No binding at 1 μM | No binding at 1 μM |
| Binding to human Neurofascin | No binding at 1 μM | No binding at 1 μM |

In vitro Functional Activity - Antibody-Drug Conjugates with cleavable linker

| | | |
|---|---|---|
| Cytotoxicity for cells | EC50: 0.008 nM | EC50: 0.03 nM |
| overexpressing human L1CAM | | |
| (HEK293T) | | |
| Cytotoxicity for cells with | EC50: 0.03 nM | EC50: 0.4 nM |
| endogenous L1CAM (MCF-7) | | |
| Cytotoxicity for cells with | EC50: 3.3 nM | EC50: 12.4 nM |
| endogenous LICAM (MDA- | | |
| MB-231) | | |

In vitro Functional Activity - Antibody-Drug Conjugates with non-cleavable linker

| | | |
|---|---|---|
| Cytotoxicity for cells | Moderate | Moderate |
| overexpressing human L1CAM | | |
| (HEK293T) | | |
| Cytotoxicity for cells with | Weak | Weak |
| endogenous L1CAM (MCF-7) | | |
| Cytotoxicity for cells with | Nonspecific killing only | Nonspecific killing only |
| endogenous LICAM (MDA- | | |
| MB-231) | | |

TABLE 83
In vivo efficacy.

| | | |
|---|---|---|
| PARAMETER | TDI-Y-004 | TDI-Y-005 |

Mouse Models - Antibody-Drug Conjugates with cleavable linker

| | | |
|---|---|---|
| Efficacy in MDA-MB-231 | >90% tumor control | >90% tumor control |
| subcutaneous xenograft model | 100% survival | 100% survival |
| | No tumor regrowth up to 60 | No tumor regrowth up to 60 days |
| | days after last dose | after last dose |
| Efficacy in MDA-MB-231 tail | >85% tumor control | >85% tumor control |
| vein injection model | | |

Mouse Models - Antibody-Drug Conjugates with non-cleavable linker

| | | |
|---|---|---|
| Efficacy in MDA-MB-231 | 100% tumor control, complete | 100% tumor control, |
| subcutaneous xenograft model | regression | complete regression |
| | 100% survival | 100% survival |
| | No tumor recurrence up to 60 | No tumor recurrence up to 60 days |
| | days after last dose | after last dose |
| Efficacy in MDA-MB-231 tail | >99% tumor control, complete | >99% tumor control, |
| vein injection model | regression | complete regression |
| | 100% survival | 100% survival |
| | No tumor recurrence up to 48 | No tumor recurrence up to 48 days |
| | days after last dose | after last dose |

### Example 13—Humanization of Murine Antibody 76H12

To humanize the variable regions of murine mAb 76H12, the complementarity-determining regions (CDR) were grafted onto human V_{H} and V_{L} acceptor frameworks and select human-to-murine framework substitutions were introduced as needed to fully recapitulate L1CAM binding affinity. IGHV1-46 and IGKV1-39 human germline acceptor frameworks were selected based on sequence identity to the V_{H} and V_{L} regions of 76H12 and on their frequency of usage in natural human immune repertoires. For V_{H} and V_{L} sequence downstream of CDR3, human IGHJ4 and IGKJ2 J-region sequences were used in humanized designs. Four humanized V_{H} sequences (Table 84) and four humanized V_{L} sequences (Table 85) were designed.

TABLE 84
VH sequences of 76H12 and humanized variants

| VH Identifier | Sequence |
|---|---|
| 76H12_H | QVQLQQPGSDLVRPGTSVKLSCKASGYTFTSYWMHWVKQRPGQGLEWIGN |
| | VYPGSGSTKYDEKFKTKATLTVDTSSNTAYMELSSLTSEDSAVYYCTRPG |
| | NYEGFAYWGQGTLVTVSA [SEQ ID NO: 122] |
| h76H12_Hv0 | QVQLVQSGAEVKKPGASVKVSCKASGYTFTSYWMHWVRQAPGQGLEWMGN |
| | VYPGSGSTKYAQKFQGRVTMTRDTSTSTVYMELSSLRSEDTAVYYCARPG |
| | NYEGFAYWGQGTLVTVSS [SEQ ID NO: 128] |
| h76H12_Hv1 | QVQLVQSGAEVKKPGASVKVSCKASGYTFTSYWMHWVRQAPGQGLEWIGN |
| | VYPGSGSTKYAQKFQGRATLTVDTSTSTVYMELSSLRSEDTAVYYCARPG |
| | NYEGFAYWGQGTLVTVSS [SEQ ID NO: 130] |
| h76H12_Hv2 | QVQLVQSGAEVKKPGASVKVSCKASGYTFTSYWMHWVRQAPGQGLEWIGN |
| | VYPGSGSTKYAQKFQGRATLTVDTSTSTAYMELSSLRSEDTAVYYCTRPG |
| | NYEGFAYWGQGTLVTVSS [SEQ ID NO: 134] |
| h76H12_Hv3 | QVQLVQSGAEVKKPGASVKVSCKASGYTFTSYWMHWVRQAPGQGLEWIGN |
| | VYPGSGSTKYAQKFQGRATLTVDTSTNTAYMELSSLRSEDTAVYYCTRPG |
| | NYEGFAYWGQGTLVTVSS [SEQ ID NO: 135] |

76H12_H corresponds to the sequence of VH from parental (murine) 76H12. Underlined residues represent CDRs as defined for the purpose of humanization. This is a custom CDR definition distinct from the IMGT definition used elsewhere. Bold residues in humanized variants represent framework point mutations to match the 76H12_H sequence.

TABLE 85
VL sequences of 76H12 and humanized variants

| VL | |
|---|---|
| Identifier | Sequence |
| 76H12_L | NIVLTQSPASLAVSLGQRATISCRASESVDSYGSSFMHWYQQKPGQPPKL |
| | LIYLASNLESGVPARFSGSGSRTDFTLTIDPVEADDAATYYCQQNNEDPW |
| | TFGGGTKLEIK [SEQ ID NO: 123] |
| h76H12_Lv0 | DIQMTQSPSSLSASVGDRVTITCRASESVDSYGSSFMHWYQQKPGKAPKL |
| | LIYLASNLESGVPSRFSGSGSGTDFTLTISSLQPEDFATYYCQQNNEDPW |
| | TFGQGTKLEIK [SEQ ID NO: 136] |
| h76H12_Lv1 | DIQLTQSPSSLSASVGDRVTITCRASESVDSYGSSFMHWYQQKPGKPPKL |
| | LIYLASNLESGVPSRFSGSGSGTDFTLTISSLQPEDFATYYCQQNNEDPW |
| | TFGQGTKLEIK [SEQ ID NO: 131] |
| h76H12_Lv2 | DIQLTQSPSSLSASVGDRVTITCRASESVDSYGSSFMHWYQQKPGKPPKL |
| | LIYLASNLESGVPSRFSGSGSRTDFTLTISSLQPEDFATYYCQQNNEDPW |
| | TFGQGTKLEIK [SEQ ID NO: 132] |
| h76H12_Lv3 | NIQLTQSPSSLSASVGDRVTITCRASESVDSYGSSFMHWYQQKPGKPPKL |
| | LIYLASNLESGVPSRFSGSGSGTDFTLTISSLQPEDFATYYCQQNNEDPW |
| | TFGQGTKLEIK [SEQ ID NO: 129] |

76H12_L corresponds to the sequence of VL from parental (murine) 76H12. Underlined residues represent CDRs as defined for the purpose of humanization. This is a custom CDR definition distinct from the IMGT definition used elsewhere. Bold residues in humanized variants represent framework point mutations to match the 76H12_L sequence.

To evaluate monovalent binding by humanized 76H12 variants to LCAM, Fab proteins were generated (Table 86) with V_{H} sequences fused to CH1 from human IgG1 and V_{L} fused to the constant region of human kappa light chain.

TABLE 86
Design of chimeric and humanized 76H12 Fab variants

| Fab Construct | VH sequence | VL sequence |
|---|---|---|
| 1 (parental mouse | 76H12_H | 76H12_L |
| 76H12 V-regions) | | |
| 2 | h76H12_Hv0 | h76H12_Lv3 |
| 3 | h76H12_Hv1 | h76H12_Lv1 |
| 4 | h76H12_Hv1 | h76H12_Lv2 |
| 5 | h76H12_Hv1 | h76H12_Lv3 |
| 6 | h76H12_Hv2 | h76H12_Lv1 |
| 7 | h76H12_Hv2 | h76H12_Lv2 |
| 8 | h76H12_Hv2 | h76H12_Lv3 |
| 9 | h76H12_Hv3 | h76H12_Lv1 |
| 10 | h76H12_Hv3 | h76H12_Lv2 |
| 11 | h76H12_Hv3 | h76H12_Lv3 |
| 12 | h76H12_Hv3 | h76H12_Lv0 |

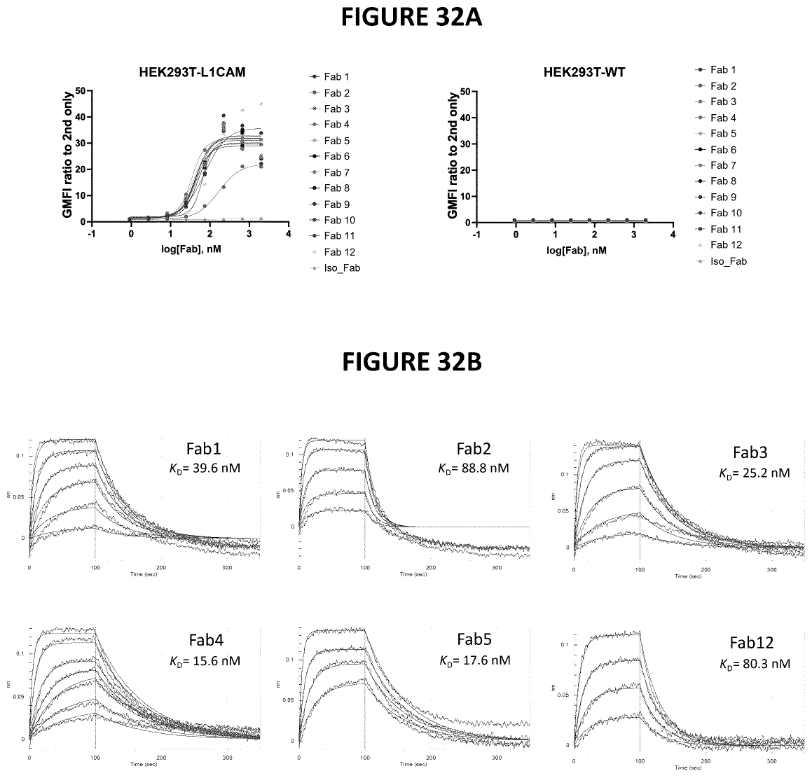

A total of 11 humanized 76H12 Fab variants (Fabs 2-12) were generated in addition to a chimeric 76H12 Fab (Fab 1) containing the parental murine VH and VL sequences. The chimeric Fab served as the benchmark for comparative assessment of humanized Fab variants. Humanized Fabs were first assessed by flow cytometry for binding to 293T cells engineered to overexpress L1CAM (FIG. 32A). All humanized variants bound to the L1CAM overexpressing cells, with most binding comparably to the chimeric 76H12 Fab. None of the Fabs bound to parental 293T cells that do not express L1CAM. Based on these data, the chimeric Fab and a subset of humanized variants were further assessed for binding to recombinant L1CAM protein by bilayer interferometry (FIG. 32B). Here, recombinant L1CAM-Fc fusion protein was immobilized onto anti-Human IgG Fc Capture (AHC) biosensors and binding of Fabs was measured kinetically over a 3-200 nM concentration range. Kinetic data were fitted to a 1:1 binding model to compute association (kₒₙ) and dissociation (k_{off}) rate constants, in addition to equilibrium dissociation constants (K_{D}) as shown in Table 87. Three of the humanized Fab constructs (Fabs 3, 4 and 5) met the criteria of binding to L1CAM with equivalent or better affinity than the chimeric Fab (Fab 1). From these, Fab 3 represents the preferred humanized variant given higher identity to human germline IGHV1-46 and IGKV1-39 sequences resulting from fewer human-to-murine framework back mutations.

TABLE 87
Binding of chimeric and humanized 76H12 Fab variants to LICAM by BLI.

| Fab Construct | VH sequence | VL sequence | kon (M−1s−1) | Koff (s−1) | KD (M) |
|---|---|---|---|---|---|
| 1 (chimeric) | 76H12_H | 76H12_L | 5.86E+05 | 2.32E−02 | 3.96E−08 |
| 2 (humanized) | h76H12_Hv0 | h76H12_Lv3 | 7.97E+05 | 7.06E−02 | 8.88E−08 |
| 3 (humanized) | h76H12_Hv1 | h76H12_Lv1 | 7.81E+05 | 1.97E−02 | 2.52E−08 |
| 4 (humanized) | h76H12_Hv1 | h76H12_Lv2 | 9.22E+05 | 1.43E−02 | 1.56E−08 |
| 5 (humanized) | h76H12_Hv1 | h76H12_Lv3 | 9.57E+05 | 1.68E−02 | 1.76E−08 |
| 12 (humanized) | h76H12_Hv3 | h76H12_Lv0 | 3.95E+05 | 3.18E−02 | 8.03E−08 |

### Example 14—Effects of 13F04 Against Human Metastatic Lung Adenocarcinoma (LUAD)

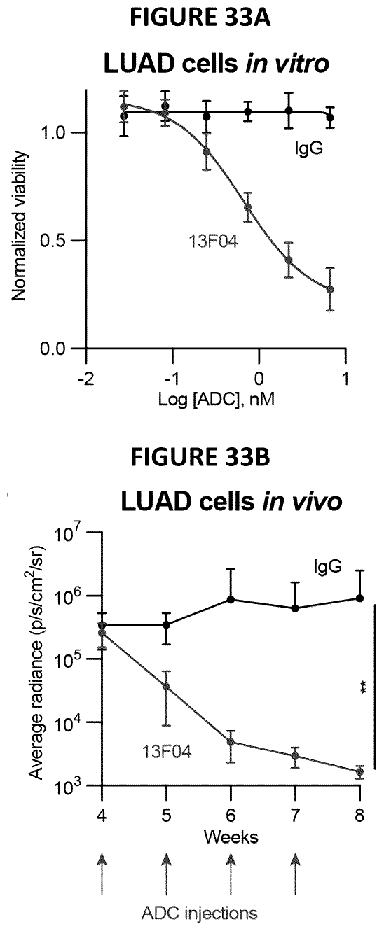

H23 is a cell line derived from KRAS-mutant, p53 mutant LUAD adenocarcinoma prior to treatment (https://www.atcc.org/products/crl-5800). H23 cell cultures were treated with ADCs generated with L1CAM mAb 13F04 or IgG control conjugated to PNU-159682. In vitro cell viability was assessed 96 h later using CellTiterGlo reagent and was normalized to DMSO control. As seen in FIG. 33A, the presently disclosed anti-L1CAM antibody significantly reduced in vitro viability.

Next, Ru631, a LUAD patient-derived xenograft, was developed. LUAD organoids were grown from Ru631 to enrich for LUAD stem-like cells. Cell suspensions from these organoids were injected to immunodeficient NSG mice via tail vein to generate lung metastatic colonies. After 4 weeks, animals were administrated 4 weekly doses of 13F04 or IgG ADCs (1 mg/kg) and the tumor growth was monitored using BLI measurements. Notably, as seen in FIG. 33B, the presently disclosed anti-L1CAM antibody significantly reduced in vivo viability. Overall, the presently disclosed antibodies showed significant activity both in vitro and in vivo.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An anti-L1CAM antibody or an antigen-binding fragment thereof, comprising:
(a) a heavy chain variable region comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, a CDR3 comprising the amino acid sequence set forth in SEQ ID NO: 4 or a conservative modification thereof; and a light chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
(b) a heavy chain variable region comprising CDR1 comprising the amino acid sequence set forth in SEQ ID NO: 12 or a conservative modification thereof, a CDR2 comprising an amino acid sequence set forth in SEQ ID NO: 13 or a conservative modification thereof, a CDR3 comprising the amino acid sequence set forth in SEQ ID NO: 14 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof;
(c) a heavy chain variable region comprising CDR1 comprising the amino acid sequence set forth in SEQ ID NO: 12 or a conservative modification thereof, a CDR2 comprising an amino acid sequence set forth in SEQ ID NO: 13 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof;
(d) a heavy chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, a CDR3 comprising the amino acid sequence set forth in SEQ ID NO: 37 or a conservative modification thereof; and a light chain variable region comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof;
(e) a heavy chain variable region comprising CDR1 comprising the amino acid sequence set forth in SEQ ID NO: 43 or a conservative modification thereof, a CDR2 comprising an amino acid sequence set forth in SEQ ID NO: 44 or a conservative modification thereof, a CDR3 comprising the amino acid sequence set forth in SEQ ID NO: 45 or a conservative modification thereof; and a light chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof;
(f) a heavy chain variable region comprising CDR1 comprising the amino acid sequence set forth in SEQ ID NO: 43 or a conservative modification thereof, a CDR2 comprising an amino acid sequence set forth in SEQ ID NO: 44 or a conservative modification thereof, a CDR3 comprising the amino acid sequence set forth in SEQ ID NO: 45 or a conservative modification thereof; and a light chain variable region comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 40 or a conservative modification thereof;
(g) a heavy chain variable region comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, a CDR3 comprising the amino acid sequence set forth in SEQ ID NO: 56 or a conservative modification thereof; and a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(h) a heavy chain variable region comprising CDR1 comprising the amino acid sequence set forth in SEQ ID NO: 62 or a conservative modification thereof, a CDR2 comprising an amino acid sequence set forth in SEQ ID NO: 63 or a conservative modification thereof, a CDR3 comprising the amino acid sequence set forth in SEQ ID NO: 64 or a conservative modification thereof; and a light chain variable region comprising a CDR1 comprising the amino acid sequence set forth in SEQ ID NO: 65 or a conservative modification thereof, a CDR2 comprising the amino acid sequence set forth in SEQ ID NO: 58 or a conservative modification thereof, and a CDR3 comprising the amino acid sequence set forth in SEQ ID NO: 66 or a conservative modification thereof;
(i) a heavy chain variable region comprising CDR1 comprising the amino acid sequence set forth in SEQ ID NO: 69 or a conservative modification thereof, a CDR2 comprising an amino acid sequence set forth in SEQ ID NO: 70 or a conservative modification thereof, a CDR3 comprising the amino acid sequence set forth in SEQ ID NO: 71 or a conservative modification thereof; and a light chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof;
(j) a heavy chain variable region comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, a CDR3 comprising the amino acid sequence set forth in SEQ ID NO: 83 or a conservative modification thereof; and a light chain variable region comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof;
(k) a heavy chain variable region comprising CDR1 comprising the amino acid sequence set forth in SEQ ID NO: 93 or a conservative modification thereof, a CDR2 comprising an amino acid sequence set forth in SEQ ID NO: 94 or a conservative modification thereof, a CDR3 comprising the amino acid sequence set forth in SEQ ID NO: 95 or a conservative modification thereof; and a light chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof;
(l) a heavy chain variable region comprising CDR1 comprising the amino acid sequence set forth in SEQ ID NO: 105 or a conservative modification thereof, a CDR2 comprising an amino acid sequence set forth in SEQ ID NO: 106 or a conservative modification thereof, a CDR3 comprising the amino acid sequence set forth in SEQ ID NO: 107 or a conservative modification thereof; and a light chain variable region comprising a CDR1 comprising the amino acid sequence set forth in SEQ ID NO: 108 or a conservative modification thereof, a CDR2 comprising the amino acid sequence set forth in SEQ ID NO: 39 or a conservative modification thereof, and a CDR3 comprising the amino acid sequence set forth in SEQ ID NO: 109 or a conservative modification thereof; or
(m) a heavy chain variable region comprising a CDR1 comprising the amino acid sequence set forth in SEQ ID NO: 116 or a conservative modification thereof, a CDR2 comprising the amino acid sequence set forth in SEQ ID NO: 117 or a conservative modification thereof, a CDR3 comprising the amino acid sequence set forth in SEQ ID NO: 118 or a conservative modification thereof; and a light chain variable region comprising a CDR1 comprising the amino acid sequence set forth in SEQ ID NO: 119 or a conservative modification thereof, a CDR2 comprising the amino acid sequence set forth in SEQ ID NO: 120, and a CDR3 comprising the amino acid sequence set forth in SEQ ID NO: 121 or a conservative modification thereof.

2. The anti-L1CAM antibody or an antigen-binding fragment thereof of claim 1, wherein:
(a) the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) the heavy chain variable region comprises CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 26; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(d) the heavy chain variable region comprises a CDR1 comprising the amino acid sequence set forth in SEQ ID NO: 35, a CDR2 comprising the amino acid sequence set forth in SEQ ID NO: 36, a CDR3 comprising the amino acid sequence set forth in SEQ ID NO: 37; and the light chain variable region comprises a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40v;
(e) the heavy chain variable region comprises CDR1 comprising the amino acid sequence set forth in SEQ ID NO: 43, a CDR2 comprising an amino acid sequence set forth in SEQ ID NO: 44, a CDR3 comprising the amino acid sequence set forth in SEQ ID NO: 45; and the light chain variable region comprises a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(f) the heavy chain variable region comprises CDR1 comprising the amino acid sequence set forth in SEQ ID NO: 43, a CDR2 comprising an amino acid sequence set forth in SEQ ID NO: 44, a CDR3 comprising the amino acid sequence set forth in SEQ ID NO: 45; and the light chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 40;
(g) the heavy chain variable region comprises a CDR1 comprising the amino acid sequence set forth in SEQ ID NO: 54, a CDR2 comprising the amino acid sequence set forth in SEQ ID NO: 55, a CDR3 comprising the amino acid sequence set forth in SEQ ID NO: 56; and the light chain variable region comprises a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) the heavy chain variable region comprises CDR1 comprising the amino acid sequence set forth in SEQ ID NO: 62, a CDR2 comprising an amino acid sequence set forth in SEQ ID NO: 63, a CDR3 comprising the amino acid sequence set forth in SEQ ID NO: 64; and the light chain variable region comprises a CDR1 comprising the amino acid sequence set forth in SEQ ID NO: 65, a CDR2 comprising the amino acid sequence set forth in SEQ ID NO: 58, and a CDR3 comprising the amino acid sequence set forth in SEQ ID NO: 66;
(i) the heavy chain variable region comprises CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and
the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(j) the heavy chain variable region comprises a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
(k) the heavy chain variable region comprises CDR1 comprising the amino acid sequence set forth in SEQ ID NO: 93, a CDR2 comprising an amino acid sequence set forth in SEQ ID NO: 94, a CDR3 comprising the amino acid sequence set forth in SEQ ID NO: 95; and the light chain variable region comprises a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
(l) the heavy chain variable region comprises CDR1 comprising the amino acid sequence set forth in SEQ ID NO: 105, a CDR2 comprising an amino acid sequence set forth in SEQ ID NO: 106, a CDR3 comprising the amino acid sequence set forth in SEQ ID NO: 107; and the light chain variable region comprises a CDR1 comprising the amino acid sequence set forth in SEQ ID NO: 108, a CDR2 comprising the amino acid sequence set forth in SEQ ID NO: 39, and a CDR3 comprising the amino acid sequence set forth in SEQ ID NO: 109; or
(m) the heavy chain variable region comprises a CDR1 comprising the amino acid sequence set forth in SEQ ID NO: 116, a CDR2 comprising the amino acid sequence set forth in SEQ ID NO: 117, a CDR3 comprising the amino acid sequence set forth in SEQ ID NO: 118; and the light chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

3. The anti-L1CAM antibody or antigen-binding fragment thereof of claim 1 or 2, comprising:
(a) the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprising a CDR1 comprises the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or
(c) the heavy chain variable region comprises a CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

4. The anti-L1CAM antibody or antigen-binding fragment thereof of any one of claims 1-3, wherein the heavy chain variable region comprises a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and the light chain variable region comprising a CDR1 comprises the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

5. The anti-L1CAM antibody or antigen-binding fragment thereof of any one of claims 1-3, wherein the heavy chain variable region comprises a CDR1 comprising the amino acid sequence set forth in SEQ ID NO: 12, a CDR2 comprising an amino acid sequence set forth in SEQ ID NO: 13, a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

6. The anti-L1CAM antibody or antigen-binding fragment thereof of any one of claims 1-3, wherein the heavy chain variable region comprises a CDR1 comprising the amino acid sequence set forth in SEQ ID NO: 69, a CDR2 comprising an amino acid sequence set forth in SEQ ID NO: 70, a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

7. The anti-L1CAM antibody or an antigen-binding fragment thereof of any one of claims 1-6, comprising
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 22, SEQ ID NO: 24, SEQ ID NO: 25, SEQ ID NO: 27, SEQ ID NO: 28, SEQ ID NO: 29, SEQ ID NO: 33, SEQ ID NO: 41, SEQ ID NO: 49, SEQ ID NO: 60, SEQ ID NO: 67, SEQ ID NO: 75, SEQ ID NO: 87, SEQ ID NO: 99, SEQ ID NO: 110, SEQ ID NO: 122, SEQ ID NO: 128, SEQ ID NO: 130, SEQ ID NO: 134, or SEQ ID NO: 135; or
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 9, SEQ ID NO: 19, SEQ ID NO: 23, SEQ ID NO: 30, SEQ ID NO: 31, SEQ ID NO: 32, SEQ ID NO: 34, SEQ ID NO: 42, SEQ ID NO: 50, SEQ ID NO: 53, SEQ ID NO: 61, SEQ ID NO: 68, SEQ ID NO: 76, SEQ ID NO: 88, SEQ ID NO: 100, SEQ ID NO: 111, SEQ ID NO: 123, SEQ ID NO: 129, SEQ ID NO: 131, SEQ ID NO: 132, or SEQ ID NO: 133.

8. The anti-L1CAM antibody or an antigen-binding fragment thereof of any one of claims 1-7, comprising:
(a) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 9;
(b) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 18, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(c) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(d) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(e) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(f) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(g) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(h) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 23;
(i) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(j) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(k) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(l) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(m) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(n) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 30;
(o) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(p) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(q) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(r) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(s) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(t) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 31;
(u) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(v) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(w) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(x) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(y) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(z) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 32;
(aa) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ab) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ac) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 25, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ad) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 27, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ae) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(af) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 29, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 19;
(ag) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 33, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 34;
(ah) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 41, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 42;
(ai) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 50;
(aj) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 53;
(ak) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 60, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 61;
(al) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 67, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 68;
(am) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 75, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 76;
(an) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 87, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 88;
(ao) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 99, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 100;
(ap) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 110, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 111;
(aq) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 122, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 123;
(ar) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 128, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 129;
(as) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131;
(at) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132;
(au) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 130, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133;
(av) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131;
(aw) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132;
(ax) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 134, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133;
(ay) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 131;
(az) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 132;
(ba) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 133; and
(bb) a heavy chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 135, and a light chain variable region comprising an amino acid sequence that is at least about 80%, at least about 85%, at least about 90%, at least about 95%, at least about 96%, at least about 97%, at least about 98%, at least about 99%, at least about 100% identical to the amino acid sequence set forth in SEQ ID NO: 136.

9. The antibody or antigen-binding fragment thereof of any one of claims 1-8, wherein
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 22, and the light chain variable region comprises the amino acid sequence set forth in SEQ ID NO: 23;
(d) the heavy chain variable region comprises the amino acid sequence set forth in SEQ ID NO: 24, and the light chain variable region comprises the amino acid sequence set forth in SEQ ID NO: 23;
(e) the heavy chain variable region comprises the amino acid sequence set forth in SEQ ID NO: 25, and the light chain variable region comprises the amino acid sequence set forth in SEQ ID NO: 23;
(f) the heavy chain variable region comprises the amino acid sequence set forth in SEQ ID NO: 27, and the light chain variable region comprises the amino acid sequence set forth in SEQ ID NO: 23;
(g) the heavy chain variable region comprises the amino acid sequence set forth in SEQ ID NO: 28, and the light chain variable region comprises the amino acid sequence set forth in SEQ ID NO: 23;
(h) the heavy chain variable region comprises the amino acid sequence set forth in SEQ ID NO: 29, and the light chain variable region comprises the amino acid sequence set forth in SEQ ID NO: 23;
(i) the heavy chain variable region comprises the amino acid sequence set forth in SEQ ID NO: 22, and the light chain variable region comprises the amino acid sequence set forth in SEQ ID NO: 30;
(j) the heavy chain variable region comprises the amino acid sequence set forth in SEQ ID NO: 24, and the light chain variable region comprises the amino acid sequence set forth in SEQ ID NO: 30;
(k) the heavy chain variable region comprises the amino acid sequence set forth in SEQ ID NO: 25, and the light chain variable region comprises the amino acid sequence set forth in SEQ ID NO: 30;
(l) the heavy chain variable region comprises the amino acid sequence set forth in SEQ ID NO: 27, and the light chain variable region comprises the amino acid sequence set forth in SEQ ID NO: 30;
(m) the heavy chain variable region comprises the amino acid sequence set forth in SEQ ID NO: 28, and the light chain variable region comprises the amino acid sequence set forth in SEQ ID NO: 30;
(n) the heavy chain variable region comprises the amino acid sequence set forth in SEQ ID NO: 29, and the light chain variable region comprises the amino acid sequence set forth in SEQ ID NO: 30;
(o) the heavy chain variable region comprises the amino acid sequence set forth in SEQ ID NO: 22, and the light chain variable region comprises the amino acid sequence set forth in SEQ ID NO: 31;
(p) the heavy chain variable region comprises the amino acid sequence set forth in SEQ ID NO: 24, and the light chain variable region comprises the amino acid sequence set forth in SEQ ID NO: 31;
(q) the heavy chain variable region comprises the amino acid sequence set forth in SEQ ID NO: 25, and the light chain variable region comprises the amino acid sequence set forth in SEQ ID NO: 31;
(r) the heavy chain variable region comprises the amino acid sequence set forth in SEQ ID NO: 27, and the light chain variable region comprises the amino acid sequence set forth in SEQ ID NO: 31;
(s) the heavy chain variable region comprises the amino acid sequence set forth in SEQ ID NO: 28, and the light chain variable region comprises the amino acid sequence set forth in SEQ ID NO: 31;
(t) the heavy chain variable region comprises the amino acid sequence set forth in SEQ ID NO: 29, and the light chain variable region comprises the amino acid sequence set forth in SEQ ID NO: 31;
(u) the heavy chain variable region comprises the amino acid sequence set forth in SEQ ID NO: 22, and the light chain variable region comprises the amino acid sequence set forth in SEQ ID NO: 32;
(v) the heavy chain variable region comprises the amino acid sequence set forth in SEQ ID NO: 24, and the light chain variable region comprises the amino acid sequence set forth in SEQ ID NO: 32;
(w) the heavy chain variable region comprises the amino acid sequence set forth in SEQ ID NO: 25, and the light chain variable region comprises the amino acid sequence set forth in SEQ ID NO: 32;
(x) the heavy chain variable region comprises the amino acid sequence set forth in SEQ ID NO: 27, and the light chain variable region comprises the amino acid sequence set forth in SEQ ID NO: 32;
(y) the heavy chain variable region comprises the amino acid sequence set forth in SEQ ID NO: 28, and the light chain variable region comprises the amino acid sequence set forth in SEQ ID NO: 32;
(z) the heavy chain variable region comprises the amino acid sequence set forth in SEQ ID NO: 29, and the light chain variable region comprises the amino acid sequence set forth in SEQ ID NO: 32;
(aa) the heavy chain variable region comprises the amino acid sequence set forth in SEQ ID NO: 22, and the light chain variable region comprises the amino acid sequence set forth in SEQ ID NO: 19;
(ab) the heavy chain variable region comprises the amino acid sequence set forth in SEQ ID NO: 24, and the light chain variable region comprises the amino acid sequence set forth in SEQ ID NO: 19;
(ac) the heavy chain variable region comprises the amino acid sequence set forth in SEQ ID NO: 25, and the light chain variable region comprises the amino acid sequence set forth in SEQ ID NO: 19;
(ad) the heavy chain variable region comprises the amino acid sequence set forth in SEQ ID NO: 27, and the light chain variable region comprises the amino acid sequence set forth in SEQ ID NO: 19;
(ae) the heavy chain variable region comprises the amino acid sequence set forth in SEQ ID NO: 28, and the light chain variable region comprises the amino acid sequence set forth in SEQ ID NO: 19;
(af) the heavy chain variable region comprises the amino acid sequence set forth in SEQ ID NO: 29, and the light chain variable region comprises the amino acid sequence set forth in SEQ ID NO: 19;
(ag) the heavy chain variable region comprising the amino acid sequence set forth in SEQ ID NO: 33, and the light chain variable region comprising the amino acid sequence set forth in SEQ ID NO: 34;
(ah) the heavy chain variable region comprising the amino acid sequence set forth in SEQ ID NO: 41, and the light chain variable region comprising the amino acid sequence set forth in SEQ ID NO: 42;
(ai) the heavy chain variable region comprising the amino acid sequence set forth in SEQ ID NO: 49, and the light chain variable region comprising the amino acid sequence set forth in SEQ ID NO: 50;
(aj) the heavy chain variable region comprising the amino acid sequence set forth in SEQ ID NO: 49, and the light chain variable region comprising the amino acid sequence set forth in SEQ ID NO: 53;
(ak) the heavy chain variable region comprising the amino acid sequence set forth in SEQ ID NO: 60, and the light chain variable region comprising the amino acid sequence set forth in SEQ ID NO: 61;
(al) the heavy chain variable region comprising the amino acid sequence set forth in SEQ ID NO: 67, and the light chain variable region comprising the amino acid sequence set forth in SEQ ID NO: 68;
(am) the heavy chain variable region comprising the amino acid sequence set forth in SEQ ID NO: 75, and the light chain variable region comprising the amino acid sequence set forth in SEQ ID NO: 76;
(an) the heavy chain variable region comprising the amino acid sequence set forth in SEQ ID NO: 87, and the light chain variable region comprising the amino acid sequence set forth in SEQ ID NO: 88;
(ao) the heavy chain variable region comprising the amino acid sequence set forth in SEQ ID NO: 99, and the light chain variable region comprising the amino acid sequence set forth in SEQ ID NO: 100;
(ap) the heavy chain variable region comprising the amino acid sequence set forth in SEQ ID NO: 110, and the light chain variable region comprising the amino acid sequence set forth in SEQ ID NO: 111;
(aq) the heavy chain variable region comprising the amino acid sequence set forth in SEQ ID NO: 122, and the light chain variable region comprising the amino acid sequence set forth in SEQ ID NO: 123;
(ar) the heavy chain variable region comprising the amino acid sequence set forth in SEQ ID NO: 128, and the light chain variable region comprising the amino acid sequence set forth in SEQ ID NO: 129;
(as) the heavy chain variable region comprising the amino acid sequence set forth in SEQ ID NO: 130, and the light chain variable region comprising the amino acid sequence set forth in SEQ ID NO: 131;
(at) the heavy chain variable region comprising the amino acid sequence set forth in SEQ ID NO: 130, and the light chain variable region comprising the amino acid sequence set forth in SEQ ID NO: 132;
(au) the heavy chain variable region comprising the amino acid sequence set forth in SEQ ID NO: 130, and the light chain variable region comprising the amino acid sequence set forth in SEQ ID NO: 133;
(av) the heavy chain variable region comprising the amino acid sequence set forth in SEQ ID NO: 134, and the light chain variable region comprising the amino acid sequence set forth in SEQ ID NO: 131;
(aw) the heavy chain variable region comprising the amino acid sequence set forth in SEQ ID NO: 134, and the light chain variable region comprising the amino acid sequence set forth in SEQ ID NO: 132;
(ax) the heavy chain variable region comprising the amino acid sequence set forth in SEQ ID NO: 134, and the light chain variable region comprising the amino acid sequence set forth in SEQ ID NO: 133;
(ay) the heavy chain variable region comprising the amino acid sequence set forth in SEQ ID NO: 135, and the light chain variable region comprising the amino acid sequence set forth in SEQ ID NO: 131;
(az) the heavy chain variable region comprising the amino acid sequence set forth in SEQ ID NO: 135, and the light chain variable region comprising the amino acid sequence set forth in SEQ ID NO: 132;
(ba) the heavy chain variable region comprising the amino acid sequence set forth in SEQ ID NO: 135, and the light chain variable region comprising the amino acid sequence set forth in SEQ ID NO: 133; or
(bb) the heavy chain variable region comprising the amino acid sequence set forth in SEQ ID NO: 135, and the light chain variable region comprising the amino acid sequence set forth in SEQ ID NO: 136.

10. The anti-L1CAM antibody or antigen-binding fragment thereof of any one of claims 1-9, wherein the antibody comprises a comprises a heavy chain constant region and/or a light chain constant region.

11. The anti-L1CAM antibody or antigen-binding fragment thereof of claim 10, wherein:
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence set forth in SEQ ID NO: 144, SEQ ID NO: 145, SEQ ID NO: 146, or SEQ ID NO: 147; and/or
(b) the light chain constant region comprises an amino acid sequence that is at least about 80%, at least about 85%, at least about 90%, at least about 95%, at least about 96%, at least about 97%, at least about 98%, at least about 99% at least about 100% identical to the amino acid sequence set forth in SEQ ID NO: 148.

12. The anti-L1CAM antibody or antigen-binding fragment thereof of claim 10 or 11, wherein:
(a) the heavy chain constant region comprises the amino acid sequence set forth in SEQ ID NO: 144, SEQ ID NO: 145, SEQ ID NO: 146, or SEQ ID NO: 147; and/or
(b) the light chain constant region comprises the amino acid sequence set forth in SEQ ID NO: 148.

13. The anti-L1CAM antibody or antigen-binding fragment thereof of any one of claims 1-12, wherein the antibody comprises a human variable region framework region.

14. The anti-L1CAM antibody or antigen-binding fragment thereof of any one of claims 1-13, which is a fully human or an antigen-binding fragment thereof.

15. The anti-L1CAM antibody or antigen-binding fragment thereof of any one of claims 1-12, which is a chimeric antibody or an antigen-binding fragment thereof.

16. The anti-L1CAM antibody or antigen-binding fragment thereof of any one of claims 1-12, which is a humanized antibody or an antigen-binding fragment thereof.

17. The anti-L1CAM antibody or antigen-binding fragment thereof of any one of claims 1-16, wherein the antigen-binding fragment is a Fab, Fab′, F(ab′)₂, variable fragment (Fv), or single chain variable region (scFv).

18. The anti-L1CAM antibody or antigen-binding fragment thereof of claim 17, wherein the antigen-binding fragment is an scFv.

19. An anti-L1CAM antibody or an antigen-binding fragment thereof comprising
(a) a heavy chain variable region comprising a CDR1, a CDR2, and a CDR3 of the heavy chain variable region comprising the amino acid sequence set forth in SEQ ID NO: 8, and a light chain variable region comprising a CDR1, a CDR2, and a CDR3 of the light chain variable region comprising the amino acid sequence set forth in SEQ ID NO: 9;
(b) a heavy chain variable region comprising a CDR1, a CDR2, and a CDR3 of the heavy chain variable region comprising the amino acid sequence set forth in SEQ ID NO: 18, and a light chain variable region comprising a CDR1, a CDR2, and a CDR3 of the light chain variable region comprising the amino acid sequence set forth in SEQ ID NO: 19;
(c) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 23;
(d) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 23;
(e) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 23;
(f) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 23;
(g) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 23;
(h) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 23;
(i) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 30;
(j) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 30;
(k) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 30;
(l) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 30;
(m) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 30;
(n) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 30;
(o) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 31;
(p) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 31;
(q) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 31;
(r) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 31;
(s) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 31;
(t) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 31;
(u) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 32;
(v) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 32;
(w) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 32;
(x) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 32;
(y) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 32;
(z) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 32;
(aa) a heavy chain variable region comprising a CDR1, a CDR2, and a CDR3 of the heavy chain variable region comprising the amino acid sequence set forth in SEQ ID NO: 22, and a light chain variable region comprising a CDR1, a CDR2, and a CDR3 of the light chain variable region comprising the amino acid sequence set forth in SEQ ID NO: 19;
(ab) a heavy chain variable region comprising a CDR1, a CDR2, and a CDR3 of the heavy chain variable region comprising the amino acid sequence set forth in SEQ ID NO: 24, and a light chain variable region comprising a CDR1, a CDR2, and a CDR3 of the light chain variable region comprising the amino acid sequence set forth in SEQ ID NO: 19;
(ac) a heavy chain variable region comprising a CDR1, a CDR2, and a CDR3 of the heavy chain variable region comprising the amino acid sequence set forth in SEQ ID NO: 25, and a light chain variable region comprising a CDR1, a CDR2, and a CDR3 of the light chain variable region comprising the amino acid sequence set forth in SEQ ID NO: 19;
(ad) a heavy chain variable region comprising a CDR1, a CDR2, and a CDR3 of the heavy chain variable region comprising the amino acid sequence set forth in SEQ ID NO: 27, and a light chain variable region comprising a CDR1, a CDR2, and a CDR3 of the light chain variable region comprising the amino acid sequence set forth in SEQ ID NO: 19;
(ae) a heavy chain variable region comprising a CDR1, a CDR2, and a CDR3 of the heavy chain variable region comprising the amino acid sequence set forth in SEQ ID NO: 28, and a light chain variable region comprising a CDR1, a CDR2, and a CDR3 of the light chain variable region comprising the amino acid sequence set forth in SEQ ID NO: 19;
(af) a heavy chain variable region comprising a CDR1, a CDR2, and a CDR3 of the heavy chain variable region comprising the amino acid sequence set forth in SEQ ID NO: 29, and a light chain variable region comprising a CDR1, a CDR2, and a CDR3 of the light chain variable region comprising the amino acid sequence set forth in SEQ ID NO: 19;
(ag) the heavy chain variable region comprising the amino acid sequence set forth in SEQ ID NO: 33, and the light chain variable region comprising the amino acid sequence set forth in SEQ ID NO: 34;
(ah) the heavy chain variable region comprising the amino acid sequence set forth in SEQ ID NO: 41, and the light chain variable region comprising the amino acid sequence set forth in SEQ ID NO: 42;
(ai) the heavy chain variable region comprising the amino acid sequence set forth in SEQ ID NO: 49, and the light chain variable region comprising the amino acid sequence set forth in SEQ ID NO: 50;
(aj) the heavy chain variable region comprising the amino acid sequence set forth in SEQ ID NO: 49, and the light chain variable region comprising the amino acid sequence set forth in SEQ ID NO: 53;
(ak) the heavy chain variable region comprising the amino acid sequence set forth in SEQ ID NO: 60, and the light chain variable region comprising the amino acid sequence set forth in SEQ ID NO: 61;
(al) the heavy chain variable region comprising the amino acid sequence set forth in SEQ ID NO: 67, and the light chain variable region comprising the amino acid sequence set forth in SEQ ID NO: 68;
(am) the heavy chain variable region comprising the amino acid sequence set forth in SEQ ID NO: 75, and the light chain variable region comprising the amino acid sequence set forth in SEQ ID NO: 76;
(an) the heavy chain variable region comprising the amino acid sequence set forth in SEQ ID NO: 87, and the light chain variable region comprising the amino acid sequence set forth in SEQ ID NO: 88;
(ao) the heavy chain variable region comprising the amino acid sequence set forth in SEQ ID NO: 99, and the light chain variable region comprising the amino acid sequence set forth in SEQ ID NO: 100;
(ap) the heavy chain variable region comprising the amino acid sequence set forth in SEQ ID NO: 110, and the light chain variable region comprising the amino acid sequence set forth in SEQ ID NO: 111;
(aq) the heavy chain variable region comprising the amino acid sequence set forth in SEQ ID NO: 122, and the light chain variable region comprising the amino acid sequence set forth in SEQ ID NO: 123;
(ar) the heavy chain variable region comprising the amino acid sequence set forth in SEQ ID NO: 128, and the light chain variable region comprising the amino acid sequence set forth in SEQ ID NO: 129;
(as) the heavy chain variable region comprising the amino acid sequence set forth in SEQ ID NO: 130, and the light chain variable region comprising the amino acid sequence set forth in SEQ ID NO: 131;
(at) the heavy chain variable region comprising the amino acid sequence set forth in SEQ ID NO: 130, and the light chain variable region comprising the amino acid sequence set forth in SEQ ID NO: 132;
(au) the heavy chain variable region comprising the amino acid sequence set forth in SEQ ID NO: 130, and the light chain variable region comprising the amino acid sequence set forth in SEQ ID NO: 133;
(av) the heavy chain variable region comprising the amino acid sequence set forth in SEQ ID NO: 134, and the light chain variable region comprising the amino acid sequence set forth in SEQ ID NO: 131;
(aw) the heavy chain variable region comprising the amino acid sequence set forth in SEQ ID NO: 134, and the light chain variable region comprising the amino acid sequence set forth in SEQ ID NO: 132;
(ax) the heavy chain variable region comprising the amino acid sequence set forth in SEQ ID NO: 134, and the light chain variable region comprising the amino acid sequence set forth in SEQ ID NO: 133;
(ay) the heavy chain variable region comprising the amino acid sequence set forth in SEQ ID NO: 135, and the light chain variable region comprising the amino acid sequence set forth in SEQ ID NO: 131;
(az) the heavy chain variable region comprising the amino acid sequence set forth in SEQ ID NO: 135, and the light chain variable region comprising the amino acid sequence set forth in SEQ ID NO: 132;
(ba) the heavy chain variable region comprising the amino acid sequence set forth in SEQ ID NO: 135, and the light chain variable region comprising the amino acid sequence set forth in SEQ ID NO: 133; or
(bb) the heavy chain variable region comprising the amino acid sequence set forth in SEQ ID NO: 135, and the light chain variable region comprising the amino acid sequence set forth in SEQ ID NO: 136.

20. An anti-L1CAM antibody or an antigen-binding fragment thereof comprising:
(a) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 77 or SEQ ID NO: 78;
(b) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 89 or SEQ ID NO: 90;
(c) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 101 or SEQ ID NO: 102;
(d) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 112 or SEQ ID NO: 113; or
(e) a single chain variable region (scFv) comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 124 or SEQ ID NO: 125.

21. The anti-L1CAM antibody or an antigen-binding fragment thereof of claim 20, wherein:
(a) the scFv comprises the amino acid sequence set forth in SEQ ID NO: 77 or SEQ ID NO: 78;
(b) the scFv comprises the amino acid sequence set forth in SEQ ID NO: 89 or SEQ ID NO: 90;
(c) the scFv comprises the amino acid sequence set forth in SEQ ID NO: 101 or SEQ ID NO: 102;
(d) the scFv comprises the amino acid sequence set forth in SEQ ID NO: 112 or SEQ ID NO: 113; or
(e) the scFv comprises the amino acid sequence set forth in SEQ ID NO: 124 or SEQ ID NO: 125.

22. An anti-L1CAM antibody or an antigen-binding fragment thereof comprising a heavy chain variable region comprising a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

23. An anti-L1CAM antibody or an antigen-binding fragment thereof comprising a heavy chain variable region comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

24. An anti-L1CAM antibody or an antigen-binding fragment thereof comprising a heavy chain variable region comprising a CDR1 comprising the amino acid sequence set forth in SEQ ID NO: 69, a CDR2 comprising the amino acid sequence set forth in SEQ ID NO: 70, a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

25. An anti-L1CAM antibody or an antigen-binding fragment thereof comprising a heavy chain variable region comprising a CDR1 comprising the amino acid sequence set forth in SEQ ID NO: 81, a CDR2 comprising the amino acid sequence set forth in SEQ ID NO: 82, a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

26. An anti-L1CAM antibody or an antigen-binding fragment thereof comprising a heavy chain variable region comprising a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 95; and a light chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98.

27. An anti-L1CAM antibody or an antigen-binding fragment thereof comprising a heavy chain variable region comprising a heavy chain variable region comprising the amino acid sequence set forth in SEQ ID NO: 8, and a light chain variable region comprising the amino acid sequence set forth in SEQ ID NO: 9.

28. An anti-L1CAM antibody or an antigen-binding fragment thereof comprising a heavy chain variable region comprising a heavy chain variable region comprising the amino acid sequence set forth in SEQ ID NO: 18, and a light chain variable region comprising the amino acid sequence set forth in SEQ ID NO: 19.

29. An anti-L1CAM antibody or an antigen-binding fragment thereof comprising a heavy chain variable region comprising a heavy chain variable region comprising the amino acid sequence set forth in SEQ ID NO: 75, and a light chain variable region comprising the amino acid sequence set forth in SEQ ID NO: 76.

30. An anti-L1CAM antibody or an antigen-binding fragment thereof comprising a heavy chain variable region comprising a heavy chain variable region comprising the amino acid sequence set forth in SEQ ID NO: 87, and a light chain variable region comprising the amino acid sequence set forth in SEQ ID NO: 88.

31. An anti-L1CAM antibody or an antigen-binding fragment thereof comprising a heavy chain variable region comprising a heavy chain variable region comprising the amino acid sequence set forth in SEQ ID NO: 99, and a light chain variable region comprising the amino acid sequence set forth in SEQ ID NO: 100.

32. An anti-L1CAM antibody or an antigen-binding fragment thereof comprising the scFv comprises the amino acid sequence set forth in SEQ ID NO: 77 or SEQ ID NO: 78.

33. An anti-L1CAM antibody or an antigen-binding fragment thereof comprising the scFv comprises the amino acid sequence set forth in SEQ ID NO: 89 or SEQ ID NO: 90.

34. An anti-L1CAM antibody or an antigen-binding fragment thereof comprising the scFv comprises the amino acid sequence set forth in SEQ ID NO: 101 or SEQ ID NO: 102.

35. An antibody or an antigen-binding fragment thereof, which cross-competes for binding to L1CAM with an anti-L1CAM antibody or an antigen-binding fragment thereof of any one of claims 1-34.

36. An antibody or an antigen-binding fragment thereof, which binds to the same epitope region on L1CAM with an anti-L1CAM antibody or an antigen-binding fragment thereof of any one of claims 1-34.

37. A composition comprising the anti-L1CAM antibody or antigen-binding fragment thereof of any one of claims 1-36.

38. The composition of claim 37, which is a pharmaceutical composition that further comprises a pharmaceutically acceptable carrier.

39. An immunoconjugate comprising the anti-L1CAM antibody or antigen-binding fragment thereof of any one of claims 1-36, linked to a therapeutic agent.

40. The immunoconjugate of claim 39, wherein the therapeutic agent is a drug, a cytotoxin, or a radioactive isotope.

41. The immunoconjugate of claim 39 or 40, wherein the therapeutic agent is a compound selected from the group consisting of dolastatins, maytansinoids, duocarmycins, anthracyclines, camptothecins, and amatoxins.

42. The immunoconjugate of claim 41, wherein the therapeutic agent is selected from the group consisting of monomethyl auristatin E, ABZ038, duocarmycin TM, PNU159682, SN38, and α-Amanitin.

43. The immunoconjugate of claim 41 or 42, wherein the therapeutic agent is PNU159682.

44. The immunoconjugate of any one of claims 39-43 comprising a linker.

45. The immunoconjugate of claim 44, wherein the linker is a cleavable linker or a non-cleavable linker.

46. The immunoconjugate of any one of claims 39-45, comprising a molecule of formula:

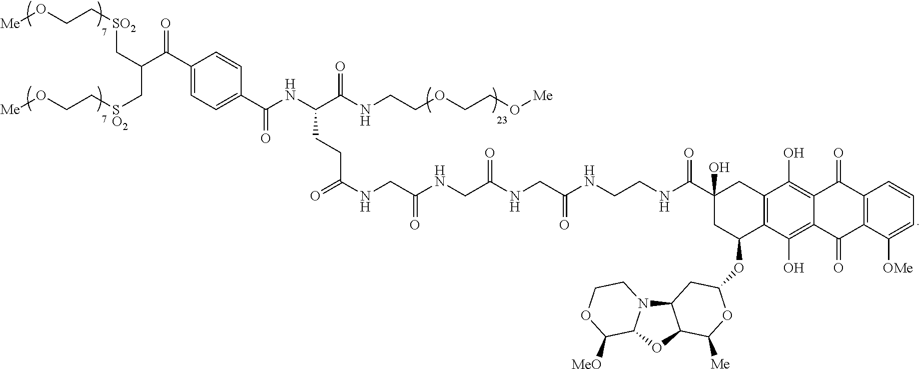

47. The immunoconjugate of any one of claims 39-46, comprising a molecule of formula:

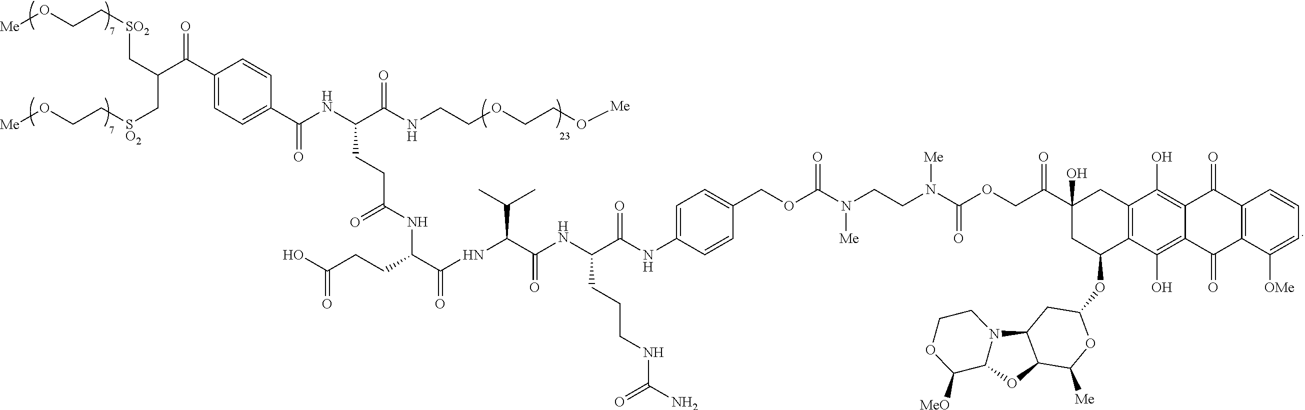

48. A composition comprising the immunoconjugate of any one of claims 39-47.

49. The composition of claim 48, which is a pharmaceutical composition that further comprises a pharmaceutically acceptable carrier.

50. A multi-specific molecule comprising the antibody or antigen-binding fragment thereof of any one of claims 1-36, linked to one or more functional moieties.

51. The multi-specific molecule of claim 50, wherein the one or more functional moieties have a different binding specificity than the antibody or antigen binding fragment thereof.

52. A composition comprising the multi-specific molecule of claim 50 or 51.

53. The composition of claim 52, which is a pharmaceutical composition that further comprises a pharmaceutically acceptable carrier.

54. A nucleic acid that encodes an antibody or antigen-binding fragment thereof of any one of claims 1-36.

55. A nucleic acid that encodes a heavy chain variable region of an antibody or antigen-binding fragment thereof of any one of claims 1-36.

56. A nucleic acid that encodes a light chain variable region of an antibody or antigen-binding fragment thereof of any one of claims 1-36.

57. A vector comprising the nucleic acid of any one of claims 54-56.

58. A host cell comprising the vector of claim 57.

59. A method for detecting L1CAM in a whole cell, a tissue, or a blood sample, comprising:
(a) contacting a cell, tissue or blood sample with the antibody or antigen-binding fragment thereof of any one of claims 1-36, wherein the antibody or antigen-binding fragment thereof comprises a detectable label; and
(b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of L1CAM in the cell, tissue or blood sample.

60. A method of treating or ameliorating a disease or disorder associated with L1CAM in a subject, comprising administering to the subject the antibody or antigen-binding fragment thereof of any one of claims 1-36, the immunoconjugate of any one of claims 39-47, the multi-specific molecule of claim 50 or 51, or the composition of any one of claims 37, 38, 48, 49, 52, or 53.

61. The method of claim 60, wherein the disease or disorder is a tumor.

62. A method of reducing tumor burden in a subject, comprising administering to the subject an antibody or antigen-binding fragment thereof of any one of claims 1-36, the immunoconjugate of any one of claims 39-47, the multi-specific molecule of claim 50 or 51, or the composition of any one of claims 37, 38, 48, 49, 52, or 53.

63. The method of claim 62, wherein the method reduces the number of the tumor cells, reduces the tumor size, and/or eradicates the tumor in the subject.

64. A method of treating and/or preventing a tumor in a subject, comprising administering to the subject an antibody or antigen-binding fragment thereof of any one of claims 1-36, the immunoconjugate of any one of claims 39-47, the multi-specific molecule of claim 50 or 51, or the composition of any one of claims 37, 38, 48, 49, 52, or 53.

65. A method of increasing or lengthening survival of a subject having a tumor, comprising administering to the subject an antibody or antigen-binding fragment thereof of any one of claims 1-36, the immunoconjugate of any one of claims 39-47, the multi-specific molecule of claim 50 or 51, or the composition of any one of claims 37, 38, 48, 49, 52, or 53.

66. The method of claim 65, wherein the method reduces or eradicates tumor burden in the subject.

67. The method of any one of claims 60-66, wherein the tumor is cancer.

68. The method of any one of claims 60-67, wherein the tumor is a metastatic cancer.

69. The method of claim 68, wherein metastatic cancer is an advanced metastatic cancer.

70. The method of any one of claims 60-69, wherein the subject is a human.

71. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of any one of claims 1-36, the immunoconjugate of any one of claims 39-47, the multi-specific molecule of claim 50 or 51, or the composition of any one of claims 37, 38, 48, 49, 52, or 53.

72. The kit of claim 71, wherein the kit further comprises written instructions for using the antibody or antigen-binding fragment thereof, immunoconjugate, multi-specific molecule, or composition for treating or ameliorating a disease or disorder in a subject, reducing tumor burden in a subject, treating and/or preventing a tumor in a subject, and/or increasing or lengthening survival of a subject having a tumor.

73. The antibody or antigen fragment thereof of any one of claims 1-36, the immunoconjugate of any one of claims 39-47, the multi-specific molecule of claim 50 or 51, or the composition of any one of claims 37, 38, 48, 49, 52, or 53 for use in treating or ameliorating a disease or disorder in a subject, reducing tumor burden in a subject, treating and/or preventing a tumor in a subject, and/or increasing or lengthening survival of a subject having a tumor.

74. The antibody or antigen fragment thereof of any one of claims 1-36, the immunoconjugate of any one of claims 39-47, the multi-specific molecule of claim 50 or 51, or the composition of any one of claims 37, 38, 48, 49, 52, or 53 for treating or ameliorating a disease or disorder in a subject, reducing tumor burden in a subject, treating and/or preventing a tumor in a subject, and/or increasing or lengthening survival of a subject having a tumor.

75. Use of the antibody or antigen fragment thereof of any one of claims 1-36, the immunoconjugate of any one of claims 39-47, the multi-specific molecule of claim 50 or 51, or the composition of any one of claims 37, 38, 48, 49, 52, or 53 for the manufacturing of a medicament for treating or ameliorating a disease or disorder in a subject, reducing tumor burden in a subject, treating and/or preventing a tumor in a subject, and/or increasing or lengthening survival of a subject having a tumor.
